(19) United States
(12) Patent Application Publication
(10) Pub. No.: US 2026/0207878 A1
(43) Pub. Date: Jul. 23, 2026

(54) METHOD, APPARATUS, AND SYSTEM FOR CREATING ENVIRONMENT THROUGH AI-BASED NON-CONTACT SLEEP ANALYSIS

(71) Applicant: ASLEEP CO., LTD, Seoul (KR)

(72) Inventors: Dongheon Lee, Seoul (KR); Junki Hong, Seoul (KR); Hye Ah PARK, Seoul (KR); Hyunggug KIM, Seoul (KR); So Ra KANG, Seoul (KR); Jaehyun BAE, Seoul (KR); Sanghyun LEE, Seoul (KR); Daewoo KIM, Seoul (KR); Woojung LIM, Seoul (KR); Jinhwan JUNG, Seoul (KR); Chae Eun LEE, Seoul (KR)

(73) Assignee: ASLEEP CO., LTD, Seoul (KR)

(21) Appl. No.: 19/120,223

(22) PCT Filed: Sep. 27, 2023
(86) PCT No.: PCT/KR2023/014987

§ 371 Date: Apr. 13, 2026

(30) Foreign Application Priority Data
Oct. 13, 2022 (KR) 10-2022-0131829
Oct. 14, 2022 (KR) 10-2022-0132127
Oct. 20, 2022 (KR) 10-2022-0136013
Oct. 31, 2022 (KR) 10-2022-0143037
Nov. 1, 2022 (KR) 10-2022-0143598
Nov. 21, 2022 (KR) 10-2022-0156817
Nov. 30, 2022 (KR) 10-2022-0164613
Dec. 6, 2022 (KR) 10-2022-0168612
Dec. 27, 2022 (KR) 10-2022-0186414
Feb. 24, 2023 (KR) 10-2023-0025312
Mar. 6, 2023 (KR) 10-2023-0028993
May 24, 2023 (KR) 10-2023-0067208
May 26, 2023 (KR) 10-2023-0068718
Jun. 8, 2023 (KR) 10-2023-0073394
Jul. 12, 2023 (KR) 10-2023-0090412
Sep. 19, 2023 (KR) 10-2023-0124703
Sep. 26, 2023 (KR) 10-2023-0129880

(51) Int. Cl. A61M 21/02; A61B 5/00

(52) U.S. Cl. CPC A61M 21/02; A61B 5/4806

(57) ABSTRACT

The present invention relates to a method for controlling an environment adjustment device, comprising: an acquisition step of acquiring environment sensing information; a pre-processing step of performing pre-processing on the acquired environment sensing information; a generation step of generating sleep state information based on the pre-processed environment sensing information; and a control step of controlling the environment adjustment device based on the generated sleep state information.

### TECHNICAL FIELD

The present invention relates to a method, device, and system for environment adjustment through AI-based non-contact sleep analysis.

### BACKGROUND ART

For genuine healthcare, it is essential to monitor and manage 24 hours a day. Health monitoring and management are not simple one-to-one matches, as all elements are intricately interconnected.

Additionally, while there are various methods to maintain and improve health, such as exercise and diet, managing sleep, which accounts for more than 30% of the day, is of utmost importance.

However, despite the leisure afforded by the simple labor replacement by machines, modern individuals suffer from irregular eating habits, lifestyle habits, and stress, leading to inadequate sleep. Consequently, they suffer from sleep disorders such as insomnia, hypersomnia, sleep apnea syndrome, nightmares, night terrors, and sleepwalking.

According to the National Health Insurance Corporation, the number of sleep disorder patients in the country has increased by an average of about 8% annually from 2014 to 2018, with approximately 570,000 patients receiving treatment for sleep disorders in 2018.

As sound sleep is recognized as a crucial factor affecting physical and mental health, interest in sound sleep is increasing. However, to improve sleep disorders, it is necessary to visit specialized medical institutions directly, which requires separate examination costs, and continuous management is difficult, resulting in insufficient efforts by users for treatment.

Due to the increasingly serious sleep problems, the need for sleep health management is growing, and consequently, the Sleep Tech market, which aims to solve sleep problems through technology, is rapidly expanding.

Korean Patent Publication No. 2003-0032529 discloses a sleep-inducing device and method that receives a user's physical information and outputs vibrations and/or ultrasound of a frequency band detected through repetitive learning according to the user's physical state during sleep, thereby enabling optimal sleep induction.

However, conventional technology poses a concern of reduced sleep quality due to the discomfort caused by body-worn equipment, and requires periodic maintenance of the equipment (e.g., charging).

Additionally, the conventional sleep analysis method using wearable devices has the problem that sleep analysis is impossible if the wearable device is not properly in contact with the user's body or if the user does not wear the wearable device.

Furthermore, when multiple users sleep in the same space, the movement of non-wearable device users can interfere with the sleep analysis of wearable device users, and sleep analysis for non-wearable device users is impossible.

Moreover, conventional sleep analysis methods using wearable devices or non-contact sleep management studies utilize the variability of Heart Rate Variability (HRV) or changes in brain waves during sleep and wake states. However, the difference is not significant, limiting the accurate determination of wake time, which is fundamental to all sleep treatments.

Particularly, when using brain wave changes for the treatment of sleep disorders such as snoring, it is impossible to detect the precursor symptoms of snoring through brain wave changes, making it unusable for snoring prevention. It only detects changes in brain waves after snoring occurs, limiting its use to snoring diagnosis.

Accordingly, recent studies are being conducted to estimate a user's sleep state by monitoring the activation level of the autonomic nervous system based on breathing patterns and body movements during the night in a non-contact manner, and to create a user's sleep environment based on the estimated sleep state.

In particular, according to numerous studies on the relationship between sleep environment factors such as air quality, temperature, and humidity, and sleep, it has been confirmed that these sleep environment factors have a decisive impact on sleep quality. This implies that the sleep environment needs to be optimized to improve sleep quality.

### DETAILED DESCRIPTION OF THE INVENTION

### Technical Problem

The objective of the present invention is to provide a sleep analysis system and method that can accurately analyze the sleep of various types of users in real-time, without the need to purchase or wear a separate wearable device, and without being constrained by time and place.

Additionally, the objective of the present invention is to provide a sleep analysis system and method that can replace conventional various biometric signals solely through the user's breathing sound by simultaneously using a smart home-appliance with a built-in microphone and a smartphone, and to deeply analyze the user's sleep through artificial intelligence learning.

Furthermore, the present invention aims to provide various home-appliances to offer an optimal sleep environment related to various factors such as air quality, temperature, and/or humidity of the sleep environment, based on the sleep state information detected from the user's sleep environment.

The problems to be solved by the present invention are not limited to the tasks mentioned above, and other tasks not mentioned can be clearly understood by those skilled in the art from the following description.

### Technical Solution

According to one embodiment of the present invention, in a method for adjusting the environment of an object, the method may include acquiring environment sensing information, performing pre-processing on the acquired environment sensing information, converting the pre-processed environment sensing information into data, generating sleep state information based on the data-converted environment sensing information, and controlling an electronic device to adjust the environment of the object based on the generated sleep state information.

Additionally, according to one embodiment of the present invention, the step of controlling the electronic device may involve generating information to control the environment of the object in real-time based on the generated sleep state information.

Furthermore, in a method for adjusting the environment of an object according to one embodiment of the present invention, the environment sensing information may include sound information.

Additionally, in a method for creating an environment for an object according to an embodiment of the present invention, the sound information may include breathing sound information.

Furthermore, in a method for creating an environment for an object according to an embodiment of the present invention, the sleep state information may include sleep stage information.

Moreover, in a method for creating an environment for an object according to an embodiment of the present invention, the step of datafying the environment sensing information may further include converting the pre-processed environment sensing information into information that includes changes in frequency components over the time axis.

Here, in a method for creating an environment for an object according to an embodiment of the present invention, the information including changes in frequency components over the time axis may be a spectrogram.

Meanwhile, according to an embodiment of the present invention, an electronic device for creating an environment for an object may be provided, comprising: a sensor for acquiring environment sensing information, means for performing pre-processing on the acquired environment sensing information, means for datafying the pre-processed environment sensing information, means for generating sleep state information based on the datafied environment sensing information, and means for controlling the electronic device so that the environment of the object is created based on the generated sleep state information.

Meanwhile, according to an embodiment of the present invention, an electronic device for creating an environment for an object may be provided, comprising: a sensor for acquiring environment sensing information, means for performing pre-processing on the acquired environment sensing information, means for transmitting the datafied environment sensing information to a server, means for receiving the generated sleep state information when the server generates sleep state information based on the transmitted environment sensing information, and means for controlling the electronic device so that the environment of the object is created based on the received sleep state information.

Meanwhile, according to an embodiment of the present invention, an electronic device for creating an environment for an object may be provided, comprising: a sensor for acquiring environment sensing information, means for performing pre-processing on the acquired environment sensing information, means for transmitting the pre-processed environment sensing information to a server, means for receiving the generated sleep state information when the server datafies the transmitted environment sensing information and generates sleep state information based on the datafied environment sensing information, and means for controlling the electronic device so that the environment of the object is created based on the received sleep state information.

Here, in an electronic device for creating an environment for an object according to an embodiment of the present invention, the means for controlling the electronic device may generate information for controlling the environment of the object in real-time based on the generated sleep state information.

Additionally, in an electronic device for creating an environment for an object according to an embodiment of the present invention, the environment sensing information may include sound information.

Furthermore, in an electronic device for creating an environment for an object according to an embodiment of the present invention, the sound information may include breathing sound information.

Additionally, in an electronic device for creating an environment for an object according to an embodiment of the present invention, the sleep state information may include sleep stage information.

Furthermore, in an electronic device for creating an environment for an object according to an embodiment of the present invention, the data-processed environment sensing information may be converted into information that includes changes over the time axis of frequency components of the pre-processed environment sensing information.

Here, in an electronic device for creating an environment for an object according to an embodiment of the present invention, the information including changes over the time axis of frequency components may be a spectrogram.

Meanwhile, in an electronic device for controlling a home-appliance to create an environment for an object according to an embodiment of the present invention, there may be provided an electronic device comprising: a sensor for acquiring environment sensing information, means for performing pre-processing on the acquired environment sensing information, means for data-processing the pre-processed environment sensing information, means for generating sleep state information based on the data-processed environment sensing information, and means for controlling the home-appliance so that the environment for the object is created based on the generated sleep state information.

Meanwhile, in an electronic device for controlling a home-appliance to create an environment for an object according to an embodiment of the present invention, there may be provided an electronic device comprising: a sensor for acquiring environment sensing information, means for performing pre-processing on the acquired environment sensing information, means for data-processing the pre-processed environment sensing information, means for transmitting the data-processed environment sensing information to a server, means for receiving the generated sleep state information when the server generates sleep state information based on the transmitted environment sensing information, and means for controlling the home-appliance so that the environment for the object is created based on the received sleep state information.

Meanwhile, in an electronic device for controlling a home-appliance to create an environment for an object according to an embodiment of the present invention, there may be provided an electronic device comprising: a sensor for acquiring environment sensing information, means for performing pre-processing on the acquired environment sensing information, means for transmitting the pre-processed environment sensing information to a server, means for receiving the generated sleep state information when the server data-processes the transmitted environment sensing information and generates sleep state information based on the data-processed environment sensing information, and means for controlling the home-appliance so that the environment for the object is created based on the received sleep state information.

Meanwhile, in an electronic device for controlling a home-appliance to create an environment for an object according to an embodiment of the present invention, there may be provided an electronic device comprising: means for receiving sleep state information generated by another electronic device that acquires environment sensing information, data-processes the acquired environment sensing information, and generates sleep state information based on the data-processed environment sensing information, and means for controlling the home-appliance so that the environment for the object is created based on the received sleep state information.

Meanwhile, in an electronic device for controlling a home-appliance to create an environment for an object according to an embodiment of the present invention, there may be provided an electronic device comprising: means for receiving sleep state information from a server when another electronic device acquires environment sensing information, data-processes the acquired environment sensing information, transmits the data-processed environment sensing information to the server, and the server generates sleep state information based on the transmitted environment sensing information, and means for controlling the home-appliance so that the environment for the object is created based on the received sleep state information.

Meanwhile, in an electronic device for controlling a home-appliance to create an environment for an object according to an embodiment of the present invention, there may be provided an electronic device comprising: means for receiving sleep state information from a server when another electronic device acquires environment sensing information, transmits the acquired environment sensing information to the server, and the server data-processes the transmitted environment sensing information and generates sleep state information based on the data-processed environment sensing information, and means for controlling the home-appliance so that the environment for the object is created based on the received sleep state information.

Here, in an electronic device for controlling a home-appliance to create an environment for an object according to an embodiment of the present invention, the means for controlling the home-appliance may generate information for real-time control of the environment for the object based on the generated sleep state information.

Additionally, in an electronic device for controlling a home-appliance to create the environment of an object according to one embodiment of the present invention, the environment sensing information may include sound information.

Additionally, in an electronic device for controlling a home-appliance to create the environment of an object according to one embodiment of the present invention, the sound information may include breathing sound information.

Additionally, in an electronic device for controlling a home-appliance to create the environment of an object according to one embodiment of the present invention, the sleep state information may include sleep stage information.

Additionally, in an electronic device for controlling a home-appliance to create the environment of an object according to one embodiment of the present invention, the data-processed environment sensing information may be converted from the pre-processed environment sensing information into information that includes changes in frequency components over the time axis.

Here, in an electronic device for controlling a home-appliance to create the environment of an object according to one embodiment of the present invention, the information including changes in frequency components over the time axis may be a spectrogram.

The present invention relates to a method for controlling an environment adjustment device, comprising: an acquisition step of acquiring environment sensing information; a pre-processing step of performing pre-processing on the acquired environment sensing information; a generation step of generating sleep state information based on the pre-processed environment sensing information; and a control step of controlling the environment adjustment device based on the generated sleep state information.

The present invention relates to a method for controlling an environment adjustment device, wherein in the control step, the environment adjustment device is controlled in real-time based on the generated sleep state information.

The present invention relates to a method for controlling an environment adjustment device, wherein the generation step further includes converting the environment sensing information into information that includes changes in frequency components over the time axis.

The present invention relates to a method for controlling an environment adjustment device, wherein the control step includes: generating first environment adjustment information based on the generated sleep state information; causing the environment adjustment device to create an environment based on the first environment adjustment information; generating second environment adjustment information based on the generated user's sleep state information after the environment adjustment device has started creating the environment based on the first environment adjustment information; and causing the environment adjustment device to create an environment based on the generated second environment adjustment information.

The step of generating the first environment adjustment information includes generating the first environment adjustment information based on the sleep state information generated over a time corresponding to one or more epochs, and the step of generating the second environment adjustment information includes generating the second environment adjustment information based on the sleep state information generated over a time corresponding to one or more epochs after the environment adjustment device has started creating the environment based on the first environment adjustment information, relating to a method for controlling an environment adjustment device.

The present invention relates to an electronic device for controlling an environment adjustment device, comprising: a sensor for acquiring environment sensing information; an operation for performing pre-processing on the acquired environment sensing information; an operation for generating sleep state information based on the pre-processed environment sensing information; and an operation for controlling the environment adjustment device based on the generated sleep state information, wherein the electronic device includes a control unit.

The present invention pertains to an electronic device for controlling an environment adjustment device, wherein the control unit performs an operation to control the environment adjustment device in real-time based on the generated sleep state information.

The present invention relates to an electronic device for controlling an environment adjustment device, wherein the control unit performs an operation to convert the environment sensing information into information that includes changes over the time axis of the frequency components of the environment sensing information.

The present invention pertains to an electronic device for controlling an environment adjustment device, wherein the control unit generates first environment adjustment information based on the generated sleep state information, performs an operation to control the environment adjustment device based on the generated first environment adjustment information, and after the environment adjustment device begins to adjust the environment based on the first environment adjustment information, generates second environment adjustment information based on the generated user's sleep state information, and performs an operation to control the environment adjustment device based on the generated second environment adjustment information.

The present invention relates to an electronic device for controlling an environment adjustment device, wherein the generated first environment adjustment information is created based on the sleep state information generated over a time corresponding to one or more epochs, and the generated second environment adjustment information is created based on the sleep state information generated over a time corresponding to one or more epochs after the environment adjustment device begins to adjust the environment based on the first environment adjustment information.

The present invention pertains to an environment adjustment system comprising: an electronic device including a sensor for acquiring environment sensing information, a control unit, and a communication unit for transmitting and receiving information via a network; a server performing an operation to generate sleep state information based on the environment sensing information; and an environment adjustment device, wherein the control unit performs an operation for pre-processing the acquired environment sensing information and transmits the pre-processed environment sensing information to the server via the communication unit, the server performs an operation to generate sleep state information based on the pre-processed environment sensing information received from the electronic device, and the control unit performs an operation to receive the generated sleep state information from the server via the communication unit and controls the environment adjustment device based on the received sleep state information.

The present invention relates to an environment adjustment system, wherein the control unit performs an operation to control the environment adjustment device in real-time based on the received sleep state information.

The present invention pertains to an environment adjustment system, wherein the control unit performs an operation to control the environment adjustment device in real-time based on the received sleep state information.

The present invention relates to an environment adjustment system, wherein the control unit performs an operation to receive information from the server via the communication unit, which converts the environment sensing information into information that includes changes over the time axis of the frequency components of the environment sensing information.

The present invention pertains to an environment adjustment system, wherein the control unit performs an operation to control the environment adjustment device based on the first environment adjustment information generated based on the received sleep state information via the communication unit, and after the environment adjustment device begins to adjust the environment based on the first environment adjustment information, generates second environment adjustment information based on the received user's sleep state information via the communication unit, and performs an operation to control the environment adjustment device based on the generated second environment adjustment information.

The present invention relates to an environment adjustment system wherein the generated first environment adjustment information is created based on the sleep state information generated over a time corresponding to one or more epochs, and the generated second environment adjustment information is created based on the sleep state information generated over a time corresponding to one or more epochs after the environment adjustment device begins to adjust the environment based on the first environment adjustment information.

The present invention pertains to an environment adjustment system comprising an electronic device that includes a sensor for acquiring environment sensing information, a control unit, and a communication unit for transmitting and receiving information via a network; a server that performs operations to generate sleep state information based on the environment sensing information and to generate environment adjustment information based on the sleep state information; and an environment adjustment device controlled based on the environment adjustment information. The control unit performs operations to pre-process the acquired environment sensing information and to transmit the pre-processed environment sensing information to the server via the communication unit. The server performs operations to generate sleep state information based on the pre-processed environment sensing information received from the electronic device, to generate environment adjustment information for controlling the environment adjustment device based on the generated sleep state information, and to transmit the generated environment adjustment information to the environment adjustment device.

The present invention relates to an environment adjustment system wherein the server includes a first server and a second server. The first server performs operations to generate sleep state information based on the pre-processed environment sensing information received from the electronic device, and the second server performs operations to generate environment adjustment information for controlling the environment adjustment device based on the generated sleep state information.

The present invention pertains to an environment adjustment system wherein the server performs operations to control the environment adjustment device in real-time based on the generated sleep state information.

The present invention relates to an environment adjustment system wherein the server performs operations to convert the environment sensing information received from the electronic device into information that includes changes in the frequency components of the environment sensing information over the time axis.

The present invention pertains to an environment adjustment system wherein the server includes a first server and a second server. The first server performs operations to generate sleep state information based on the pre-processed environment sensing information received from the electronic device, and the second server performs operations to generate environment adjustment information for controlling the environment adjustment device based on the generated sleep state information.

The present invention relates to an environment adjustment system wherein the generated first environment adjustment information is created based on the sleep state information generated over a time corresponding to one or more epochs, and the generated second environment adjustment information is created based on the sleep state information generated over a time corresponding to one or more epochs after the environment adjustment device begins to adjust the environment based on the first environment adjustment information.

The present invention pertains to a method for controlling an electronic device that provides a predetermined scent in response to predetermined scent-providing information, comprising an acquisition step for acquiring environment sensing information; a pre-processing step for performing pre-processing on the acquired environment sensing information; a generation step for generating sleep state information based on the pre-processed environment sensing information; and a control step for controlling the electronic device that provides a predetermined scent based on the generated sleep state information.

The present invention relates to a method for controlling an electronic device that provides a predetermined scent in response to predetermined scent-providing information, wherein the electronic device is controlled in real-time based on the generated sleep state information.

The present invention pertains to a method for controlling an electronic device that provides a predetermined scent in response to predetermined scent-providing information, wherein the generation step further includes converting the environment sensing information into information that includes changes in the frequency components of the environment sensing information over the time axis.

The present invention relates to a method for controlling an electronic device that provides a predetermined scent in response to predetermined scent-providing information, wherein the information includes a spectrogram representing changes in frequency components over the time axis.

The present invention relates to a method for controlling an electronic device that provides a predetermined scent in response to predetermined scent-providing information, wherein the environment sensing information includes sleep sound information.

The present invention relates to a method for controlling an electronic device that provides a predetermined scent in response to predetermined scent-providing information, wherein the sleep state information includes at least one of sleep stage information, sleep stage probability information, sleep event information, and sleep event probability information.

The present invention relates to a method for controlling an electronic device that provides a predetermined scent in response to predetermined scent-providing information, further comprising the steps of: causing the electronic device to provide a first scent; generating second scent-providing information based on the generated user's sleep state information after the electronic device begins providing the first scent; and causing the electronic device to provide a second scent based on the generated second scent-providing information.

The present invention relates to a method for controlling an electronic device that provides a predetermined scent in response to predetermined scent-providing information, further comprising the step of generating second scent-providing information based on at least one of the user's sleep stage information, sleep stage probability information, sleep event information, and sleep event probability information generated after the electronic device begins providing the first scent.

The present invention relates to a method for controlling an electronic device that provides a predetermined scent in response to predetermined scent-providing information, further comprising the steps of: generating first scent-providing information based on the generated sleep state information; causing the electronic device to provide the first scent for a first period based on the generated first scent-providing information; generating second scent-providing information based on the generated user's sleep state information after the electronic device begins providing the first scent; and causing the electronic device to provide the second scent for a second period based on the generated second scent-providing information.

The present invention relates to a method for controlling an electronic device that provides a predetermined scent in response to predetermined scent-providing information, wherein the first period and the second period are multiples of a predetermined minimum time unit.

The present invention relates to a method for controlling an electronic device that provides a predetermined scent in response to predetermined scent-providing information, further comprising the step of generating second scent-providing information based on at least one of the user's sleep stage information, sleep stage probability information, sleep event information, and sleep event probability information generated after the electronic device begins providing the first scent.

The present invention relates to a method for controlling an electronic device that provides a predetermined scent in response to predetermined scent-providing information, wherein the first scent-providing information and the second scent-providing information include at least one of scent attribute information and scent provision control information.

The present invention, in the control step, includes generating the first scent-providing information based on the sleep state information generated over a time corresponding to one or more epochs, and generating the second scent-providing information based on the sleep state information generated over a time corresponding to one or more epochs after starting to provide the first scent. This invention relates to a method for controlling an electronic device that provides a predetermined scent in response to predetermined scent-providing information.

The present invention relates to a method for controlling an electronic device that provides a predetermined scent in response to predetermined scent-providing information, wherein the epoch is set to data corresponding to 30-second intervals.

The present invention relates to a method for controlling an electronic device that provides a predetermined scent in response to predetermined scent-providing information, wherein at least one of the first or second scent includes no scent, and at least one of the first scent-providing information or the second scent-providing information includes information indicating that no scent is provided.

The present invention relates to an electronic device that provides a predetermined scent in response to predetermined scent-providing information, comprising: a sensor for acquiring environment sensing information; means for performing pre-processing on the acquired environment sensing information; means for generating sleep state information based on the pre-processed environment sensing information; and means for providing a predetermined scent based on the generated sleep state information.

The present invention relates to an electronic device that provides a predetermined scent in response to predetermined scent-providing information, wherein the predetermined scent is provided in real-time based on the generated sleep state information.

The present invention relates to an electronic device that provides a predetermined scent in response to predetermined scent-providing information, wherein the means for generating sleep state information based on the pre-processed environment sensing information converts the environment sensing information into information including changes over the time axis of the frequency components of the environment sensing information.

The present invention relates to an electronic device that provides a predetermined scent in response to predetermined scent-providing information, wherein the information including changes over the time axis of the frequency components is a spectrogram.

The present invention relates to an electronic device that provides a predetermined scent in response to predetermined scent-providing information, wherein the environment sensing information includes sleep sound information.

The present invention relates to an electronic device that provides a predetermined scent in response to predetermined scent-providing information, wherein the sleep state information includes at least one of sleep stage information, sleep stage probability information, sleep event information, and sleep event probability information.

The present invention relates to an electronic device that provides a predetermined scent in response to predetermined scent-providing information, wherein the means for providing a predetermined scent based on the generated sleep state information provides the first scent to the user and provides the second scent based on the generated user's sleep state information after starting to provide the first scent.

The present invention relates to an electronic device that provides a predetermined scent in response to predetermined scent-providing information, wherein, in the case of providing the second scent, the device provides the second scent based on at least one of the user's sleep stage information, sleep stage probability information, sleep event information, and sleep event probability information generated after starting to provide the first scent.

The present invention pertains to an electronic device that provides a predetermined scent in response to predetermined scent-providing information, wherein the means for providing the predetermined scent based on the generated sleep state information provides the first scent for a first period based on the generated sleep state information, and provides the second scent for a second period based on the user's sleep state information generated after starting to provide the first scent.

The present invention relates to an electronic device that provides a predetermined scent in response to predetermined scent-providing information, wherein the first time and the second time are multiples of a predetermined minimum time unit.

The present invention pertains to an electronic device that provides a predetermined scent in response to predetermined scent-providing information, wherein, in the case of providing the second scent, the device provides the second scent based on at least one of the user's sleep stage information, sleep stage probability information, sleep event information, and sleep event probability information generated after starting to provide the first scent.

The present invention relates to an electronic device that provides a predetermined scent in response to predetermined scent-providing information, wherein the first scent and the second scent are at least one of a scent based on scent attribute information and a scent based on scent-provision control information.

The present invention pertains to an electronic device that provides a predetermined scent in response to predetermined scent-providing information, wherein, in the case of providing the first scent, the device provides the first scent based on the sleep state information generated for a time corresponding to one or more epochs, and provides the second scent based on the sleep state information generated for a time corresponding to one or more epochs after starting to provide the first scent.

The present invention relates to an electronic device that provides a predetermined scent in response to predetermined scent-providing information, wherein the epoch is set to data corresponding to 30-second units.

The present invention pertains to an electronic device that provides a predetermined scent in response to predetermined scent-providing information, wherein at least one of the first scent or the second scent includes a non-scent.

The present invention relates to an electronic device that provides a predetermined scent in response to predetermined scent-providing information, comprising: a sensor for acquiring environment sensing information; means for performing pre-processing on the acquired environment sensing information; means for transmitting the pre-processed environment sensing information to a server; means for receiving sleep state information generated based on the transmitted environment sensing information from the server; and means for providing a predetermined scent based on the received sleep state information.

The present invention relates to an electronic device that provides a predetermined scent in response to predetermined scent-providing information, wherein the means for providing the predetermined scent provides the predetermined scent in real-time based on the received sleep state information.

The present invention relates to an electronic device that provides a predetermined scent in response to predetermined scent-providing information, wherein the means for receiving sleep state information generated based on the environment sensing information transmitted from the server receives the converted information when the server converts the transmitted environment sensing information into information including changes over the time axis of the frequency components of the environment sensing information.

The present invention relates to an electronic device that provides a predetermined scent in response to predetermined scent-providing information, wherein the information including changes over the time axis of the frequency components is a spectrogram.

The present invention relates to an electronic device that provides a predetermined scent in response to predetermined scent-providing information, wherein the environment sensing information includes sleep sound information.

The present invention relates to an electronic device that provides a predetermined scent in response to predetermined scent-providing information, wherein the sleep state information includes at least one of sleep stage information, sleep stage probability information, sleep event information, and sleep event probability information.

The present invention relates to an electronic device that provides a predetermined scent in response to predetermined scent-providing information, wherein the means for providing the predetermined scent based on the received sleep state information provides a first scent, and after starting to provide the first scent, provides a second scent based on the received user's sleep state information.

The present invention relates to an electronic device that provides a predetermined scent in response to predetermined scent-providing information, wherein in the case of providing the second scent, the second scent is provided based on at least one of the user's sleep stage information, sleep stage probability information, sleep event information, and sleep event probability information received after starting to provide the first scent.

The present invention relates to an electronic device that provides a predetermined scent in response to predetermined scent-providing information, wherein the means for providing the predetermined scent based on the received sleep state information provides a first scent for a first time period and, after starting to provide the first scent, provides a second scent for a second time period based on the received user's sleep state information.

The present invention relates to an electronic device that provides a predetermined scent in response to predetermined scent-providing information, wherein the first time period and the second time period are multiples of a predetermined minimum time unit.

The present invention relates to an electronic device that provides a predetermined scent in response to predetermined scent-providing information, wherein in the case of providing the second scent, the second scent is provided based on at least one of the user's sleep stage information, sleep stage probability information, sleep event information, and sleep event probability information received after starting to provide the first scent to the user.

The present invention relates to an electronic device that provides a predetermined scent in response to predetermined scent-providing information, wherein the first scent-providing information and the second scent-providing information include at least one of scent attribute information and scent provision control information.

The present invention pertains to an electronic device that provides a predetermined scent in response to predetermined scent-providing information, wherein, when providing the first scent, the device provides the first scent based on the sleep state information received for a time corresponding to one or more epochs, and provides the second scent based on the sleep state information received for a time corresponding to one or more epochs after the provision of the first scent has commenced.

The present invention relates to an electronic device that provides a predetermined scent in response to predetermined scent-providing information, wherein the epoch is set as data corresponding to 30-second units.

The present invention pertains to an electronic device that provides a predetermined scent in response to predetermined scent-providing information, wherein at least one of the first scent or the second scent includes a non-scent, and the scent-providing information includes information indicating that no scent is provided.

The present invention relates to an electronic device for controlling a home-appliance that provides a predetermined scent in response to predetermined scent-providing information, comprising: a sensor for acquiring environment sensing information; means for performing pre-processing on the acquired environment sensing information; means for generating sleep state information based on the pre-processed environment sensing information; and means for controlling the home-appliance that provides a predetermined scent based on the generated sleep state information.

The present invention pertains to an electronic device for controlling a home-appliance that provides a predetermined scent in response to predetermined scent-providing information, comprising: a sensor for acquiring environment sensing information; means for performing pre-processing on the acquired environment sensing information; means for transmitting the pre-processed environment sensing information to a server; means for receiving sleep state information generated based on the transmitted environment sensing information from the server; and means for controlling the home-appliance that provides a predetermined scent based on the received sleep state information.

The present invention relates to an electronic device for controlling a home-appliance that provides a predetermined scent in response to predetermined scent-providing information, wherein another electronic device acquires environment sensing information, performs pre-processing on the acquired environment sensing information, and generates sleep state information based on the pre-processed environment sensing information, comprising: a receiving unit for receiving the generated sleep state information; and means for controlling the home-appliance that provides a predetermined scent based on the received sleep state information.

The present invention pertains to an electronic device for controlling a home-appliance that provides a predetermined scent in response to predetermined scent-providing information, wherein another electronic device acquires environment sensing information, performs pre-processing on the acquired environment sensing information, and transmits the pre-processed environment sensing information to a server, comprising: a receiving unit for receiving sleep state information generated based on the transmitted environment sensing information from the server; and means for controlling the home-appliance that provides a predetermined scent based on the received sleep state information.

To solve the above task, a light-modulation device for adjusting a user's sleep environment is provided, comprising: a sensing unit for acquiring the user's sound information; a transmitting unit for transmitting the acquired user's sound information to a server; a receiving unit for receiving sleep state information generated by the server based on the transmitted user's sound information; a control unit for generating light-modulation information based on the received sleep state information; and a light source unit for emitting adjusted light based on the generated light-modulation information.

To solve the above task, the receiving unit receives the user's average sleep onset latency information generated by the server based on the transmitted user's sound information, and the control unit generates light-modulation information based on the user's average sleep onset latency information, thereby providing a light-modulation device for adjusting the user's sleep environment.

To address the present task, the control unit provides a light-modulation device that creates a user's sleep environment by generating light-modulation information based on the set time information.

To address the present task, when the server determines that the user has fallen asleep and generates sleep state information, the receiving unit receives the sleep state information, and the control unit provides a light-modulation device that creates a user's sleep environment by generating light-modulation information based on the sleep state information.

To address the present task, the control unit provides a light-modulation device that creates a user's sleep environment by generating light-modulation information according to the light-modulation information generated based on the user's average sleep onset time information, controlling the amount of light emitted by the light source unit to decrease or to 0 lux upon reaching the average sleep onset delay time.

To address the present task, the control unit provides a light-modulation device that creates a user's sleep environment by generating light-modulation information to control the light emitted by the light source unit below a threshold when the user falls asleep faster than the user's average sleep onset delay time.

To address the present task, the control unit provides a light-modulation device that creates a user's sleep environment by generating light-modulation information to maintain the light emitted by the light source unit below a first threshold when the user falls asleep later than the user's average sleep onset delay time, and controlling it below a second threshold upon the user's sleep onset.

To address the present task, when the server generates sleep state information based on the transmitted user's sound information and generates the user's biological rhythm information based on the generated sleep state information, the receiving unit provides a light-modulation device that creates a user's sleep environment by receiving the generated biological rhythm information from the server.

To address the present task, the control unit provides a light-modulation device that creates a user's sleep environment by generating light-modulation information so that the biological rhythm information received by the receiving unit from the server conforms to predetermined biological time information.

To address the present task, when the receiving unit receives predetermined alarm time information, the control unit provides a light-modulation device that creates a user's sleep environment by generating light-modulation information to increase the amount of light at a predetermined gradient from a set predetermined brightness to a user-set brightness starting before the critical time of the alarm time.

To address the present task, when the receiving unit receives predetermined alarm time information and the user's REM sleep is detected between the alarm time and the critical time, the control unit provides a light-modulation device that creates a user's sleep environment by generating light-modulation information to increase the amount of light at a predetermined gradient from a set predetermined brightness to a user-set brightness after a predetermined time has elapsed from the point when the user's REM sleep is detected.

To address the present task, when the receiving unit receives predetermined alarm time information and the user's REM sleep is not detected between the alarm time information and the critical time, the control unit provides a light-modulation device that creates a user's sleep environment by generating light-modulation information to increase the amount of light at a predetermined gradient from a set predetermined brightness to a user-set brightness before the critical time of the alarm time.

To address the present task, the control unit provides a light-modulation device that creates a user's sleep environment by generating light-modulation information to cause the light source unit to emit light above a threshold value if the user's wake state is not detected for a threshold time after the alarm time.

To address the present task, when the receiving unit receives predetermined alarm time information, the control unit provides a light-modulation device that creates a user's sleep environment by generating light-modulation information to increase the amount of light from a predetermined brightness to a user-set brightness at a predetermined gradient, based on the generated user's biological rhythm information, starting from a threshold time before the alarm time.

To address the present task, in a light-modulation device that creates a user's sleep environment, it includes: a sensing unit that acquires the user's sound information; a transmitting unit that transmits the acquired user's sound information to a server; a receiving unit that receives the generated light-modulation information based on the sleep state information generated by the server from the transmitted user's sound information; and a light source unit that emits adjusted light based on the generated light-modulation information.

To address the present task, in a light-modulation device that creates a user's sleep environment, it includes: a sensing unit that acquires the user's sound information; a control unit that generates sleep state information based on the acquired user's sound information and generates light-modulation information based on the generated sleep state information; and a light source unit that emits adjusted light based on the generated light-modulation information.

To address the present task, in a light-modulation device that creates a user's sleep environment, it includes: a sensing unit that acquires the user's sound information; a transmitting unit that transmits the acquired user's sound information to a first server; a receiving unit that receives sleep state information generated by a second server based on the transmitted sound information from the first server, generates light-modulation information based on the acquired sound information, and receives the light-modulation information; and a light source unit that emits adjusted light based on the received light-modulation information.

To address the present task, in a device for controlling the light of a light source device having a light source unit, it includes: a sensing unit that acquires sound information; a memory unit where an application can be recorded; and a processor unit where the application can be executed. The application is configured to generate sleep state information based on the sound information acquired by the sensing unit, generate light-modulation information based on the generated sleep state information, and transmit the light-modulation information to the light source unit.

To address the present task, in a device for controlling the light of a light source device having a light source unit, it includes: a sensing unit that acquires sound information; a memory unit where a first application and a second application can be recorded; and a processor unit where the first application and the second application can be executed. The first application is configured to generate sleep state information based on the sound information acquired by the sensing unit, and the second application is configured to generate light-modulation information based on the sleep state information generated by the first application and transmit the light-modulation information to the light source unit.

To address the present task, in a recording medium where a program is recorded, it includes: a step of pre-processing the sound information acquired by the sensing unit to acquire sleep sound information; a step of transmitting the acquired sleep sound information to a server through a transmitting unit; a first receiving step of receiving the generated sleep state information from the server through a receiving unit when the server generates sleep state information based on the transmitted user's sleep sound information; a control step of generating light-modulation information based on the received sleep state information; and an emission step of emitting adjusted light through the light source unit based on the generated light-modulation information.

To address the present task, the receiving step includes receiving the generated user's average sleep onset latency information when the server generates the user's average sleep onset latency information based on the transmitted user's sound information, and the control step includes providing a recording medium where a program is recorded to generate light-modulation information based on the user's average sleep onset latency information.

To address the present task, the control step includes providing a recording medium where a program is recorded to generate light-modulation information based on the set time information.

To address the present task, when the server determines that the user is falling asleep and generates sleep state information, the receiving step involves receiving the sleep state information, and the control step provides a recording medium on which a program is recorded that generates light-modulation information based on the sleep state information.

To address the present task, the control step provides a recording medium on which a program is recorded that generates light-modulation information based on the user's average sleep latency information. This program controls the light emitted by the light source unit to decrease when the average sleep latency is reached or to maintain a predetermined brightness.

To address the present task, the control step provides a recording medium on which a program is recorded that generates light-modulation information to control the light emitted by the light source unit below a threshold if the user is detected to fall asleep before reaching the user's average sleep latency.

To address the present task, a heated-water mattress for creating a user's sleep environment is provided, comprising: a sensing unit for acquiring the user's sound information; a transmitting unit for transmitting the acquired user's sound information to a server; a receiving unit for receiving the generated sleep state information from the server when the server generates sleep state information based on the transmitted user's sound information; a control unit for generating thermal control information based on the received sleep state information; and thermal control means for adjusting the heat to achieve the adjusted temperature based on the generated thermal control information.

To address the present task, when the receiving unit receives sleep state information from the server indicating that the user is before sleep onset, the control unit provides a heated-water mattress for creating a user's sleep environment by generating thermal control information based on the user-set temperature.

To address the present task, when the receiving unit receives sleep state information from the server indicating that the user is in sleep latency, or receives user-set sleep latency, the control unit provides a heated-water mattress for creating a user's sleep environment by generating thermal control information set to a temperature below or above a predetermined temperature based on the received user's sleep state information or the received user-set sleep latency.

To address the present task, further comprising a user body temperature measurement unit, when the receiving unit receives sleep state information from the server indicating that the user is in sleep latency and receives user body temperature drop information from the user body temperature measurement unit, the control unit provides a heated-water mattress for creating a user's sleep environment by generating thermal control information set to a temperature above a predetermined temperature from the user-set temperature.

To address the present task, when the server generates sleep state information based on the transmitted user's sound information and generates the user's biological rhythm information based on the generated sleep state information, the receiving unit provides a heated-water mattress for creating a user's sleep environment by receiving the generated biological rhythm information from the server.

To address the present task, when the receiving unit receives sleep state information from the server indicating that the user is in sleep latency, the control unit provides a heated-water mattress for creating a user's sleep environment by generating thermal control information with a change from the user-set temperature to a predetermined temperature based on the biological rhythm information received by the receiving unit from the server.

To address the present task, when the receiving unit receives sleep state information from the server indicating the first deep sleep, the control unit provides a heated-water mattress for creating a user's sleep environment by maintaining the set thermal control information until a predetermined time thereafter.

To address the present task, the receiving unit provides a heated-water mattress that creates a user's sleep environment by maintaining the preset temperature control information until the receiving unit receives sleep state information from the server indicating the first REM sleep.

To address the present task, when the receiving unit receives sleep state information from the server indicating REM sleep, the receiving unit, based on the sleep state information received from the server, provides a heated-water mattress that creates a user's sleep environment by generating temperature control information that induces a predetermined temperature change during the REM sleep.

To address the present task, when the receiving unit receives sleep state information from the server indicating a wake state, REM state, or light sleep state, or sleep state information indicating a point between the REM state and the light sleep state, the control unit provides a heated-water mattress that creates a user's sleep environment by generating temperature control information that increases the temperature by a predetermined amount between the wake state detection point, the REM state detection point, or the light sleep state detection point and the user's desired wake time, or between the REM state detection point and the light sleep state detection point and the user's desired wake time.

To address the present task, the temperature control information that increases the temperature by the predetermined amount provides a heated-water mattress that creates a user's sleep environment, wherein the control unit generates first temperature control information when the receiving unit receives sleep state information from the server indicating the user is in a light sleep state at the user's desired wake time, and generates second temperature control information when the user is in a deep sleep state.

To address the present task, the system further includes a user body temperature measurement unit, and when the receiving unit receives sleep state information from the server indicating a wake state and receives user body temperature information from the user body temperature measurement unit indicating the user's body temperature is higher than a predetermined temperature, the control unit provides a heated-water mattress that creates a user's sleep environment by generating temperature control information that lowers the temperature by a predetermined amount.

To address the present task, the system includes a sensing unit for acquiring the user's sound information; a transmitting unit for transmitting the acquired user's sound information to the server; a receiving unit for receiving the generated sleep state information from the server based on the transmitted user's sound information, and generating temperature control information based on the generated sleep state information; and thermal control means for adjusting the heat to achieve the adjusted temperature based on the generated temperature control information, thereby providing a heated-water mattress that creates a user's sleep environment.

To address the present task, in the heated-water mattress that creates a user's sleep environment, the system includes a sensing unit for acquiring the user's sound information; a control unit for generating sleep state information based on the acquired user's sound information and generating temperature control information based on the generated sleep state information; and thermal control means for adjusting the heat to achieve the adjusted temperature based on the generated temperature control information, thereby providing a heated-water mattress that creates a user's sleep environment.

To address the present task, in the heated-water mattress that creates a user's sleep environment, the system includes a sensing unit for acquiring the user's sound information; a transmitting unit for transmitting the acquired sound information to the first server; a receiving unit for receiving sleep state information generated by the second server based on the transmitted sound information from the first server, generating temperature control information based on the received sleep state information, and receiving the temperature control information; and thermal control means for adjusting the heat to achieve the adjusted temperature based on the received temperature control information, thereby providing a heated-water mattress that creates a user's sleep environment.

To address the present task, in a device for controlling the thermal control means of a heated-water mattress with thermal control means, the system includes a sensing unit for acquiring the user's sound information; a memory unit where an application can be recorded; and a processor unit where the application can be executed. The processor unit generates sleep state information based on the sound information acquired from the sensing unit through the application, generates temperature control information based on the generated sleep state information, and transmits the generated temperature control information to the heated-water mattress, thereby providing a device for controlling the thermal control means of a heated-water mattress with thermal control means.

To address the present task, in a device for controlling the thermal control means of a heated-water mattress with thermal control means, the system includes a sensing unit for acquiring sound information; a memory unit where a first application and a second application can be recorded; and a processor unit where the first application and the second application can be executed. The processor unit generates sleep state information based on the sound information acquired from the sensing unit through the first application, generates temperature control information based on the sleep state information generated by the first application through the second application, and transmits the temperature control information to the heated-water mattress, thereby providing a device for controlling the thermal control means of a heated-water mattress with thermal control means.

To address the present task, a program recorded on a recording medium includes the steps of: pre-processing the sound information acquired from the sensing unit to obtain sleep sound information; transmitting the obtained sleep sound information to a server via the transmitting unit; receiving, via the receiving unit, the sleep state information generated by the server based on the transmitted user's sleep sound information; generating temperature control information based on the received sleep state information in a control step; and adjusting the heat to achieve a regulated temperature based on the generated temperature control information in a thermal control step. The program recorded on the recording medium is provided to perform these steps.

To address the present task, a device for controlling the thermal control means of a heated-water mattress with thermal control means includes a memory unit where an application can be recorded; and a processor unit where the application can be executed. The processor unit acquires the user's sound information through the application, transmits the acquired user's sound information to the first server via the application, receives the sleep state information generated by the first server based on the transmitted user's sound information via the application, transmits the received sleep state information to the second server, and receives the temperature control information generated by the second server based on the received sleep state information via the application. The device is provided to control the thermal control means of the heated-water mattress.

To address the present task, a device for controlling the thermal control means of a heated-water mattress with thermal control means includes a sensing unit for acquiring the user's sound information; a memory unit where an application can be recorded; and a processor unit where the application can be executed. The processor unit transmits the sound information acquired from the sensing unit to the first server via the application, receives the sleep state information obtained based on the sound information acquired from the sensing unit from the first server, transmits the obtained sleep state information to the second server, receives the temperature control information obtained based on the received sleep state information from the second server, and transmits the received temperature control information to the heated-water mattress. The device is provided to control the thermal control means of the heated-water mattress.

The cosmetic recommendation method according to the present invention includes the steps of: calculating the user's sleep indicator; generating cosmetic information corresponding to the calculated sleep indicator; and displaying the generated cosmetic information.

The step of generating the cosmetic information may include generating recommended cosmetic information based on a lookup table where cosmetic information corresponding to the sleep indicator is recorded.

Additionally, the step of generating the cosmetic information may include learning to generate cosmetic information corresponding to multiple sleep indicator information to create a cosmetic recommendation model; and inputting the sleep indicator information into the cosmetic recommendation model to output recommended cosmetic information as a result.

Meanwhile, the cosmetic verification method according to the present invention includes the steps of: receiving environment sensing information from a user terminal of a user who used a predetermined cosmetic; acquiring at least one of the user's sleep state information and sleep stage information based on the environment sensing information; generating a verification indicator for the predetermined cosmetic using at least one of the sleep state information and sleep stage information; and verifying the effect of the predetermined cosmetic on sleep quality based on the verification indicator.

The step of acquiring the sleep state information may include generating an inference model trained with environment sensing information as input; and extracting the sleep state information as a result by inputting the environment sensing information received from the user terminal into the inference model.

A method for recommending sleep-related products according to an embodiment of the present invention includes the steps of: acquiring user sleep information from one or more sensor devices; calculating the user's sleep indicator based on the acquired user sleep information; and providing the generated sleep-related product information.

Here, the user sleep information acquired from the one or more sensor devices includes the user's sleep sound information.

Additionally, the step of generating sleep-related product recommendation information according to an embodiment of the present invention may further include generating a lookup table in which sleep-related product information corresponding to the calculated sleep indicator is recorded.

Alternatively, the method for recommending sleep-related products according to an embodiment of the present invention may further include the step of receiving verification indicators of sleep-related products, and in the step of generating sleep-related product recommendation information, the recommendation information may be generated based on the received verification indicators of sleep-related products and the calculated sleep indicator.

Alternatively, the method for recommending sleep-related products according to an embodiment of the present invention may further include receiving an input action from the user, wherein the input action includes at least one of swiping a sleep-related product, entering a keyword, or selecting a keyword, and in the step of generating sleep-related product recommendation information, the recommendation information may be generated based on the calculated sleep indicator and the input action.

Alternatively, the method for recommending sleep-related products according to an embodiment of the present invention may further include receiving statistical information according to the user's attributes, wherein the user's attributes include at least one of the user's gender, age group, occupation, living area, race, presence of pets, environmental factors, or non-environmental factors, and in the step of generating sleep-related product recommendation information, the recommendation information may be generated based on the calculated sleep indicator and the received statistical information according to the user's attributes.

Alternatively, the step of generating sleep-related product recommendation information according to an embodiment of the present invention may further include generating a sleep-related product recommendation model by learning to generate recommendation information based on multiple sleep indicators, and the calculated sleep indicator may be input into the sleep-related product recommendation model to output the recommendation information as a result.

Additionally, the step of calculating the user's sleep indicator based on the acquired user sleep information according to an embodiment of the present invention may further include converting the frequency components of the audio information included in the user's sleep information into information reflecting changes over the time axis, and acquiring at least one of the user's sleep state information and sleep stage information based on the converted information.

Here, in the step of calculating the user's sleep indicator based on the acquired user sleep information, the converted information may be a visualization of changes over the time axis of the frequency components of the audio information.

Alternatively, in the step of calculating the user's sleep indicator based on the acquired user sleep information, the converted information may be a spectrogram.

Meanwhile, the method for verifying sleep-related products according to an embodiment of the present invention includes acquiring user sleep information from one or more sensor devices; acquiring at least one of the user's sleep intention information, sleep state information, and sleep stage information based on the acquired user sleep information; and verifying the impact of the sleep-related product on sleep based on at least one of the user's sleep intention information, sleep state information, and sleep stage information.

Here, the user sleep information acquired from one or more sensor devices includes the user's sleep sound information.

Herein, the step of verifying the impact of the sleep-related products on sleep according to an embodiment of the present invention further includes generating a verification indicator for the sleep-related products. The verification indicator may be generated in the form of a lookup table or a numerical indicator related to sleep.

Additionally, the numerical indicator related to sleep according to an embodiment of the present invention is characterized by being calculated based on a lookup table or based on the numerical representation of sleep analysis results. The numerical indicator related to sleep may be calculated based on at least one of the user's sleep onset latency, sleep onset time, wake-up time, total sleep time, and sleep time for each sleep stage when using the sleep-related products.

Herein, the numerical representation of the sleep analysis results according to an embodiment of the present invention is a comprehensive sleep score with a maximum of 100 points, calculated using a predefined formula. The predefined formula may be a formula that calculates the comprehensive score by substituting scores corresponding to each sleep stage of the user using the sleep-related products, based on scores corresponding to each sleep stage information.

Meanwhile, the step of generating the verification indicator according to an embodiment of the present invention further includes receiving the user's subjective judgment indicator, which is calculated based on at least one of a string value, a numerical value, or the user's input action. The verification indicator for the sleep-related products generated in the step of generating the verification indicator may be created by additionally considering the received user's subjective judgment indicator.

Furthermore, the method for verifying sleep-related products according to an embodiment of the present invention includes acquiring at least one of the user's sleep intention information, sleep state information, and sleep stage information based on the acquired user's sleep information. This step involves converting the changes over the time axis of frequency components of audio information included in the user's sleep information into information, and acquiring at least one of the user's sleep state information and sleep stage information based on the converted information.

Herein, in the step of acquiring at least one of the user's sleep intention information, sleep state information, and sleep stage information based on the acquired user's sleep information, the converted information may be visualized to represent the changes over the time axis of the frequency components of the audio information.

Alternatively, in the step of acquiring at least one of the user's sleep intention information, sleep state information, and sleep stage information based on the acquired user's sleep information, the converted information may be a spectrogram.

The method for providing environment adjustment information for sleep according to the present invention to achieve the above objective includes the steps of: a smart home-appliance acquiring sleep sound information related to the user's sleep in real-time through a microphone module; a user terminal receiving the acquired sleep sound information, converting it into a spectrogram, performing analysis, and determining the user's sleep stage in real-time; and the user terminal outputting a control signal to control the operation of the smart home-appliance in real-time according to events occurring at each determined sleep stage. The step of outputting the control signal includes the smart home-appliance responding to the control signal to provide the sleep environment to the user.

The determined sleep stages in the method for providing environment adjustment information for sleep according to the present invention to achieve the above objective include detecting when the user enters the bedroom, when the user lies on the bed, when sleep onset is detected, when deep sleep entry is detected, when sleep apnea occurrence is detected, when awakening during sleep is detected, when REM sleep occurrence around alarm time is detected, and when wake-up is detected.

The case where the user lying on the bed is detected in the method for providing environment adjustment information for sleep according to the present invention to achieve the above objective is characterized by including the step where the sleep onset button is pressed by the user lying on the bed, and the user's sleep intention is estimated by the user terminal.

The smart home-appliance according to the method for providing environment adjustment information for sleep according to the present invention, aimed at achieving the aforementioned objective, is characterized by including at least one of an air conditioner, humidifier, dehumidifier, smart speaker, air purifier, smart TV, robotic vacuum cleaner, lighting, smart bed, clothing care device, smart diffuser, washing machine, dryer, water purifier, and refrigerator.

The air conditioner according to the method for providing environment adjustment information for sleep according to the present invention, aimed at achieving the aforementioned objective, is characterized by setting the airflow and the brightness of the display unit when the user's lying on the bed is detected, switching the type of wind to indirect wind, and setting the temperature to reduce the time to fall asleep based on the user's personal records.

The air conditioner according to the method for providing environment adjustment information for sleep according to the present invention, aimed at achieving the aforementioned objective, is characterized by setting the temperature suitable for the user through past matching data on the correlation between the user's sleep quality and temperature when the falling asleep is detected.

The air conditioner according to the method for providing environment adjustment information for sleep according to the present invention, aimed at achieving the aforementioned objective, is characterized by setting the temperature to protect the user's neck and nose when the occurrence of sleep apnea is detected, setting the temperature to allow the user to re-enter sleep when awakening during sleep is detected, and setting the temperature and airflow to assist the user's alertness after waking when waking is detected.

The humidifier and the dehumidifier according to the method for providing environment adjustment information for sleep according to the present invention, aimed at achieving the aforementioned objective, are characterized by setting the humidity suitable for each user or to a humidity level that can reduce the time to fall asleep based on the user's personal records when the user's lying on the bed is detected.

The humidifier and the dehumidifier according to the method for providing environment adjustment information for sleep according to the present invention, aimed at achieving the aforementioned objective, are characterized by activating in a low-noise state when falling asleep is detected, determining the occurrence of sleep apnea and awakening during sleep when deep sleep entry is detected, and increasing the bedroom humidity to alleviate the symptoms of sleep apnea when sleep apnea is determined.

The humidifier and the dehumidifier according to the method for providing environment adjustment information for sleep according to the present invention, aimed at achieving the aforementioned objective, are characterized by setting the humidity to allow the user to re-enter sleep when awakening during sleep is determined.

The smart speaker according to the method for providing environment adjustment information for sleep according to the present invention, aimed at achieving the aforementioned objective, is characterized by playing sleep-inducing sounds or predetermined falling asleep content based on the user's personal records when the user's lying on the bed is detected.

The smart speaker according to the method for providing environment adjustment information for sleep according to the present invention, aimed at achieving the aforementioned objective, is characterized by determining the occurrence of sleep apnea and awakening during sleep when deep sleep entry is detected, and playing falling asleep content without voice when awakening during sleep is determined.

The air purifier according to the method for providing environment adjustment information for sleep according to the present invention, aimed at achieving the aforementioned objective, is characterized by reducing the LED brightness and decreasing the noise and airflow generated during operation when the user's lying on the bed is detected.

In accordance with the present invention, the method for providing environment adjustment information for sleep, the air purifier is characterized by switching to a rapid purification mode by increasing the airflow when the entry into deep sleep is detected.

In accordance with the present invention, the method for providing environment adjustment information for sleep, when the air purifier is of a table type, is characterized by changing the color of the mood light according to the quality of sleep upon detecting the user's awakening.

In accordance with the present invention, the method for providing environment adjustment information for sleep, the smart TV is characterized by displaying statistics of the user's recent sleep quality and the target sleep for the day based on these statistics when the user's lying on the bed is detected.

In accordance with the present invention, the method for providing environment adjustment information for sleep, the smart TV is characterized by displaying a predetermined sleep report on the screen upon detecting the user's awakening.

In accordance with the present invention, the method for providing environment adjustment information for sleep, the robot vacuum cleaner is characterized by detecting the user's falling asleep and operating in automatic cleaning mode, and increasing the freedom of cleaning area and cleaning time when the entry into deep sleep is detected.

In accordance with the present invention, the method for providing environment adjustment information for sleep, the robot vacuum cleaner is characterized by temporarily pausing operation if cleaning is in progress when awakening during sleep is detected, and returning to the charging dock when REM sleep occurrence is detected around the alarm time.

In accordance with the present invention, the method for providing environment adjustment information for sleep includes the steps of: the smart watch acquiring sleep sound information related to the user's sleep in real-time through a microphone module; the user terminal receiving the acquired sleep sound information, converting it into a spectrogram, and performing analysis to determine the user's sleep stage in real-time; and outputting a control signal from the user terminal to control the operation of the smart watch in real-time according to events occurring at each determined sleep stage; wherein the step of outputting the control signal includes the smart watch responding to the control signal to provide the sleep environment to the user.

In accordance with the present invention, the method for providing environment adjustment information for sleep, the smart watch is characterized by providing a service for falling asleep through vibration when the user's lying on the bed is detected, and adjusting the intensity of the vibration inversely proportional to the determination degree of the user's falling asleep state, or adjusting the vibration duration according to the average falling asleep time for each individual when falling asleep is detected.

In accordance with the present invention, the method for providing environment adjustment information for sleep, the service for falling asleep is characterized by including a breathing guide and a meditation guide.

According to the method for providing environment adjustment information for sleep according to the present invention, the smart watch, upon detecting the occurrence of sleep apnea, provides a gentle vibration to the user when sleep apnea is detected or predicted to occur, thereby interrupting the user's sleep apnea. Additionally, when REM sleep is detected around the alarm time, it provides a wake-up alarm through vibration or provides an alarm at a time when the user is more likely to wake up based on the user's personal sleep records.

According to the method for providing environment adjustment information for sleep according to the present invention, the smart watch, upon detecting awakening, provides a predetermined sleep report through the screen at the user's awakening time.

Meanwhile, the information on the method for providing environment adjustment information for sleep according to the present invention for achieving the other objectives can be stored in a computer-readable recording medium.

Specific details of other embodiments are included in the “Detailed Description of the Invention” and the accompanying “Drawings.”

The advantages and/or features of the present invention, and methods of achieving them, will become apparent by referring to various embodiments described in detail below in conjunction with the accompanying drawings.

However, the present invention is not limited to the configurations of each embodiment disclosed below but may be implemented in various different forms. Each embodiment disclosed herein is provided to make the disclosure of the present invention complete and to fully convey the scope of the present invention to those skilled in the art to which the present invention pertains. The present invention should be defined only by the scope of the claims.

### Effects of the Invention

According to one embodiment of the present invention, it is possible to predict the user's wake time and/or sleep state information, allowing for convenient and accurate analysis of various users' sleep at home without being restricted by time and place.

Furthermore, there is no need for the user to wear wearable devices during sleep analysis, thereby increasing the user's physical freedom during sleep time.

Furthermore, by collecting polysomnography results globally, sleep sound data can be established, and sound AI can be verified across various races, ages, genders, and measurement environments, thereby creating a new standard for home environment sleep tracking.

Additionally, an AI sleep stage analysis model can be developed by learning various ambient noises, including routine noises occurring in the user's sleep environment and abnormal or intermittent noises.

Moreover, by utilizing smartphone sound data and smart speaker sound data collected simultaneously with polysomnography from numerous clinical subjects over an extended period, a sound AI and wireless communication sensing clinical data set can be established.

Furthermore, using smart home-appliances and smartphones, a user's sleep can be analyzed in depth, allowing for not only individual sleep analysis but also multi-person sleep analysis.

Additionally, in the event of a user's sleep disorder, it can be appropriately alleviated, and when multiple people are sleeping in the same space, an alarm for alleviating the sleep disorder can be delivered only to the user experiencing the disorder, thereby preventing disturbance to others' sleep.

Moreover, using smart home-appliances and/or smartphones, the user's physical activity state can be monitored in real-time 24 hours a day.

According to one embodiment of the present invention, an optimized sleep environment can be provided to improve the quality of the user's sleep through sleep state information detected in relation to the user's sleep environment.

In particular, by optimizing the sleep environment concerning various factors such as air quality, temperature, and/or humidity of the sleep environment, the quality of sleep can be significantly enhanced.

The effects of the present invention are not limited to the effects mentioned above, and other effects not mentioned will be clearly understood by those skilled in the art from the following description.

### BRIEF DESCRIPTION OF THE DRAWINGS

FIG. 1(a) illustrates a conceptual diagram of a system in which various aspects of a computing device for creating a sleep environment based on sleep state information related to an embodiment of the present invention can be implemented.

FIG. 1(b) illustrates a conceptual diagram of a system in which various aspects of a sleep environment adjustment device related to another embodiment of the present invention can be implemented.

FIG. 1(c) illustrates a conceptual diagram of a system in which various aspects of different electronic devices related to another embodiment of the present invention can be implemented.

FIG. 2a illustrates a block diagram of a computing device for creating a sleep environment based on sleep state information according to an embodiment of the present invention.

FIG. 2b is a block diagram for explaining an environment adjustment device equipped with sleep state information generation means according to an embodiment of the present invention.

FIG. 2c is a block diagram for explaining an environment adjustment device that receives sleep state information from a server and controls the environment adjustment unit according to an embodiment of the present invention.

FIG. 2d is a block diagram for explaining a home-appliance control device equipped with sleep state information generation means according to an embodiment of the present invention.

FIG. 2e is a block diagram for explaining an environment adjustment device that receives sleep state information from a server and controls a home-appliance according to an embodiment of the present invention.

FIG. 2f is a block diagram for explaining an environment adjustment device that receives sleep state information from another electronic device and controls a home-appliance according to an embodiment of the present invention.

FIG. 2g is a block diagram for explaining an environment adjustment device in which another electronic device senses environment sensing information, receives sleep state information from a server, and controls a home-appliance according to an embodiment of the present invention.

FIG. 2h is a block diagram illustrating an environment adjustment device that receives sleep state information from a first server and environment adjustment information from a second server to control a home-appliance, according to an embodiment of the present invention.

FIG. 2i is a block diagram illustrating an environment adjustment device that controls a home-appliance by receiving environment adjustment information generated by a second server, which receives sleep state information from a first server, according to an embodiment of the present invention.

FIG. 2j is a block diagram illustrating the control of an environment adjustment device through a network, according to an embodiment of the present invention.

FIG. 3 is a diagram comparing the results of polysomnography (PSG result) and the analysis results using an AI algorithm according to the present invention (AI result).

FIG. 4 is a diagram comparing the results of polysomnography (PSG result) with the analysis results using an AI algorithm according to the present invention (AI result) in relation to sleep apnea and hypopnea.

FIG. 5 is an illustrative diagram explaining the process of acquiring sleep sound information from environment sensing information related to an embodiment of the present invention.

FIG. 6(a) is an illustrative diagram explaining a method for obtaining a spectrogram corresponding to sleep sound information related to an embodiment of the present invention.

FIG. 6(b) is a conceptual diagram explaining a privacy protection method using mel spectrogram conversion for sleep sound information extracted from a user in the sleep analysis method according to the present invention.

FIG. 7 is an illustrative diagram exemplarily showing time-specific environment adjustment information according to a user's sleep state related to an embodiment of the present invention.

FIG. 8 is an exemplary flowchart illustrating a method for providing a sleep environment adjustment method according to sleep state information related to an embodiment of the present invention.

FIG. 9 is a schematic diagram illustrating one or more network functions related to an embodiment of the present invention.

FIG. 10 shows an exemplary block diagram of a sleep environment adjustment device related to an embodiment of the present invention.

FIG. 11(a) illustrates an exemplary block diagram of a receiving module and a transmitting module related to an embodiment of the present invention.

FIG. 11(b) is a block diagram showing the configuration of a smart home-appliance within an AI-based non-contact sleep analysis system according to the present invention.

FIG. 12 is an exemplary diagram for explaining a second sensor unit that detects whether a user is located in a predefined area related to an embodiment of the present invention.

FIG. 13 is a flowchart illustratively showing the process of generating sleep state information through an automatic sleep measurement mode related to an embodiment of the present invention.

FIG. 14 is a flowchart illustratively showing the process of creating an environment that induces a user's sleep entry related to an embodiment of the present invention.

FIG. 15 is a flowchart illustratively showing the process of changing a user's sleep environment during sleep and just before waking up related to an embodiment of the present invention.

FIGS. 16(a) and 16(b) are conceptual diagrams for explaining the operation of an air conditioner according to an embodiment of the present invention.

FIGS. 16(c) and 16(d) are conceptual diagrams for explaining the operation of an air purifier according to an embodiment of the present invention.

FIG. 17(a) is a block diagram illustrating the configuration of an air conditioner according to an embodiment of the present invention.

FIG. 17(b) is a block diagram illustrating the configuration of an air purifier according to an embodiment of the present invention.

FIG. 18 is a diagram for explaining an example of the air conditioner shown in FIGS. 16 and 17.

FIG. 19 is a diagram for explaining another example of the air conditioner shown in FIGS. 16 and 17.

FIGS. 20 to 21 are diagrams for explaining yet another example of the air conditioner shown in FIGS. 16 and 17.

FIG. 22 is a diagram for explaining yet another example of the air conditioner shown in FIGS. 16 and 17.

FIGS. 23(a) and (b) are diagrams for explaining a method of operating the indoor unit 500″) shown in FIGS. 20 to 21 in sleep mode through the display unit 570″) of the indoor unit 500″).

FIGS. 23(c) and (d) are diagrams of the display unit 4000 for explaining the sleep mode of the air purifier 700′) according to an embodiment of the present invention.

FIG. 24(a) is a diagram for explaining a method of operating the indoor unit 500′, 500″, 500″′, 500″″) shown in FIGS. 18 to 22 in sleep mode through the remote control 600 according to an embodiment of the present invention.

FIG. 24(b) is a diagram showing an example of the display unit 4000 of the air purifier 700′) according to an embodiment of the present invention.

FIGS. 25(a) and 25(b) are diagrams for explaining a method of driving the indoor unit 500′, 500″, 500″′, 500″″) shown in FIGS. 18 to 22 into sleep mode through the user terminal 10 according to an embodiment of the present invention.

FIG. 25(c) is a diagram showing the screen of a first application for remotely controlling the air purifier 700″) from the user terminal 10 according to an embodiment of the present invention.

FIG. 25(d) is a diagram showing the screen of an application for controlling the sleep mode of the air purifier 700″) according to an embodiment of the present invention.

FIG. 26 is a diagram for explaining a method of automatically driving the indoor unit or air purifier into sleep mode.

FIGS. 27 to 28 are diagrams for explaining the timing of the sleep mode operation shown in FIG. 26.

FIGS. 29(a) and 29(b) are diagrams for explaining an example of the air purifier shown in FIGS. 16 and 17.

FIG. 30 is a diagram showing a state in which some parts of the cover 1100, 2100 of the air purifier 700′) shown in FIG. 29 are removed.

FIG. 31(a) is a diagram for explaining another example of the air purifier shown in FIGS. 16 and 17.

FIG. 31(b) is a diagram for explaining the timing of the sleep mode operation of the air purifier 700″′) shown in FIG. 26.

FIG. 32(a) is a diagram for explaining sleep stage analysis using a spectrogram in the sleep analysis method according to the present invention.

FIG. 32(b) is a diagram for explaining the determination of sleep disorders using a spectrogram in the sleep analysis method according to the present invention.

FIG. 33(a) is a diagram illustrating the experimental process for verifying the performance of the sleep analysis method according to the present invention.

FIG. 33(b) is a graph verifying the performance of the sleep analysis method according to the present invention, comparing the polysomnography (PSG) results with the analysis results using the AI algorithm according to the present invention.

FIG. 34 is a table verifying the accuracy of the sleep analysis method according to the present invention, showing experimental result data analyzed based on age, gender, BMI, and presence of disease.

FIG. 35 is a conceptual diagram illustrating an embodiment of the sleep analysis method according to the present invention, showing the case using a smart speaker and a smartphone for ease of understanding.

FIG. 36(a) is a flowchart explaining the method for preventing and alleviating sleep disorders using an AI-based non-contact sleep analysis system according to an embodiment of the present invention.

FIG. 36(b) is a flowchart explaining the method for preventing and alleviating sleep disorders using an AI-based non-contact sleep analysis system according to another embodiment of the present invention.

FIG. 37 is a diagram explaining the traffic response method when the sleep analysis method according to the present invention is performed in the cloud.

FIG. 38 is a conceptual diagram for explaining single-user sleep analysis and multi-user sleep analysis in the sleep analysis method according to the present invention.

FIG. 39 is a flowchart for explaining the operation of the AI-based non-contact sleep analysis method according to the present invention.

FIG. 40 is a flowchart illustrating an embodiment of various smart home-appliances used in the sleep analysis method according to the present invention.

FIG. 41 is a table illustrating an example of the operation in the sleep preparation stage of a specific scenario of multiple smart home-appliances operating in a time-series manner according to the user's sleep stages using the sleep analysis method of the present invention.

FIG. 42 is a table illustrating an example of the operation in the stage from falling asleep to before deep sleep, connected in time-series succession to FIG. 41 in the scenario.

FIG. 43 is a table illustrating an example of the operation in the stage from after deep sleep to before wake-up detection, connected in time-series succession to FIG. 42 in the scenario.

FIG. 44 is a table illustrating an example of the operation in the wake-up stage, connected in time-series succession to FIG. 43 in the scenario.

FIG. 45 is a conceptual diagram illustrating the training method using only sleep polysomnography microphone data (S) in a hospital environment according to a conventional sleep analysis method, for comparison with the sleep analysis method of the present invention.

FIG. 46 is a conceptual diagram of a method for generating an AI sleep analysis model by reflecting various sounds in a home environment according to the sleep analysis method of the present invention, as shown in the training method of FIG. 45.

FIG. 47 is a table verifying the performance of the sleep analysis method according to the present invention, trained by dividing into nine groups according to the types of residential noise.

FIG. 48 is a schematic diagram for explaining the 24-hour monitoring process of a user by the AI-based non-contact sleep analysis system and sleep analysis method according to the present invention.

FIG. 49 is a table comparing the smart home-appliances and sleep analysis method according to the present invention with the products and devices of existing world-leading sleep tech companies, showing the mean per class results.

FIG. 50 is a block diagram illustrating the operation of an AI-based non-contact sleep analysis system according to an embodiment of the present invention.

FIG. 51 is a block diagram illustrating the operation among components of an AI-based non-contact sleep analysis system according to an embodiment of the present invention.

FIG. 52 is a table describing the position where the environment adjustment device is placed, and, for each detailed product, the activation status according to sleep state information, and exemplary operations in sleep mode and wake mode.

FIG. 53 is a block diagram illustrating the operation of a system implementing the method of providing sleep environment adjustment information according to the present invention.

FIG. 54 is a conceptual diagram illustrating the operation of a wearable device within the system shown in FIG. 53.

FIG. 55 is a conceptual diagram illustrating the operation of a wearable device interfacing with a smartphone according to the present invention.

FIG. 56 is a table showing the operation of an air conditioner and a humidifier/dehumidifier among the major smart home-appliances with frequent operation transitions according to one embodiment, utilizing the method of providing sleep environment adjustment information of the present invention in accordance with the user's sleep stage flow.

FIG. 57 is a table showing the operation of a smart speaker and an air purifier among the major smart home-appliances according to an embodiment of the present invention.

FIG. 58 is a table showing the operation of a smart TV and a robotic vacuum cleaner among the major smart home-appliances according to an embodiment of the present invention.

FIG. 59 is a flowchart illustrating the operation of a smart watch within the system shown in FIG. 53 according to another embodiment of the present invention.

FIG. 60 is a table illustrating the operation of a smart watch according to another embodiment of the present invention.

FIG. 61a is a conceptual diagram illustrating a sleep-related product recommendation system according to the present invention.

FIG. 61b is a conceptual diagram illustrating a sleep-related product verification system according to the present invention.

FIG. 61c is a conceptual diagram illustrating a sleep-related product recommendation/verification system according to another embodiment of the present invention.

FIG. 62 is a diagram illustrating the experimental process for verifying the performance of the sleep analysis method according to the present invention.

FIG. 63 is a graph verifying the performance of the sleep analysis method according to the present invention, comparing the results of polysomnography (PSG result) with the analysis results using the AI algorithm according to the present invention (AI result).

FIG. 64 is a graph verifying the performance of the sleep analysis method according to the present invention, comparing the results of polysomnography (PSG result) with the analysis results using the AI algorithm according to the present invention (AI result) in relation to sleep apnea and hypopnea.

FIG. 65a is a flowchart illustrating the sleep-related product recommendation method according to the present invention.

FIG. 65b is a flowchart illustrating the sleep-related product verification method according to the present invention.

FIG. 66 is a diagram for explaining the overall structure of the sleep analysis model according to an embodiment of the present invention.

FIG. 67 is a diagram for explaining a feature extraction model and a feature classification model according to an embodiment of the present invention.

FIGS. 68 to 69 are tables for explaining a lookup table in which information on sleep-related products recommended in response to sleep indicators is recorded.

FIG. 70a is a table for explaining a lookup table in which composition information is recorded when the sleep-related product is a composition.

FIG. 70b is a table for explaining a lookup table in which fiber component information is recorded when the sleep-related product is composed of fiber components.

FIG. 71a is a table for explaining a case where a verification indicator for sleep-related products is displayed as a score.

FIGS. 71b and 71c are exemplary diagrams for explaining a case where a verification indicator for sleep-related products is displayed as a numerical evaluation related to the user's sleep according to an embodiment of the present invention.

FIG. 72 is a table for explaining the acquisition of sleep state information from multiple users to generate verification indicators for multiple users.

FIG. 73 is a table for explaining a lookup table in which information on recommended sleep-related products obtained through sleep evaluation statistics of multiple users for multiple sleep-related products is recorded.

FIG. 74 is a diagram showing a graphical user interface for receiving user input actions to calculate subjective judgment indicators according to an embodiment of the present invention.

FIG. 75 is a block diagram for explaining a cosmetic recommendation method according to an embodiment of the present invention.

FIG. 76 is a block diagram illustrating a cosmetic verification method according to an embodiment of the present invention.

FIG. 77 is a conceptual diagram showing a system of a heated-water mattress for creating a sleep environment.

FIG. 78 is a conceptual diagram showing another embodiment of a system of a heated-water mattress 30-1 for creating a sleep environment.

FIG. 79 is a configuration diagram illustrating the operation among components of an AI-based non-contact sleep analysis system according to a heated-water mattress for creating a sleep environment.

FIG. 80 is a diagram illustrating a heated-water mattress where learning and inference are performed on a server according to a heated-water mattress for creating a sleep environment.

FIG. 81 is a diagram illustrating a heated-water mattress where learning, inference, and temperature control are performed on a server according to a heated-water mattress for creating a sleep environment.

FIG. 82 is a diagram illustrating a heated-water mattress where learning, inference, and temperature control are performed on the heated-water mattress itself according to a heated-water mattress for creating a sleep environment.

FIG. 83 is a diagram illustrating a heated-water mattress where learning and inference are performed on a first server, and temperature control is performed on a second server according to a heated-water mattress for creating a sleep environment.

FIG. 84 is a diagram illustrating a device for controlling the thermal control means of a heated-water mattress, which includes a processor unit capable of executing an application.

FIG. 85 is a block diagram illustrating a light-modulation device that receives sleep state information from a server according to an embodiment of the present invention.

FIG. 86 is a conceptual diagram illustrating a light-modulation method for creating a user's sleep environment according to an embodiment of the present invention.

FIG. 87 is a flowchart illustrating a method for analyzing sleep state information, which includes the process of combining sleep sound information and sleep environment information into multimodal data according to an embodiment of the present invention.

FIG. 88 is a flowchart illustrating a method for analyzing sleep state information, which includes the step of combining each inferred sleep sound information and sleep environment information into multimodal data according to an embodiment of the present invention.

FIG. 89 is a flowchart illustrating a method for analyzing sleep state information, which includes the step of combining inferred sleep sound information with sleep environment information into multimodal data according to an embodiment of the present invention.

FIG. 90 is a diagram for explaining consistency training according to an embodiment of the present invention.

FIG. 91 is a diagram for explaining a user modeling method in the emotional modeling method according to the present invention, where the user checks preferred scents by swiping.

FIG. 92 is a diagram for explaining a user modeling method in the emotional modeling method according to the present invention, where the user inputs text related to preferred scents.

FIG. 93 is a diagram for explaining a user modeling method in the emotional modeling method according to the present invention, where the user selects keywords related to preferred scents.

FIG. 94 is a diagram for explaining a user modeling method in the emotional modeling method according to the present invention, where feedback is received from the user after waking up regarding the provided scents.

FIG. 95 is a diagram for explaining a scent-providing device that provides scents to a user during sleep according to the present invention.

### EMBODIMENTS FOR IMPLEMENTING THE INVENTION

The advantages and features of the present invention, as well as methods for achieving them, will become apparent by referring to the embodiments described in detail below in conjunction with the accompanying drawings. However, the present invention is not limited to the embodiments disclosed herein and may be implemented in various other forms. The embodiments are provided to ensure the completeness of the disclosure of the present invention and to fully convey the scope of the invention to those skilled in the art to which the invention pertains. The present invention is defined only by the scope of the claims.

In describing the embodiments disclosed herein, detailed descriptions of related known technologies may be omitted if it is determined that they could obscure the essence of the embodiments disclosed herein. Furthermore, the accompanying drawings are provided merely to facilitate understanding of the embodiments disclosed herein and do not limit the technical ideas disclosed herein. It should be understood that all modifications, equivalents, and substitutes included within the spirit and scope of the present invention are encompassed.

The terms used herein are for the purpose of describing the embodiments and are not intended to limit the present invention.

Unless otherwise defined, all terms (including technical and scientific terms) used herein can be understood in a manner commonly understood by those skilled in the art to which the present invention pertains. Additionally, terms generally defined in dictionaries are not to be ideally or excessively interpreted unless explicitly defined otherwise.

In this specification, the singular form includes the plural form unless specifically stated otherwise. The terms “comprises” and/or “comprising” as used in the specification do not exclude the presence or addition of one or more other components besides the mentioned components. Throughout the specification, the same reference numerals refer to the same components, and “and/or” includes each and every combination of the mentioned components.

Although “first,” “second,” etc., are used to describe various components, these components are not limited by these terms. These terms are merely used to distinguish one component from another. Therefore, a first component mentioned below may also be a second component within the technical spirit of the present invention.

The terms “unit” or “module” as used in the specification refer to hardware components such as software, FPGA, or ASIC, and “unit” or “module” performs certain roles. However, “unit” or “module” is not limited to software or hardware. “Unit” or “module” may be configured to be on addressable storage media and may be configured to reproduce one or more processors. Therefore, as an example, “unit” or “module” includes software components, object-oriented software components, class components, and task components, as well as processes, functions, attributes, procedures, subroutines, segments of program code, drivers, firmware, microcode, circuits, data, databases, data structures, tables, arrays, and variables. The functions provided within components and “units” or “modules” may be combined into fewer components and “units” or “modules” or further separated into additional components and “units” or “modules.”

In this specification, a computer refers to any type of hardware device that includes at least one processor, and it can be understood to encompass software configurations operating on the hardware device according to the embodiment. For example, a computer can be understood to include smartphones, tablet PCs, desktops, laptops, and user clients and applications running on each device, but is not limited thereto.

Furthermore, the “smart home-appliance” described below is a device equipped with a microphone capable of detecting a user's breathing sound and collecting audio data, and may include smart speakers, smart TVs, smart lighting, smart mattresses, and the like.

Additionally, the “SleepTrack App” may refer to an application that delivers a user's sleep report to a smartphone using PUI, VUI, and GUI, and operates smart home-appliances based on the report results.

Additionally, “research interaction” may refer to the research and development of new products aimed at improving the quality of a user's sleep within categories such as fragrance, cosmetics, food, health functional foods, and hormones.

Furthermore, “research interaction of the SleepTrack app” refers to the development of sleep environment adjustment services and new products based on sleep analysis conducted by the SleepTrack app to improve sleep quality.

Moreover, “Sleep Management App Interaction” may refer to the interaction between traditional sleep industries capable of sleep storytelling, such as sports, hotels, cram schools, and the military, and sleep management apps capable of sleep analysis without hardware solutions.

Additionally, “interaction from research interaction to sleep management app” refers to the interaction between new products without digital components and sleep management apps capable of sleep analysis without hardware solutions.

Those skilled in the art should further recognize that various exemplary logical blocks, configurations, modules, circuits, means, logics, and algorithm steps described in connection with the embodiments disclosed herein can be implemented as electronic hardware, computer software, or combinations of both. To clearly illustrate the interchangeability of hardware and software, various exemplary components, blocks, configurations, means, logics, modules, circuits, and steps have been generally described in terms of their functionality above.

Whether such functionality is implemented as hardware or software depends upon the specific application and design constraints imposed on the overall system. Skilled artisans may implement the described functionality in varying ways for each particular application. However, such implementation decisions should not be interpreted as causing a departure from the scope of the present invention.

Hereinafter, embodiments of the present invention will be described in detail with reference to the accompanying drawings.

Each step described herein is described as being performed by a computer, but the subject of each step is not limited thereto, and at least some of the steps may be performed by different devices depending on the embodiment.

### 027 Cases

### Overall Configuration

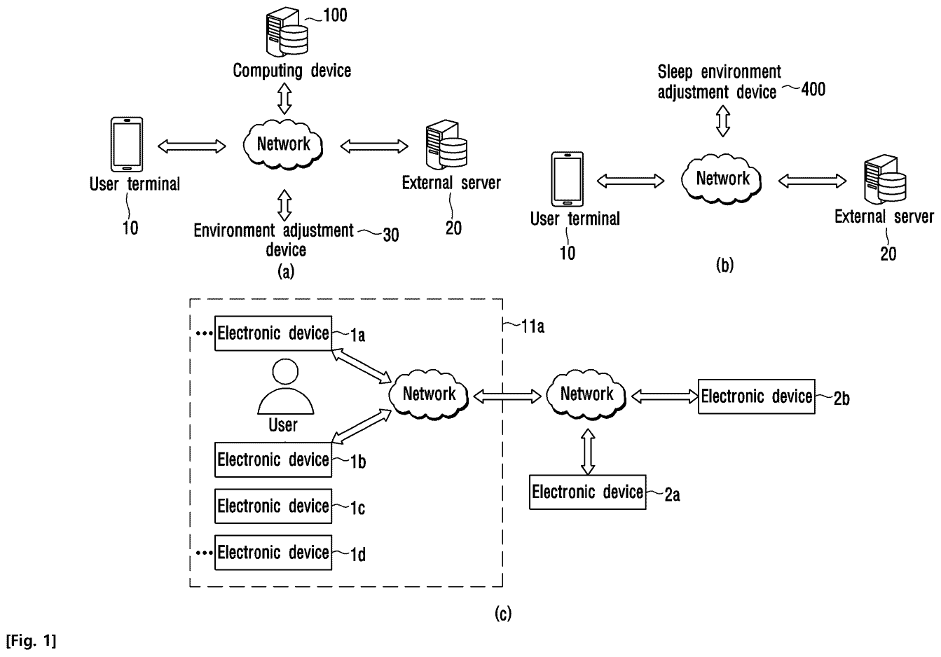

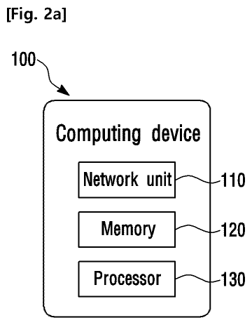

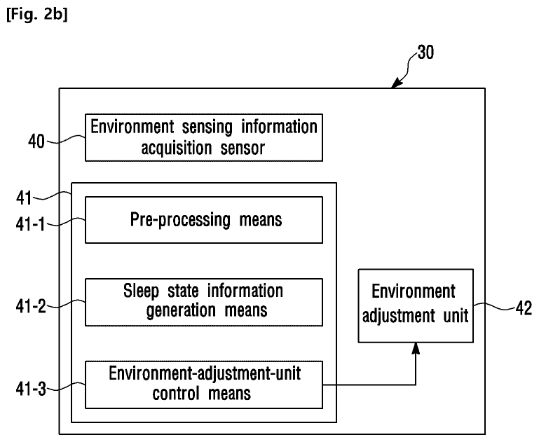

FIG. 1(a) illustrates a conceptual diagram showing a system in which various aspects of a computing device for creating a sleep environment based on sleep state information according to an embodiment of the present invention can be implemented. The system according to embodiments of the present invention may include a computing device 100, a user terminal 10, an external server 20, an environment adjustment device 30, and a network. Here, the system for implementing the method of creating a sleep environment based on the sleep state information shown in FIG. 1(a) is according to one embodiment, and its components are not limited to the embodiment shown in FIG. 1, and may be added, changed, or deleted as necessary.

Meanwhile, FIG. 1(b) illustrates a conceptual diagram of a system in which various aspects of a sleep environment adjustment device related to another embodiment of the present invention can be implemented.

The system according to the embodiments of the present invention may include a sleep environment adjustment device 400, a user terminal 10, an external server 20, and a network. Here, the system for implementing a method to create a sleep environment based on the sleep state information shown in FIG. 1(b) is according to one embodiment, and its components are not limited to the embodiment shown in FIG. 1(b) and may be added, modified, or deleted as necessary.

First, the system according to the embodiment shown in FIG. 1(a) will be described.

As shown in FIG. 1(a), the present invention allows a computing device 100, a user terminal 10, an external server 20, and an environment adjustment device 30 to transmit and receive data for the system according to embodiments of the present invention through a network.

The network according to the embodiments of the present invention may use various wired communication systems such as Public Switched Telephone Network (PSTN), xDSL (x Digital Subscriber Line), RADSL (Rate Adaptive DSL), MDSL (Multi Rate DSL), VDSL (Very High Speed DSL), UADSL (Universal Asymmetric DSL), HDSL (High Bit Rate DSL), and Local Area Network (LAN). Additionally, the network presented here may use various wireless communication systems such as CDMA (Code Division Multi Access), TDMA (Time Division Multi Access), FDMA (Frequency Division Multi Access), OFDMA (Orthogonal Frequency Division Multi Access), SC-FDMA (Single Carrier-FDMA), and other systems.

The network according to the embodiments of the present invention can be configured regardless of its communication mode, whether wired or wireless, and can be composed of various communication networks such as Personal Area Network (PAN) and Wide Area Network (WAN). Furthermore, the network may be the well-known World Wide Web (WWW) and may also use wireless transmission technologies for short-range communication, such as Infrared Data Association (IrDA) or Bluetooth. The technologies described herein can be used not only in the networks mentioned above but also in other networks.

According to one embodiment of the present invention, the user terminal 10 is a terminal that can receive information related to the user's sleep through information exchange with the computing device 100, meaning a terminal possessed by the user. For example, the user terminal 10 may be a terminal related to a user who wishes to improve health through information related to their sleep habits. The user can acquire monitoring information related to their sleep through the user terminal 10. The sleep-related monitoring information may include, for example, sleep state information related to the time the user fell asleep, the duration of sleep, and the time of waking up, or sleep stage information related to changes in sleep stages during sleep. Specifically, sleep stage information may mean information on how the user's sleep changed to light sleep, normal sleep, deep sleep, or REM sleep at each point during the user's last 8 hours of sleep. The specific description of the aforementioned sleep stage information is merely exemplary and the present invention is not limited thereto.

Meanwhile, FIG. 1(c) illustrates a conceptual diagram of a system in which various aspects of different electronic devices related to another embodiment of the present invention can be implemented.

The electronic devices shown in FIG. 1(c) can perform at least one of the operations performed by various devices according to the embodiments of the present invention.

For example, the operations performed by various devices according to the embodiments of the present invention may include acquiring environment sensing information, training a sleep analysis model, inferring a sleep analysis model, acquiring sleep state information, controlling an electronic device, displaying sleep state information, and displaying environment adjustment information.

Alternatively, for example, operations may include receiving information related to a user's sleep, transmitting or receiving environment sensing information, determining environment sensing information, processing or refining data, processing or providing services, analyzing sleep states, constructing a training data set based on information related to a user's sleep, storing information on acquired data or multiple training data for training a neural network, generating environment adjustment information, determining environment adjustment information, operating an environment adjustment module based on environment adjustment information, transmitting or receiving various information, and exchanging data for the system according to embodiments of the present invention through a network.

The electronic devices depicted in FIG. 1(c) may individually perform the operations executed by various devices according to embodiments of the present invention, but may also perform one or more operations simultaneously or in a time-series manner.

Referring to FIG. 1(c), electronic devices 1a to 1d may be electronic devices within the range of a predefined area 11a, which is an area capable of acquiring object state information such as information on a user's movement or breathing.

Meanwhile, referring to FIG. 1(c), electronic devices 1a and 1d may be devices composed of a combination of two or more electronic devices.

Meanwhile, referring to FIG. 1(c), electronic devices 1a and 1b may be electronic devices connected to a network within the predefined area 11a.

Meanwhile, referring to FIG. 1(c), electronic devices 1c and 1d may be electronic devices not connected to a network within the predefined area 11a.

Meanwhile, referring to FIG. 1(c), electronic devices 2a to 2b) may be electronic devices outside the range of the predefined area 11a.

Meanwhile, referring to FIG. 1(c), there may be a network interacting with electronic devices within the range of the predefined area 11a, and a network interacting with electronic devices outside the range of the predefined area 11a.

Here, the network interacting with electronic devices within the range of the predefined area 11a may serve the role of transmitting and receiving information for controlling smart home-appliances.

Additionally, the network interacting with electronic devices within the predefined area 11a may be, for example, a short-range network or a local network. Here, the network interacting with electronic devices within the predefined area 11a may also be, for example, a long-range network or a global network.

A detailed description of the operation of the networks shown in FIG. 1(c) is the same as that described through the diagrams of FIG. 1(a) or FIG. 1(b), and thus redundant descriptions will be omitted.

Meanwhile, referring to FIG. 1(c), electronic devices connected through a network outside the predefined area 11a may be one or more, and in this case, the electronic devices may distribute data processing among themselves or perform one or more operations separately.

Alternatively, if there is more than one electronic device connected through a network outside the predefined area 11a, the electronic devices may operate independently of each other.

Hereinafter, with reference to FIG. 1(c), various aspects according to an embodiment of the present invention will be described, but the present invention is not limited thereto.

For example, according to one embodiment of the present invention, within an electronic device equipped with environment sensing and control functions, the steps of acquiring environment sensing information, performing pre-processing on the acquired environment sensing information, converting the sound information included in the pre-processed environment sensing information into a spectrogram, generating sleep state information based on the converted spectrogram, and controlling the electronic device to adjust the environment based on the generated sleep state information may be performed.

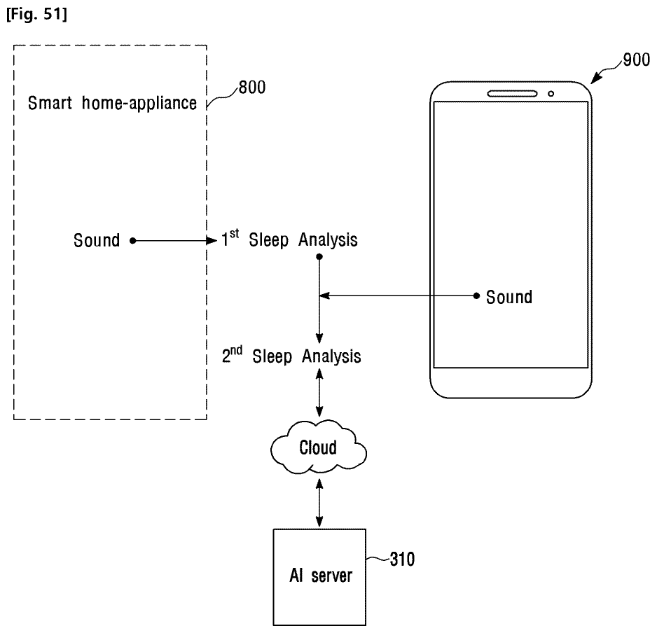

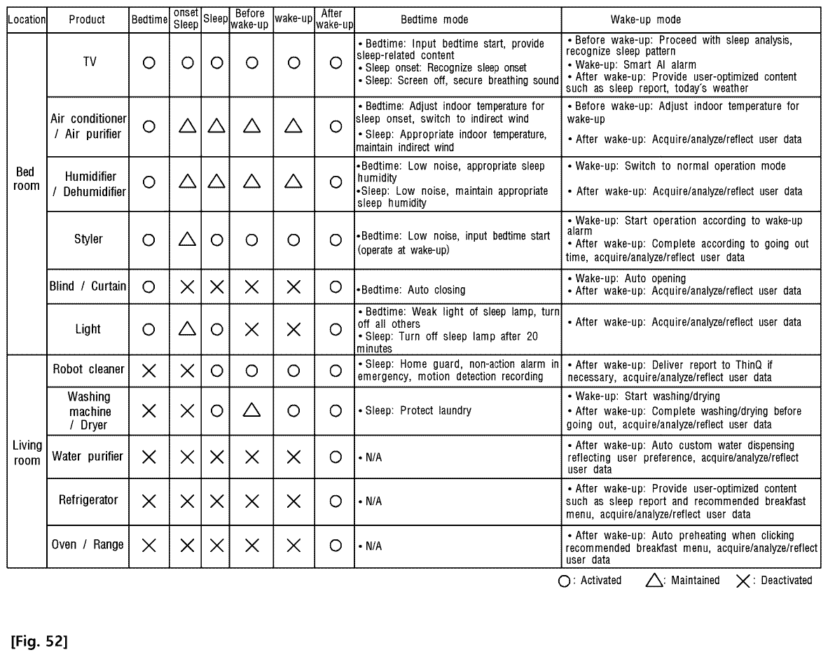

Alternatively, as shown in FIG. 51, according to one embodiment of the present invention, within an electronic device equipped with environment sensing and control functions, the steps of acquiring environment sensing information, performing pre-processing on the acquired environment sensing information, converting the sound information included in the pre-processed environment sensing information into a spectrogram, transmitting the converted spectrogram to an AI server 310, and when the AI server 310 generates sleep state information through learning or inference based on the transmitted spectrogram, receiving the sleep state information generated by the AI server 310, and controlling the electronic device to adjust the environment based on the received sleep state information may be performed.

Alternatively, according to one embodiment of the present invention, there is an electronic device for controlling a home-appliance to adjust the environment, and the steps of acquiring environment sensing information in the electronic device, performing pre-processing on the acquired environment sensing information, converting the sound information included in the pre-processed environment sensing information into a spectrogram, generating sleep state information based on the converted spectrogram, and controlling the home-appliance to adjust the environment based on the generated sleep state information may be performed.

Alternatively, according to one embodiment of the present invention, there is an electronic device for controlling a home-appliance to adjust the environment, and the steps of acquiring environment sensing information in the electronic device, performing pre-processing on the acquired environment sensing information, converting the sound information included in the pre-processed environment sensing information into a spectrogram, transmitting the converted spectrogram to an AI server 310, and when the AI server 310 generates sleep state information through learning or inference based on the transmitted spectrogram, receiving the sleep state information generated by the AI server 310, and controlling the home-appliance to adjust the environment based on the received sleep state information may be performed.

Alternatively, according to one embodiment of the present invention, there is an electronic device for controlling a home-appliance to adjust the environment, where another electronic device acquires environment sensing information, converts the sound information included in the acquired environment sensing information into a spectrogram, and generates sleep state information based on the converted spectrogram. The steps of receiving sleep state information from the other electronic device, and controlling the home-appliance to adjust the environment based on the received sleep state information may be performed. Here, the other electronic device refers to a device different from the electronic device controlling the home-appliance and may correspond to one or more other electronic devices. If there are multiple other electronic devices, the steps of acquiring environment sensing information, converting the sound information included in the environment sensing information into a spectrogram, and generating sleep state information may be performed independently.

For example, according to one embodiment of the present invention, there is an electronic device for controlling a home-appliance to create an environment. Another electronic device acquires environment sensing information, converts the sound information included in the acquired environment sensing information into a spectrogram, and transmits the converted spectrogram to an AI server 310. When the AI server 310 generates sleep state information based on the transmitted spectrogram, the electronic device receives the sleep state information generated by the AI server 310 and controls the home-appliance to create an environment based on the received sleep state information. The description regarding the other electronic device is the same as previously described, and thus redundant description will be omitted.

The various embodiments of the present invention described above illustrate that the acquisition of environment sensing information, pre-processing of environment sensing information, conversion of the spectrogram, generation of sleep state information, and control of electronic devices or home-appliances (e.g., smart home-appliances) do not necessarily occur within the same electronic device. These operations can occur across multiple devices, and they may occur in a time-series manner, simultaneously, or independently and individually. Therefore, the present invention is not limited to the various embodiments described above.

Hereinafter, the various operations according to the present invention will be described with specific examples. However, as previously mentioned, the examples of electronic devices described below are merely illustrative for clear understanding and do not limit the electronic devices performing specific operations.

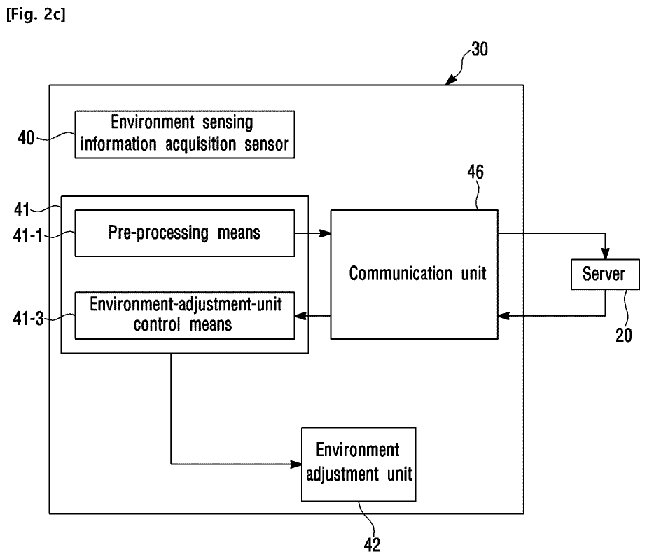

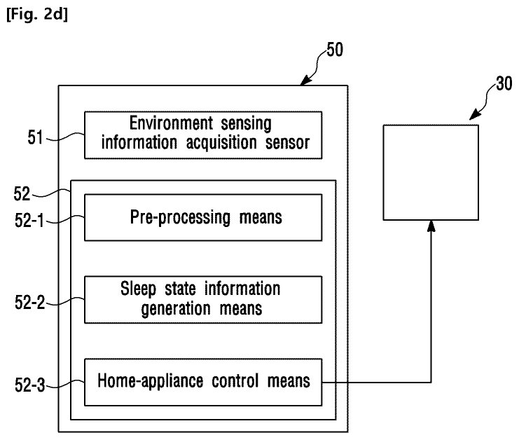

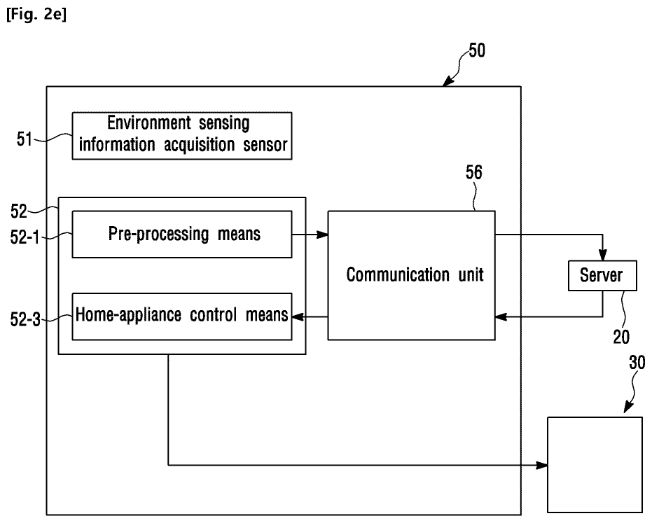

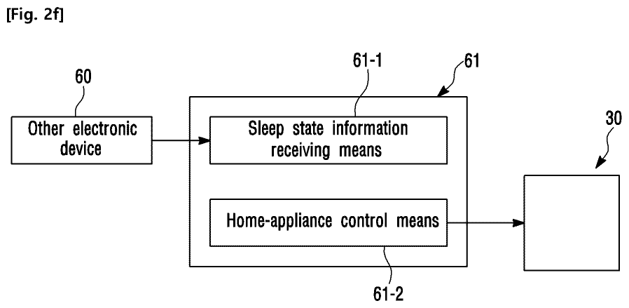

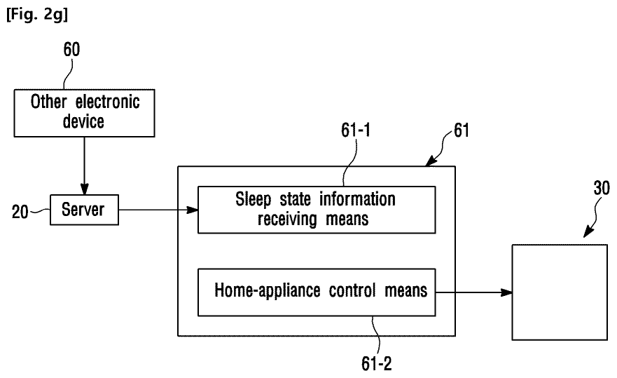

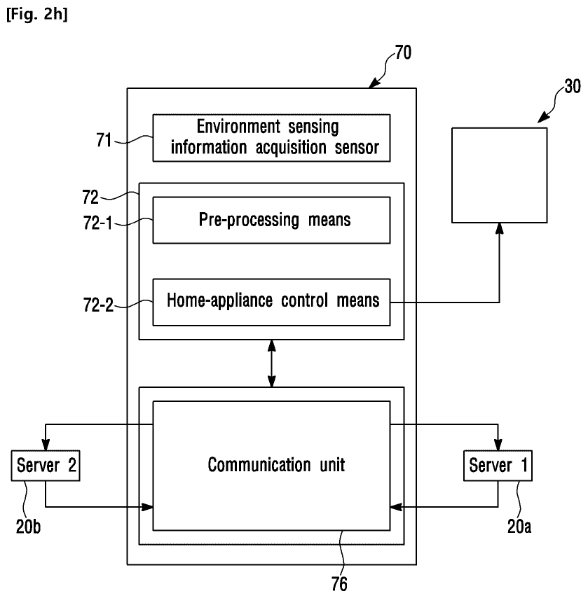

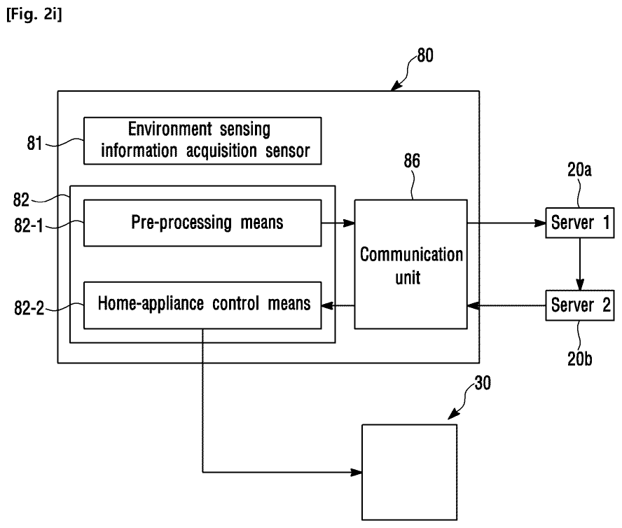

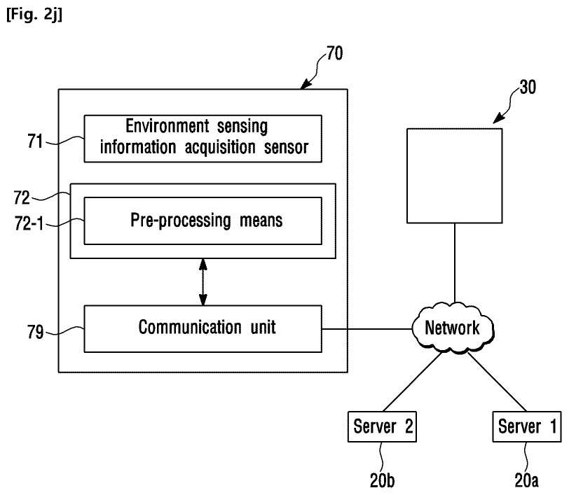

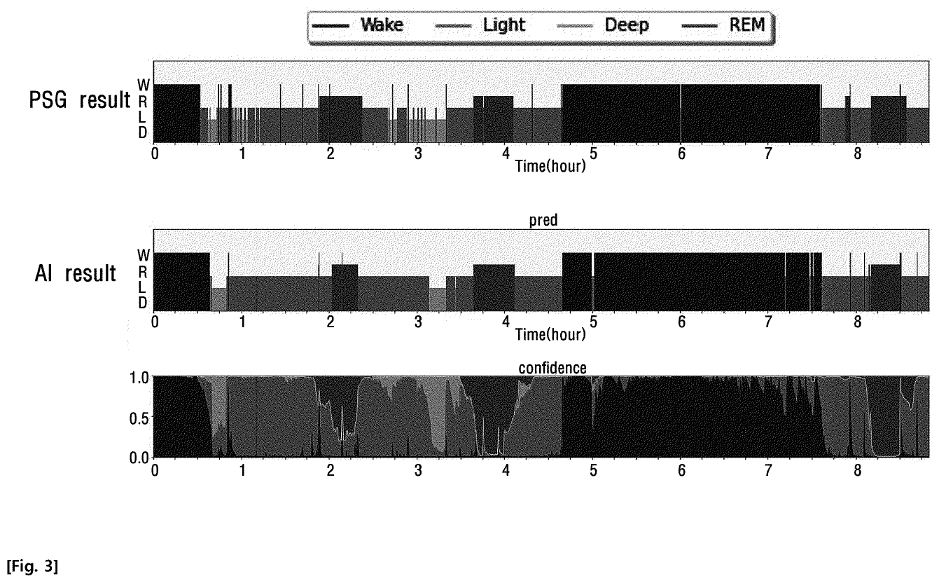

### Description of FIGS. 2b to 2i

FIG. 2b is a block diagram illustrating an environment adjustment device equipped with a sleep state information generation means according to an embodiment of the present invention.

According to one embodiment of the present invention, the environment adjustment device 30 may include an environment adjustment information acquisition sensor 40, a control unit 41, and an environment adjustment unit 42. Specifically, the control unit 41 may include pre-processing means 41-1, sleep state information generation means 41-2, and environment-adjustment-unit control means 41-3.

According to the present invention, the environment adjustment information acquisition sensor 40 of the environment adjustment device 30 can acquire environment sensing information from a user. The pre-processing means 41-1 can perform pre-processing on the environment sensing information acquired by the environment adjustment information acquisition sensor 40. The sleep state information generation means 41-2 can generate sleep state information based on the pre-processed environment sensing information from the pre-processing means 41-1. The environment-adjustment-unit control means 41-3 can control the environment adjustment unit 42 to provide a predetermined scent based on the generated sleep state information.

The environment-adjustment-unit control means 41-3 can control the environment adjustment unit 42 to provide a predetermined scent in real-time based on the generated sleep state information.

The sleep state information generation means 41-2 can convert the environment sensing information into information that includes changes over the time axis of frequency components of the environment sensing information. Specifically, the information including changes over the time axis of frequency components may be a spectrogram 300.

FIG. 2c is a block diagram illustrating an environment adjustment device that receives sleep state information from a server to control the environment adjustment unit according to an embodiment of the present invention.

According to an embodiment of the present invention, the environment adjustment device 30 may include an environment sensing information acquisition sensor 40, a control unit 41, an environment adjustment unit 42, and a communication unit 46. Specifically, the control unit 41 may include pre-processing means 41-1 and environment-adjustment-unit control means 41-3.

According to the present invention, the environment sensing information acquisition sensor 40 of the environment adjustment device 30 can acquire environment sensing information from the user. The pre-processing means 41-1 can perform pre-processing on the environment sensing information acquired from the environment sensing information acquisition sensor 40. The communication unit 46 can transmit the pre-processed environment sensing information from the pre-processing means 41-1 to the server 20. Accordingly, the communication unit 46 can receive sleep state information generated by the server 20. The environment-adjustment-unit control means 41-3 can control the environment adjustment unit 42 to provide a predetermined scent based on the received sleep state information.

The environment-adjustment-unit control means 41-3 can control the environment adjustment unit 42 to provide a predetermined scent in real-time based on the received sleep state information.

The communication unit 46 can receive converted information when the server 20 converts the environment sensing information into information that includes changes in the frequency components over the time axis. Specifically, the information including changes in the frequency components over the time axis may be a spectrogram 300.

FIG. 2d is a block diagram illustrating a home-appliance control device equipped with a sleep state information generation means according to an embodiment of the present invention.

According to an embodiment of the present invention, the electronic device 50 for controlling a home-appliance may include an environment sensing information acquisition sensor 51 and a control unit 52. Specifically, the control unit 52 may include pre-processing means 52-1, sleep state information generation means 52-2, and home-appliance control means 52-3.

According to the present invention, the environment sensing information acquisition sensor 51 of the electronic device 50 for controlling a home-appliance can acquire environment sensing information from the user. The pre-processing means 52-1 can perform pre-processing on the environment sensing information acquired from the environment sensing information acquisition sensor 51. The sleep state information generation means 52-2 can generate sleep state information based on the pre-processed environment sensing information from the pre-processing means 52-1. Accordingly, the home-appliance control means 52-3 can control the environment adjustment device 30 to provide a predetermined scent based on the generated sleep state information.

The home-appliance control means 52-3 can control the environment adjustment device 30 to provide a predetermined scent in real-time based on the generated sleep state information.

The sleep state information generation means 52-2 can convert the environment sensing information into information that includes changes in the frequency components over the time axis. Specifically, the information including changes in the frequency components over the time axis may be a spectrogram 300.

FIG. 2e is a block diagram illustrating an environment adjustment device that controls a home-appliance by receiving sleep state information from a server according to an embodiment of the present invention.

According to an embodiment of the present invention, an electronic device 50 for controlling a home-appliance may include an environment sensing information acquisition sensor 51, a control unit 52, and a communication unit 56. Specifically, the control unit 52 may include pre-processing means 52-1 and home-appliance control means 52-3.

According to the present invention, the environment sensing information acquisition sensor 51 of the electronic device 50 for controlling a home-appliance can acquire environment sensing information from a user. The pre-processing means 52-1 can perform pre-processing on the environment sensing information acquired from the environment sensing information acquisition sensor 51. The communication unit 56 can transmit the pre-processed environment sensing information from the pre-processing means 52-1 to a server 20. Accordingly, the communication unit 56 can receive sleep state information generated by the server 20. The home-appliance control means 52-3 can control the environment adjustment device 30 to provide a predetermined scent based on the received sleep state information.

The home-appliance control means 52-3 can control the environment adjustment device 30 to provide a predetermined scent in real-time based on the received sleep state information.

The communication unit 56 can receive converted information when the server 20 converts the environment sensing information into information including changes in frequency components over the time axis. Specifically, the information including changes in frequency components over the time axis may be a spectrogram 300.

FIG. 2f is a block diagram illustrating an environment adjustment device for controlling a home-appliance by receiving sleep state information from another electronic device according to an embodiment of the present invention.

According to an embodiment of the present invention, an electronic device 61 for controlling a home-appliance may include sleep state information receiving means 61-1 and home-appliance control means 61-2.

According to the present invention, another electronic device 60 can acquire environment sensing information from a user. The other electronic device 60 can perform pre-processing on the acquired environment sensing information. The other electronic device 60 can generate sleep state information based on the pre-processed environment sensing information.

Accordingly, the sleep state information receiving means 61-1 of the electronic device 61 for controlling a home-appliance can receive sleep state information from the other electronic device 60. Consequently, the home-appliance control means 61-2 can control the environment adjustment device 30 to provide a predetermined scent based on the received sleep state information.

The home-appliance control means 61-2 can control the environment adjustment device 30 in real-time based on the received sleep state information.

The other electronic device 60 can convert the environment sensing information into information including changes in frequency components over the time axis. Accordingly, the sleep state information receiving means 61-1 of the electronic device 61 for controlling a home-appliance can receive the converted information. Specifically, the information including changes in frequency components over the time axis may be a spectrogram 300.

FIG. 2g is a block diagram illustrating an environment adjustment device for controlling a home-appliance by sensing environment sensing information and receiving sleep state information from a server according to an embodiment of the present invention.

According to an embodiment of the present invention, an electronic device 61 for controlling a home-appliance may include a sleep state information receiving means 61-1 and a home-appliance control means 61-2.

According to the present invention, another electronic device 60 can acquire environment sensing information from a user. The other electronic device 60 can perform pre-processing on the acquired environment sensing information. The other electronic device 60 can transmit the pre-processed environment sensing information to a server 20. Accordingly, the sleep state information receiving means 61-1 of the electronic device 61 for controlling a home-appliance can receive sleep state information from the server 20. Consequently, the home-appliance control means 61-2 can control the environment adjustment device 30 to provide a predetermined scent based on the received sleep state information.

The home-appliance control means 61-2 can control the environment adjustment device 30 to provide a predetermined scent based on the received sleep state information.

The server 20 can convert the environment sensing information into information that includes changes in the frequency components of the environment sensing information over the time axis. Accordingly, the sleep state information receiving means 61-1 of the electronic device 61 for controlling a home-appliance can receive the converted information. Specifically, the information including changes in the frequency components over the time axis may be a spectrogram 300.

FIG. 2h is a block diagram illustrating an environment adjustment device that receives sleep state information from a first server and environment adjustment information from a second server to control a home-appliance according to an embodiment of the present invention.

According to an embodiment of the present invention, a home-appliance control device 70 includes an environment adjustment information acquisition sensor 71, a control unit 72, and a communication unit 76. Specifically, the control unit 72 may include pre-processing means 72-1 and home-appliance control means 72-2.

According to the present invention, the environment adjustment information acquisition sensor 71 of the home-appliance control device 70 can acquire environment sensing information from a user. The pre-processing means 72-1 can perform pre-processing on the environment sensing information acquired by the environment adjustment information acquisition sensor 71. The communication unit 76 can transmit the pre-processed environment sensing information from the pre-processing means 72-1 to a first server 20a. Accordingly, the first server 20a generates sleep state information, and the communication unit 76 receives the sleep state information. The communication unit 76 then transmits the sleep state information to a second server 20b, which generates environment adjustment information based on the received sleep state information. The communication unit 76 receives the environment adjustment information from the second server 20b, and the home-appliance control means 72-2 generates environment adjustment device control information based on the received environment adjustment information to control the environment adjustment device 30. The communication unit 76 can receive the converted information when the first server 20a converts the environment sensing information into information that includes changes in the frequency components over the time axis. Specifically, the information including changes in the frequency components over the time axis may be a spectrogram 300.

FIG. 2i is a block diagram illustrating an environment adjustment device that receives sleep state information from a first server and generates environment adjustment information from a second server to control a home-appliance according to an embodiment of the present invention.

According to an embodiment of the present invention, a home-appliance control device 80 includes an environment adjustment information acquisition sensor 81, a control unit 82, and a communication unit 86. Specifically, the control unit 82 may include pre-processing means 82-1 and home-appliance control means 82-2.

According to the present invention, the environment adjustment information acquisition sensor 81 of the home-appliance control device 80 can acquire environment sensing information from a user. The pre-processing means 82-1 can perform pre-processing on the environment sensing information acquired by the environment adjustment information acquisition sensor 81. The communication unit 86 can transmit the pre-processed environment sensing information from the pre-processing means 82-1 to a first server 20a. Accordingly, the first server 20a generates sleep state information, and the first server 20a transmits the sleep state information to a second server 20b, which generates environment adjustment information based on the received sleep state information. The communication unit 86 receives the environment adjustment information from the second server 20b, and the home-appliance control means 82-2 generates environment adjustment device control information based on the received environment adjustment information to control the environment adjustment device 30. The communication unit 86 can receive the converted information when the first server 20a converts the environment sensing information into information that includes changes in the frequency components over the time axis. Specifically, the information including changes in the frequency components over the time axis may be a spectrogram 300.

FIG. 2j is a block diagram illustrating the control of an environment adjustment device through a network according to an embodiment of the present invention.

According to an embodiment of the present invention, the home-appliance control device 70 includes an environment sensing information acquisition sensor 71, a control unit 72, and a communication unit 79. Specifically, the control unit 72 may include pre-processing means 72-1.

The pre-processing means 72-1 of the control unit 72 performs pre-processing on the environment sensing information and can transmit the pre-processed environment sensing information through the communication unit 79 that sends and receives information over the network.

The first server 20a can receive the pre-processed environment sensing information via the network and generate sleep state information. The network that receives the generated sleep state information can transmit it to the second server 20b. Accordingly, the second server 20b can generate environment adjustment information based on the received sleep state information and control the environment adjustment device 30 in real-time through the network.

### Acquisition of Environment Sensing Information

In an embodiment, the environment sensing information of the present invention can be acquired through an electronic device (e.g., user terminal 10, etc.). Environment sensing information may refer to sensing information acquired from the space where the user is located.

Environment sensing information may be sensing information acquired in a non-contact manner related to the user's activity or sleep.

For example, environment sensing information may be sleep sound information acquired from a bedroom where the user is sleeping. According to an embodiment, the environment sensing information acquired through the user terminal 10 may serve as the foundational information for acquiring the user's sleep state information in the present invention. Specifically, sleep state information related to whether the user is before sleep, during sleep, or after sleep can be acquired through environment sensing information related to the user's activity.

For example, environment sensing information may include the user's breathing and movement information. To this end, the user terminal 10 may be equipped with a radar sensor as a motion sensor. The user terminal 10 can process signals of the user's movement and distance measured through the radar sensor to generate discrete waveforms (breathing information) corresponding to the user's breathing.

For example, environment sensing information may include measurements obtained through sensors measuring the temperature, humidity, and lighting levels of the bedroom. To this end, the user terminal 10 may be equipped with sensors measuring the temperature, humidity, and lighting levels of the bedroom.

Such a user terminal 10 may refer to any form of entity within a system having a mechanism for communication with a computing device 100. For example, such a user terminal 10 may include a personal computer (PC), notebook, mobile terminal, smartphone, tablet PC, AI speaker, AI TV, and wearable device, and may include any type of terminal capable of connecting to wired/wireless networks. Additionally, the user terminal 10 may include any server implemented by at least one of an agent, Application Programming Interface (API), and plug-in. Furthermore, the user terminal 10 may include an application source and/or client application.

According to one embodiment of the present invention, the external server 20 may be a server that stores information on a plurality of training data for training a neural network. The plurality of training data may include, for example, health examination information or sleep examination information. For instance, the external server 20 may be at least one of a hospital server and an information server, and may store information related to multiple sleep polysomnography records, electronic health records, and electronic medical records. For example, the sleep polysomnography records may include information on the breathing and movements of a sleep examination subject during sleep and information on sleep diagnosis results (e.g., sleep stage information) corresponding to such information. The information stored in the external server 20 can be utilized as training data, validation data, and test data for training the neural network in the present invention.

The computing device 100 of the present invention can receive health examination information or sleep examination information from the external server 20 and build a training data set based on such information. By performing training on one or more network functions through the training data set, the computing device 100 can generate a sleep analysis model for acquiring sleep state information corresponding to environment sensing information. A detailed description of the configuration for building the training data set for neural network training and the learning method using the training data set will be provided later.

According to the present invention, the external server 20 may be a digital device equipped with a processor and memory, such as a laptop computer, notebook computer, desktop computer, web pad, or mobile phone. The external server 20 may be a web server that processes services. The types of servers mentioned above are merely examples and the present invention is not limited thereto.

According to one embodiment of the present invention, the environment adjustment device 30 can adjust the user's sleep environment. Specifically, the environment adjustment device 30 may include one or more environment adjustment modules and can adjust the user's sleep environment by operating an environment adjustment module related to at least one of air quality, illumination, temperature, wind direction, humidity, and sound in the space where the user is located, based on environment adjustment information received from the computing device 100.

Additionally, in an embodiment such as that shown in FIG. 1(c), at least one of the electronic devices depicted in FIG. 1(c) may perform the aforementioned operations.

According to one embodiment of the present invention, the environment adjustment device 30 can be implemented as a TV providing images and videos and generating sound, an air purifier controlling air quality, a lighting device controlling light quantity (illumination), a heating/cooling device controlling temperature, an air conditioner controlling temperature and humidity, a humidifier/dehumidifier controlling humidity, an audio/speaker controlling sound, a styler managing clothing, blinds or curtains, a robot or vacuum cleaner, a washing machine or dryer, a water purifier, an oven or range, etc.

The environment adjustment information may be a signal generated from the computing device 100 based on the determination of the user's sleep state information. For example, the environment adjustment information may include information on lowering or increasing illumination. If the environment adjustment device 30 is a lighting device, the environment adjustment information may include control information to gradually increase the illumination from 0 lux to 250 lux with 3000K white light starting 30 minutes before the predicted wake-up time.

For a specific example, if the environment adjustment device 30 is an air purifier or air conditioner, the environment adjustment information may include various information related to temperature and/or humidity control, removal of fine dust (fine dust, ultrafine dust, extremely fine dust), removal of harmful gases, allergy care operation, deodorization/sterilization operation, dehumidification/humidification control, blower intensity control, air purifier or air conditioner operation noise control, LED lighting, management of smog-causing substances (SO2, NO2, removal of household odors, etc., based on the user's real-time sleep state. Additionally, if the environment adjustment device 30 is an air conditioner, the environment adjustment information may include temperature and humidity control of the sleep space, blower intensity control, operation noise control, LED lighting, etc., based on the user's real-time sleep state.

As an additional example, the environment adjustment information may include control information for adjusting at least one of temperature, humidity, wind direction, or sound. The specific description of the aforementioned environment adjustment information is merely exemplary, and the present invention is not limited thereto.

According to one embodiment of the present invention, the one or more environment adjustment modules included in the environment adjustment device 30 may include, for example, at least one of an illumination control module, a temperature control module, a wind direction control module, a humidity control module, and a sound control module. However, it is not limited thereto, and the one or more environment adjustment modules may further include various environment adjustment modules that can bring changes to the user's sleep environment. That is, the environment adjustment device 30 can adjust the user's sleep environment by operating one or more environment adjustment modules based on the environment control signal from the computing device 100.

According to an embodiment of the present invention, the computing device 100 can acquire the user's sleep state information and adjust the user's sleep environment based on the sleep state information. Specifically, the computing device 100 can acquire sleep state information related to whether the user is before, during, or after sleep based on environment sensing information, and adjust the sleep environment of the space where the user is located according to the sleep state information. For example, if the computing device 100 acquires sleep state information indicating that the user is before sleep, it can generate environment adjustment information related to the intensity and illumination of light to induce sleep (e.g., white light at 3000K, 30 lux illumination), air quality (fine dust concentration, harmful gas concentration, air humidity, air temperature, etc.). The computing device 100 can transmit the environment adjustment information related to the intensity and illumination of light and air quality to the environment adjustment device 30. In this case, the environment adjustment device 30 can adjust the intensity and illumination of light in the space where the user is located to appropriate levels for inducing sleep (e.g., white light at 3000K with 30 lux illumination) based on the environment adjustment information received from the computing device 100. That is, the environment adjustment information generated by the computing device 100 can be delivered to a lighting device, an embodiment of the environment adjustment device 30, to adjust the illumination within the sleep space.

Additionally, the computing device 100 can generate environment adjustment information related to various aspects such as fine dust removal, harmful gas removal, allergy care operation, deodorization/sterilization operation, dehumidification/humidification control, blower intensity control, operation noise control of the environment adjustment device 30, and LED lighting based on the user's sleep state information.

Furthermore, in an embodiment such as that shown in FIG. 1(c), at least one of the electronic devices depicted in FIG. 1(c) may perform the aforementioned operations.

For example, the environment adjustment information generated by the computing device 100 can be delivered to an air purifier or air conditioner, embodiments of the environment adjustment device 30, to adjust the temperature, humidity, or air quality within an indoor space, vehicle, or sleep space.

Hereinafter, for convenience in explaining the operation of smart home-appliances, the terms ‘sleep mode’ and ‘wake mode’ will be used. ‘Sleep mode’ refers to a concept that includes the operation modes of smart home-appliances during the user's preparation for sleep, falling asleep, and sleeping stages, while ‘wake mode’ refers to a concept that includes the operation modes of smart home-appliances during the user's pre-wake, waking, and post-wake stages.

### Description of FIG. 52

FIG. 52 is a table describing the location where the environment adjustment device is placed, and for each specific product, the activation status according to sleep state information, and exemplary operations in sleep mode and wake mode. Specifically, it describes the location where the environment adjustment device 30 is placed and, for each specific product of the environment adjustment device 30, the activation status according to sleep state information (before sleep, falling asleep, sleeping, pre-wake, waking, post-wake), and exemplary operations in sleep mode and wake mode. The environment adjustment information may include control information that allows operations to be performed in each product's activation status, sleep mode, and wake mode.

The specific descriptions related to the aforementioned sleep state information and environment adjustment information are merely examples, and the present invention is not limited thereto.

### Description of Environment Sensing Information

According to an embodiment of the present invention, the environment sensing information utilized by the computing device 100 for sleep state analysis may include information acquired in a non-invasive manner during the user's activity or sleep in a space. For specific examples, the environment sensing information may include sounds generated by the user's tossing and turning during sleep, sounds related to muscle movements, or sounds related to the user's breathing during sleep. Alternatively, the environment sensing information may include movement and distance information related to the user's movements during sleep, and breathing information generated based on this.

According to an embodiment, the environment sensing information may include sleep sound information, which may refer to acoustic information related to movement patterns and breathing patterns occurring during a user's sleep. Alternatively, the environment sensing information may include sleep movement information, which refers to information related to movement patterns and breathing patterns occurring during a user's sleep.

In the embodiment, the environment sensing information can be acquired through a user terminal 10 possessed by the user. For example, environment sensing information related to the user's activities in a space can be acquired through a microphone module provided in the user terminal 10. Alternatively, environment sensing information related to the user's activities in a space can be acquired through a radar sensor provided in the user terminal 10.

Generally, the microphone module provided in the user terminal 10 possessed by the user should be configured as a MEMS (Micro-Electro Mechanical Systems) due to the relatively small size of the user terminal 10. Although such a microphone module can be manufactured in a very compact form, it may have a lower signal-to-noise ratio (SNR) compared to a condenser microphone or a dynamic microphone. A low signal-to-noise ratio means that the ratio of noise, which is the sound not intended to be identified, is high compared to the sound intended to be identified, making it difficult to identify the sound (i.e., it is unclear).

In the present invention, the environment sensing information subject to analysis may include sleep sound information, which is acoustic information related to the user's breathing and movement acquired during sleep. Since this sleep sound information pertains to very small sounds (i.e., sounds that are difficult to distinguish) such as the user's breathing and movement, and is acquired along with other sounds during the sleep environment, it may be very difficult to detect and analyze when acquired through the aforementioned microphone module with a low signal-to-noise ratio.

According to an embodiment of the present invention, the computing device 100 can acquire sleep state information based on the environment sensing information obtained from the user terminal 10. Specifically, the computing device 100 can convert and/or adjust the environment sensing information, which is acquired unclearly with a lot of noise, into analyzable data, and perform learning on an artificial neural network using the converted and/or adjusted data. Once pre-learning on the artificial neural network is completed, the trained neural network (e.g., an acoustic analysis model) can acquire the user's sleep state information based on the data (e.g., spectrogram) obtained in response to the sleep sound information (e.g., converted and/or adjusted).

In the embodiment, the sleep state information may include not only information related to whether the user is sleeping but also sleep stage information related to changes in the user's sleep stages during sleep. For a specific example, the sleep state information may include sleep stage information indicating that the user was in REM sleep at a first time point and in light sleep at a second time point different from the first time point. In this case, through the sleep state information, it can be acquired that the user fell into relatively deep sleep at the first time point and took lighter sleep at the second time point.

That is, the computing device 100 can process sleep sound information with a low signal-to-noise ratio, acquired through commonly distributed user terminals (e.g., AI speakers, bedroom IoT devices, mobile phones, etc.) for sound collection, into data suitable for analysis, and provide sleep state information related to changes in sleep stages by processing the processed data. This allows for monitoring sleep states in a general home environment without the need for a contact microphone on the user's body for clear sound acquisition or purchasing additional devices with a high signal-to-noise ratio, thereby enhancing convenience through software updates alone.

Although the computing device 100 and the environment adjustment device 30 are depicted as separate entities in FIG. 1(a), according to an embodiment of the present invention, the environment adjustment device 30 may be included within the computing device 100, performing sleep state measurement and environment adjustment operations as a single integrated device.

Additionally, in the embodiment shown in FIG. 1(c), at least one of the electronic devices depicted in FIG. 1(c) may perform the aforementioned operations.

In the embodiment, the computing device 100 may be a terminal or a server, and any form of device may be included. The computing device 100 may be a digital device equipped with a processor and memory, such as a laptop computer, notebook computer, desktop computer, web pad, or mobile phone, possessing computational capabilities. The computing device 100 may be a web server processing services. The types of servers mentioned above are merely examples, and the present invention is not limited thereto.

According to one embodiment of the present invention, the computing device 100 may be a server providing cloud computing services. More specifically, the computing device 100 may be a server that provides cloud computing services, which is a type of internet-based computing that processes information on computers connected to the internet rather than on the user's computer. The cloud computing service may store data on the internet, allowing users to access necessary data or programs via internet connection without installing them on their own computers, and enabling easy sharing and transmission of stored data through simple operations and clicks.

Additionally, the cloud computing service not only stores data on internet servers but also allows users to perform desired tasks using the functions of web-based applications without installing separate programs, and provides a service where multiple users can share and work on documents simultaneously. Furthermore, the cloud computing service may be implemented in at least one form among IaaS (Infrastructure as a Service), PaaS (Platform as a Service), SaaS (Software as a Service), virtual machine-based cloud servers, and container-based cloud servers. That is, the computing device 100 of the present invention may be implemented in at least one form of the aforementioned cloud computing services. The specific description of the aforementioned cloud computing services is merely exemplary and may include any platform for constructing the cloud computing environment of the present invention.

### Overall Configuration of the Computing Device

The specific configuration, technical features, and effects according to the technical features of the computing device 100 of the present invention will be described with reference to the accompanying drawings.

FIG. 2 illustrates a block diagram of a computing device for creating a sleep environment based on sleep state information related to one embodiment of the present invention.

As shown in FIG. 2, the computing device 100 may include a network unit 110, a memory 120, and a processor 130. It is not limited to the components included in the aforementioned computing device 100. That is, additional components may be included, or some of the aforementioned components may be omitted depending on the implementation aspects of the embodiments of the present invention.

According to one embodiment of the present invention, the computing device 100 may include a network unit 110 that transmits and receives data with a user terminal 10, an external server 20, and an environment adjustment device 30. The network unit 110 may transmit and receive data for performing a sleep environment creation method according to sleep state information in accordance with one embodiment of the present invention with other computing devices, servers, etc.

That is, the network unit 110 may provide communication functions between the computing device 100 and the user terminal 10, the external server 20, and the environment adjustment device 30. For example, the network unit 110 may receive sleep examination records and electronic health records for multiple users from a hospital server. In another example, the network unit 110 may receive environment sensing information related to the space where the user is active from the user terminal 10. In yet another example, the network unit 110 may transmit environment adjustment information to the environment adjustment device 30 to adjust the environment of the space where the user is located. Additionally, the network unit 110 may allow information transfer between the computing device 100 and the user terminal 10 and the external server 20 by calling procedures to the computing device 100.

The network unit 110 according to one embodiment of the present invention may use various wired communication systems such as Public Switched Telephone Network (PSTN), xDSL (x Digital Subscriber Line), RADSL (Rate Adaptive DSL), MDSL (Multi Rate DSL), VDSL (Very High Speed DSL), UADSL (Universal Asymmetric DSL), HDSL (High Bit Rate DSL), and Local Area Network (LAN).

Furthermore, the network unit 110 presented in this specification may use various wireless communication systems that can be realized currently and in the future, such as mobile communication systems like 4G, 5G (LTE), and satellite communication systems like Starlink.

In the present invention, the network unit 110 can be configured regardless of its communication mode, such as wired or wireless, and can be composed of various communication networks, including a Personal Area Network (PAN) and a Wide Area Network (WAN). Additionally, the network may be the well-known World Wide Web (WWW) and may utilize wireless transmission technologies used for short-range communication, such as Infrared Data Association (IrDA) or Bluetooth. The technologies described herein can be used not only with the aforementioned networks but also with other networks.

According to one embodiment of the present invention, the memory 120 can store a computer program for performing a sleep environment adjustment method according to sleep state information in accordance with an embodiment of the present invention. The stored computer program can be read and executed by the processor 130. Furthermore, the memory 120 can store any form of information generated or determined by the processor 130 and any form of information received by the network unit 110. Additionally, the memory 120 can store data related to the user's sleep. For example, the memory 120 may temporarily or permanently store input/output data (e.g., environment sensing information related to the user's sleep environment, sleep state information corresponding to the environment sensing information, or environment adjustment information according to the sleep state information).

According to one embodiment of the present invention, the memory 120 may include at least one type of storage medium, such as a flash memory type, hard disk type, multimedia card micro type, card-type memory (e.g., SD or XD memory), Random Access Memory (RAM), Static Random Access Memory (SRAM), Read-Only Memory (ROM), Electrically Erasable Programmable Read-Only Memory (EEPROM), Programmable Read-Only Memory (PROM), magnetic memory, magnetic disk, or optical disk. The computing device 100 may operate in association with web storage that performs the storage function of the memory 120 on the internet. The description of the memory above is merely exemplary, and the present invention is not limited thereto.

When the computer program is loaded into the memory 120, it may include one or more instructions that cause the processor 130 to perform methods/operations according to various embodiments of the present invention. That is, by executing one or more instructions, the processor 130 can perform methods/operations according to various embodiments of the present invention.

In one embodiment, the computer program may include one or more instructions to perform a sleep environment adjustment method according to sleep state information, comprising the steps of acquiring the user's sleep state information, generating environment adjustment information based on the sleep state information, and transmitting the environment adjustment information to the environment adjustment device.

According to one embodiment of the present invention, the processor 130 may be composed of one or more cores and may include processors for data analysis and deep learning, such as a central processing unit (CPU), a general-purpose graphics processing unit (GPGPU), or a tensor processing unit (TPU).

The processor 130 can read the computer program stored in the memory 120 to perform data processing for machine learning according to one embodiment of the present invention. According to one embodiment of the present invention, the processor 130 can perform operations for training a neural network. The processor 130 can perform calculations for neural network training, such as processing input data for deep learning (DL), feature extraction from input data, error calculation, and weight updates of the neural network using backpropagation.

Additionally, at least one of the CPU, GPGPU, and TPU of the processor 130 can process the training of network functions. For example, the CPU and GPGPU can together process the training of network functions and data classification using network functions. Furthermore, in one embodiment of the present invention, the processors of multiple computing devices can be used together to process the training of network functions and data classification using network functions. Additionally, the computer program executed in the computing device according to one embodiment of the present invention may be executable by the CPU, GPGPU, or TPU.

In this specification, the network function can be used interchangeably with artificial neural networks and neural networks. The network function may include one or more neural networks, and in this case, the output of the network function may be an ensemble of the outputs of one or more neural networks.

In this specification, the model may include a network function. The model may include one or more network functions, and in this case, the output of the model may be an ensemble of the outputs of one or more network functions.

The processor 130 can provide a sleep analysis model according to an embodiment of the present invention by reading a computer program stored in the memory 120. According to an embodiment of the present invention, the processor 130 can perform calculations to derive environment adjustment information based on sleep state information. According to an embodiment of the present invention, the processor 130 can perform calculations to train the sleep analysis model. The sleep analysis model will be described in more detail below.

According to the present invention, sleep information related to the quality of a user's sleep can be inferred based on the sleep analysis model. Environment sensing information acquired from the user in real-time or periodically is input into the sleep analysis model as input values, resulting in the output of data related to the user's sleep.

The training of such a sleep analysis model and the inference based thereon can be performed by the computing device 100 shown in FIG. 1(a). That is, both training and inference can be designed to be performed by the computing device 100. However, in other embodiments, training may be performed on the computing device 100, while inference may be performed on the user terminal 10. Additionally, training may be performed on the computing device 100, while inference may be performed on an environment adjustment device 30 implemented as various smart home-appliances (such as air conditioners, TVs, lighting, refrigerators, air purifiers, etc.). In another embodiment, it may be performed by the sleep environment adjustment device 400 shown in FIG. 1(b). That is, both training and inference can be performed by the sleep environment adjustment device 400.

Alternatively, in the case of an embodiment such as FIG. 1(c), at least one of the electronic devices shown in FIG. 1(c) can perform at least one of the aforementioned operations.

According to an embodiment of the present invention, the processor 130 can generally handle the overall operation of the computing device 100. By processing signals, data, information, etc., input or output through the components examined above, or by running applications stored in the memory 120, the processor 130 can provide or process appropriate information or functions to the user terminal.

According to an embodiment of the present invention, the processor 130 can acquire the user's sleep state information. The acquisition of sleep state information according to an embodiment of the present invention may involve acquiring or loading sleep state information stored in the memory 120. Additionally, the acquisition of sleep state information may involve receiving or loading data from other storage media, other computing devices, or separate processing modules within the same computing device based on wired/wireless communication means.

Additionally, in the case of an embodiment such as FIG. 1(c), at least one of the electronic devices shown in FIG. 1(c) can perform at least one of the aforementioned operations.

### Sleep State Information

In one embodiment, sleep state information may include information related to whether the user is sleeping. Specifically, sleep state information may include at least one of the first sleep state information indicating the user is before sleep, the second sleep state information indicating the user is during sleep, and the third sleep state information indicating the user is after sleep. In other words, if the first sleep state information is inferred concerning the user, the processor 130 can determine that the user is in a state before sleep (i.e., before going to bed). If the second sleep state information is inferred, it can be determined that the user is in a state during sleep. If the third sleep state information is acquired, it can be determined that the user is in a state after sleep (i.e., upon waking).

Such sleep state information can be characterized by being acquired based on environment sensing information. Environment sensing information may include sensing information acquired in a non-contact manner from the space where the user is located.

According to one embodiment, the processor 130 can acquire environment sensing information. Specifically, the environment sensing information can be acquired through the user terminal 10 possessed by the user. For example, environment sensing information related to the space in which the user is active can be acquired through the user terminal 10 possessed by the user, and the processor 130 can receive the environment sensing information from the user terminal 10. The environment sensing information may be sound information acquired in a non-contact manner during the user's daily life. For instance, the environment sensing information may include various sound information acquired according to the user's lifestyle, such as sound information related to cleaning, sound information related to cooking, sound information related to watching TV, and sleep sound information acquired during sleep. In the embodiment, the sleep sound information acquired during the user's sleep may include sounds generated by the user's tossing and turning, sounds related to muscle movements, or sounds related to the user's breathing during sleep. That is, the sleep sound information in the present invention may refer to sound information related to movement patterns and breathing patterns during the user's sleep.

### Sleep Analysis Information and Sleep Stage Information

In sleep analysis, various information such as sleep onset time, wake-up time, and total sleep time is analyzed, and according to one embodiment, the processor 130 can extract sleep stage information. The sleep stage information can be extracted based on the user's environment sensing information. Sleep stages can be divided into NREM (non-REM) sleep and REM (Rapid Eye Movement) sleep, and NREM sleep can be further divided into multiple stages (e.g., two stages of Light and Deep, or four stages from N1 to N4. The setting of sleep stages may be defined as general sleep stages, but can also be arbitrarily set to various sleep stages depending on the designer. Through sleep stage analysis, not only the quality of sleep related to sleep can be predicted, but also sleep disorders (e.g., sleep apnea) and their underlying causes (e.g., snoring).

In sleep analysis, changes in sleep stages are analyzed, and a hypnogram can be generated to identify the changes in the analyzed sleep stages, thereby identifying the user's sleep cycle.

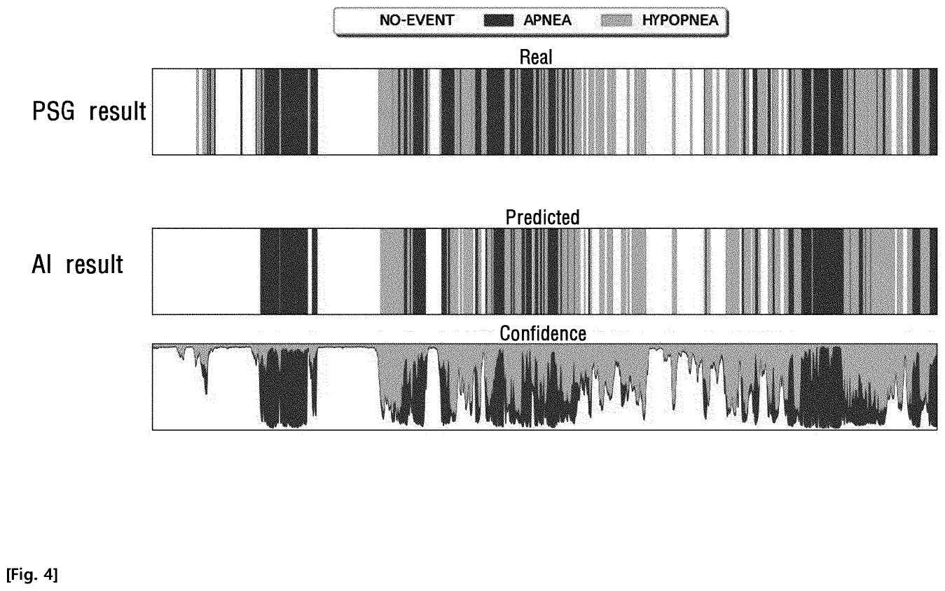

FIG. 3 is a diagram comparing the results of polysomnography (PSG result) and the analysis results using the AI algorithm according to the present invention (AI result).

As shown in FIG. 3, the sleep stage information acquired according to the present invention not only closely matches polysomnography but also includes more precise and meaningful information related to sleep stages (Wake, Light, Deep, REM). The hypnogram shown at the bottom of FIG. 3 indicates the probability of belonging to one of the four classes (Wake, Light, Deep, REM) every 30 seconds when predicting sleep stages based on user sound information. Here, the four classes represent the states of being awake, lightly asleep, deeply asleep, and in REM sleep, respectively.

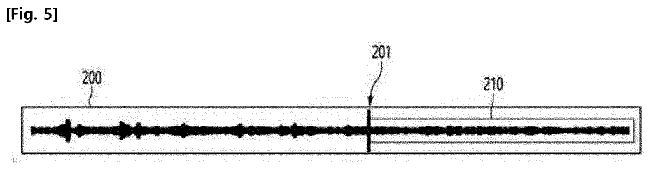

FIG. 4 is a diagram comparing the results of polysomnography (PSG result) and the analysis results using the AI algorithm according to the present invention (AI result) in relation to sleep apnea and hypopnea. The hypnogram shown at the bottom of FIG. 4 indicates the probability of belonging to one of the two conditions (sleep apnea, hypopnea) every 30 seconds when predicting sleep disorders based on user sound information.

By using the sleep stage information according to the present invention, as shown in FIG. 4, the sleep stage information acquired according to the present invention not only closely matches polysomnography but also includes more precise analysis information related to apnea and hypopnea.

According to the present invention, the processor 130 can generate environment adjustment information based on the sleep stage information. For example, if the sleep stage is in the Light stage or N1 stage, environment adjustment information can be generated to control environment adjustment devices (such as air conditioners, lighting, air purifiers) to induce deep sleep.

For instance, a part of the smart home-appliance 800 according to an embodiment of the present invention can be set to sound an alarm if REM sleep is detected within 30 minutes of the wake-up time set by the user.

This is because waking up during REM sleep can result in feeling more refreshed. Through the sleep management app of the present invention, REM can be detected in real-time during the user's sleep, and auditory or tactile stimuli can be delivered to the user to wake them within this time frame.

Additionally, some smart home-appliances 800 according to an embodiment of the present invention can detect periods of respiratory instability based on sleep sound information during the user's sleep and provide vibrotactile stimulation to guide the user back to stable breathing.

Generally, if sleep apnea persists, the sympathetic nervous system is activated, which can lead to cardiovascular diseases later on. Therefore, when periods of respiratory instability are detected in real-time during the user's sleep through the sleep management app of the present invention, auditory and tactile stimuli can be delivered to the user via some smart home-appliances 800 according to an embodiment of the present invention to cease the user's respiratory instability.

Obstructive sleep apnea can be selectively identified in stages based on body movement information or the user's posture information.

In Sleep Analysis, the quality of sleep, sleep stages, and the presence of sleep apnea are analyzed based on sleep sound information. Sleep sound information may refer to sound information related to breathing occurring during the user's sleep.

Sleep analysis involves pre-processing the user's sleep sound information and analyzing the user's sleep stages through AI algorithms, with specific analysis methods to be described in more detail below.

### Singularity Identification According to Predefined Pattern Detection

According to one embodiment of the present invention, the processor 130 can acquire sleep state information based on environment sensing information. Specifically, the processor 130 can identify singularities where information of a predefined pattern in the environment sensing information is detected. Here, the information of the predefined pattern may relate to breathing and movement patterns related to sleep. For example, in a wake state, all nervous systems are activated, leading to irregular breathing patterns and frequent body movements. Additionally, due to the lack of relaxation of the neck muscles, breathing sounds may be minimal.

Conversely, when the user is asleep, the autonomic nervous system stabilizes, resulting in regular breathing changes, reduced body movements, and potentially louder breathing sounds. That is, the processor 130 can identify the point in time when sound information related to regular breathing, minimal body movement, or minimal breathing sounds, which are predefined patterns, is detected as a singularity in the environment sensing information. Furthermore, the processor 130 can acquire sleep sound information based on the environment sensing information obtained relative to the identified singularity. The processor 130 can identify singularities related to the user's sleep timing in the time-series environment sensing information and acquire sleep sound information based on these singularities.

FIG. 5 is an exemplary diagram for explaining the process of acquiring sleep sound information 210 from environment sensing information 200 related to an embodiment of the present invention.

Referring to FIG. 5 as a specific example, the processor 130 can identify a singularity 201 related to the point in time when a predefined pattern is identified from the environment sensing information 200. Based on the identified singularity, the processor 130 can acquire sleep sound information 210 by utilizing the acoustic information obtained after the singularity. The waveform and singularity related to sound in FIG. 5 are merely illustrative for understanding the present invention and do not limit the invention.

In other words, by identifying singularities related to the user's sleep from the environment sensing information, the processor 130 can extract and acquire only the sleep sound information from a vast amount of acoustic information (i.e., environment sensing information) based on the singularity. This automation of the process of recording the user's sleep time provides convenience and simultaneously contributes to improving the accuracy of the acquired sleep sound information.

Additionally, in an embodiment, the processor 130 can acquire sleep state information related to whether the user is before sleep or during sleep based on the singularity 201 identified from the environment sensing information 200. Specifically, if the singularity 201 is not identified, the processor 130 can determine that the user is before sleep, and if the singularity 201 is identified, it can determine that the user is during sleep after the singularity 201. Furthermore, after identifying the singularity 201, the processor 130 can identify the point in time when a predefined pattern is not observed (e.g., wake-up time) and determine that the user is after sleep, i.e., has woken up, if such a point in time is identified.

Thus, the processor 130 can acquire sleep state information related to whether the user is before, during, or after sleep based on whether the singularity 201 is identified in the environment sensing information 200 and whether the predefined pattern is continuously detected after the singularity is identified.

Alternatively, in the case of an embodiment such as (c) of FIG. 1, at least one of the electronic devices shown in (c) of FIG. 1 can perform at least one of the aforementioned operations.

Furthermore, in a smart home-appliance 800 according to an embodiment of the present invention, the processor 830 can identify a singularity 201 related to the point in time when a predefined pattern is identified from the environment sensing information 200.

According to the present invention, the processor 830 can acquire sleep sound information 210 based on the acoustic information obtained after the identified singularity 201.

The waveform and singularity related to sound in FIG. 5 are merely illustrative for understanding the present invention and do not limit the invention.

In other words, by identifying a singularity 201 related to the user's sleep from the acoustic information, the processor 830 included in the smart home-appliance 800 according to an embodiment of the present invention can extract and acquire only the sleep sound information 210 from a vast amount of environment sensing information (i.e., acoustic information) based on the singularity 201.

This automation of the process of recording the user's sleep time provides convenience and simultaneously contributes to improving the accuracy of the acquired sleep sound information.

In addition, in the embodiment, the processor 830 can acquire sleep state information related to whether the user is before sleep or during sleep based on the singularity 201 identified from the environment sensing information 200. Specifically, if the singularity 201 is not identified, the processor 830 may determine that the user is before sleep, and if the singularity 201 is identified, it may determine that the user is during sleep after the singularity 201.

Furthermore, after the singularity 201 is identified, the processor 830 can identify a point in time when a predefined pattern is not observed (e.g., the waking time), and if this point in time is identified, it can determine that the user has woken up after sleep.

That is, the processor 830 can acquire sleep state information related to whether the user is before, during, or after sleep based on whether the singularity 201 is identified in the environment sensing information 200 and whether a predefined pattern is continuously detected after the singularity is identified.

Meanwhile, the processor 830 can acquire sleep state information based on sleep sound information rather than environment sensing information 200.

In the present invention, since the user's sleep state information is pre-identified using sleep sound information during the primary sleep analysis, the reliability of the analysis regarding the sleep state can be further enhanced.

The sleep analysis method according to the present invention generates an inference model through deep learning of the environment sensing information, and the inference model extracts the user's sleep state and sleep stage.

Briefly, the environment sensing information 200, including sleep sound information, is converted into a spectrogram, and an inference model is generated based on the spectrogram.

At this time, in the sleep analysis using sound information, the protection of the user's privacy cannot be overlooked, and the present invention utilizes a process of pre-processing the environment sensing information 200 to protect the user's privacy.

As described above, an inference model for extracting the user's sleep state and sleep stage is generated through deep learning of the environment sensing information 200.

Briefly, the environment sensing information 200, including sound information, is converted into a spectrogram, and an inference model can be generated based on the spectrogram.

As described above, the inference model can be implemented in the computing device 100 shown in FIG. 1(a) or the sleep environment adjustment device 400 shown in FIG. 1(b).

Subsequently, environment sensing information, which includes user sound information acquired through the user terminal 10, is input into the inference model to output sleep state information and/or sleep stage information as a result. At this time, learning and inference may be performed by the same entity, but they may also be performed by separate entities. That is, both learning and inference can be performed by the computing device 100 in FIG. 1(a) or the environment adjustment device 400 in FIG. 1(b). Learning may be conducted on the computing device 100, while inference may be performed on the user terminal 10. Alternatively, learning may be conducted on the computing device 100, while inference may be performed on the environment adjustment device 30 implemented as smart home-appliances such as an air conditioner, TV, lighting, refrigerator, air purifier, etc.

Alternatively, in the embodiment shown in FIG. 1(c), at least one of the electronic devices depicted in FIG. 1(c) can perform at least one of the aforementioned operations.

### Sleep Analysis Model and Sleep Analysis Method

According to one embodiment of the present invention, sleep stage information may be characterized as being acquired through a sleep analysis model that analyzes the user's sleep stages based on environment sensing information. That is, the sleep stage information of the present invention can be acquired through the sleep analysis model.

According to one embodiment of the present invention, the processor 130 or processor 830 can acquire environment sensing information and, based on this information, acquire sleep sound information. In this case, sleep sound information relates to sounds acquired during the user's sleep, such as sounds generated by the user's movements during sleep, sounds related to muscle movements, or sounds related to the user's breathing during sleep.

Hereinafter, the sleep analysis method according to an embodiment of the present invention will be described with reference to the drawings.

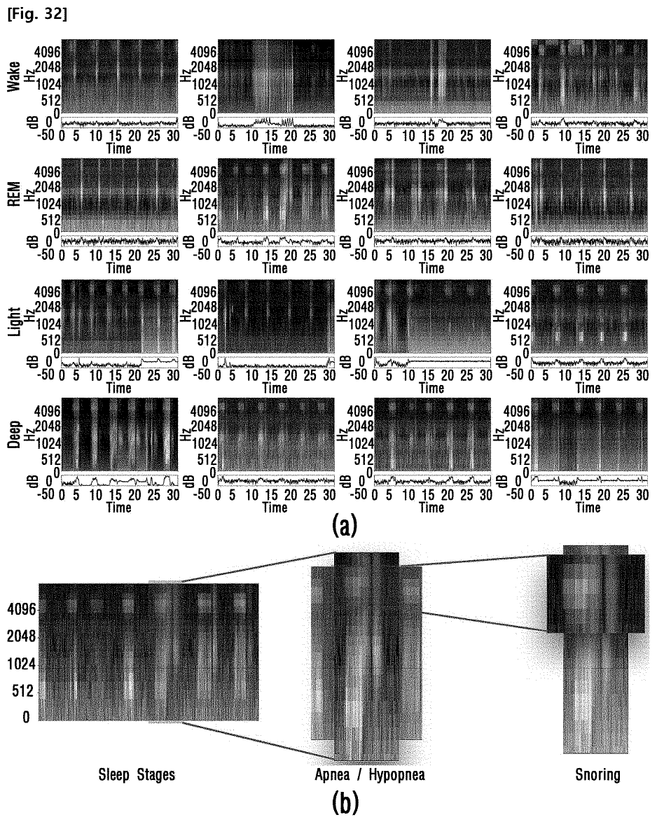

FIG. 32(a) is a diagram for explaining sleep stage analysis using a spectrogram in the sleep analysis method according to the present invention.

FIG. 32(b) is a diagram for explaining the determination of sleep disorders using a spectrogram in the sleep analysis method according to the present invention.

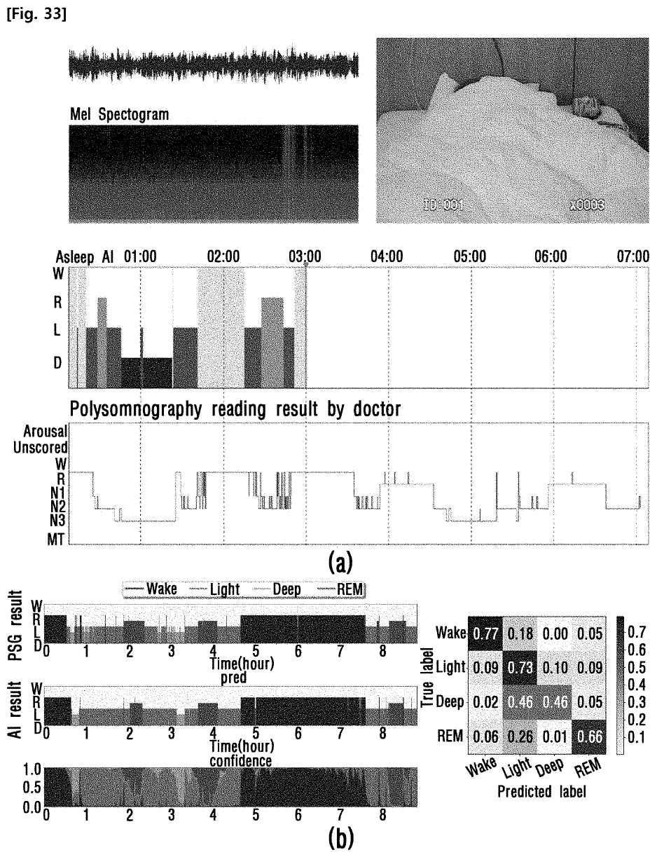

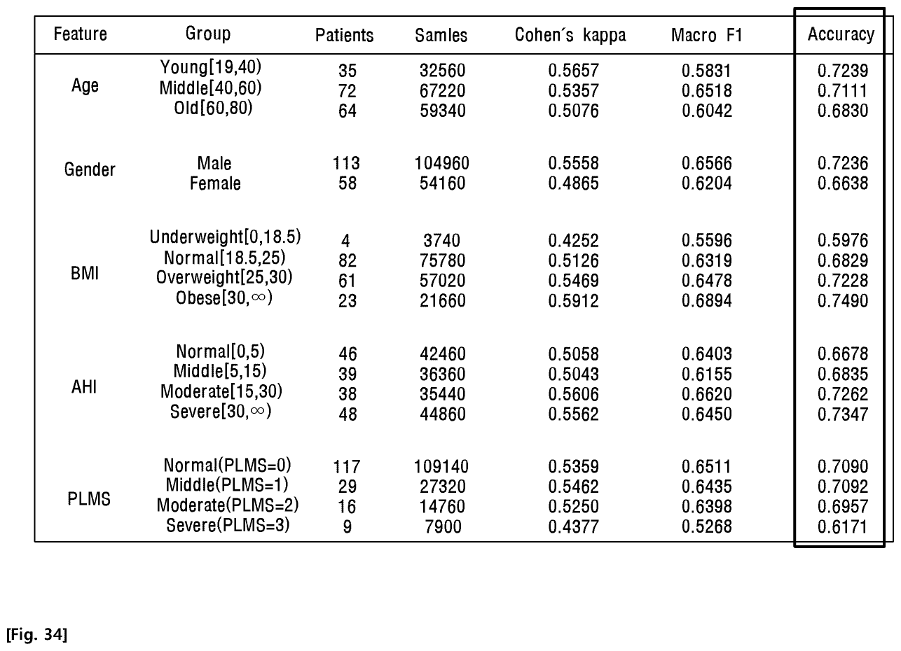

FIG. 33(a) is a diagram showing the experimental process for verifying the performance of the sleep analysis method according to the present invention.

FIG. 33(b) is a graph verifying the performance of the sleep analysis method according to the present invention, comparing the polysomnography results (PSG result) with the analysis results using the AI algorithm according to the present invention (AI result).

As illustrated in FIG. 32(a), when the user's sleep sound information is input, the corresponding sleep stage (Wake, REM, Light, Deep) can be immediately inferred.

In addition, a secondary analysis based on the sleep sound information can extract the points in time when sleep disorders (such as sleep apnea or hyperpnea) or snoring occur, through singularities in the Mel spectrum corresponding to the sleep stage.

As shown in FIG. 32(b), in a single Mel spectrogram, the breathing pattern is analyzed, and if characteristics corresponding to sleep apnea or hyperpnea events are detected, the corresponding point in time can be determined as the occurrence of a sleep disorder. At this time, the process may further include classifying the event as snoring rather than sleep apnea or hyperpnea through frequency analysis.

As depicted in FIG. 33(a), the user's sleep video and sleep sound are acquired in real-time, and the acquired sleep sound information is immediately converted into a spectrogram.

During this process, pre-processing of the sleep sound information may be performed. The spectrogram is input into the sleep analysis model, where the sleep stage is immediately analyzed.

When compared with the results of polysomnography (PSG), it was confirmed that the results of the sleep analysis model, which uses sleep sound information as input, are highly accurate.

The hypnogram shown at the bottom of FIG. 33(a) indicates the probability of which of the four classes (Wake, Light, Deep, REM) the user belongs to every 30 seconds when predicting sleep stages based on the user's sleep sound information. Here, the four classes represent the states of being awake, lightly asleep, deeply asleep, and in REM sleep, respectively.

As illustrated in FIG. 33(b), the sleep analysis results obtained according to the present invention not only closely match polysomnography but also include more precise and meaningful information related to sleep stages (Wake, Light, Deep, REM).

### Generation and Acquisition of the Spectrogram

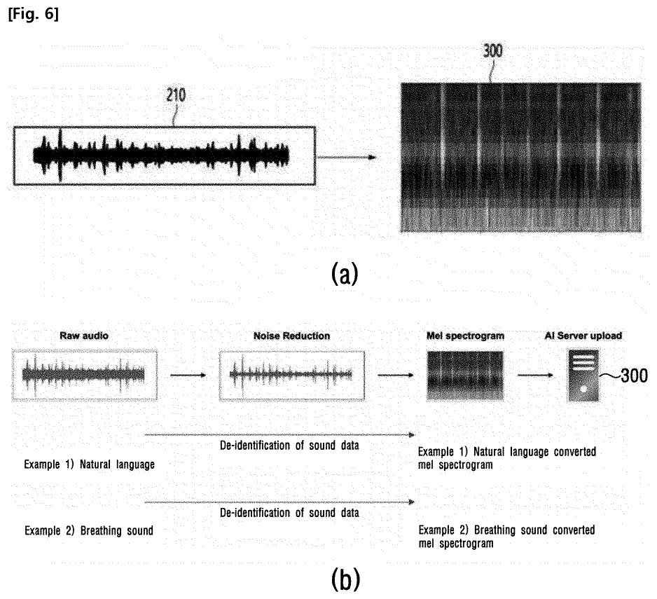

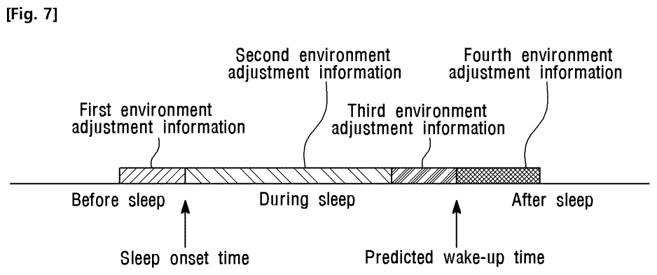

FIG. 6(a) is an exemplary diagram for explaining a method of acquiring a spectrogram corresponding to sleep sound information related to an embodiment of the present invention.

According to the present invention, a sleep analysis model can be generated using a spectrogram created based on sleep sound information. If the sleep sound information expressed as audio data is used as is, the amount of information is very large, leading to a significant increase in computational load and time, and the inclusion of unwanted signals reduces computational precision. Additionally, if all of the user's audio signals are transmitted to a server, there may be concerns about privacy infringement. The present invention reduces computational load and time and promotes the protection of individual privacy by removing noise from the sleep sound information, converting it into a spectrogram (Mel spectrogram), and training the spectrogram to generate a sleep analysis model.

According to an embodiment of the present invention, the processor 130 or processor 830 can generate a spectrogram 300 corresponding to the sleep sound information 210, as illustrated in FIG. 6(a).

The raw data, which serves as the basis for generating the spectrogram 300, can be received as sleep sound information. This raw data may be acquired through the user terminal from the start time to the end time input by the user, or from the time of user terminal operation (e.g., alarm setting) to the time corresponding to the terminal operation (e.g., alarm setting time).

Alternatively, the acquisition time may be automatically selected based on the user's sleep pattern, or determined automatically based on sound (e.g., user's voice, breathing sounds, sounds from surrounding devices such as TV, washing machine) or changes in illumination to capture the user's intended sleep time.

Although not shown in FIG. 6(a), the process may further include pre-processing the input raw data. The pre-processing includes a noise reduction process for the raw data, where noise (e.g., white noise) contained in the raw data is removed. The noise reduction process can be performed using algorithms such as spectral gating and spectral subtraction to eliminate background noise. Furthermore, the present invention can employ a deep learning-based noise reduction algorithm to perform the noise removal process, specifically utilizing a noise reduction algorithm specialized for the user's breathing and respiratory sounds through deep learning.

Notably, the invention can generate a spectrogram based solely on the amplitude, excluding the phase from the raw data, which not only protects privacy but also reduces data volume, thereby enhancing processing speed.

The processor 130 or processor 830 according to an embodiment of the present invention can perform a fast Fourier transform on the sleep sound information 210 to generate a spectrogram 300 corresponding to the sleep sound information 210.

The spectrogram 300 is intended to visualize and comprehend sound or waves, combining features of a waveform and a spectrum. The spectrogram 300 may represent amplitude differences as variations in print density or display color according to changes along the time axis and frequency axis.

The pre-processed raw data related to sound can be segmented into 30-second intervals and converted into a mel spectrogram. Consequently, a 30-second mel spectrogram may have dimensions of 20 frequency bins by 1201 time steps. In the present invention, the split-cat method is used to preserve the amount of information by converting the rectangular mel spectrogram into a square shape.

Meanwhile, the present invention can utilize a method of simulating breathing sounds measured in various home environments by adding various noises occurring in a home environment to clean breathing sounds. Since sound has an additive property, it can be combined. However, adding original audio signals such as mp3 or pcm and converting them into a mel spectrogram can consume significant computing resources.

Therefore, the present invention proposes a method of converting breathing sounds and noise into mel spectrograms separately and then combining them. This allows for the simulation of breathing sounds measured in various home environments, which can be used for training deep learning models to ensure robustness in diverse home environments.

In the present invention, the sleep sound information 210 pertains to sounds related to breathing and body movements acquired during the user's sleep time, which may be very faint. Accordingly, the processor 130 or processor 830 can convert the sleep sound information into a spectrogram 300 to perform sound analysis. In this case, as previously described, the spectrogram 300 includes information showing how the frequency spectrum of sound changes over time, allowing for the easy identification of breathing or movement patterns related to relatively faint sounds, thereby enhancing analysis efficiency.

Alternatively, in the case of an embodiment such as FIG. 1(c), at least one of the electronic devices shown in FIG. 1(c) can perform the aforementioned operations.

According to one embodiment, each spectrogram can be configured to have a frequency spectrum of different concentrations according to various sleep stages. Specifically, merely the change in the energy level of sleep sound information may make it difficult to predict whether it is at least one of an awake state, REM sleep state, light sleep state, or deep sleep state. However, by converting sleep sound information into a spectrogram, changes in the spectrum of each frequency can be easily detected, enabling analysis corresponding to small sounds (e.g., breathing and body movements).

Additionally, the processor 130 or processor 830 can process the spectrogram 300 as input to the sleep analysis model to acquire sleep stage information. Here, the sleep analysis model is a model for acquiring sleep stage information related to changes in the user's sleep stages, and it can output sleep stage information by using sleep sound information acquired during the user's sleep as input. In an embodiment, the sleep analysis model may include a neural network model configured through one or more network functions.

### Network Functions and Neural Network

In an embodiment of the present invention, the sleep analysis model may include a neural network model configured through one or more network functions. The sleep analysis model is composed of one or more network functions, and each network function can generally be composed of a set of interconnected computational units referred to as ‘nodes.’ These ‘nodes’ may also be referred to as ‘neurons.’ Each network function is configured to include at least one or more nodes. The nodes (or neurons) constituting one or more network functions can be interconnected by one or more ‘links.’

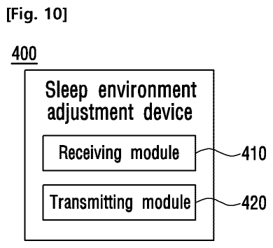

FIG. 9 is a schematic diagram illustrating one or more network functions related to an embodiment of the present invention.

A deep neural network (DNN) may refer to a neural network that includes multiple hidden layers in addition to an input layer and an output layer. By using a deep neural network, latent structures of data can be identified.

That is, latent structures of photos, text, videos, voice, and music (for example, identifying what object is in a photo, the content and emotion of text, the content and emotion of voice, etc.) can be identified. A deep neural network may include a convolutional neural network (CNN), recurrent neural network (RNN), autoencoder, generative adversarial networks (GAN), restricted Boltzmann machine (RBM), deep belief network (DBN), Q network, U network, Siamese network, etc. The description of the aforementioned deep neural network is merely exemplary, and the present invention is not limited thereto.

In an embodiment of the present invention, the network function may include an autoencoder. An autoencoder may be a type of artificial neural network that outputs data similar to the input data. An autoencoder may include at least one hidden layer, and an odd number of hidden layers may be arranged between the input and output layers.

The number of nodes in each layer may be reduced from the number of nodes in the input layer to an intermediate layer called a bottleneck layer (encoding), and then expanded symmetrically from the bottleneck layer to the output layer (symmetric to the input layer). The nodes of the dimension reduction layer and the dimension restoration layer may be symmetric or asymmetric.

The autoencoder according to an embodiment of the present invention can perform nonlinear dimension reduction. The number of input and output layers may correspond to the number of sensors remaining after the pre-processing of input data. In the autoencoder structure, the number of nodes in the hidden layer included in the encoder may decrease as it moves away from the input layer.

The number of nodes in the bottleneck layer (the layer with the fewest nodes located between the encoder and decoder) may be maintained above a certain number (for example, more than half of the input layer) to ensure that a sufficient amount of information is transmitted, as having too few nodes may result in inadequate information transfer.

The neural network can be trained using at least one method among supervised learning, unsupervised learning, and semi-supervised learning. The training of the neural network aims to minimize the error of the output.

In the training of the neural network, the process involves repeatedly inputting training data into the neural network, calculating the error between the neural network's output for the training data and the target, and backpropagating the error from the output layer towards the input layer to update the weights of each node in the neural network in a direction that reduces the error.

In supervised learning, labeled training data, where each piece of training data has a labeled correct answer, is used (i.e., labeled training data). In unsupervised learning, the training data may not have labeled correct answers. For example, in supervised learning related to data classification, the training data may be data where each piece of training data is labeled with a category.

The error can be calculated by inputting labeled training data into the neural network and comparing the neural network's output (category) with the label of the training data. In another example, in unsupervised learning related to data classification, the error can be calculated by comparing the input training data with the neural network's output.

The calculated error is backpropagated in the neural network in the reverse direction (i.e., from the output layer towards the input layer), and according to the backpropagation, the connection weights of each node in each layer of the neural network can be updated. The change in the connection weights of each node being updated can be determined by the learning rate.

The computation of the neural network for the input data and the backpropagation of the error can constitute a learning cycle (epoch). The learning rate can be applied differently depending on the number of repetitions of the learning cycle of the neural network.

For example, a high learning rate can be used in the early stages of training the neural network to quickly achieve a certain level of performance, thereby increasing efficiency, and a low learning rate can be used in the later stages to improve accuracy.

In the training of the neural network, the training data is generally a subset of the actual data (i.e., the data intended to be processed using the trained neural network), and thus, there may exist learning cycles where the error for the training data decreases but the error for the actual data increases.

Overfitting is a phenomenon where the error for the actual data increases due to excessive learning on the training data. For example, a neural network trained to recognize cats by showing it yellow cats may not recognize cats of colors other than yellow, which is a type of overfitting.

Overfitting can act as a cause that increases the error of machine learning algorithms. To prevent such overfitting, various optimization methods can be employed. Methods such as increasing the training data, applying regularization, or using dropout, which omits some nodes of the network during the training process, can be applied to prevent overfitting.

Throughout this specification, the terms computational model, neural network, network function, and neural network can be used interchangeably (hereinafter referred to as a neural network for consistency). The data structure may include a neural network.

The data structure including the neural network can be stored on a computer-readable medium. The data structure including the neural network may also include data input to the neural network, weights of the neural network, hyperparameters of the neural network, data acquired from the neural network, activation functions associated with each node or layer of the neural network, and loss functions for training the neural network.

The data structure including the neural network may include any of the components disclosed above. That is, the data structure including the neural network may be configured to include all or any combination of data input to the neural network, weights of the neural network, hyperparameters of the neural network, data acquired from the neural network, activation functions associated with each node or layer of the neural network, and loss functions for training the neural network. In addition to the aforementioned components, the data structure including the neural network may include any other information that determines the characteristics of the neural network.

Furthermore, the data structure may include all forms of data used or generated in the computational process of the neural network and is not limited to the aforementioned details. The computer-readable medium may include a computer-readable recording medium and/or a computer-readable transmission medium. The neural network may generally be composed of a set of interconnected computational units, commonly referred to as nodes. These nodes may also be referred to as neurons. The neural network is configured to include at least one or more nodes.

Within the neural network, one or more nodes connected via links can form a relative relationship of input nodes and output nodes. The concept of input nodes and output nodes is relative; any node in an output node relationship with respect to one node may be in an input node relationship with another node, and vice versa.

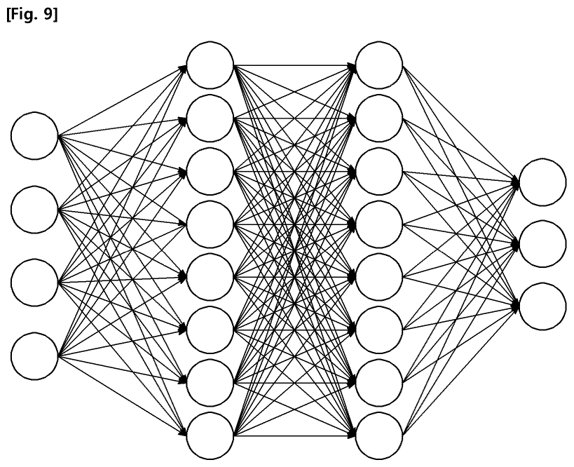

As described above, the input node to output node relationship can be generated around the link. As shown in FIG. 8, one input node can be connected to one or more output nodes via links, and vice versa.

In the relationship of input nodes and output nodes connected through a link, the value of the output node can be determined based on the data input to the input node. Here, the node interconnecting the input node and the output node can have a weight.

The weight can be variable and can be adjusted by the user or algorithm to perform the desired function of the neural network. For example, if one output node is interconnected with one or more input nodes by respective links, the output node can determine its value based on the values input to the connected input nodes and the weights set on the links corresponding to each input node.

As described above, the neural network forms an input node and output node relationship within the neural network by interconnecting one or more nodes through one or more links. The characteristics of the neural network can be determined by the number of nodes and links within the neural network, the relationships between the nodes and links, and the values of the weights assigned to each link.

For example, if there are two neural networks with the same number of nodes and links, but with different weight values between the links, the two neural networks may be recognized as different from each other.

Some of the nodes constituting the neural network can form a layer based on their distances from the initial input node. For instance, a set of nodes that are at a distance of n from the initial input node can form the n-th layer.

The distance from the initial input node can be defined by the minimum number of links that must be traversed to reach the respective node from the initial input node.

However, this definition of a layer is arbitrary for the purpose of explanation, and the order of layers within the neural network can be defined in a manner different from the aforementioned method. For example, the layers of nodes may also be defined by their distance from the final output node.

The initial input node may refer to one or more nodes within the neural network where data is directly input without passing through links in relation to other nodes. Alternatively, within the neural network, it may refer to nodes that do not have other input nodes connected by links in terms of the relationship between nodes based on links.

Similarly, the final output node may refer to one or more nodes within the neural network that do not have output nodes in relation to other nodes. Additionally, a hidden node refers to nodes constituting the neural network that are neither the initial input node nor the final output node. In one embodiment of the present invention, the neural network may have more nodes in the input layer than in the hidden layer closer to the output layer, and the number of nodes may decrease as it progresses from the input layer to the hidden layer.

The neural network may include one or more hidden layers. The hidden nodes of a hidden layer can take the output of the previous layer and the output of surrounding hidden nodes as input. The number of hidden nodes per hidden layer may be the same or different.

The number of nodes in the input layer can be determined based on the number of data fields of the input data and may be the same as or different from the number of hidden nodes. The input data entered into the input layer can be processed by the hidden nodes of the hidden layer and output by the fully connected layer (FCL), which is the output layer.

### Feature Extraction Model and Feature Classification Model

According to one embodiment of the present invention, the sleep analysis model may include a feature extraction model that extracts one or more features per predetermined epoch and a feature classification model that classifies each feature extracted through the feature extraction model into one or more sleep stages to generate sleep stage information.

According to an embodiment, the feature extraction model can analyze the time-series frequency patterns of the spectrogram 300 to extract features related to breathing sounds and breathing patterns.

In one embodiment, the feature extraction model may be configured through a part of a pre-trained neural network model (e.g., an autoencoder) using a training data set. Here, the training data set may consist of a plurality of spectrograms and a plurality of sleep stage information corresponding to each spectrogram.

In one embodiment, the feature extraction model may be configured through a proprietary deep learning model (e.g., an autoencoder) trained using a training data set. The feature extraction model can be trained using supervised or unsupervised learning methods. The feature extraction model can be trained to output data similar to the input data through the training data set.

Specifically, during the encoding process through the encoder, only the core feature data (or features) of the input spectrogram can be learned through the hidden layer, while the remaining information can be discarded. In this case, during the decoding process through the decoder, the output data of the hidden layer may be an approximation of the input data (i.e., the spectrogram) rather than a perfect copy. That is, the autoencoder can be trained to adjust the weights so that the output data and the input data become as similar as possible.

Each of the plurality of spectrograms included in the training data set can be tagged with sleep stage information. Each of the plurality of spectrograms can be input into the feature extraction model, and the output corresponding to each spectrogram can be stored in association with the tagged sleep stage information.

Specifically, when the first training data sets (i.e., a plurality of spectrograms) tagged with the first sleep stage information (e.g., light sleep) are used as input, the features related to the output for the corresponding input can be stored in association with the first sleep stage information. In an embodiment, one or more features related to the output can be represented in a vector space.

In this case, the feature data output corresponding to each of the first training data sets can be located relatively close in the vector space since they are outputs through spectrograms related to the first sleep stage. That is, the training can be performed so that a plurality of spectrograms corresponding to each sleep stage output similar features.

In the case of the encoder, it can be trained to extract features that allow the decoder to effectively reconstruct the input data. Therefore, as the feature extraction model is implemented through the encoder of the trained autoencoder, it can extract features (i.e., a plurality of features) that allow effective reconstruction of the input data (i.e., the spectrogram).

Through the aforementioned training process, the encoder constituting the feature extraction model can extract features corresponding to the spectrogram 300 when the spectrogram 300 (e.g., a spectrogram converted in response to sleep sound information) is used as input.

In an embodiment, the processor 130 or processor 830 can process the spectrogram 300 generated in response to the sleep sound information 210 as input to the feature extraction model to extract features. Here, since the sleep sound information 210 is time-series data acquired during the user's sleep, the processor 130 or processor 830 can divide the spectrogram 300 into predetermined epochs. For example, the processor 130 or processor 830 can divide the spectrogram 300 corresponding to the sleep sound information 210 into 30-second intervals to acquire a plurality of spectrograms. For instance, if sleep sound information is acquired during a user's 7-hour (i.e., 420 Minute) sleep, the processor 130 or processor 830 can divide the spectrogram into 30-second intervals to acquire 840 spectrograms.

Alternatively, in the embodiment as shown in FIG. 1(c), at least one of the electronic devices depicted in FIG. 1(c) may perform at least one of the aforementioned operations. The specific numerical descriptions regarding the sleep time, the division time unit of the spectrogram, and the number of divisions are merely exemplary and do not limit the present invention.

According to an embodiment of the present invention, the processor 130 or processor 830 may process each of the divided plurality of spectrograms as input to a feature extraction model to extract a plurality of features corresponding to each of the plurality of spectrograms. For example, if the number of the plurality of spectrograms is 840, the number of features extracted by the feature extraction model may also be 840. The specific numerical descriptions related to the number of spectrograms and the plurality of features are merely exemplary and do not limit the present invention.

Additionally, the processor 130 or processor 830 may process the plurality of features output through the feature extraction model as input to a feature classification model to acquire sleep stage information. In an embodiment, the feature classification model may be a neural network model designed to predict sleep stages corresponding to the features.

For example, the feature classification model may be configured to include a fully connected layer and may classify features into at least one of the sleep stages. For instance, if the feature classification model receives a first feature corresponding to a first spectrogram as input, it may classify the first feature as light sleep.

The feature classification model may perform multi-epoch classification by inputting spectrograms related to multiple epochs to predict sleep stages of multiple epochs. Multi-epoch classification refers to estimating multiple sleep stages (e.g., changes in sleep stages over time) at once by inputting spectrograms corresponding to multiple epochs (i.e., combinations of spectrograms each corresponding to 30 seconds), rather than providing sleep stage analysis information for a single epoch spectrogram (i.e., a single spectrogram corresponding to 30 seconds).

For example, since the breathing pattern changes more slowly compared to brainwave signals or other biological signals, it may be necessary to observe how the pattern changes at past and future points in time for accurate sleep stage estimation. Specifically, the feature classification model may perform predictions for 20 spectrograms located in the middle by inputting 40 spectrograms (e.g., 40 spectrograms each corresponding to 30 seconds). That is, while examining all spectrograms from 1 to 40, it may predict sleep stages through classification corresponding to spectrograms from 10 to 20. The specific numerical descriptions regarding the number of spectrograms are merely exemplary and do not limit the present invention.

In other words, in the process of estimating sleep stages, instead of performing sleep stage prediction corresponding to each single spectrogram, the method may improve the accuracy of the output by utilizing spectrograms corresponding to multiple epochs as input to consider information related to both past and future.

As described above, the processor 130 or processor 830 may acquire a spectrogram based on sleep sound information. In this case, the conversion to a spectrogram may be intended to facilitate the analysis of breathing or movement patterns related to relatively small sounds. Additionally, the processor 130 or processor 830 may generate sleep stage information based on the acquired spectrogram using a sleep analysis model configured to include a feature extraction model and a feature classification model. In this case, since the sleep analysis model can perform sleep stage prediction by inputting spectrograms corresponding to multiple epochs to consider information related to both past and future, it can output more accurate sleep stage information.

That is, the processor 130 or processor 830 may output sleep stage information corresponding to sleep sound information by utilizing the sleep analysis model as described above.

Alternatively, in the embodiment as shown in FIG. 1(c), at least one of the electronic devices depicted in FIG. 1(c) may perform the aforementioned operation.

According to an embodiment, the sleep stage information may pertain to information related to the stages of sleep that change during a user's sleep. For example, the sleep stage information may indicate how the user's sleep transitioned among light sleep, normal sleep, deep sleep, or REM sleep at each point during the user's 8-hour sleep the previous night. The specific description of the aforementioned sleep stage information is merely exemplary and does not limit the present invention.

### Method for Protecting User Privacy

FIG. 6(b) is a conceptual diagram illustrating a privacy protection method using Mel spectrogram conversion of sleep sound information extracted from a user in the sleep analysis method according to the present invention.

As shown in FIG. 6(b), the sound information extracted from the user, or the raw data extracted therefrom, which is the sleep sound information, undergoes a pre-processing step of noise reduction. In the noise reduction process, noise (e.g., white noise) included in the raw data is removed.

The noise reduction process can be performed using algorithms such as spectral gating and spectral subtraction to remove background noise.

Furthermore, in the present invention, the noise removal process can be performed using a deep learning-based noise reduction algorithm. The deep learning-based noise reduction algorithm can utilize a noise reduction algorithm specialized in the user's breathing sounds, that is, learned through the user's breathing sounds.

Subsequently, the noise-removed raw data is converted into a Mel spectrogram. Here, the Mel spectrogram refers to a series of simplified vectors in the frequency domain for a given input sentence (text).

At this time, a method can be used to generate the Mel spectrogram based solely on the amplitude, excluding the phase from the raw data, which not only protects privacy but also reduces data volume to improve processing speed. However, in other embodiments, it is also possible to generate the Mel spectrogram using both phase and amplitude.

The present invention generates a sleep analysis model using the Mel spectrogram 300 created based on the sleep sound information 210. If the sleep sound information expressed as audio data is used as is, the amount of information is very large, leading to a significant increase in computational load and time, and the inclusion of unwanted signals can degrade computational accuracy. Moreover, if all of the user's audio signals are transmitted to an external server 20 or AI server 310, there is a risk of privacy infringement.

By removing noise from the sleep sound information using the aforementioned method, converting it into a Mel spectrogram, and training the Mel spectrogram to generate a sleep analysis model, the present invention can reduce computational load and time while also promoting the protection of individual privacy.

At this time, the de-identification of sound data can be performed on natural language and breathing sounds, which can be converted into a natural language transformation mel spectrogram and a breathing sound transformation mel spectrogram, respectively. In the sleep analysis according to the present invention, only the information necessary for the analysis model is utilized to enhance computational speed and reduce computational load.

### Verification of Accuracy and Embodiments of the Sleep Analysis Method

FIG. 34 is a table verifying the accuracy of the sleep analysis method according to the present invention, showing experimental result data analyzed based on age, gender, BMI, and the presence of diseases.

FIG. 34 is a conceptual diagram illustrating an embodiment of the sleep analysis method according to the present invention, specifically when using a smart speaker and a smartphone for ease of understanding.

Unlike the polysomnography method in hospitals, the sleep analysis method according to the present invention allows for the on/off control of lighting during the examination and the free adjustment of indoor temperature and humidity.

That is, beyond the verification of fixed hospital environments, it allows for verification in various real-world situations to achieve a significant competitive edge, enabling convenient and flexible sleep analysis in diverse environments outside of hospitals using only a smart home-appliance 800 and a smartphone 900.

As a result, as shown in FIG. 34, it was confirmed that the experimental results consistently showed high accuracy across a wide range of ages, genders, BMIs, and target groups with sleep apnea and limb movement disorders.

As shown in FIG. 34, for ease of understanding, the smart home-appliance 800 is assumed to be a smart speaker 804, but it is not limited thereto. That is, the smart home-appliance 800 can be implemented as a tablet personal computer, mobile phone, video phone, e-book reader, desktop personal computer, laptop personal computer, netbook computer, workstation, server, personal digital assistant, portable multimedia player, MP3 player, mobile medical device, camera, or wearable device (e.g., smart glasses, head-mounted device (HMD), electronic clothing, electronic bracelet, electronic necklace, appcessory, electronic tattoo, smart watch), smart mirror, kiosk, etc.

Furthermore, the smart home-appliance 800 can be implemented as smart home appliances such as a TV, digital video disk player, audio system, refrigerator, air conditioner, vacuum cleaner, oven, microwave, washing machine, air purifier, set-top box, home automation control panel, security control panel, TV box, game console, electronic dictionary, electronic key, camcorder, or electronic frame, various medical devices, home robots, internet of things devices (e.g., light bulbs, various sensors, electric or gas meters, sprinkler devices, fire alarms, thermostats, street lights, toasters, exercise equipment, hot water tanks, heaters, boilers, etc.). Additionally, the smart home-appliance 800 can be implemented as part of furniture or a building/structure, an electronic board, an electronic signature receiving device, a projector, etc., and may be one or more combinations of the various devices mentioned above.

Alternatively, in the case of an embodiment like (c) of FIG. 1, at least one of the electronic devices shown in (c) of FIG. 1 may correspond to one or more combinations of the various devices described above.

Therefore, the sleep analysis method according to the present invention allows for convenient and simple deep sleep analysis of the user through smart home-appliances 800 such as a smartphone 900 or a smart speaker 804, without being constrained by time and place, even outside of a hospital setting.

### Configuration of the Non-Contact Sleep Analysis System

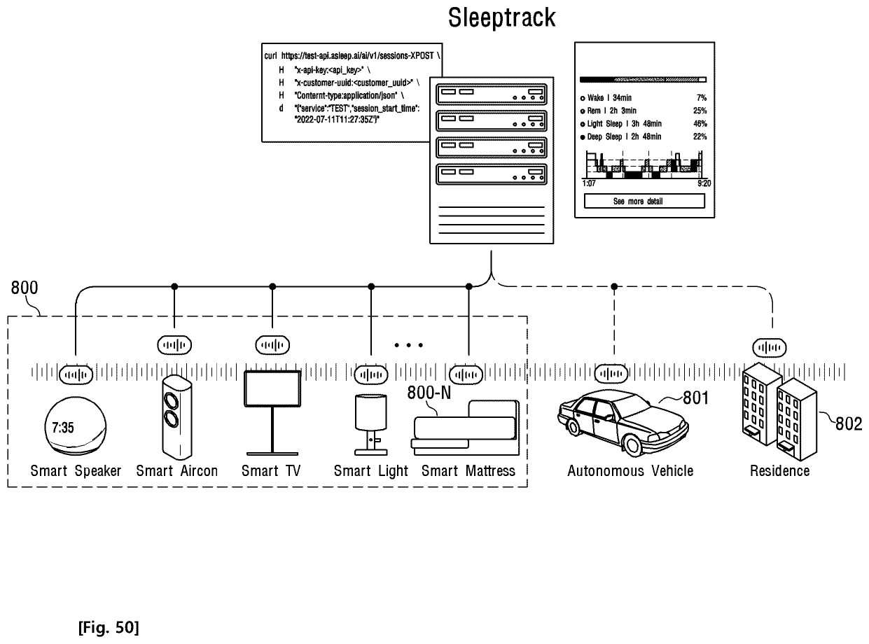

FIG. 50 is a configuration diagram for explaining the operation of an AI-based non-contact sleep analysis system according to the present invention, which includes one or more smart home-appliances 800, a SleepTrack app, an autonomous vehicle 801, and a living space 802.

FIG. 51 is a configuration diagram for explaining the operation among components of the AI-based non-contact sleep analysis system according to the present invention, which includes smart home-appliances 800, a smartphone 900, and an AI server 310.

As shown in FIG. 50, the smart home-appliance 800 according to the present invention can perform more universal and precise sleep analysis by acquiring the user's sleep sound information through an embedded microphone and utilizing it to conduct sleep analysis (non-contact sleep analysis).

That is, it can verify various real-world situations beyond the environmental verification of hospital sleep studies, accurately detecting events such as insomnia, sleep apnea, and hypopnea in real-time, and providing sleep diagnostic solutions for a wide range of ages, genders, races, BMI, and health conditions.

As seen in FIG. 51, the smart home-appliance 800 and the smartphone 900 work in conjunction to perform the user's sleep analysis. The smart home-appliance 800 and the smartphone 900 can be paired via Bluetooth or connected through other wireless communication methods.

According to the present invention, the smartphone 900 can perform sleep analysis based on the user's sleep sound information acquired from the smart home-appliance 800.

In this case, the user's sleep sound information may be acquired from the smart home-appliance 800 and transmitted to the smartphone 900, or it may be independently acquired through a microphone embedded in the smartphone 900.

That is, in the embodiment shown in FIG. 51, sleep stage analysis is conducted through non-contact sleep stage analysis using the smart home-appliance 800 and the smartphone 900. The user can check the sleep stage analysis results derived from the smartphone 900 on the screen of the smartphone 900.

As described above, even when the user does not wear the smart home-appliance 800, it is necessary for the smart home-appliance 800 to be appropriately positioned around the user to receive at least a part of the input signal for sleep analysis (e.g., body movement information) or the input signal for sleep analysis (sleep sound information).

In particular, to extract body movement information, it may be preferable to position the device in an area capable of detecting the user's movements (e.g., under the pillow, on top of the mattress).

On the other hand, since sound is transmitted in a radial direction, when using only sleep sound information, there is an advantage of being able to collect and analyze information regardless of the user's position, or the distance or angle between the user and the smart home-appliance 800.

Therefore, the smart home-appliance 800 of the present invention does not necessarily need to be worn by the user. If it is appropriately positioned within a predetermined radius (e.g., 4-5 m) within the user's sleep space, regardless of the user's position, distance, or angle, the sleep stage analysis described above becomes possible. The specific numerical description of the radius is merely exemplary and does not limit the present invention.

In one embodiment, when the smart home-appliance 800 is not worn by the user, it can emit a predetermined signal to prompt the user to place the smart home-appliance 800 closer to them so that it can receive the input signal for sleep analysis (sleep sound information). The predetermined signal may be vibration, alarm, text, LED, etc.

The radius between the user and the smart home-appliance 800 may be extracted by the smart home-appliance 800 or by the smartphone 900.

That is, since the user's sleep space is fixed, the position of the smart home-appliance 800 can be tracked to determine whether the smart home-appliance 800 is positioned appropriately.

Meanwhile, the smart home-appliance 800 may correspond to a sleep-related product (device) used for the user's sleep, rather than a device wearable by the user.

For example, as one of the smart home-appliances 800, a smart speaker 804 may be used. The smart speaker 804 may include an internal sound sensor to measure various sound information.

The smart speaker 804 can perform a primary sleep analysis using the sound information acquired through the sound sensor. The smart speaker 804 may be paired with a smartphone 900, allowing the information measured by the smart speaker 804 or the primary sleep analysis results analyzed by the smart speaker 804 to be transmitted to the smartphone 900. In this case, the smart speaker 804 may include a communication module.

Additionally, as one of the smart home-appliances 800, a smart mattress may be utilized. The smart mattress can include an acoustic sensor internally to measure various sleep sound information.

The smart mattress can perform a primary sleep analysis using the sleep sound information. The smart mattress can be paired with a smartphone 900 to transmit the information measured by the smart mattress or the primary sleep analysis results analyzed by the smart mattress to the smartphone 900. In this case, the smart mattress may include a communication module.

Meanwhile, the smart mattress may include various modules (temperature control module, infrared irradiation module, cooling module) for temperature regulation, and the temperature can be adjusted based on the final sleep stage analysis results. This enhances the quality of the user's sleep.

Furthermore, the aforementioned smart speaker 804 or smart mattress may include a vibration module or an alarm module to alleviate and improve sleep disorders, as will be described later. That is, if sleep apnea, snoring, sleep hyperventilation, REM sleep, etc., are detected, the vibration module or alarm module of the smart speaker 804 or smart mattress can be activated to deliver tactile or auditory stimuli to the user.

Additionally, in autonomous vehicles 801 or recently constructed living spaces 802, one or more smart devices may be integrated with the SleepTrack app, allowing the AI-based non-contact sleep analysis system according to the present invention to be established and operated.

Alternatively, in the case of an embodiment like that shown in FIG. 1(c), at least one of the electronic devices depicted in FIG. 1(c) can perform at least one of the aforementioned operations.

The descriptions of the types of smart home-appliances or spaces mentioned above are merely exemplary, and the present invention is not limited thereto.

Smart Home-Appliances within the Sleep Analysis System

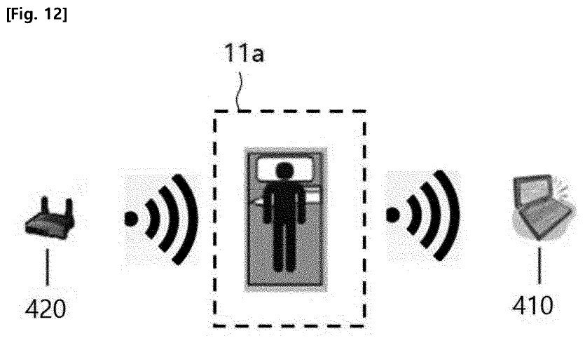

FIG. 11(b) is a block diagram illustrating the configuration of a smart home-appliance within the AI-based non-contact sleep analysis system according to the present invention.

The smart home-appliance 800 according to the present invention includes a communication unit 810, a sensing unit 820, a processor 830, a memory unit 840, and an alarm unit 850. Various other configurations to perform the functions of the smart home-appliance 800 may also be included.

In other words, according to the implementation aspects of the embodiments of the present invention, additional configurations may be further included, or some of the configurations may be omitted, or two or more configurations may be integrated into one configuration.

The communication unit 810 performs data transmission and reception with a smartphone 900 or an AI server 310 through a wireless communication network. The wireless communication network may include short-range wireless communication networks such as Z-wave, Zigbee, Wi-Fi, Bluetooth (BLE), LTE-M, LoRa (Long Range), Narrowband Internet of Things (NB-IoT), and Infrared Data Association (IrDA). Additionally, the wireless communication network may include 2G mobile communication networks such as Wireless LAN (WLAN), Wireless Broadband (Wibro), Wi-Fi (wireless fidelity), WiMax (world interoperability for microwave access), GSM (global system for mobile communication), or CDMA (code division multiple access), 3G mobile communication networks such as WCDMA (wideband code division multiple access) or CDMA2000, 3.5G mobile communication networks such as HSDPA (high speed downlink packet access) or HSUPA (high speed uplink packet access), and 4G, 5G, 6G mobile communication networks such as LTE (long term evolution) network or LTE-Advanced network, but are not limited thereto.

According to one embodiment of the present invention, the sensing unit 820 may include a microphone module for extracting sleep sound information of the user. The microphone module may be configured as a MEMS (Micro-Electro Mechanical Systems) to be applied to small devices. Such a microphone module can be manufactured in a very small size and may have a very low SNR (Signal Noise Ratio) compared to a condenser microphone or a dynamic microphone.

In this case, the sleep sound information is information of sound signals during sleep, which closely interacts with sleep itself and can be acquired without separately wearing wearable devices such as a smart watch or smart ring.

According to one embodiment of the present invention, the sensing unit 820 may include a pressure sensor, grip sensor, color sensor, IR (infrared) sensor, temperature sensor, humidity sensor, and illuminance sensor.

According to one embodiment of the present invention, the memory 840 may store a computer program for performing sleep analysis, and the stored computer program may be read and executed by the processor 830 described later. Additionally, the memory 840 may store any form of information generated or determined by the processor 830 and any form of information received by the communication unit 810. Furthermore, the memory 840 may store data related to the user's sleep.

For example, the memory 840 may temporarily or permanently store input/output data.

According to the present invention, the memory 840 may be implemented as at least one type of storage medium such as a flash memory type, hard disk type, multimedia card micro type, card type memory (e.g., SD or XD memory), RAM (Random Access Memory), SRAM (Static Random Access Memory), ROM (Read-Only Memory), EEPROM (Electrically Erasable Programmable Read-Only Memory), PROM (Programmable Read-Only Memory), magnetic memory, magnetic disk, or optical disk, but is not limited thereto.

According to the present invention, when the computer program is loaded into the memory 840, it may include one or more instructions that cause the processor 830 to perform methods/operations according to various embodiments of the present invention. That is, the processor 830 may perform methods/operations according to various embodiments of the present invention by executing one or more instructions.

According to one embodiment of the present invention, the processor 830 may be composed of one or more cores and may include processors for data analysis and deep learning, such as a central processing unit (CPU) of a smart home-appliance, a general-purpose graphics processing unit (GPGPU), or a tensor processing unit (TPU).

According to one embodiment of the present invention, the processor 830 can perform data processing for machine learning according to an embodiment of the present invention by reading a computer program stored in the memory 840. In one embodiment of the present invention, the processor 830 can perform operations for training a neural network.

According to the present invention, the processor 830 can perform calculations for training a neural network, such as processing input data for learning in deep learning (DL), feature extraction from input data, error calculation, and weight updates of the neural network using backpropagation.

Additionally, at least one of the CPU, GPGPU, and TPU of the processor 830 can process the learning of network functions.

For example, the CPU and GPGPU can jointly process the learning of network functions and data classification using network functions. Furthermore, in one embodiment of the present invention, the processors of multiple smart home-appliances can be used together to process the learning of network functions and data classification using network functions.

Additionally, the computer program executed in the smart home-appliance 800 according to one embodiment of the present invention can be a program executable by a CPU, GPGPU, or TPU.

According to the present invention, the network function can be used interchangeably with artificial neural networks and neural networks. The network function may include one or more neural networks, in which case the output of the network function can be an ensemble of the outputs of one or more neural networks.

According to the present invention, the model (inference model) may include a network function. The model may include one or more network functions, in which case the output of the model can be an ensemble of the outputs of one or more network functions.

According to the present invention, the processor 830 can provide a sleep analysis model according to one embodiment of the present invention by reading a computer program stored in the memory 840. In one embodiment of the present invention, the processor 830 can perform user sleep analysis based on sleep sound information using the sleep analysis model.

That is, a user's breathing during sleep contains a wealth of information for sleep analysis, including not only body movements and breathing sounds during sleep but also various sleep disorders (e.g., sleep apnea, hypopnea, snoring), which contain a lot of information. By utilizing artificial intelligence (AI), high accuracy can be expected.

As shown in FIG. 32(b), during the sleep stage, the user's breathing patterns and regularity, movement sounds during sleep, and breathing sounds are measured, and recovery breathing sounds after apnea events and unstable breathing sounds during hypopnea events can be measured.

Furthermore, when the frequency pattern of breathing sounds is analyzed, it is possible to fundamentally predict the causes of snoring or sleep apnea.

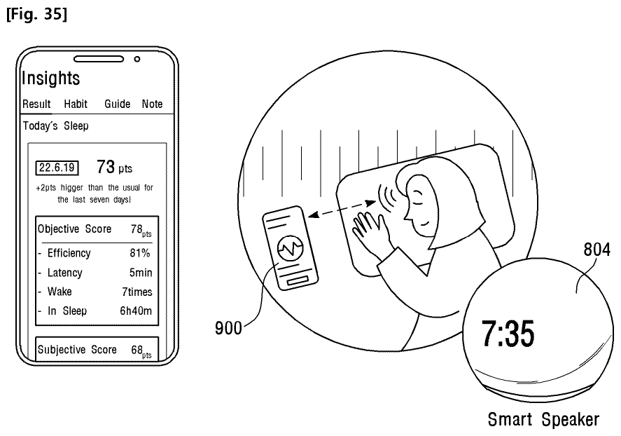

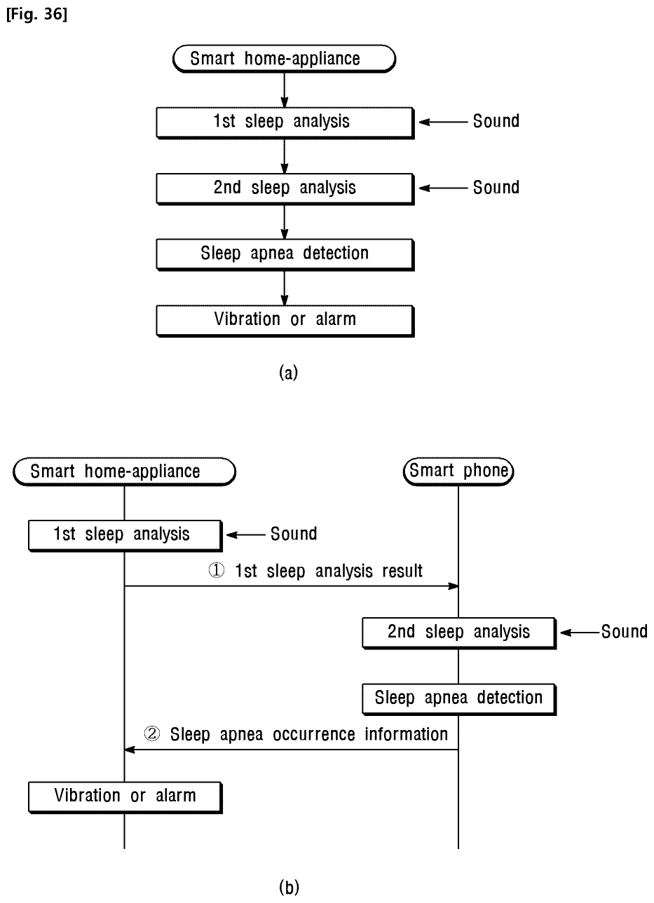

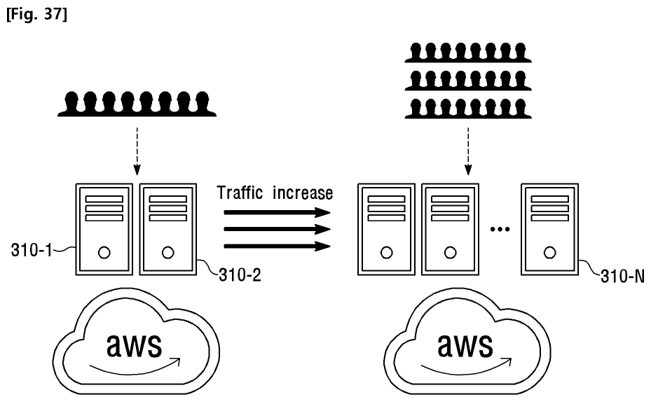

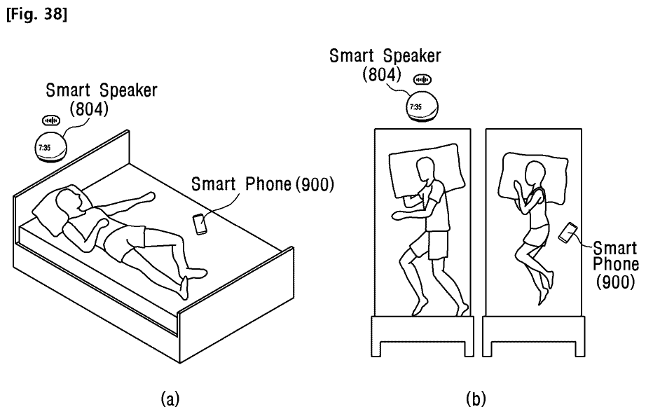

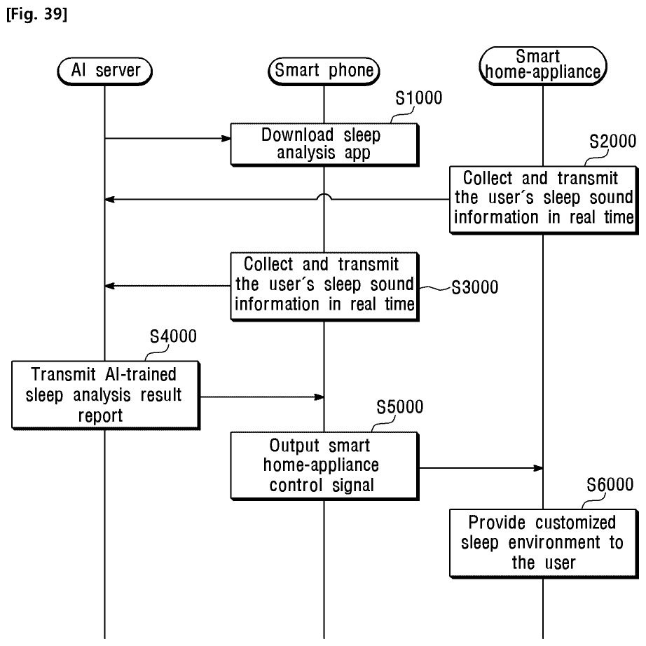

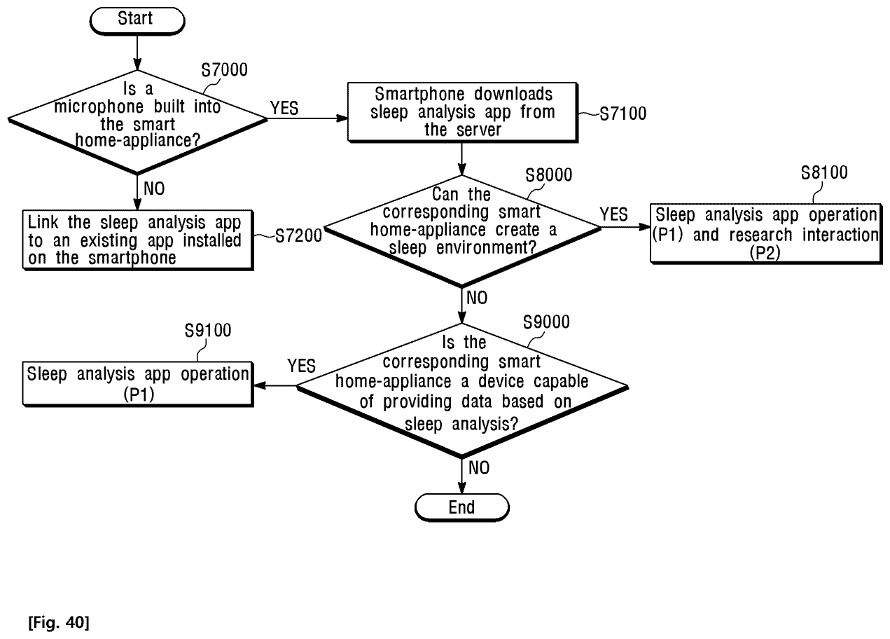

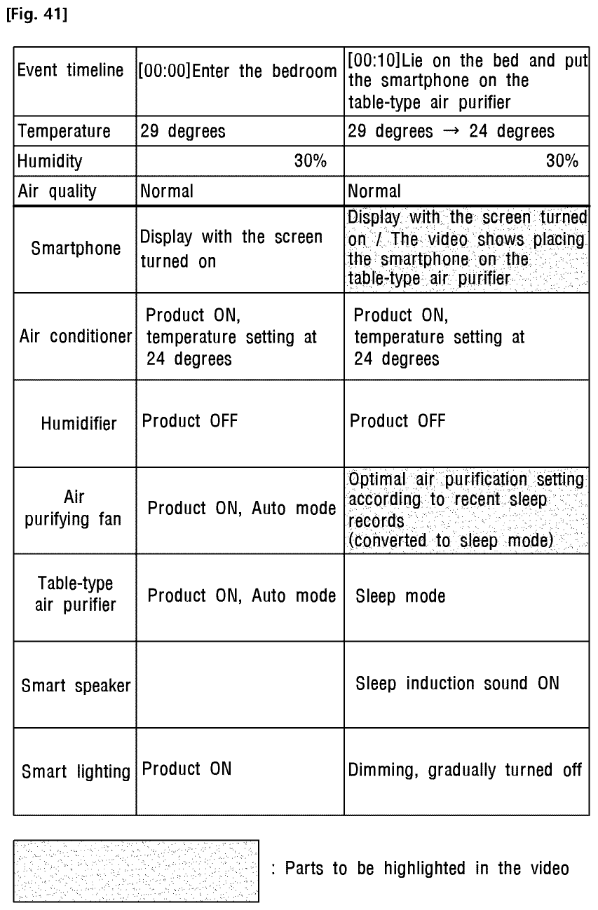

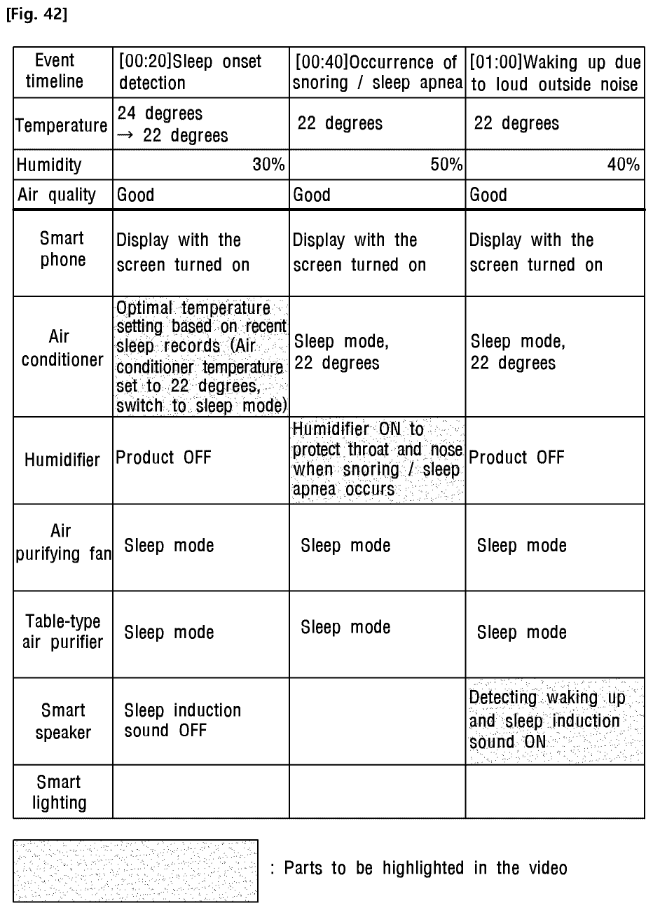

In particular, breathing sounds during sleep, which are the user's breathing sounds during sleep, can be conveniently measured outside of a hospital through various smart home-appliances 800 such as a smartphone 900 or a smart speaker 804, as shown in FIG. 35.

According to one embodiment of the present invention, the processor 830 can perform calculations to train the sleep analysis model. Based on the sleep analysis model, sleep information related to the user's sleep stages, sleep quality, occurrence of sleep disorders, etc., can be inferred. Sleep sound information acquired from the user in real-time or periodically is input as input data into the sleep analysis model, resulting in the output of data related to the user's sleep (data concerning sleep stages, sleep quality, occurrence of sleep disorders, etc.).

Meanwhile, the smart home-appliance 800 according to the present invention may further include an alarm unit 850. The alarm unit 850 is a means to provide tactile or auditory feedback to the user in the event of sleep disorders such as sleep apnea during primary and secondary sleep analysis.

For example, the alarm unit 850 can be implemented as an actuator generating vibrations, a vibration module, a haptic module, or as a speaker module generating sound or audio.

In the present invention, sleep state information may relate to whether the user is sleeping. Specifically, sleep state information may include at least one of the first sleep state information indicating the user is before sleep, the second sleep state information indicating the user is during sleep, and the third sleep state information indicating the user is after sleep.

In other words, if the first sleep state information is inferred regarding the user, the processor 830 can determine that the user is in a state before sleep (i.e., before going to bed). If the second sleep state information is inferred, it can be determined that the user is in a state during sleep, and if the third sleep state information is acquired, it can be determined that the user is in a state after sleep (i.e., upon waking).

Such sleep state information can be acquired based on environment sensing information. Environment sensing information may be sensing information acquired in a non-contact manner from the space where the user is located.

For example, the processor 830 can extract sleep state information based on environment sensing information acquired from the sensing unit 820, such as sound information related to cleaning, sound information related to cooking, sound information related to watching TV, and sleep sound information acquired during sleep.

In this case, the sleep sound information acquired during the user's sleep may include sounds generated by the user's tossing and turning, sounds related to muscle movements, or breathing sounds during sleep. That is, in the present invention, sleep sound information may refer to sound information related to the user's breathing patterns during sleep.

According to the present invention, sleep stages can be classified into NREM (non-REM) sleep and REM (Rapid Eye Movement) sleep, and NREM sleep can be further divided into multiple stages (e.g., two stages of Light and Deep, or four stages from N1 to N4. The setting of sleep stages may be defined based on generally accepted sleep stages, but can also be arbitrarily set in various ways depending on the designer.

According to one embodiment of the present invention, through sleep stage analysis, not only the quality of sleep but also sleep disorders (e.g., sleep apnea) and their underlying causes (e.g., snoring) can be predicted.

According to the present invention, the processor 830 can acquire sleep state information based on sound information obtained from the smart home-appliance 800.

Specifically, the processor 830 can identify singularities where information of a predefined pattern is detected in the sound information.

Here, the information of the predefined pattern may relate to breathing patterns associated with sleep. For example, in the wake state, all nervous systems are activated, leading to irregular breathing patterns and significant body movement.

Additionally, because the relaxation of neck muscles does not occur, breathing sounds may be minimal. In contrast, when the user is sleeping, the autonomic nervous system stabilizes, causing breathing to change regularly, and breathing sounds may increase.

That is, the processor 830 can identify the point in time when sound information related to regular breathing and minimal breathing sounds, which are predefined pattern sound information, is detected as a singularity. Furthermore, the processor 830 can acquire sleep sound information based on the sound information obtained with reference to the identified singularity 201.

The processor 830 can identify singularities related to the user's sleep timing from the time-series sound information and acquire sleep sound information based on these singularities.

Additionally, according to one embodiment of the present invention, in the case of an embodiment like (c) of FIG. 1, at least one of the electronic devices shown in (c) of FIG. 1 may perform at least one of the aforementioned operations.

Comparison of conventional sleep analysis methods and the sleep analysis method of the present invention

FIG. 45 is a conceptual diagram illustrating the training method in the case of using only sleep polysomnography microphone data (S) in a hospital environment according to conventional sleep analysis methods, for comparison with the sleep analysis method of the present invention.

FIG. 46 is a conceptual diagram illustrating a method for generating an AI sleep analysis model by reflecting various sounds in a home environment according to the sleep analysis method of the present invention, in the training method shown in FIG. 45.

Here, waveform (a) represents the waveform of sleep polysomnography microphone data (S) in a hospital environment, waveform (b) represents the waveform of various noise data (N) occurring in a home environment, and waveform (c) is the combined waveform of waveform (a) and waveform (b).

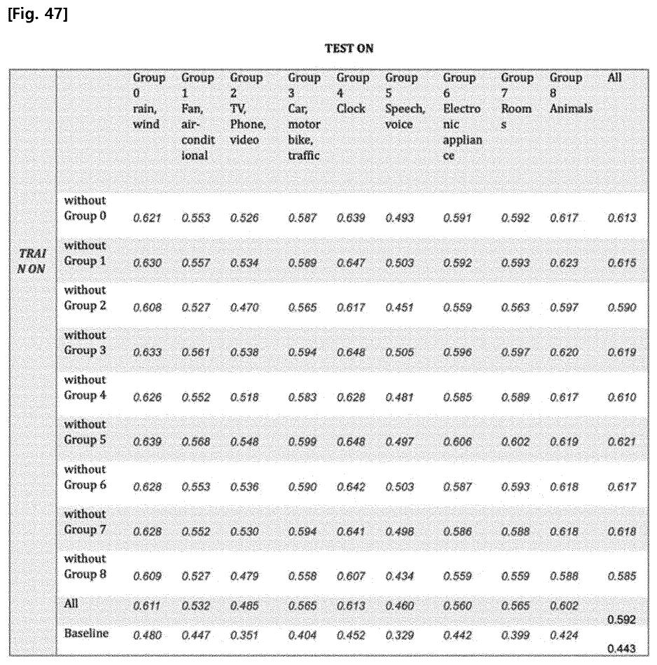

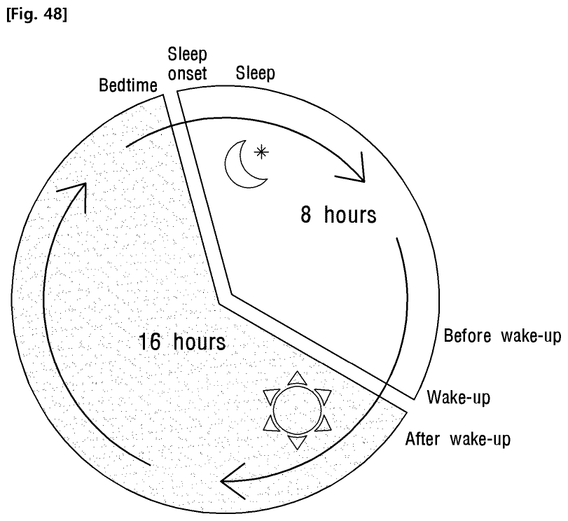

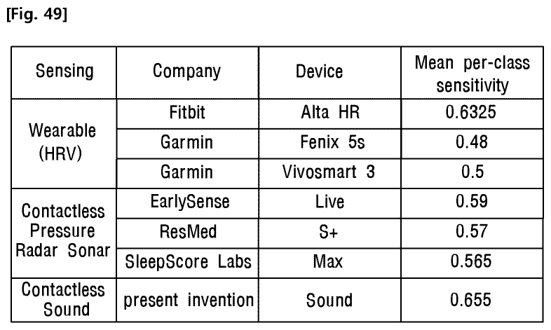

FIG. 47 is a table verifying the performance of the sleep analysis method according to the present invention, trained by dividing the performance into nine groups according to the type of residential noise, and it is experimental result data tested on groups 0 to 8.

The types of residential noise are as follows: Group 1 includes rain sound and wind sound; Group 2 includes fan sound and air conditioner sound; Group 3 includes TV sound, telephone sound, and video recorder sound; Group 4 includes car sound, motorbike sound, and other vehicle sounds; Group 5 includes clock sound; Group 6 includes human conversation sound and voice; Group 7 includes electronic device sound; Group 8 includes inter-room/inter-floor noise; and Group 9 includes pet sound.

As seen in FIG. 45, the conventional training method using only sleep polysomnography microphone data (S) collected in a hospital environment involves inputting the sleep polysomnography microphone data (S) collected in the hospital, processing it through the first AI sleep analysis model, and generating and feeding back a label for sleep analysis and diagnosis reflecting classification loss.

In contrast, the training method using home sleep polysomnography microphone data (H) is as follows.

First, as shown in FIG. 46, the sleep polysomnography microphone data (S) used in the conventional training method (a) with only hospital environment data is combined with various noise data (N) occurring in the home environment and inputted.

When this combined data (S+N) is inputted and processed through the second AI sleep analysis model, consistency loss occurs.

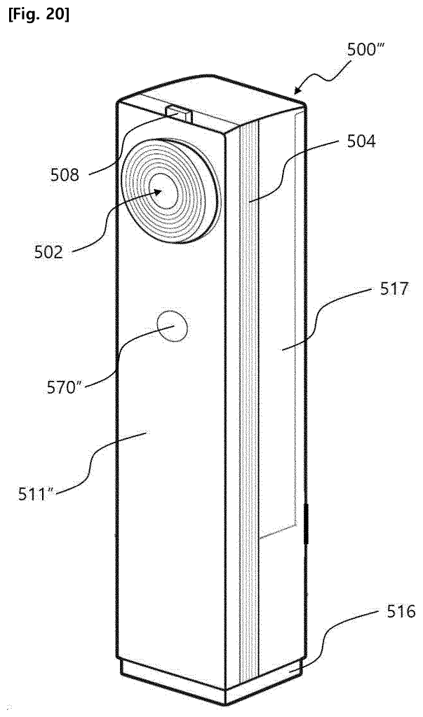

When the classification loss generated by the training method shown in FIG. 20 is added and reflected in this consistency loss, the third AI sleep analysis model is generated.

At this time, the first and second AI sleep analysis models impose a correlation between each other's output data.

### User's 24-Hour Monitoring Processor and Mean Per Class Result Comparison

FIG. 48 is a schematic diagram illustrating the 24-hour monitoring process of a user according to the AI-based non-contact sleep analysis system and sleep analysis method of the present invention.

FIG. 49 is a table comparing the mean per class results of the smart home-appliance and sleep analysis method according to the present invention with the products and devices of existing world-leading sleep tech companies.

In conventional cases where only a smart watch was used to analyze patterns of user activity, rest, and sleep, there was a problem of sleep analysis being interrupted when the smart watch was removed during sleep. The present invention allows for seamless real-time monitoring of all user activities, even when the smart watch is removed during sleep, by utilizing a smart phone 900 in conjunction with a smart home-appliance 800.

For example, when the smart watch is removed, placed on a charger, or mounted on a charging pad, the smart phone 900 is automatically activated, allowing continuous analysis of user activity, rest, and sleep. In this case, the smart phone 900 can be activated when it is time to sleep, even when not adjacent to the smart home-appliance 800.

This method ensures the continuity of user activity measurement, including sleep. For instance, as shown in FIG. 48, 24-hour data can be acquired through the smart phone 900. This data can then be processed into various reports and provided to the user.

The user can start sleep recording by touching the screen of the smart phone 900 and receive a sleep analysis result report (such as bedtime, sleep onset latency, sleep duration, time taken to wake after alarm, etc.) analyzed in the aforementioned manner. Alarms (such as alarms with gradually increasing sound tailored to individual sleep stages) can be automatically generated according to sleep stages, and all-day care services such as user profiling (sleep information, preferred content, content recommendations based on age/gender/occupation, etc.), and recommendations for personalized sleep/exercise/diet/cosmetics/behavioral regulations optimized for individual sleep patterns can be provided.

The present invention displays weight/blood pressure and sleep apnea, insomnia, or exercise and insomnia as sleep measurement records, which can motivate users to change behaviors to improve their health. That is, the present invention can naturally enhance user compliance with behavior changes.

For example, if a user is overweight, sleep apnea commonly occurs, and weight loss can help improve sleep apnea. Thus, the present invention can be linked with a healthcare app's diet, exercise, and weight tracking.

In other words, the history of sleep apnea allows for behavioral intervention with real-time sleep apnea detection and accuracy provided by the present invention.

Additionally, since sleep apnea can be a cause of hypertension, the present invention enables blood pressure tracking management when respiratory instability intervals occur regularly.

Specifically, weight loss can help lower blood pressure in the human body, and upon successful weight loss, the Pittsburgh Sleep Quality Index (PSQI) can be utilized to objectively compare the quality of sleep before and after the weight loss.

Furthermore, exercise (excluding within 3 hours before bedtime) can aid in alleviating insomnia, thereby increasing the time spent exposed to natural light during outdoor activities and improving the user's mood.

Additionally, it becomes possible to receive recommendations for various exercise programs from healthcare applications.

Moreover, the present invention can display the correlation between stress levels and sleep, or premenstrual syndrome and insomnia, to the user, which can lead the user to reassess their health condition.

By indicating the correlation between stress levels and sleep quality, an element of interest is added, and based on the user's stress levels and degree of depression, it becomes possible to complete psychiatric-related questionnaires provided by healthcare applications.

Additionally, in cases where insomnia is reported as a symptom of premenstrual syndrome, the sleep efficiency can be annotated in the menstrual cycle tracking calendar to allow for the comparison of sleep data, enabling the user to check their health status related to physiological phenomena.

Meanwhile, one of the important aspects of sleep stage analysis is determining whether the user wakes up during sleep and whether genuine awakening occurs. Specifically, it is crucial to accurately analyze the WAKE stage, and sleep sound information is a highly useful factor in detecting whether the user is in the genuine WAKE stage.

In conventional polysomnography, EEG measurements merely confirmed changes in brain waves when the user was awake. However, the sleep stage analysis of the present invention utilizes sleep sound information to indicate precursor signals (such as sound patterns and movement patterns) before the user wakes up (before reaching the WAKE stage), allowing for the prediction and detection of the WAKE stage.

According to the AI sleep stage analysis model trained on a multitude of data, the determination of the WAKE stage, particularly based on sleep sound information, becomes more precise. Additionally, while a user waking from sleep may follow their body's biorhythm, it can also be influenced by external factors such as ambient noise and disturbances.

The present invention enables the AI sleep stage analysis model to be constructed by learning the user's sleep environment, including various ambient noises such as routine noises occurring in the surrounding space and abnormal or intermittent noises. As a result, the WAKE stage can be predicted and detected more clearly and reliably.

As shown in FIG. 49, compared to the solutions of existing world-leading sleep tech companies, the accuracy of Wake detection has improved by 43% compared to existing wearables and by 52% compared to existing non-contact methods. In terms of average accuracy for Wake/Sleep, results showed an improvement of 16% over existing wearables and 20% over existing non-contact methods.

Furthermore, in terms of average accuracy for Wake/NREM/REM 3C), results showed an improvement of 15% over existing wearables and 25% over existing non-contact methods.

Additionally, the sleep analysis of the present invention, utilizing sleep sound information, possesses high versatility as it can be performed by anyone with a device that includes a microphone, and can be applied to various devices.

The sleep analysis method, sleep disorder alleviation and prevention method, sleep disorder improvement method, and monitoring method according to the present invention can be provided by a server offering cloud computing services. More specifically, these methods can be executed by a server providing cloud computing services, which processes information on a computer connected to the internet rather than on the user's computer.

In the embodiment shown in FIGS. 50 and 51, various sleep sound information acquired from a smart home-appliance 800 and a smartphone 900 is transmitted to the AI server 310. The AI server 310 performs sleep analysis using this information and can transmit the results back to the smart home-appliance 800 and smartphone 900.

According to another embodiment of the present invention, various sleep sound information acquired from the smartphone 900 can be converted into a spectrogram on the smartphone 900 and transmitted to the AI server 310. In this case, the AI server 310 can perform sleep analysis using the spectrogram.

According to yet another embodiment of the present invention, various sleep sound information acquired from the smartphone 900 can be converted into a spectrogram by the AI server 310, which then performs sleep analysis using the spectrogram.

Cloud computing services can store data on the internet, allowing users to access necessary data or programs via internet connection without installing them on their computers. Users can easily share and transfer stored data with simple operations and clicks. Additionally, cloud computing services not only store data on internet servers but also allow users to perform desired tasks using application functions provided on the web without installing separate programs. Multiple users can simultaneously share and work on documents.

Cloud computing services can be implemented in at least one form of IaaS (Infrastructure as a Service), PaaS (Platform as a Service), SaaS (Software as a Service), virtual machine-based cloud servers, and container-based cloud servers. That is, the smart home-appliance 800 of the present invention can be implemented in at least one form of the aforementioned cloud computing services. The specific description of the aforementioned cloud computing services is merely exemplary and may include any platform for constructing the cloud computing environment of the present invention.

The sleep analysis method, sleep disorder alleviation and prevention method, sleep disorder improvement method, and monitoring method according to the present invention can be implemented in the form of program instructions executable through various computer means and can be recorded on a computer-readable medium. The computer-readable recording medium may include program instructions, data files, data structures, etc., either alone or in combination.

The program instructions recorded on the medium may be specifically designed and configured for the present invention or may be those known and available to those skilled in computer software. Examples of computer-readable recording media include magnetic media such as hard disks, floppy disks, and magnetic tapes; optical media such as CD-ROMs and DVDs; magneto-optical media such as floptical disks; and hardware devices specially configured to store and execute program instructions, such as ROM, RAM, and flash memory.

Examples of program instructions include machine code, such as that generated by a compiler, as well as high-level language code executable by a computer using an interpreter or the like. The aforementioned hardware devices may be configured to operate as one or more software modules to perform the operations of the present invention, and vice versa.

Alternatively, in the case of an embodiment such as that shown in FIG. 1(c), at least one of the electronic devices depicted in FIG. 1(c) may perform at least one of the aforementioned operations.

### Generation of Environment Adjustment Information

According to an embodiment of the present invention, the processor 130 or processor 830 can generate environment adjustment information based on sleep state information and/or sleep stage information.

Sleep state information relates to whether the user is sleeping and may include at least one of the first sleep state information indicating the user is before sleep, the second sleep state information indicating the user is during sleep, and the third sleep state information indicating the user is after sleep. The step of generating environment adjustment information will be described in detail using processor 130 as an example.

According to the embodiment, processor 130 can generate first environment adjustment information based on the first sleep state information. Specifically, when processor 130 acquires the first sleep state information indicating the user is before sleep, it can generate first environment adjustment information based on the said first sleep state information.

According to the embodiment, the first environment adjustment information may relate to the intensity and illumination of light that naturally induces sleep. Specifically, the first environment adjustment information may be control information to supply white light at 3000K with an illumination of 30 lux from the sleep induction point until the second sleep state information is acquired.

According to the embodiment, the sleep induction point can be determined by processor 130. Specifically, processor 130 can determine the sleep induction point through information exchange with the user's user terminal 10. For example, the user can set the desired sleep time through the user terminal 10 and transmit it to processor 130. Processor 130 can determine the sleep induction point based on the time the user wishes to sleep, as received from the user terminal 10. For instance, processor 130 can determine the sleep induction point as 20 minutes before the time the user wishes to sleep. For example, if the user sets the desired sleep time as 11:00, processor 130 can determine 10:40 as the sleep induction point. The specific numerical values for the aforementioned times are merely exemplary and do not limit the present invention.

According to an embodiment, the processor 130 can acquire the user's sleep intention information based on the environment sensing information and determine the sleep induction timing based on the sleep intention information. The sleep intention information may represent the user's intention to sleep as a quantitative value. For example, the higher the user's sleep intention, the closer the sleep intention information is to 10, and the lower the sleep intention, the closer the sleep intention information is to 0.

Alternatively, in an embodiment such as that shown in FIG. 1(c), at least one of the electronic devices depicted in FIG. 1(c) may perform at least one of the aforementioned operations.

The specific numerical description of the aforementioned sleep intention information is merely exemplary and the present invention is not limited thereto.

### Sleep Intention Information Acquisition

According to one embodiment of the present invention, the processor 130 or processor 830 can acquire sleep intention information based on the environment sensing information. Alternatively, in an embodiment such as that shown in FIG. 1(c), at least one of the electronic devices depicted in FIG. 1(c) may acquire the sleep intention information. Hereinafter, the step of acquiring sleep intention information will be described in detail using processor 130 as an example.

In one embodiment, the processor 130 can identify the types of sounds included in the environment sensing information. Additionally, the processor 130 can calculate the sleep intention information based on the number of identified sound types. The processor 130 may calculate lower sleep intention information as the number of sound types increases, and higher sleep intention information as the number of sound types decreases. For example, if the environment sensing information includes three types of sounds (e.g., vacuum cleaner noise, TV noise, and user voice), the processor 130 may calculate the sleep intention information as 2 points. Conversely, if the environment sensing information includes one type of sound (e.g., washing machine), the processor 130 may calculate the sleep intention information as 6 points. The specific numerical description of the types of sounds included in the environment sensing information and the sleep intention information is merely exemplary and the present invention is not limited thereto.

That is, the processor 130 can acquire sleep intention information related to how much the user intends to sleep based on the number of types of sounds included in the environment sensing information. For example, the more types of sounds identified, the lower the user's sleep intention, resulting in sleep intention information with a lower score.

In an embodiment, the processor 130 can pre-match different intention scores to each of the multiple sound information and create or record an intention score table. For example, a first sound information related to a washing machine may be matched with an intention score of 2 points, a second sound information related to a humidifier noise may be pre-matched with an intention score of 5 points, and a third sound information related to a voice may be matched with an intention score of 1 point. The processor 130 may pre-match relatively high intention scores to sound information related to the user's sleep (e.g., sounds generated by user activity such as vacuuming, dishwashing, voice sounds) and pre-match relatively low intention scores to sound information unrelated to the user's sleep (e.g., sounds unrelated to user activity such as vehicle noise, rain sounds) to create an intention score table. The specific numerical description of the intention scores matched to each sound information is merely exemplary and the present invention is not limited thereto.

The processor 130 can acquire sleep intention information based on the environment sensing information and the intention score table. Specifically, the processor 130 can record the intention score matched to the identified sound at the time when at least one of the multiple sounds included in the intention score table is identified in the environment sensing information. For example, if a vacuum cleaner sound is identified at a first time point during the real-time acquisition of environment sensing information, the processor 130 can match and record the intention score of 2 points associated with the vacuum cleaner sound at the first time point. The processor 130 can match and record the intention score matched to the identified sound at each time point whenever various sounds are identified during the environment sensing information acquisition process.

In an embodiment, the processor 130 can acquire sleep intention information based on the sum of the intention scores acquired over a predetermined time (e.g., 10 minutes). Specifically, the higher the intention score acquired over 10 minutes, the higher the sleep intention information that can be acquired, and the lower the intention score acquired over 10 minutes, the lower the sleep intention information that can be acquired. The specific numerical description of the predetermined time is merely exemplary and the present invention is not limited thereto.

That is, the processor 130 can acquire sleep intention information related to how likely the user intends to sleep, based on the characteristics of the sound included in the environment sensing information. For example, the more sounds related to the user's activities are identified, the sleep intention information indicating a low sleep intention of the user (i.e., sleep intention information with a low score) may be output.

### Determination of Environment Adjustment Information and Operation of Smart Home-Appliances

According to an embodiment of the present invention, the processor 130 or processor 830 can determine environment adjustment information based on sleep state information and/or sleep intention information.

Furthermore, based on the environment adjustment information, various smart home-appliances 800 according to an embodiment of the present invention can operate.

Alternatively, in the case of an embodiment such as (c) of FIG. 1, at least one of the electronic devices shown in (c) of FIG. 1 may perform at least one of the aforementioned operations. Hereinafter, the determination of environment adjustment information and the operation of smart home-appliances will be described in detail using drawings and the like.

### Overall Operation

FIG. 8 illustrates an exemplary flowchart for providing a sleep environment adjustment method according to sleep state information related to an embodiment of the present invention.

According to an embodiment of the present invention, the method may include a step S100 of acquiring the user's sleep state information.

According to an embodiment of the present invention, the method may include a step S200 of generating environment adjustment information based on the sleep state information.

According to an embodiment of the present invention, the method may include a step S300 of transmitting the environment adjustment information to the environment adjustment device 30.

The steps illustrated in FIG. 8 described above may have their order changed as needed, and at least one or more steps may be omitted or added. That is, the aforementioned steps are merely one embodiment of the present invention, and the scope of rights of the present invention is not limited thereto.

FIG. 39 is a flowchart for explaining the operation of a non-contact sleep analysis method based on AI according to the present invention.

FIG. 40 is a flowchart illustrating embodiments of various smart home-appliances used in the sleep analysis method according to the present invention.

Referring to FIGS. 50, 51, and 39, the overall operation of the AI-based non-contact sleep analysis method according to the present invention is schematically described as follows.

A sleep analysis app may be downloaded to a smartphone 900 (S1000).

At least one or more smart home-appliances 800 may collect the user's sleep sound information in real-time and transmit it to the server 310 (S2000).

The smartphone 900 may simultaneously collect the user's sleep sound information in real-time and transmit it to the server 310 (S3000).

The server 310 may transmit a sleep analysis result report, learned by AI, to the smartphone 900 (S4000).

The smartphone 900 may output a control signal to control the operation of at least one or more smart home-appliances 800 (S5000).

At least one or more smart home-appliances 800 may provide a customized sleep environment to the user (S6000).

Next, with reference to FIGS. 50, 51, and 40, the detailed operation of the AI-based contactless sleep analysis method according to the present invention is described as follows.

First, it can be determined whether a microphone is embedded in the smart home-appliance 800 (S7000).

If affirmative, the sleep analysis app according to the present invention (hereinafter referred to as the sleeptrack app) can be downloaded to the smartphone 900 (S7100). If negative, the sleeptrack app can be integrated with an existing app installed on the smartphone 900 (S7200).

Here, the features of the sleeptrack app are as follows.

It is a sleep analysis app that detects the user's real-time sleep stage information and periods of respiratory instability. It includes a database capable of storing weekly and monthly sleep quality indicators and sleep environment information, and utilizes a dashboard to derive service insights through usage sessions and sleep statistics, thereby calculating a highly accurate sleep stage graph (hypnogram), sleep evaluation indicators, and respiratory instability indicators for a single night.

Additionally, the sleeptrack app enables seamless monitoring and data collection between daily life and sleep in a contactless manner, without the need to wear a separate wearable device.

Through this, not only can the freedom of the body during sleep be increased, but also the wake time, which is the foundation of all sleep therapies, can be accurately matched, allowing for convenient and accurate analysis of various types of users' sleep at home, regardless of time and place.

Furthermore, the purposes of the sleeptrack app are as follows.

Based on real-time sleep tracking, it intervenes in the user's sleep to create the optimal sleep environment for the user based on sleep analysis results, providing user-specific sleep pattern analysis reports, as well as alarms tailored to individual sleep stages, sleep hygiene guides, and sleep/wake sound content.

Additionally, it can recommend content that forms behavior correction and sleep routines optimized for individual sleep patterns, such as personalized exercises and dietary habits, based on user profiles including sleep information, preferred content, sleep BTI, and recommended content responsiveness according to age group, gender, and occupation.

Meanwhile, in step S7100, it is determined whether the smart home-appliance can create a sleep environment (S8000). Here, the sleep environment may include temperature, humidity, light, sound, the position of the head and body, scent, and more.

In step S8000, if affirmative, the SleepTrack app is activated, and research interactions may be generated (S810). If negative, it is determined whether the device can provide customer value based on sleep analysis through various user interfaces (e.g., PUI, VUI, and/or GUI) (S9000).

In step S9000, if affirmative, the SleepTrack app is activated (S9100). If negative, the operation may be terminated as the introduction of the SleepTrack app would be meaningless.

Exemplarily, smart home-appliances reaching step S8100 may include air conditioners and/or air purifiers for temperature control, humidifiers and/or dehumidifiers for humidity control, blinds and/or curtains, lights for light control, smart speakers 804 for sound control, smart beds for adjusting the user's head and body position, smart diffusers for scent control, and smart devices with healthcare apps installed.

Additionally, smart home-appliances reaching step S9100 may include TVs, clothing care devices, robotic vacuum cleaners, washing machines and/or dryers, refrigerators, and smart devices with healthcare apps installed.

Furthermore, application fields that can reach “Sleep Management App Interaction” beyond steps S8100 and S9100 may include industries related to scent, cosmetics, health functional foods, traditional sleep industry, sports, hotels, cram schools, fire stations, and government agencies.

Here, the “Sleep Management App” refers to a type of sleep management app capable of sleep analysis without hardware solutions.

Additionally, the “SleepTrack App” may refer to a sleep analysis app that delivers the user's sleep report to the user's smartphone 900 in real-time through PUI, VUI, and/or GUI, and operates smart home-appliances 800 based on the report results.

The steps illustrated in the aforementioned drawings may have their order changed as needed, and at least one step may be omitted or added. That is, the aforementioned steps are merely one embodiment of the present invention, and the scope of rights of the present invention is not limited thereto.

Hereinafter, the step of determining environment adjustment information will be described in detail by dividing it into sleep state and sleep stage using the processor 130 as an example. Additionally, examples of smart home-appliances 800 operating according to environment adjustment information will be described in detail. However, the following description is not limited to the embodiments described, and the present invention is not limited thereto.

### Determination of Sleep Induction Timing

According to an embodiment, the processor 130 can determine the sleep induction timing based on sleep intention information. Alternatively, in the case of an embodiment such as that shown in FIG. 1(c), at least one of the electronic devices depicted in FIG. 1(c) may also determine the sleep induction timing. Specifically, the processor 130 can identify the point at which the sleep intention information exceeds a predetermined threshold score as the sleep induction timing. That is, when high sleep intention information is acquired, the processor 130 can identify this as the appropriate timing for sleep induction, i.e., the sleep induction timing.

As described above, the processor 130 can determine the user's sleep induction timing. According to an embodiment, when the processor 130 acquires the first sleep state information indicating that the user is before sleep, it can generate the first environment adjustment information to adjust the light from the sleep induction timing to the point where the second sleep state information is acquired (supplying white light at 3000K with an illumination of 30 lux).

### First Environment Adjustment Information and Operation of Smart Home-Appliance Based on Pre-Sleep State

According to an embodiment of the present invention, when the user's state is in the pre-sleep state, the processor 130 can generate the first environment adjustment information to adjust the light from the predicted timing when the user is preparing for sleep (e.g., sleep induction timing) to the point of falling asleep (i.e., when the second sleep state information is acquired), and decide to transmit the first environment adjustment information to the environment adjustment device 30.

Alternatively, in the case of an embodiment such as that shown in FIG. 1(c), at least one of the electronic devices depicted in FIG. 1(c) may perform at least one of the aforementioned operations.

Accordingly, from 20 minutes before the user falls asleep (e.g., sleep induction timing) to the moment of falling asleep, white light at 3000K can be supplied with an illumination of 30 lux. This light is excellent for melatonin secretion before the user falls asleep and naturally induces sleep, thereby enhancing the user's sleep efficiency.

Additionally, according to an embodiment, when the user's state is in the pre-sleep state, the processor 130 can generate the first environment adjustment information to control the smart home-appliance from the predicted timing when the user is preparing for sleep (e.g., sleep induction timing) to the point of falling asleep (i.e., when the second sleep state information is acquired).

Specifically, the first environment adjustment information can be generated to pre-remove fine dust and harmful gases or control the indoor temperature and humidity for sleep induction until a predetermined time before the user's sleep (e.g., 20 minutes prior). Additionally, the first environment adjustment information may include controlling the smart home-appliance to induce a level of noise (white noise) conducive to sleep just before sleep, adjusting the blower intensity of smart home-appliances such as air purifiers or air conditioners to below a predefined intensity, reducing the intensity of LEDs, or converting direct airflow to indirect airflow. Furthermore, the first environment adjustment information may include information to control smart home-appliances to execute dehumidification/humidification based on temperature and humidity information within the sleep space. Moreover, the first environment adjustment information may include control information to adjust personalized temperature, humidity, blower intensity, and noise based on the operation history of smart home-appliances such as air purifiers or air conditioners and the acquired sleep state (quality of sleep).

According to an embodiment of the present invention, when the user's state is in the pre-sleep state, smart home-appliances can operate according to the first environment adjustment information from the predicted timing when the user is preparing for sleep (e.g., sleep induction timing) to the point of falling asleep (i.e., when the second sleep state information is acquired). Various operations of smart home-appliances will be explained below as examples.

For example, at the stage where the user is preparing for sleep, such as the predicted time when the user is preparing for sleep or the time when the user intends to sleep, the lights installed in the bedroom, living room, kitchen, bathroom, etc., can detect the user's presence through an embedded motion sensor. Additionally, a healthcare app can initiate the user's sleep measurement.

A TV according to an embodiment of the present invention can provide user-optimized sleep content. Alternatively, it can set the screen-off time. Here, the user-optimized sleep content may include mindfulness, guided imagery, ASMR, counting numbers backward, counting sheep, etc.

An air conditioner and/or air purifier according to an embodiment of the present invention can adjust the indoor temperature for the user's sleep onset. Additionally, the type of air provided can be converted to indirect airflow.

A humidifier and/or dehumidifier according to an embodiment of the present invention can be activated in a low-noise state. It can also maintain an appropriate humidity level.

A refrigerator according to an embodiment of the present invention can recommend sleep-promoting foods (e.g., warm milk, chamomile, etc.) based on the analysis of the user's personal bedtime or guide the user to avoid late-night snacks.

A clothing management device according to an embodiment of the present invention can switch to a low-noise mode or have the sleep start time set so that it operates immediately upon waking.

Blinds and/or curtains according to an embodiment of the present invention can automatically close, and sleep lights among the lights can be switched to a dim light. All other lights can be set to turn off.

Additionally, according to an embodiment of the present invention, at the time the user falls asleep, the healthcare app can recognize the user's sleep onset. The TV can continue to provide sound-related content from the user-optimized sleep content, and the screen can be set to turn off.

### Second Environment Adjustment Information and Operation of Smart Home-Appliances Based on the Second Sleep State

According to an embodiment of the present invention, the processor 130 can generate second environment adjustment information based on the second sleep state information.

For example, the processor 130 can identify the time when the user is entering sleep, i.e., the sleep onset time, through the second sleep state information, and generate second environment adjustment information based on this.

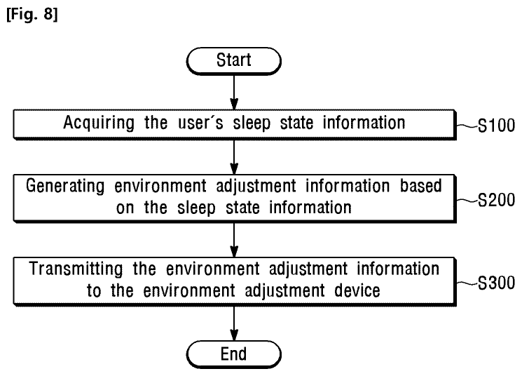

For instance, as illustrated in FIG. 7, the processor 130 may generate second environment adjustment information to minimize light from the onset of sleep or control smart home-appliances to sleep mode to optimize temperature and humidity, thereby creating an atmosphere akin to a quiet darkroom. This second environment adjustment information has the effect of enhancing the quality of sleep by allowing the user to fall into a deep sleep.

In an embodiment, the processor 130 can generate external environment adjustment information based on sleep stage information. In this embodiment, the sleep stage information may include information regarding the user's sleep stage changes acquired in a time-series manner through the analysis of sleep sound information.

Alternatively, in the case of an embodiment such as (c) of FIG. 1, at least one of the electronic devices shown in (c) of FIG. 1 may perform the above operation.

The second environment adjustment information may be control information that minimizes illumination to create a darkroom environment without light. For example, if there is light interference during sleep, the probability of fragmented sleep increases, making it difficult to achieve good sleep.

Additionally, the processor 130 may generate second environment adjustment information to control smart home-appliances by lowering the brightness of the display unit to a predetermined brightness, turning off the display unit, operating at noise levels below a predetermined level, adjusting the blowing strength to below a predetermined intensity, setting the blowing temperature within a predetermined range, maintaining the humidity in the sleep space at a predetermined temperature, or maintaining gentle airflow.

According to the present invention, the second environment adjustment information may include control information for operating smart home-appliances to improve air quality in the indoor space or optimize temperature and humidity, especially when the user is in deep sleep, as there is less concern about waking up.

That is, when the processor 130 detects that the user has entered sleep (or a sleep stage) and acquires second sleep state information, it can generate second environment adjustment information to prevent light supply or control the operation of smart home-appliances. Consequently, the probability of the user achieving deep sleep increases, thereby improving sleep quality.

Furthermore, as a specific example, when the processor 130 identifies that the user has entered a sleep stage (e.g., light sleep) through the user's sleep stage information, it can generate external environment adjustment information to optimize indoor temperature and humidity, minimize illumination to create a darkroom environment, or control smart home-appliances to perform tasks such as removing fine dust/harmful gases, adjusting air temperature and humidity, lighting LEDs, controlling operation noise levels, and adjusting airflow, thereby facilitating restful sleep. By creating the optimal illumination for each sleep stage of the user, i.e., the optimal sleep environment, the user's sleep efficiency can be improved.

Additionally, the processor 130 can generate environment adjustment information to provide appropriate illumination or adjust air quality according to changes in the user's sleep stage during sleep. For example, when transitioning from light sleep to deep sleep, it may supply subtle red light, or when transitioning from REM sleep to light sleep, it may lower illumination or supply blue light, thereby generating more diverse external environment adjustment information according to sleep stage changes. This approach considers not only the situation before sleep or immediately after waking but also the entire sleep experience, thereby maximizing the user's sleep quality.

Below, various operations of smart home-appliances based on second sleep state information are described as examples.

A healthcare app according to an embodiment of the present invention can analyze a user's breathing sounds in real-time and provide stimuli such as vibrations or alarms during apnea.

A TV according to an embodiment of the present invention can turn off the screen and mute the sound.

An air conditioner and/or air purifier according to an embodiment of the present invention can maintain an appropriate indoor temperature and indirect airflow. Additionally, it can adjust the temperature upon detecting light sleep due to temperature changes.

A humidifier and/or dehumidifier according to an embodiment of the present invention can maintain a low-noise mode and appropriate humidity.

A door lock according to an embodiment of the present invention can confirm the locked state.

An outlet and/or switch according to an embodiment of the present invention can switch to a low-power mode.

Among the lights according to an embodiment of the present invention, a sleep light can be turned off at a predefined time (e.g., 15 to 25 minutes later) from the point when the user's falling asleep is recognized.

Meanwhile, during sleep mode, sleep stages can be further classified into options such as basic sleep mode, personalized sleep mode, and special care mode.

The basic sleep mode can provide an environment (air, temperature, humidity, light, scent, etc.) that creates a comfortable sleep environment by setting it as the default value for sleep mode.

The personalized sleep mode can provide a customized sleep mode based on accumulated user data according to the quality of the user's sleep.

The special care mode can develop and provide an optimized customized sleep mode for unique users experiencing discomfort during sleep, such as itching or being overweight.

Alternatively, in an embodiment such as that shown in FIG. 1(c), at least one of the electronic devices depicted in FIG. 1(c) may perform at least one of the aforementioned operations.

### Third Environment Adjustment Information and Operation of Smart Home-Appliances Based on Wake-Up Induction Timing

According to one embodiment of the present invention, the processor 130 can generate third environment adjustment information based on the wake-up induction timing. Alternatively, in an embodiment such as that shown in FIG. 1(c), at least one of the electronic devices depicted in FIG. 1(c) may also generate the third environment adjustment information.

For example, the processor 130 can identify the user's wake-up time through sleep planning information, generate a wake-up prediction time based on the identified wake-up time, and accordingly generate environment adjustment information. For instance, as shown in FIG. 7, the processor 130 can generate third environment adjustment information to gradually increase the illumination from 0 lux to 250 lux with 3000K white light based on the bed position starting 30 minutes before the wake-up prediction time. This third environment adjustment information can guide the user to wake up naturally and refreshed at the desired wake-up time.

Additionally, the processor 130 can decide to transmit the environment adjustment information to the environment adjustment device 30. That is, the processor 130 can enhance the user's sleep quality by generating external environment adjustment information based on sleep planning information, allowing the user to fall asleep easily or wake up naturally during sleep or wake-up times.

In an additional embodiment, the processor 130 can generate recommended sleep planning information based on sleep stage information. Specifically, the processor 130 can acquire information on the user's sleep stage changes (e.g., sleep cycle) through sleep stage information and set an expected wake-up time based on this information.

For example, a typical sleep cycle during the day may include light sleep, deep sleep, light sleep, and REM sleep stages. The processor 130 can determine that the time after REM sleep is when the user can wake up most refreshed and generate recommended sleep planning information by deciding the wake-up time after the REM stage. Furthermore, the processor 130 can generate environment adjustment information according to the recommended sleep planning information and decide to transmit it to the environment adjustment device 30. Therefore, the user can wake up naturally according to the recommended sleep planning information suggested by the processor 130. This recommendation by the processor 130 based on the user's sleep stage changes can minimize the user's fatigue, thereby improving the user's sleep efficiency.

As described above, the third environment adjustment information can be characterized as control information that gradually increases the illumination from 0 lux to 250 lux with 3000K white light from the wake-up induction time to the wake-up time. For example, the third environment adjustment information may relate to control information about gradually increasing the illumination starting 30 minutes before the user's wake-up time (i.e., wake-up induction time). Here, the wake-up induction time can be characterized as being determined based on the wake-up prediction time.

In one embodiment, the wake-up induction time can be characterized as being determined based on the wake-up prediction time. The wake-up prediction time may be information regarding the time the user is expected to wake up. For example, the wake-up prediction time may be 7 a.m. for the first user. The specific description of the aforementioned wake-up prediction time or numerical values is merely exemplary and not limiting to the present invention.

The third environment adjustment information may include information for controlling a smart home-appliance to induce awakening by increasing or decreasing at least one of indoor temperature, humidity, airflow intensity, noise, or vibration at the time of awakening.

Additionally, the third environment adjustment information may include control information for controlling a smart home-appliance to generate white noise to gradually induce awakening.

According to the present invention, the third environment adjustment information may include control information for maintaining the noise of the smart home-appliance below a predefined level after awakening.

Furthermore, the third environment adjustment information may include control information for controlling a smart home-appliance in conjunction with the awakening prediction time and the awakening recommendation time. The awakening recommendation time may be a time automatically extracted based on the user's sleep pattern, and the awakening prediction time will be described in detail later.

Hereinafter, various operations of smart home-appliances in awakening mode based on the awakening induction time and the awakening time will be described by way of example.

In the pre-awakening stage, a healthcare app according to an embodiment of the present invention may conduct a sleep analysis of the user and recognize the user's sleep pattern.

An air conditioner and/or air purifier according to an embodiment of the present invention may adjust the environment, such as indoor air quality, temperature, or humidity, to facilitate the user's awakening.

In the awakening stage, a healthcare app according to an embodiment of the present invention may activate a smart alarm installed in the app if the user's REM sleep is detected or if a change in the user's body temperature is sensed.

A humidifier and/or dehumidifier according to an embodiment of the present invention may switch to a general operation mode.

A clothing management device according to an embodiment of the present invention may initiate operation in accordance with a pre-set awakening alarm time during the preparation-for-sleep stage.

Blinds and/or curtains according to an embodiment of the present invention may automatically open.

In one embodiment of the present invention, a washing machine can initiate a washing operation.

Additionally, in one embodiment of the present invention, a dryer can initiate a drying operation.

In the post-awakening stage, a healthcare app according to one embodiment of the present invention can display the analyzed user's sleep report on the user's smartphone 900 and provide user-optimized content such as today's weather and major news.

An apparel management device according to one embodiment of the present invention can complete tasks such as caring for dust or wrinkles on clothing, deodorization, sterilization, and drying in accordance with a predefined departure time.

A robotic vacuum cleaner according to one embodiment of the present invention can, if necessary, transmit a report to the user's smartphone 900 at this stage, secure, analyze, and reflect user data, and complete the washing operation of the washing machine and the drying operation of the dryer before the user's departure, subsequently securing, analyzing, and reflecting user data.

A water purifier according to one embodiment of the present invention can dispense automatically customized water reflecting the user's preferences, and subsequently secure, analyze, and reflect user data.

A refrigerator according to one embodiment of the present invention can display a list of recommended and non-recommended breakfast menus and a list of recommended morning exercises on the display unit based on the analyzed user's sleep and health data installed on the front.

An oven/microwave according to one embodiment of the present invention can automatically preheat a selected menu from the recommended breakfast menus suggested by the refrigerator, and subsequently secure, analyze, and reflect user data.

Furthermore, by securing, analyzing, and reflecting user data from all smart home-appliances 800, the optimal sleep environment (temperature, humidity, air quality, illumination, etc.) based on personal data can be recommended.

Alternatively, in the case of an embodiment such as that shown in FIG. 1(c), at least one of the electronic devices depicted in FIG. 1(c) may perform at least one of the aforementioned operations.

### Determination of Wake-Up Prediction Time

In one embodiment, the wake-up prediction time may be predetermined through information exchange with the user's user terminal 10. Specifically, the user can set the desired wake-up time via the user terminal 10 and transmit it to the processor 130. That is, the processor 130 can acquire the wake-up prediction time based on the time set by the user of the user terminal 10. For example, if the user sets an alarm time through the user terminal 10, the processor 130 can determine the set alarm time as the wake-up prediction time.

In another embodiment, the wake-up prediction time may be determined based on the sleep onset time identified through the second sleep state information. Specifically, the processor 130 can ascertain the user's sleep onset time through the second sleep state information indicating that the user is asleep. The processor 130 can determine the wake-up prediction time based on the sleep onset time identified through the second sleep state information. For instance, the processor 130 can determine the wake-up prediction time as a point 8 hours after the sleep onset time, which is considered an appropriate sleep duration. For example, if the sleep onset time is 11 PM, the processor 130 can determine the wake-up prediction time as 7 AM. The specific numerical descriptions for each time mentioned above are merely exemplary and do not limit the invention. That is, the processor 130 can determine the wake-up prediction time based on the time the user falls asleep.

In yet another embodiment, the wake-up recommendation time may be determined based on the user's sleep stage information. For example, a user may wake up feeling most refreshed when waking during the REM stage. During a night's sleep, a user may cycle through light sleep, deep sleep, light sleep, and REM sleep, and waking during the REM sleep stage can result in the most refreshing wake-up. Preferably, considering the user's appropriate or desired sleep time, the wake-up recommendation time can be determined to at least satisfy the appropriate or desired sleep time.

Accordingly, the processor 130 can determine the user's wake-up prediction time through sleep stage information related to the user's sleep stages. For example, the processor 130 can determine the wake-up recommendation time as the point when the user transitions from the REM stage to another sleep stage (preferably, just before transitioning from the REM stage to another sleep stage) through the sleep stage information. That is, the processor 130 can determine the wake-up prediction time based on the sleep stage information (i.e., the REM sleep stage) where the user can wake up most refreshed.

As described above, the processor 130 can determine the user's wake-up prediction time based on at least one of user settings, sleep onset time, and sleep stage information. Additionally, when the processor 130 determines the wake-up prediction time, which is the time the user wishes to wake up, it can determine the wake-up induction time based on the wake-up prediction time. For example, the processor 130 can determine the wake-up induction time as 30 minutes before the time the user wishes to wake up. For instance, if the wake-up prediction time set by the user is 7 AM, the processor 130 can determine the wake-up induction time as 6:30 AM. The specific descriptions of the times mentioned above are merely exemplary and do not limit the invention.

That is, the processor 130 can determine the wake-up induction time by identifying the wake-up prediction time when the user's wake-up is expected, and generate the third environment adjustment information to gradually increase the supply of 3000K white light from 0 lux to 250 lux from the wake-up induction time until the wake-up time (e.g., until the user actually wakes up). The processor 130 can decide to transmit the third environment adjustment information to the environment adjustment device 30, and accordingly, the environment adjustment device 30 can perform light-related adjustment operations in the space where the user is located based on the third environment adjustment information.

Alternatively, in the embodiment shown in FIG. 1(c), at least one of the electronic devices depicted in FIG. 1(c) can perform at least one of the aforementioned operations. For example, the environment adjustment device 30 can control the light supply module to gradually increase the 3000K white light from 0 lux to 250 lux starting 30 minutes before the wake-up time. The specific numerical descriptions mentioned above are merely exemplary and do not limit the invention.

### Fourth Environment Adjustment Information and Operation of Smart Home-Appliance Based on Third Sleep State Information

According to one embodiment of the present invention, the processor 130 can acquire the fourth environment adjustment information based on the third sleep state information. Specifically, the processor 130 can acquire the user's sleep disorder information. In one embodiment, the sleep disorder information may include delayed sleep phase syndrome. Delayed sleep phase syndrome is a sleep disorder symptom where the ideal sleep time is delayed, preventing the user from falling asleep at the desired time. According to the embodiment, blue-light therapy is one treatment method for delayed sleep phase syndrome, where blue light is supplied for about 30 minutes after the user wakes up at the desired wake-up time. Repeating this blue light supply every morning can restore the circadian rhythm to its original state, preventing the user from feeling sleepy later at night compared to normal individuals.

Accordingly, the processor 130 can generate fourth environment adjustment information based on sleep disorder information and third sleep state information. For example, if the user terminal 10 acquires sleep disorder information indicating that the user has delayed sleep phase syndrome and third sleep state information indicating that the user is post-sleep (i.e., awake), the processor 130 can generate fourth environment adjustment information. In this case, the fourth environment adjustment information may be control information to supply blue light with an intensity of 300 lux, a color temperature of 221 degrees, 100% saturation, and 56% brightness for a predefined time from the wake-up time. In one embodiment, blue light with an intensity of 300 lux, a color temperature of 221 degrees, 100% saturation, and 56% brightness may be intended to treat delayed sleep phase syndrome. Specifically, if a user with delayed sleep phase syndrome wakes up at 7 AM, the processor 130 can determine the wake-up time as 7 AM based on the third sleep state information and generate fourth environment adjustment information to supply blue light with the specified parameters from 7 AM to a predefined time (e.g., 7:30 AM). Consequently, the user's circadian rhythm can be adjusted to a normal range (e.g., falling asleep around midnight and waking up around 7 AM). Thus, through the generation of fourth environment adjustment information, the quality of sleep for a user with a specific sleep disorder can be improved.

According to one embodiment of the present invention, the processor 130 can decide to transmit the environment adjustment information to the environment adjustment device. Specifically, the processor 130 can generate environment adjustment information related to light intensity adjustment and decide to transmit the information to the environment adjustment device 30, thereby controlling the light intensity adjustment operation of the environment adjustment device 30.

According to the embodiment, light can be one of the representative factors affecting sleep quality. For instance, depending on the intensity, color, and exposure level of light, it can have a positive or negative impact on sleep quality. Accordingly, the processor 130 can enhance the user's sleep quality by adjusting the light intensity. For example, the processor 130 can monitor the situation before or after the user falls asleep and perform light intensity adjustments to effectively wake the user. That is, the processor 130 can automatically adjust the light intensity by identifying the sleep state (e.g., sleep stage) to maximize sleep quality.

Alternatively, in the case of an embodiment like (c) of FIG. 1, at least one of the electronic devices shown in (c) of FIG. 1 may perform at least one of the aforementioned operations. The descriptions of the figures and times mentioned above are merely exemplary, and the present invention is not limited thereto.

### Environment Adjustment Information According to Detected Events

One embodiment of the present invention can generate environment adjustment information to control the environment adjustment device based on at least one detected event.

The generation of environment adjustment information according to one embodiment of the present invention can be performed by the computing device 100 shown in (a) of FIG. 1 or the sleep environment adjustment device 400 shown in (b) of FIG. 1.

Events can be variably preset to include at least one or more. For example, events can include at least one of the following events A to H.
A. In-room
B. In-bed
C. Fall Asleep
D. Apnea
E. Deep Sleep
F. Wake Up During Sleep
G. REM Near Alarm
H. Wake Up

Hereinafter, each event will be described in detail.

The aforementioned Event A refers to an event where the user enters a space for sleeping, such as a bedroom. Event A can be detected through a presence detection sensor.

The presence detection sensor, also known as a human-body detection sensor, may include, for example, a radar sensor, PIR motion sensor, WiFi sensing sensor, camera sensor, or ultrasonic sensor.

The presence detection sensor may be mounted on the environment adjustment device 30 or separately installed in the bedroom and connected to the environment adjustment device 30 via wired or wireless means. Alternatively, the presence detection sensor may be connected to the network of FIG. 1(a) or FIG. 1(b) to transmit detection signals to the computing device 100, sleep environment adjustment device 400, user terminal 10, or environment adjustment device 30. Additionally, the presence detection sensor may be connected to the user terminal 10 through short-range communication to transmit detection signals to the user terminal 10.

Alternatively, when the present invention is implemented in the embodiment shown in FIG. 1(c), the presence detection sensor may be present in at least one of the electronic devices depicted in FIG. 1(c).

Upon the occurrence of Event A, that is, when Event A is detected by the presence detection sensor, environment adjustment information A may be generated to automatically turn on the environment adjustment device 30. Here, environment adjustment information A may include control information for changing the environment adjustment device 30 to a specific operation mode in addition to automatically turning it on.

Event B refers to an event where the user lies on the bed. Event B can be detected through a piezoelectric sensor. The piezoelectric sensor can be mounted on a bed where the user sleeps. However, it is not limited to this, and the piezoelectric sensor can also be mounted on a sofa or massage chair where the user can sleep.

The piezoelectric sensor may be connected to the environment adjustment device 30 either wired or wirelessly. Alternatively, the piezoelectric sensor may be connected to the network shown in FIG. 1(a) or FIG. 1(b) to transmit detection signals to the computing device 100, sleep environment adjustment device 400, user terminal 10, or environment adjustment device 30. Alternatively, the piezoelectric sensor may be connected to the user terminal 10 via short-range communication to transmit detection signals to the user terminal 10.

Upon the occurrence of Event B, that is, when Event B is detected by the piezoelectric sensor, environment adjustment information B may be generated to drive the environment adjustment device 30 into sleep mode. Environment adjustment information B may include control information for the environment adjustment device 30 to facilitate the user's falling asleep. The control information may include information to reduce noise or light generated by the environment adjustment device 30. For example, if the environment adjustment device 30 is an air conditioner, it may include information to change the airflow to below a certain intensity, reduce the current airflow to below the specified intensity, convert direct airflow to indirect or no airflow, or lower the brightness of the display unit to below a predetermined level.

Additionally, it may include information to turn off or dim the lights installed in the bedroom to below a certain brightness. It may also include information to close curtains or blinds installed in the bedroom to eliminate external sleep disturbances. Furthermore, it may include information to turn on a sound device installed in the bedroom to play specific audio sources, or conversely, to turn off the sound device. It may also include information to change the motion of a motion bed installed in the bedroom to a specific motion conducive to pre-sleep reading or media viewing. Additionally, it may include information to operate a scent-providing device installed in the bedroom to emit a scent that aids in falling asleep.

Event C refers to an event where the user enters sleep onset (or ingress). As previously described, Event C can be determined by the computing device 100 or sleep environment adjustment device 400 that receives environment sensing information sensed by the user terminal 10. As shown in FIG. 7, the sleep onset (or ingress) time can be determined through the environment sensing information sensed by the user terminal 10.

Upon the occurrence of Event C, that is, when Event C is detected, environment adjustment information C may be generated to drive the environment adjustment device 30 into sleep mode. Environment adjustment information C may include control information to create the optimal bedroom sleep environment. Here, the optimal bedroom sleep environment may be optimal environment information obtained based on paired data (temperature and/or humidity & sleep quality) acquired over a predetermined period (e.g., a week or a month). For example, based on quantitative data representing the sleep quality of the user over the past week and the temperature and humidity data of the bedroom during which the quantitative data was obtained, the temperature and humidity of the bedroom where the user slept best can be determined as the optimal bedroom sleep environment.

The control information may involve setting the temperature and humidity of the bedroom to the optimal temperature and humidity using the environment adjustment device 30. It may also include information to turn off the lights installed in the bedroom. Additionally, it may include information to turn off the sound device installed in the bedroom. Furthermore, it may include information to change the motion of a motion bed installed in the bedroom to a specific motion conducive to deep sleep. Additionally, it may include information to operate a scent-providing device installed in the bedroom to emit a scent that aids in deep sleep or to turn off the scent-providing device.

Event D refers to an event where sleep apnea or respiratory depression occurs during the user's sleep. As previously described, Event D can be determined by the computing device 100 or sleep environment adjustment device 400 that receives environment sensing information sensed by the user terminal 10. As shown in FIG. 4, the occurrence time of sleep apnea or respiratory depression can be determined through the environment sensing information sensed by the user terminal 10.

Upon the occurrence of the D event, that is, when the D event is detected, D environment adjustment information for driving the sleep environment adjustment device 30 into sleep mode may be generated. The D environment adjustment information may include control information to alleviate sleep apnea or reduced breathing, or to quickly transition stopped or weak breathing to normal breathing. For example, the control information may include information to increase the set humidity or temperature, or to convert direct or indirect airflow to no airflow when the sleep environment adjustment device 30 is an air conditioner, in order to protect the airway and throat of a user experiencing sleep apnea.

Alternatively, if the sleep environment adjustment device 30 includes a humidification function, it may include information to activate this function. If the sleep environment adjustment device 30 includes a vibration function, it may include information to activate this function. Additionally, it may include information to adjust the lighting installed in the bedroom to a specific brightness and color temperature. It may also include information to turn on a sound device installed in the bedroom. Furthermore, it may include information to change the motion of a motion bed installed in the bedroom to a specific motion that assists the user's breathing. Additionally, it may include information to activate a scent-providing device installed in the bedroom to emit a scent that can alleviate sleep apnea.

The E event indicates an event where the user has entered deep sleep. As previously described, the E event can be determined by a computing device 100 or a sleep environment adjustment device 400 that has received environment sensing information sensed by the user terminal 10. As shown in FIG. 3, the point of entry into deep sleep can be determined through the environment sensing information sensed by the user terminal 10.

When the E event occurs, that is, when the E event is detected, E environment adjustment information for driving the sleep environment adjustment device 30 into sleep mode may be generated. The E environment adjustment information may include control information to change to a temperature or humidity optimized for the deep sleep stage. For example, the control information may include information to change the current bedroom temperature or humidity to the optimized temperature or humidity when the sleep environment adjustment device 30 is an air conditioner.

Here, the optimized temperature or humidity may be determined as the specific temperature or humidity at which the user sustained deep sleep the longest, using quantitative sleep reports obtained over a predetermined period. It may also include information to turn off or dim the lighting installed in the bedroom to a minimal brightness. Additionally, it may include information to turn off a sound device installed in the bedroom. Furthermore, it may include information to change the motion of a motion bed installed in the bedroom to a specific motion favorable for deep sleep. Additionally, it may include information to activate a scent-providing device installed in the bedroom to emit a scent that can maintain restful sleep.

The F event indicates an event where the user has woken up during sleep. As previously described, the F event can be determined by a computing device 100 or a sleep environment adjustment device 400 that has received environment sensing information sensed by the user terminal 10. As shown in FIG. 3, the wake-up point can be determined through the environment sensing information sensed by the user terminal 10.

When the F event occurs, that is, when the F event is detected, F environment adjustment information for driving the sleep environment adjustment device 30 into sleep mode may be generated. The F environment adjustment information may include control information to help the user fall back asleep. For example, the control information may include information to change the air conditioner's set temperature and humidity to the preferred temperature or humidity that the user typically set when falling asleep, if the sleep environment adjustment device 30 is an air conditioner.

Alternatively, the control information may include information to change the set temperature or humidity of the sleep environment adjustment device 30 to the specific temperature or humidity at which the user's time to fall asleep was shortest, using past quantitative sleep reports. It may also include information to adjust the lighting installed in the bedroom to a specific brightness and color temperature conducive to falling asleep. Additionally, it may include information to turn on or off a sound device installed in the bedroom.

Furthermore, it may include information to change the motion of a motion bed installed in the bedroom to a specific motion that aids the user's re-entry into sleep. Additionally, it may include information to activate a scent-providing device installed in the bedroom to emit a scent that aids in re-entering sleep.

The G event indicates an event where REM sleep occurs near a pre-set alarm time. As previously described, the G event can be determined by a computing device 100 or a sleep environment adjustment device 400 that has received environment sensing information sensed by the user terminal 10. As shown in FIG. 3, the REM sleep point can be determined through the environment sensing information sensed by the user terminal 10.

When the G event occurs, that is, when the G event is detected, the G environment adjustment information for driving the environment adjustment device 30 into sleep mode may be generated. The G environment adjustment information may include control information to assist the user in waking up. For example, if the environment adjustment device 30 is an air conditioner, the control information may include information for changing the set temperature or humidity of the air conditioner to a specific temperature or humidity that allows the user to wake up naturally or most refreshingly.

Alternatively, the control information may include information for changing the set temperature or humidity of the air conditioner to a specific temperature or humidity preferred by the user, using past quantitative sleep reports. Additionally, it may include information to illuminate the lighting installed in the bedroom to a specific brightness and color temperature specialized for waking up.

Furthermore, it may include information to open curtains or blinds installed in the bedroom. It may also include information to turn on a sound device installed in the bedroom to play a specific sound source. Additionally, it may include information to change the motion of a motion bed installed in the bedroom to a specific motion beneficial for waking up. Moreover, it may include information to activate a scent-providing device installed in the bedroom to emit a fragrance that facilitates a refreshing wake-up.

The H event refers to an event indicating the user's wake-up time. As described above, the H event can be determined by a computing device 100 or a sleep environment adjustment device 400 that receives environment sensing information sensed by the user terminal 10. As shown in FIG. 5, the wake-up time can be determined by identifying whether a predetermined pattern is continuously detected after a singularity 201 is identified in the environment sensing information sensed by the user terminal 10.

When the H event occurs, that is, when the H event is detected, the H environment adjustment information for driving the environment adjustment device 30 into sleep mode may be generated. The H environment adjustment information may include control information for setting the temperature of the bedroom where the user slept to the optimal temperature after waking up. If the environment adjustment device 30 is an air conditioner, the control information may include information for changing the set temperature or humidity of the air conditioner to the temperature or humidity preferred by the user at the wake-up time based on past history.

Alternatively, the control information may include suggestion information for changing the set temperature or humidity of the air conditioner to a recommended temperature or humidity after waking up through the user terminal. Additionally, it may include information to illuminate the lighting installed in the bedroom to a specific brightness and color temperature. It may also include information to turn on a sound device installed in the bedroom to display specific media or play a specific sound source. Furthermore, it may include information to open windows installed in the bedroom for ventilation. Additionally, it may include information to change the motion of a motion bed installed in the bedroom to a specific motion that helps the user get up. Moreover, it may include information to activate a scent-providing device installed in the bedroom to emit a fragrance that aids the user's movement after waking up.

Furthermore, if the embodiment of the present invention is, for example, as shown in FIG. 1(c), at least one of the electronic devices shown in FIG. 1(c) may perform at least one of the aforementioned operations.

### Sleep Plan Information

In one embodiment, the processor 130 may receive sleep plan information from the user terminal 10. The sleep plan information is information generated by the user through the user terminal 10 and may include information regarding bedtime and wake-up time. The processor 130 may generate external environment adjustment information based on the sleep plan information. For a specific example, the processor 130 may identify the user's bedtime through the sleep plan information and generate external environment adjustment information based on the identified bedtime. Alternatively, if the embodiment is as shown in FIG. 1(c), at least one of the electronic devices shown in FIG. 1(c) may perform at least one of the aforementioned operations.

For example, as shown in FIG. 7, the processor 130 may generate first environment adjustment information to provide white light of 3000K at an illuminance of 30 lux based on the bed position 20 minutes before bedtime. That is, it may create an illuminance that naturally induces the user to fall asleep in relation to bedtime. The aforementioned figures and timing are merely examples, and the present invention is not limited thereto.

### Example 1 of a Method for Preventing and Mitigating Sleep Disorders

FIG. 36(a) is a flowchart illustrating a method for preventing and mitigating sleep disorders using an AI-based non-contact sleep analysis system according to an embodiment of the present invention.

The present invention allows for the real-time analysis of a user's sleep, identifying points where sleep disorders (such as sleep apnea, sleep-related breathing disorders, and hypopnea) occur. By providing stimuli (tactile, auditory, olfactory, etc.) to the user at the moment a sleep disorder occurs, the disorder can be temporarily alleviated.

In other words, the present invention can interrupt the user's sleep disorder and reduce its frequency based on accurate event detection related to sleep disorders.

Referring to FIG. 36(a), the method for preventing and mitigating sleep disorders using a smart home-appliance 800 according to the present invention involves collecting the user's sleep sound information and performing primary and secondary sleep analyses based on this information.

The primary sleep analysis is based on the user's sleep sound information, while the secondary sleep analysis is based on the results of the primary sleep analysis and the sleep sound information, with the specific analysis methods being as described above.

As a result of the primary and secondary sleep analyses, when it is determined that sleep apnea has occurred in the user, the smart home-appliance 800 can generate at least one of tactile feedback and auditory feedback. The smart home-appliance 800 may further include an alarm unit 850 for feedback, which can be implemented as an actuator generating vibrations, a vibration module, a haptic module, or a speaker module generating sound or audio.

According to the present invention, vibrations transmitted to the body part in contact with the smart home-appliance (e.g., the entire body in the case of a smart mattress) or sounds and audio resonating near the ear (e.g., from a smart speaker 804, smartphone, smart TV, etc.) stimulate the user's brain, thereby relatively quickly alleviating sleep apnea. If such processes continue during the user's sleep, the frequency of sleep apnea is significantly reduced.

At this time, not only can a single sleep apnea event be detected, but clusters of continuously occurring sleep apnea events can also be predicted in advance. To this end, the aforementioned sleep analysis learning model can be trained to predict clusters of continuous sleep apnea events.

That is, input information based on the user's sleep sound information, as described above, undergoes a pre-processing stage and a Mel spectrogram conversion process before being input into the input layer, allowing the trained sleep analysis learning model to predict clusters of continuous sleep apnea events.

According to the present invention, if a cluster where sleep apnea events occur consecutively is predicted in advance, it is possible to prevent sleep apnea in advance or alleviate or improve it by vibrating the smartphone 900 one or more times not only at the moment when the sleep apnea event is detected but also at the pre-predicted time.

That is, the present invention allows for the analysis of sleep stages based on sleep sound information signals, thereby enabling the alleviation and improvement of sleep apnea.

According to the present invention, the pattern of tactile and auditory feedback applied to the user may be intended to reduce the frequency of sleep apnea while maintaining the user's deep sleep. This pattern can be adjusted in real-time based on the analysis results of the user's sleep stages.

Additionally, this pattern may be inferred through a deep learning model trained on big data related to the analysis results of the user's sleep stages and big data related to the frequency of sleep apnea.

Although sleep disorders such as sleep apnea and hyperventilation have been mentioned above, to improve the quality of sleep, stimuli may be delivered to the user through a smart home-appliance 800 if it is determined to be the REM sleep stage.

According to the present invention, REM sleep is a sleep stage where brain waves become rapid, and autonomic activities such as heart rate and respiration are irregular, accompanied by slight involuntary muscle spasms or rapid eye movements. It generally occurs 3 to 4 times at intervals of approximately 80 to 120 minutes, but in severe cases, it can develop into REM sleep disorder and affect the quality of sleep.

Therefore, not only for sleep disorders such as sleep apnea, hyperventilation, and snoring, but also at REM sleep points, the user can be stimulated through the smart home-appliance 800. That is, as a result of the first and second sleep analyses, when it is determined that the user has entered the REM sleep stage, the smart home-appliance 800 can generate at least one of tactile feedback and auditory feedback.

Alternatively, in the case of an embodiment such as (c) of FIG. 1, at least one of the electronic devices shown in (c) of FIG. 1 may perform at least one of the aforementioned operations.

### Example 2 of the Method for Preventing and Alleviating Sleep Disorders

FIG. 36(b) is a flowchart illustrating a method for preventing and alleviating sleep disorders using an AI-based non-contact sleep analysis system according to another embodiment of the present invention.

The embodiment shown in FIG. 36(b) assumes a scenario where sleep analysis is performed on a smart home-appliance 800 and a smartphone 900.

According to one embodiment of the present invention, the sleep analysis results may include sleep state information, sleep stage information, sleep disorder occurrence information, time information, and the like. The smartphone 900 performs sleep analysis based on sleep sound information acquired through the built-in microphone module. Hereinafter, a method by which the smartphone 900 derives the final sleep analysis result using sleep sound information (Sound) will be described.

First, the smartphone 900 may derive the final sleep analysis result using weights. Specifically, the smartphone 900 can derive a secondary sleep analysis result by applying the same weight to the primary sleep analysis result and the sleep analysis result using sleep sound information.

In another embodiment, the smartphone 900 may derive the final sleep analysis result only when the sleep stages in the primary sleep analysis result and the secondary sleep analysis result completely match, determining that the user has entered the corresponding sleep stage. In another embodiment, the smartphone 900 first performs secondary sleep analysis using sleep sound information (Sound) with the AI sleep analysis model described later, and additionally extracts AI confidence levels for each time-specific sleep stage.

According to the present invention, if the extracted confidence level is below a predefined value, the sleep stage for that time period adopts the sleep stage result derived from the primary sleep analysis.

That is, by primarily adopting the secondary sleep analysis result and additionally adopting the primary sleep analysis result, a more reliable sleep analysis result can be derived.

In yet another embodiment, the smartphone 900 first secures statistics of discrepancies between the actual analysis results and the AI sleep analysis model described later. The statistics may be input by the user or autonomously secured through data from multiple users. The smartphone 900 may primarily adopt the secondary sleep analysis result (Sound-based analysis) and additionally adopt the primary sleep analysis result in areas where discrepancies exist in the secured statistics.

According to the present invention, the learning method of the AI sleep analysis model will be described in more detail below, but briefly, by inputting two types of information (primary sleep analysis result and sleep sound information) into the deep learning input layer, an AI sleep analysis model that performs sleep analysis based on two factors can be generated.

This is merely an embodiment, and the derivation of the final sleep analysis result can be achieved in various ways.

According to one embodiment of the present invention, if the secondary sleep analysis by the smartphone 900 determines that sleep apnea has occurred, the sensing unit can immediately transmit the sleep apnea occurrence information to the processor embedded in the smartphone 900. The sleep apnea occurrence information corresponds to a trigger signal for generating at least one of tactile feedback and auditory feedback by the smart home-appliance 800. Upon receiving the sleep apnea occurrence information, the smart home-appliance 800 can stimulate the user through vibrations, sounds, or audio. This stimulation can quickly alleviate the user's sleep apnea and, through continuous monitoring and stimulation, prevent or mitigate the user's sleep apnea.

Meanwhile, unlike the embodiments shown in FIGS. 34 to 36, the primary sleep analysis may be omitted, and sleep analysis may be conducted solely by the smartphone 900. That is, based on the user's sleep sound information, the user's sleep analysis is performed using the aforementioned method, and if sleep apnea is detected as a result of the sleep analysis, the sleep apnea occurrence information is immediately transmitted to the smart home-appliance 800 linked with the smartphone 900 (e.g., smart mattress, smart speaker, etc.), allowing the smart home-appliance 800 to generate vibrations or alarms (sound, audio).

Meanwhile, the correlation between air quality and sleep is as follows.

According to one study, if a mother is exposed to poor air quality during weeks 1 to 8 of pregnancy, the sleep efficiency of the newborn is reduced, and if exposed during weeks 31 to 35, the sleep duration of the newborn is reduced. It is also known that the quality of sleep during growth is closely related to knowledge acquisition and developmental growth.

Additionally, exposure to PM 10 in the summer increases irregularity in breathing during sleep, which is also related to an increase in cardiovascular diseases and mortality rates.

Meanwhile, the correlation between temperature/humidity and sleep is as follows.

A comparative experiment on sleep states at 800 ppm and 17000 ppm of carbon dioxide revealed that the air felt more stuffy and hot at 800 ppm. Another study found that sleeping in a chamber at 28 degrees Celsius resulted in lower sleep efficiency and work efficiency the next day compared to sleeping in a chamber at 24 degrees Celsius.

Furthermore, a study showed that in an environment with a relative humidity of 80% and a temperature of 32 degrees Celsius, the frequency of waking during sleep increased, and the proportion of deep sleep decreased compared to an environment with a relative humidity of 50% and a temperature of 26 degrees Celsius.

Such stimulation to prevent or mitigate the user's sleep disorders can be generated by an environment adjustment device other than the smartphone 900 or smart speaker 804.

Here, other environment adjustment devices may include lighting, air purifiers, humidifiers, speakers (audio), clothing care devices, TVs, clocks, PCs, motion beds, mattresses, smart pillows, blinds, curtains, robots, vacuum cleaners, washing machines, dryers, water purifiers, refrigerators, ovens/ranges, etc. The user's sleep disorder occurrence information can be transmitted to the various environment adjustment devices mentioned above, and the environment adjustment devices can generate stimuli to stimulate the user.

For example, sleep disorder occurrence information can be used to stimulate the user by controlling lighting to increase brightness, generating operational sounds from an air purifier, turning on a TV, activating a clock alarm, keeping a PC on, adjusting the bed angle via a motion bed, controlling a smart pillow or smart mattress to provide tactile changes or movements, or operating various home-appliances to produce sounds, thereby interrupting or alleviating sleep disorders.

Additionally, in the embodiment shown in FIG. 1(c), at least one of the electronic devices depicted in FIG. 1(c) may perform at least one of the aforementioned operations.

### Traffic Response Method and Single/Multiple User Sleep Analysis

FIG. 37 illustrates a diagram explaining the traffic response method when the sleep analysis method according to the present invention is performed in the cloud.

FIG. 38 is a conceptual diagram for explaining single-user sleep analysis and multiple-user sleep analysis according to the sleep analysis method of the present invention. For the sake of understanding, the smart home-appliance 800 will be assumed to be a smart speaker 804 in the following description. However, this is merely for explanatory purposes and does not limit the smart home-appliance of the present invention.

The sleep analysis method according to the present invention can be provided to users through the Amazon Web Services (AWS) cloud. Since the sleep analysis method according to the present invention is primarily conducted from evening to early morning, traffic may occur during these times.

Therefore, the sleep analysis method according to the present invention may further include the steps of analyzing the time intervals during which a lot of traffic occurs, predicting events entering those time intervals, and automatically adjusting (adding, relocating, etc.) the AI server 310 at the time the event occurs. Through this, the present invention can flexibly respond to traffic that may occur at specific times.

First, in the case of single-user sleep shown in FIG. 38(a), both the smart speaker 804 and the smartphone 900 are located within the same sleep space. That is, the smart speaker 804 can acquire the sleep sound information and sleep environment information of a single user, and the smartphone 900 can acquire the sleep sound information and sleep environment information (such as brightness) of a single user. In such a single-user sleep environment, the sleep analysis method described above can be directly applied.

However, in the case of multiple-user sleep shown in FIG. 38(b), the sleep sound information acquired by the smart speaker 804 or smartphone 900 may include sleep information for multiple users, such as User 1 and User 2.

Therefore, sleep analysis for multiple users sleeping in the same sleep space undergoes a more precise process. The multiple-user sleep analysis method has been previously described with reference to FIGS. 39 to 46, so redundant descriptions will be omitted.

### Sleep Environment Adjustment Device

Hereinafter, the sleep environment adjustment device depicted in FIG. 1(b) will be described in more detail. As shown in FIG. 1(b), the sleep environment adjustment device 400, the user terminal 10, and the external server 20 can mutually transmit and receive data for the system according to embodiments of the present invention through a network.

The network according to the embodiments of the present invention is as detailed above, and thus redundant descriptions will be omitted.

According to this embodiment, the user terminal 10 is a terminal that can receive information related to the user's sleep through information exchange with the sleep environment adjustment device 400, and may refer to a terminal possessed by the user. The general configuration and function of the user terminal 10 may be as described above. The user terminal 10 can acquire sound information related to the space where the user is located. For example, the sound information may refer to sound information acquired from the space where the user is located. The sound information can be acquired in a non-contact manner in relation to the user's activity or sleep.

For instance, the sound information may be acquired from the space while the user is sleeping. According to the embodiment, the sound information acquired through the user terminal 10 may serve as foundational information for acquiring the user's sleep state information in the present invention. Specifically, sleep state information regarding whether the user is before sleep, during sleep, or after sleep can be acquired through sound information related to the user's movements or breathing. Additionally, for example, information on changes in the user's sleep stages during sleep can be acquired through sound information.

The sleep environment adjustment device 400 of the present invention can receive health examination information or sleep examination information from the external server 20 and build a learning data set based on such information. The description related to the external server 20 is detailed above, and thus will be omitted here.

According to one embodiment, the sound information utilized by the sleep environment adjustment device 400 for sleep state analysis may be acquired in a non-invasive manner during the user's activity or sleep in a space. Specifically, the sound information may include sounds generated by the user's tossing and turning, sounds related to muscle movements, or sounds related to the user's breathing during sleep. According to the embodiment, the environment sensing information may include sleep sound information, which refers to sounds related to movement patterns and breathing patterns occurring during the user's sleep.

In the embodiment, the sound information can be acquired through at least one of the user terminal 10 and the sound collection unit 414. For example, environment sensing information related to the user's activity in a space can be acquired through a microphone module provided in the user terminal 10 and the sound collection unit 414.

The configuration of the microphone module provided in the user terminal 10 or the sound collection unit 414 is the same as described above.

In the present invention, the sound information subject to analysis is related to the user's breathing and movements acquired during sleep, and pertains to very small sounds (i.e., sounds that are difficult to distinguish). Since it is acquired along with other sounds during the sleep environment, if acquired through the aforementioned microphone module with a low signal-to-noise ratio, detection and analysis can be very challenging.

According to one embodiment of the present invention, the sleep environment adjustment device 400 can acquire sleep state information based on sound information obtained through a microphone module composed of MEMS. Specifically, the sleep environment adjustment device 400 can convert and/or adjust sound information, which is acquired indistinctly with a lot of noise, into data that can be analyzed, and utilize the converted and/or adjusted data to perform training on an artificial neural network. Once pre-training on the artificial neural network is completed, the trained neural network (e.g., sound analysis model) can acquire the user's sleep state information based on data (e.g., spectrogram) obtained in response to the sound information (e.g., converted and/or adjusted). In this embodiment, the sleep state information may include not only information related to whether the user is sleeping but also sleep stage information related to changes in the user's sleep stages during sleep. For example, the sleep state information may include sleep stage information indicating that the user was in REM sleep at a first time point and in light sleep at a second time point different from the first time point. In this case, through the sleep state information, it can be determined that the user was in relatively deep sleep at the first time point and in lighter sleep at the second time point.

That is, the sleep environment adjustment device 400 can acquire sleep sound information with a low signal-to-noise ratio through commonly distributed user terminals (e.g., AI speakers, bedroom IoT devices, mobile phones, etc.) or a sound collection unit 414, process it into data suitable for analysis, and provide information on whether the user is before, during, or after sleep, as well as sleep state information related to changes in sleep stages.

In an embodiment, the sleep environment adjustment device 400 may be a terminal or server and can include any form of device. The sleep environment adjustment device 400 can be a digital device equipped with processing capabilities and memory, such as a laptop computer, notebook computer, desktop computer, web pad, or mobile phone. The sleep environment adjustment device 400 may also be a web server that processes services. The types of servers mentioned above are merely examples and the present invention is not limited thereto.

According to one embodiment of the present invention, the sleep environment adjustment device 400 may be a server providing cloud computing services. As detailed above, the description is omitted here.

Alternatively, in the case of the embodiment shown in FIG. 1(c), at least one of the electronic devices depicted in FIG. 1(c) can be implemented as the sleep environment adjustment device 400.

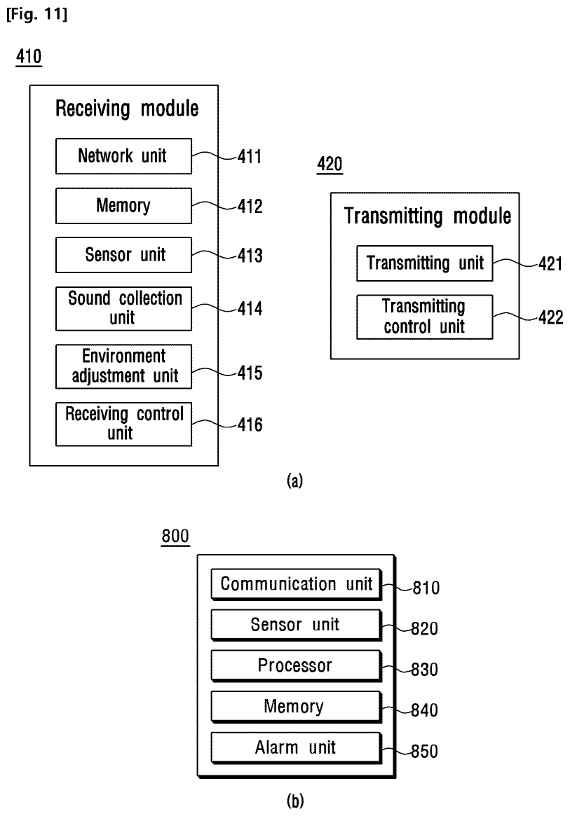

FIG. 10 illustrates an exemplary block diagram of a sleep environment adjustment device related to one embodiment of the present invention.

As shown in FIG. 10, the sleep environment adjustment device 400 may include a receiving module 410 and a transmitting module 420.

According to one embodiment of the present invention, the sleep environment adjustment device 400 may include a transmitting module 420 that transmits wireless signals and a receiving module 410 that receives the transmitted wireless signals. In one embodiment, the wireless signals may refer to signals of an orthogonal frequency division multiplexing (OFDM) method. For example, the wireless signals may be Wi-Fi-based OFDM sensing signals.

Additionally, the transmitting module 420 of the present invention can be implemented through devices such as laptops, smartphones, tablet PCs, and smart speakers 804, while the receiving module 410 can be implemented through a Wi-Fi receiver. According to the embodiment, the receiving module 410 can also be implemented through various computing devices such as laptops, smartphones, and tablet PCs. For example, the transmitting module 420 and the receiving module 410 may be equipped with wireless chips that support standards such as Wi-Fi 802.1 in, 802.1 lac, or other OFDM-supporting standards. Thus, a sleep environment adjustment device 400 that acquires object state information with high reliability through relatively low-cost equipment can be implemented.

In one embodiment, the transmitting module 420 can transmit wireless signals in one direction where the object is located, and the receiving module 410 can be provided at a predetermined separation distance from the transmitting module 420 to receive the wireless signals transmitted from the transmitting module 420. These wireless signals, being of an orthogonal frequency division multiplexing method, can be transmitted or received through multiple subcarriers.

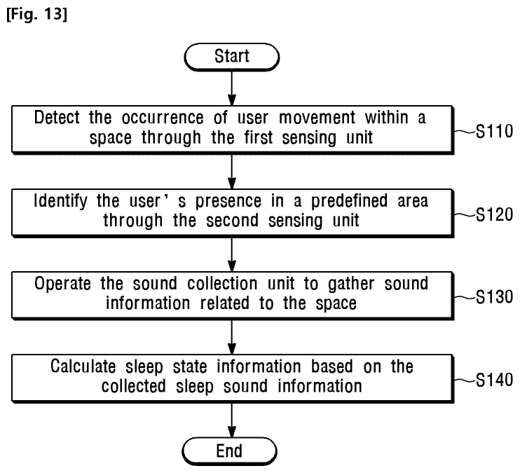

The transmitting module 420 and the receiving module 410 can be provided to have a predetermined separation distance. In this case, the predetermined separation distance may refer to the space where the object is active or located. In a specific embodiment, the transmitting module 420 and the receiving module 410 may be characterized by being provided in opposing positions based on a predefined area. Here, the predefined area 11a may refer to, for example, the area related to the position where the user sleeps, such as the area where the bed is located, as shown in FIG. 12. Alternatively, it may refer to an area where object state information such as the user's movement or breathing can be acquired. Here, the object state information is not limited to information related to the user's movement or breathing but may correspond to various information such as sound information or visual information related to the user.

The transmitting module 420 and the receiving module 410 may be provided on each side of the bed where the user sleeps. In this case, the sleep environment adjustment device 400 of the present invention can acquire object state information, such as information on whether the user is located in a predefined area and information on the user's movement or breathing, based on WiFi-based OFDM signals transmitted and received through the transmitting module 420 and the receiving module 410.

According to one embodiment, the transmitting module 420 and the receiving module 410 can transmit and receive wireless signals (e.g., OFDM signals) through one or more antennas. For example, if each of the transmitting module 420 and the receiving module 410 is equipped with three antennas, channel state information related to a total of 192 channels (i.e., 3×64 can be acquired per frame through the three antennas and 64 subcarriers. The specific numerical description of the antennas and subcarriers mentioned above is merely exemplary and not limiting to the present disclosure.

According to one embodiment, multiple transmitting modules 420 and receiving modules 410 may be provided. More specifically, three transmitting modules and four receiving modules can be provided at predetermined separation distances. In this case, the wireless signals transmitted and received by each of the multiple transmitting modules and receiving modules may differ from each other.

In an embodiment, the wireless signal received through the receiving module 410 may be a wireless signal that has passed through a channel corresponding to the predefined area and may include information representing the characteristics of that channel. The receiving module 410 can acquire channel state information from the wireless signal. The channel state information represents characteristics related to the channel associated with the space where the user is located and can be calculated based on the wireless signal transmitted from the transmitting module 420 and received through the receiving module.

Specifically, the wireless signal transmitted from the transmitting module 420 can pass through a specific channel (i.e., the space where the user is located) and be received through the receiving module 410. In this case, the wireless signal may be transmitted through multiple subcarriers corresponding to each multi-path. Accordingly, the wireless signal received through the receiving module 410 may be a signal reflecting the user's movement in the predefined area 11a. The processor can acquire channel state information related to the channel characteristics experienced by the wireless signal as it passes through the channel (i.e., the space where the user is located) from the received wireless signal. This channel state information may consist of amplitude and phase. That is, the sleep environment adjustment device 400 can acquire channel state information related to the characteristics of the space (i.e., the predefined area) between the transmitting module 420 and the receiving module 410 based on the wireless signal transmitted from the transmitting module 420 and the wireless signal received through the receiving module 410 (i.e., the signal reflecting the object's movement).

According to an embodiment, when the receiving module 410 receives the wireless signal transmitted from the transmitting module 420, it can be characterized by detecting the user's movement based on the received wireless signal. The receiving module 410 can acquire information on whether the user is located in the predefined area through changes in the channel state information. According to an embodiment, during the process of transmitting and receiving wireless signals through the transmitting module 420 and the receiving module 410, the channel state information acquired when the user is located between the transmitting module 420 and the receiving module 410 or not may differ. In a specific embodiment, the transmitting module 420 and the receiving module 410 can be arranged to maximize the difference in channel state information acquired when the user is located in the area between the transmitting module 420 and the receiving module 410 (i.e., the predefined area) and when the user is not located there. In an additional embodiment, a directional patch antenna can be provided corresponding to each of the transmitting module 420 and the receiving module 410. Here, the directional patch antenna may be an antenna module composed of m×n patches (i.e., m horizontal patches and n vertical patches). For example, the antenna pre-beam can be set to increase the signal difference when the user is located between the transmitting module 420 and the receiving module 410 or not. The beam width of the antenna is preset to be optimal, and the transmitting module 420 and the receiving module 410 can be arranged so that the user's lying position is in the direction of transmitting and receiving signals using this directional patch antenna. That is, a wireless link can be directly secured in Line-of-Sight between the directional patch antennas of the transmitting module 420 and the receiving module 410. Through this configuration, the antennas of each module can operate as directional antennas to form a wireless link corresponding to a narrower area (e.g., the predefined area).

That is, a wireless link can be formed between the antennas of the transmitting module 420 and the receiving module 410, and when the user is located between this wireless link, the user's body obstructs the wireless link, causing distortion and significant changes in the signal level (i.e., channel state information). In the embodiment, changes in the signal level can be detected through changes in the RSSI (Received Signal Strength Indicator) and CSI (Channel State Information), and accordingly, the receiving module 410 can determine whether the user is located in the predefined area 11a through these changes.

In the embodiment, information on whether the user is located in the predefined area 11a can be used to determine the operation of the environment adjustment unit 415 or to understand the user's sleep intention.

According to one embodiment of the present invention, the receiving module 410 can calculate the user's sleep state information and adjust the user's sleep environment based on the sleep state information. Specifically, the receiving module 410 can acquire sleep state information related to whether the user is before, during, or after sleep based on the acquired sensing information and adjust the sleep environment of the space where the user is located according to the sleep state information. For example, if the receiving module 410 acquires sleep state information indicating that the user is before sleep, it can generate environment adjustment information related to the intensity and illumination of light to induce sleep (e.g., white light of 3000K, illumination of 30 lux) based on the sleep state information. Additionally, the receiving module 410 can adjust the intensity and illumination of light in the space where the user is located to appropriate levels for inducing sleep (e.g., white light of 3000K at 30 lux) based on the environment adjustment information related to the intensity and illumination of light for inducing sleep.

Furthermore, if the receiving module 410 acquires sleep state information indicating that the user is before sleep, it can generate environment adjustment information for controlling smart home-appliances from the predicted time when the user is preparing for sleep (e.g., sleep induction time) until the time the user falls asleep (i.e., the time when the second sleep state information is acquired).

Specifically, environment adjustment information can be generated to pre-remove fine dust and harmful gases up to a predetermined time before the user falls asleep (e.g., 20 minutes prior), or to control the indoor temperature and humidity to be optimized according to the season or user, or to control the illumination.

Additionally, the environment adjustment information may include information to control various smart home-appliances to induce sleep just before sleep by causing noise (white noise) to a degree that can induce sleep, adjusting the blowing strength to be below a predefined intensity, lowering the intensity of LEDs, or converting direct airflow to indirect airflow.

Furthermore, the first environment adjustment information may include control information to adjust at least one of various environments such as personalized indoor temperature, indoor humidity, blowing strength, or noise, according to the operation history of the smart home-appliance and the acquired sleep state (e.g., sleep quality).

The specific descriptions related to the aforementioned sleep state information and environment adjustment information are merely examples, and the present invention is not limited thereto.

FIG. 11(a) illustrates an exemplary block configuration diagram of a receiving module and a transmitting module related to an embodiment of the present invention.

As shown in FIG. 11(a), the receiving module 410 may include a network unit 411, a memory unit 412, a sensor unit 413, a sound collection unit 414, an environment adjustment unit 415, and a receiving control unit 416. The receiving module 410 is not limited to the aforementioned components. That is, additional components may be included, or some of the aforementioned components may be omitted, depending on the implementation aspects of the embodiments of the present invention.

According to an embodiment of the present invention, the transmitting module 420 may include a transmitting unit 421 for transmitting wireless signals and a transmitting control unit 422 for controlling the wireless signal transmission operation of the transmitting unit 421, as shown in FIG. 11(a). In the embodiment, the transmitting control unit 422 can determine the timing at which the wireless signal is transmitted through the transmitting unit 421. For example, the transmitting control unit 422 can control the transmitting unit 421 to transmit a wireless signal in response to the initiation of the sleep measurement mode.

According to an embodiment of the present invention, the receiving module 410 may include a network unit 411 for transmitting and receiving data with the transmitting module 420, user terminal 10, and external server 20. The network unit 411 can transmit and receive data, etc., for performing a sleep environment adjustment method according to sleep state information according to an embodiment of the present invention with other computing devices, servers, etc. That is, the network unit 411 can provide communication functions between the receiving module 410, transmitting module 420, user terminal 10, and external server 20. For example,
the network unit 411 can receive sleep examination records and electronic health records for multiple users from a hospital server. In another example, the network unit 411 can receive sound information related to the space where the user is active from the user terminal 10. In yet another example, the network unit 411 can transmit environment adjustment information to the environment adjustment unit 415 to adjust the environment of the space where the user is located. Additionally, the network unit 411 can allow information delivery between the sleep environment adjustment device 400, user terminal 10, and external server 20 by calling procedures to the sleep environment adjustment device 400.

According to one embodiment of the present invention, the network unit 411 may be configured as any one or a combination of the various wired and wireless communication systems described above.

In one embodiment of the present invention, the memory 412 may store a computer program for performing a sleep environment adjustment method based on sleep state information according to an embodiment of the present invention. The stored computer program may be read and executed by the receiving control unit 416. Additionally, the memory 412 may store any form of information generated or determined by the receiving control unit 416 and any form of information received by the network unit 411. Furthermore, the memory 412 may store data related to the user's sleep. For example, the memory 412 may temporarily or permanently store input/output data (e.g., sleep sound information related to the user's sleep environment, sleep state information corresponding to the sound information, or environment adjustment information according to the sleep state information).

According to one embodiment of the present invention, the memory 412 may include at least one type of storage medium such as flash memory type, hard disk type, multimedia card micro type, card type memory (e.g., SD or XD memory), RAM (Random Access Memory), SRAM (Static Random Access Memory), ROM (Read-Only Memory), EEPROM (Electrically Erasable Programmable Read-Only Memory), PROM (Programmable Read-Only Memory), magnetic memory, magnetic disk, or optical disk. The sleep environment adjustment device 400 may operate in association with web storage that performs the storage function of the memory 412 on the internet. The description of the memory above is merely exemplary, and the present invention is not limited thereto.

When the computer program is loaded into the memory 412, it may include one or more instructions that cause the receiving control unit 416 to perform methods/operations according to various embodiments of the present invention. That is, the receiving control unit 416 may perform methods/operations according to various embodiments of the present invention by executing one or more instructions.

According to one embodiment of the present invention, the receiving module 410 may include a sensing unit 413 that acquires one or more pieces of sensing information related to a space. In the present invention, a space refers to a living space where the user resides, for example, a bedroom where the user sleeps.

According to the embodiment, the sensing unit 413 may include a first sensing unit that detects the user's movement in a space. The first sensing unit may be equipped with at least one of a PIR sensor (Passive Infrared Sensor) and an ultrasonic sensor. The PIR sensor can detect the user's movement within the detection range by sensing changes in infrared radiation emitted from the user's body. For example, the PIR sensor can detect the user's movement in the bedroom by identifying infrared radiation of 8 μm~14 μm emitted from the user's body. The ultrasonic sensor can detect the movement of an object by emitting sound waves and sensing the signals reflected back from a specific object. For example, the ultrasonic sensor can emit sound waves within the bedroom space and detect the user's movement inside the bedroom through the sound waves reflected from the user's body as the user enters the bedroom.

Additionally, in the embodiment, the sensing unit 413 may include a second sensing unit that detects whether the user is located in a predefined area of the space based on wireless signals. The second sensing unit can receive wireless signals transmitted from the transmitting module 420 and detect whether the user is located in the predefined area based on the received wireless signals. In the embodiment, the predefined area is related to the area within the space where the user lies down to sleep, for example, the area where the bed is located. Specifically, in the present invention, the space may refer to the bedroom interior space, and the predefined area may refer to the space where the bed is located.

In the embodiment, the second sensing unit may be characterized by being provided in a position opposite to the transmitting module 420 based on the predefined area. For example, the transmitting module 420 and the second sensing unit may be provided on each side of the bed where the user sleeps. In this case, the sleep environment adjustment device 400 of the present invention can acquire information on whether the user is located in the predefined area and object state information regarding the user's movement or breathing based on wifi-based OFDM signals transmitted and received through the transmitting module 420 and the receiving module.

According to one embodiment, if the receiving module 410 determines that the user is located in the predefined area through the second sensing unit, it may allow the operation of the environment adjustment unit 415. In other words, the receiving module 410 may allow the operation of the environment adjustment unit 415 only when the user is detected to be located in the predefined area 11a. That is, the receiving module 410 may control the operation of the environment adjustment unit 415 to perform environment adjustment operations by generating environment adjustment information only when the user is located in the predefined area. The environment adjustment unit 415 may not perform operations to change the sleep environment if the user is not located in a specific position.

In an additional embodiment, the sensing unit 413 may include one or more environment sensing modules to acquire indoor environment information related to at least one of the user's body temperature, indoor temperature, indoor airflow, indoor humidity, and indoor illumination concerning the user's sleep environment. Indoor environment information, as information related to the user's sleep environment, may serve as a reference for considering the influence of external factors on the user's sleep through sleep state information related to changes in the user's sleep stage. The one or more environment sensing modules may include, for example, at least one sensor module among a temperature sensor, airflow sensor, humidity sensor, sound sensor, and illumination sensor. However, it is not limited thereto, and various sensors capable of measuring external environments that may affect the user's sleep may also be included.

According to an embodiment of the present invention, the receiving module 410 may include a sound collection unit 414. The sound collection unit 414 is configured to include a small microphone module and can acquire information on sounds occurring in the space where the user is sleeping. In one embodiment, the microphone module provided in the sound collection unit 414 may be composed of a MEMS (Micro-Electro Mechanical Systems) of relatively small size. Such a microphone module is advantageous in terms of cost and can be manufactured in a very compact size, but may have a lower signal-to-noise ratio (SNR) compared to a condenser microphone or a dynamic microphone. A low signal-to-noise ratio means that the ratio of noise, which is the sound not intended to be identified, to the sound intended to be identified is high, indicating that sound identification is not easy (i.e., unclear). The information subject to analysis in the present invention may be sleep sound information, which is acoustic information related to the user's breathing and movements acquired during sleep. This sleep sound information pertains to very subtle sounds such as the user's breathing and movements, and since it is acquired along with other sounds during the sleep environment, detection and analysis can be very difficult when acquired through the aforementioned microphone module with a low signal-to-noise ratio. Accordingly, the receiving control unit 416 may process the sleep sound information with a low signal-to-noise ratio into data for processing and/or analysis.

According to an embodiment of the present invention, the receiving module 410 may include an environment adjustment unit 415. The environment adjustment unit 415 can adjust the user's sleep environment. Specifically, the environment adjustment unit 415 can adjust at least one of air quality, illumination, temperature, wind direction, humidity, and sound in the space where the user is located, based on environment adjustment information. The environment adjustment information may be a signal generated from the receiving control unit 416 based on the determination of the user's sleep state information. For example, the environment adjustment information may include information on lowering or increasing illumination. More specifically, the environment adjustment information may include control information to gradually increase white light of 3000K from 0 lux to 250 lux starting 30 minutes before the predicted wake-up time. Additionally, the environment adjustment information may include control information for adjusting at least one of temperature, humidity, wind direction, or sound. The environment adjustment information may include various information related to fine dust removal, harmful gas removal, allergy care operation, deodorization/sterilization operation, indoor temperature control, dehumidification control, humidification control, blower intensity control, wind direction selection and control, operation noise control, vibration control, LED lighting control, etc., based on the user's real-time sleep state. The specific description of the aforementioned environment adjustment information is merely exemplary and the present invention is not limited thereto.

According to the present invention, the environment adjustment unit 415 can perform control over at least one of illumination control, temperature control, wind direction control, humidity control, and sound control. However, it is not limited thereto, and the environment adjustment unit can perform various control operations that can bring changes to the user's sleep environment. That is, the environment adjustment unit 415 can adjust the user's sleep environment by performing various control operations based on the environment control signal from the receiving control unit 416.

In an additional embodiment, the environment adjustment unit 415 may be implemented through linkage via the Internet of Things (IoT). Specifically, the environment adjustment unit 415 may be implemented through linkage with various devices that can bring changes to the indoor environment related to the space where the user is located. For example, the environment adjustment unit 415 may be implemented as various smart home-appliances such as a smart air conditioner, smart heater, smart air purifier, smart boiler, smart window, smart humidifier, smart dehumidifier, and smart lighting based on linkage through the Internet of Things. The specific description of the aforementioned environment adjustment unit is merely exemplary and the present invention is not limited thereto.

According to an embodiment of the present invention, the receiving control unit 416 may be composed of one or more cores and may include processors for data analysis and deep learning, such as a central processing unit (CPU), a general-purpose graphics processing unit (GPGPU), and a tensor processing unit (TPU) of a computing device.

According to the present invention, the receiving control unit 416 can perform data processing for machine learning according to an embodiment of the present invention by reading a computer program stored in the memory 412.

According to an embodiment of the present invention, the receiving control unit 416 can perform calculations for training a neural network. The receiving control unit 416 can perform calculations for neural network training, such as processing input data for learning in deep learning (DL), feature extraction from input data, error calculation, and weight updates of the neural network using backpropagation.

Additionally, at least one of the CPU, GPGPU, and TPU of the receiving control unit 416 can process the learning of network functions. For example, the CPU and GPGPU can together process the learning of network functions and data classification using network functions. Furthermore, in an embodiment of the present invention, the processors of multiple computing devices can be used together to process the learning of network functions and data classification using network functions. Additionally, the computer program executed in the computing device according to an embodiment of the present invention may be a CPU, GPGPU, or TPU executable program.

In this specification, the network function can be used interchangeably with an artificial neural network or neural network. The network function may include one or more neural networks, and in this case, the output of the network function may be an ensemble of the outputs of one or more neural networks.

In this specification, the model may include a network function. The model may include one or more network functions, and in this case, the output of the model may be an ensemble of the outputs of one or more network functions.

According to the present invention, the receiving control unit 416 can provide a sleep analysis model according to an embodiment of the present invention by reading a computer program stored in the memory 412. In one embodiment of the present invention, the receiving control unit 416 can perform calculations to derive environment adjustment information based on sleep state information. In another embodiment of the present invention, the receiving control unit 416 can perform calculations to train the sleep analysis model.

According to an embodiment of the present invention, the receiving control unit 416 can generally handle the overall operation of the sleep environment adjustment device 400. By processing signals, data, and information input or output through the components examined above, or by running applications stored in the memory 412, the receiving control unit 416 can provide or process appropriate information or functions to the user terminal.

According to an embodiment of the present invention, the receiving control unit 416 can acquire sound information related to the space where the user is sleeping. The acquisition of sound information, according to an embodiment of the present invention, may involve obtaining or loading sound information stored in the memory 412. Additionally, the acquisition of sound information may involve receiving or loading data from other storage media, other computing devices, or separate processing modules within the same computing device based on wired/wireless communication means.

In one embodiment, the receiving control unit 416 can acquire sleep sound information from living environment sound information. Here, the living environment sound information may be sound information acquired during the user's daily life. For example, living environment sound information may include various sound information acquired according to the user's lifestyle, such as sound information related to cleaning, cooking, or watching TV.

Specifically, the receiving control unit 416 can identify singularities where information of a predefined pattern is detected in the living environment sound information. Here, the information of the predefined pattern may relate to breathing and movement patterns associated with sleep. For instance, in a wake state, all nervous systems are activated, leading to irregular breathing patterns and frequent body movements. Additionally, due to the lack of relaxation of neck muscles, breathing sounds may be minimal. Conversely, when the user is sleeping, the autonomic nervous system stabilizes, resulting in regular breathing changes, reduced body movements, and potentially increased breathing sounds. That is, the receiving control unit 416 can identify the point in time when sound information of a predefined pattern related to regular breathing, minimal body movement, or minimal breathing sounds is detected in the living environment sound information as a singularity. Furthermore, based on the living environment sound information acquired at the identified singularity, the receiving control unit 416 can acquire sleep sound information. The receiving control unit 416 can identify singularities related to the user's sleep timing from time-series acquired living environment sound information and acquire sleep sound information based on these singularities.

For a specific example, the receiving control unit 416 can identify singularities related to the timing when a predefined pattern is identified from the living environment sound information. Additionally, based on the sound information acquired after the identified singularity, the receiving control unit 416 can acquire sleep sound information.

In other words, by identifying singularities related to the user's sleep from the living environment sound information, the receiving control unit 416 can extract and acquire only the sleep sound information from a vast amount of sound information. In other words, it can acquire only the sound related to sleep (i.e., sleep sound information) from the sounds occurring in a space. This can automate the process of recording the user's sleep time, providing convenience and simultaneously contributing to the improvement of the accuracy of the acquired sleep sound information.

According to an embodiment, the receiving control unit 416 can derive sleep state information based on sound information. Specifically, the receiving control unit 416 can derive sleep state information based on the user's sleep sound information acquired through the sound collection unit 414.

In one embodiment, the sleep state information may include information related to whether the user is sleeping. Specifically, the sleep state information may include at least one of the first sleep state information indicating the user is before sleep, the second sleep state information indicating the user is during sleep, and the third sleep state information indicating the user is after sleep. In other words, if the first sleep state information is acquired concerning the user, the receiving control unit 416 can determine that the user is in a state before sleep (i.e., before going to bed). If the second sleep state information is acquired, it can determine that the user is in a state during sleep, and if the third sleep state information is acquired, it can determine that the user is in a state after sleep (i.e., upon waking).

This sleep state information is characterized by being acquired based on sleep sound information. The sleep sound information may include sound information acquired in a non-contact manner during the user's sleep in the space where the user is located.

According to one embodiment, the receiving control unit 416 can calculate sleep state information based on the collected sound information (S140). In this embodiment, the receiving control unit 416 can acquire sleep state information related to whether the user is before sleep or during sleep based on singularities identified from the sound information. Specifically, if no singularity is identified, the receiving control unit 416 may determine that the user is before sleep, and if a singularity is identified, it may determine that the user is during sleep after the singularity. Additionally, the receiving control unit 416 can identify a point in time when a predefined pattern is not observed after a singularity is identified (e.g., waking time), and if such a point is identified, it can determine that the user is after sleep, i.e., has woken up.

That is, the receiving control unit 416 can acquire sleep state information related to whether the user is before, during, or after sleep based on whether a singularity is identified in the sound information and whether a predefined pattern is continuously detected after the singularity is identified.

According to one embodiment of the present invention, the receiving control unit 416 can generate environment adjustment information based on sensing information and sleep state information. Specifically, the receiving control unit 416 can generate environment adjustment information based on sensing information acquired through the sensing unit 413 and sleep state information obtained from sound analysis results. The receiving control unit 416 generates environment adjustment information based on sensing information and sleep state information and transmits the generated environment adjustment information to the environment adjustment unit 415, thereby controlling the sleep environment change operation of the environment adjustment unit 415.

In the embodiment, the receiving control unit 416 can generate environment adjustment information based on sleep state information. Sleep state information is information related to whether the user is sleeping, and may include at least one of first sleep state information indicating the user is before sleep, second sleep state information indicating the user is during sleep, and third sleep state information indicating the user is after sleep.

To explain in more detail, the receiving control unit 416 can generate first environment adjustment information based on the first sleep state information. Specifically, if the receiving control unit 416 acquires first sleep state information indicating the user is before sleep, it can generate first environment adjustment information based on the first sleep state information. That is, if the user's sleep state is before sleep, the receiving control unit 416 can generate first environment adjustment information to supply predefined white light for a certain period.

According to the embodiment, the sleep induction time can be determined by the receiving control unit 416. Specifically, the receiving control unit 416 can determine the sleep induction time through information exchange with the user's user terminal 10. For example, the user can set the desired sleep time and wake-up time through the user terminal 10 to generate sleep plan information, and transmit the generated sleep plan information to the receiving control unit 416. In this case, the sleep plan information may include desired sleep time information and desired wake-up time information. The receiving control unit 416 can identify the sleep induction time based on the desired sleep time information. For example, the receiving control unit 416 can determine a time 20 minutes before the user's desired sleep time (i.e., desired sleep time) as the sleep induction time. For instance, if the user's desired sleep time is set to 11:00, the receiving control unit 416 can identify 10:40 as the sleep induction time. The specific numerical description of the aforementioned time is merely exemplary and the present invention is not limited thereto.

Additionally, according to the embodiment, the receiving control unit 416 can acquire the user's sleep intention information based on living environment sound information and determine the sleep induction time based on the sleep intention information. Sleep intention information may be information that quantitatively represents the user's intention to sleep. For example, the higher the user's sleep intention, the closer the sleep intention information is to 10, and the lower the sleep intention, the closer the sleep intention information is to 0. The specific numerical description of the aforementioned sleep intention information is merely exemplary and the present invention is not limited thereto.

The receiving control unit 416 can acquire sleep intention information based on living environment sound information. According to one embodiment, the receiving control unit 416 can identify the types of sounds included in the living environment sound information. Additionally, the receiving control unit 416 can calculate sleep intention information based on the number of identified types of sounds. The receiving control unit 416 can calculate lower sleep intention information as the number of types of sounds increases, and higher sleep intention information as the number of types of sounds decreases. For example, if the living environment sound information includes three types of sounds (e.g., vacuum cleaner sound, TV sound, and user voice), the receiving control unit 416 can calculate the sleep intention information as 2 points. Additionally, for example, if the living environment sound information includes one type of sound (e.g., washing machine), the receiving control unit 416 can calculate the sleep intention information as 6 points. The specific numerical description of the types of sounds included in the living environment sound information and the sleep intention information is merely exemplary and the present invention is not limited thereto.

That is, the receiving control unit 416 can acquire sleep intention information related to how much the user intends to sleep based on the number of types of sounds included in the living environment sound information. For example, the more types of sounds identified, the lower the user's sleep intention, resulting in sleep intention information (i.e., low score sleep intention information) being output.

In an embodiment, the receiving control unit 416 can generate an intention score table by pre-matching different intention scores to each of a plurality of sound information. For example, a first sound information related to a washing machine may be pre-matched with an intention score of 2 points, a second sound information related to a humidifier sound may be pre-matched with an intention score of 5 points, and a third sound information related to a voice may be matched with an intention score of 1 point. The receiving control unit 416 can pre-match relatively high intention scores to sound information related to the user's sleep (e.g., sounds generated as the user is active, such as vacuum cleaner, dishwashing, voice sounds, etc.) and pre-match relatively low intention scores to sound information unrelated to the user's sleep (e.g., sounds unrelated to the user's activities, such as vehicle noise, rain sounds, etc.) to generate the intention score table. The specific numerical descriptions of the intention scores matched to each sound information mentioned above are merely exemplary, and the present invention is not limited thereto.

The receiving control unit 416 can acquire sleep intention information based on the living environment sound information and the intention score table. Specifically, the receiving control unit 416 can record the intention score matched to the identified sound at the time when at least one of the plurality of sounds included in the intention score table is identified from the living environment sound information. For example, if a vacuum cleaner sound is identified at a first time point during the process of acquiring living environment sound information in real-time, the receiving control unit 416 can record the intention score of 2 points matched to the vacuum cleaner sound at the first time point. The receiving control unit 416 can record the intention score matched to the identified sound at the corresponding time point whenever various sounds are identified during the process of acquiring living environment sound information.

In an embodiment, the receiving control unit 416 can acquire sleep intention information based on the sum of the intention scores acquired over a predetermined time (e.g., 10 minutes). For example, the higher the intention score acquired over 10 minutes, the higher the sleep intention information that can be acquired, and the lower the intention score acquired over 10 minutes, the lower the sleep intention information that can be acquired. The specific numerical description of the predetermined time mentioned above is merely exemplary, and the present invention is not limited thereto.

That is, the receiving control unit 416 can acquire sleep intention information related to how much the user intends to sleep based on the characteristics of the sounds included in the living environment sound information. For example, the more sounds related to the user's activities are identified, the sleep intention information indicating that the user's sleep intention is low (i.e., low score sleep intention information) can be output.

According to an embodiment, the receiving control unit 416 can determine the sleep induction time based on the sleep intention information. Specifically, the receiving control unit 416 can identify the time when the sleep intention information exceeds a predetermined threshold score as the sleep induction time. That is, when high sleep intention information is acquired, the receiving control unit 416 can identify this as the appropriate time for sleep induction, i.e., the sleep induction time.

Additionally, in an embodiment, the receiving control unit 416 can calculate sleep intention weighting information based on the sensing information acquired through the sensing unit 413. Specifically, the receiving control unit 416 can determine that the user has a high sleep intention when the user's movement is detected in a space through the first sensing unit and the user is identified to be located in a predefined area through the second sensing unit, and accordingly, calculate sleep intention weighting information related to 1. If the receiving control unit 416 detects no movement of the user in the space and predefined area through the first and second sensing units and the user is not located there, it can determine that the user does not have a sleep intention and calculate sleep intention weighting information related to 0. That is, the receiving control unit 416 can calculate sleep intention weighting information related to 1 when the user is detected to be located in a specific space (e.g., bed space) through the sensing unit 413, and calculate sleep intention weighting information related to 0 when the user is detected not to be located in the specific space. In other words, the receiving control unit 416 can calculate sleep intention weighting information related to 0 or 1 depending on whether the user is located in the space and predefined area.

According to an embodiment, the receiving control unit 416 can determine the sleep induction time based on the sensing information and sleep state information. Specifically, the receiving control unit 416 can determine the sleep induction time based on the sensing information acquired through the sensing unit 413 and the sleep state information obtained from sound analysis results. The receiving control unit 416 can determine the sleep induction time based on the sleep intention information calculated from the living environment sound information and the sleep intention weighting information calculated from the sensing information.

For example, final sleep intention information can be acquired through the sleep intention information and sleep intention weighting information, and the time when the final sleep intention information exceeds a certain threshold can be determined as the sleep induction time. For instance, the receiving control unit 416 can calculate the final sleep intention information by multiplying the sleep intention information and the sleep intention weighting information. Specifically, if the sleep intention information calculated based on the living environment sound information is ‘9’ and the sleep intention weighting information calculated based on the sensing information is ‘0’, the final sleep intention information can be calculated as 0, and the receiving control unit 416 can determine that it does not exceed the predetermined threshold (e.g., 8.

In another example, if the sleep intention information is ‘9’ and the sleep intention weighting information is ‘1’, the final sleep intention information can be calculated as 9, and the receiving control unit 416 can determine that it exceeds the predetermined threshold (e.g., 8 and decide the corresponding time as the sleep induction time. The specific numerical descriptions of the sleep intention information, sleep intention weighting information, and final sleep intention information mentioned above are merely exemplary, and the present invention is not limited thereto. As described above, even if high sleep intention information is acquired through sound information, the final sleep intention information can change depending on whether the user is located in a certain position. For example, even if high sleep intention information (e.g., 10 is calculated based on the living environment sound information, if the user is not located in a certain position, the final sleep intention information becomes 0, and it can be finally determined that the user's sleep intention is low.

As described above, the receiving control unit 416 can determine the user's sleep induction time. Accordingly, when the receiving control unit 416 acquires the first sleep state information indicating that the user is before sleep, it can generate the first environment adjustment information to adjust the light from the sleep induction time to the time when the second sleep state information is acquired, supplying white light at 3000K with an illumination of 30 lux.

That is, the receiving control unit 416 can generate first environment adjustment information to adjust the light from the point in time when the user's state is predicted to be preparing for sleep (e.g., sleep induction point) until the point the user falls asleep (i.e., when the second sleep state information is acquired), and can decide to transmit the first environment adjustment information to the environment adjustment unit 415.

According to one embodiment of the present invention, the receiving control unit 416 can generate second environment adjustment information based on the second sleep state information. The second environment adjustment information may be control information to create a darkroom environment by minimizing illumination. That is, when the user's sleep state is during sleep, the receiving control unit 416 can minimize illumination to create a darkroom environment.

Specifically, when the receiving control unit 416 detects that the user has entered sleep (or a sleep stage) (i.e., when acquiring the second sleep state information), it can generate control information, namely, the second environment adjustment information, to prevent light from being supplied. Consequently, the probability of the user achieving deep sleep increases, thereby improving the quality of sleep.

According to one embodiment of the present invention, the receiving control unit 416 can generate third environment adjustment information based on the wake-up induction point.

Specifically, when the user's sleep state is during sleep, the receiving control unit 416 can generate third environment adjustment information to gradually increase the illumination of white light from the wake-up induction point until the user's wake-up point.

In one embodiment, the wake-up induction point may be determined based on the desired wake-up time information.

The desired wake-up time information may be information regarding the wake-up time desired by the user. For example, the desired wake-up time information acquired from the first user may relate to 7 AM. The specific description of the wake-up prediction point mentioned above is merely exemplary and does not limit the present invention.

In one embodiment, the desired wake-up time information may be acquired through information exchange with the user's user terminal 10. The user can set the desired sleep and wake-up times through the user terminal 10 and transmit them to the receiving control unit 416. The receiving control unit 416 can acquire the desired wake-up time information based on the wake-up time set by the user on the user terminal 10.

In another embodiment, the wake-up induction point may be determined based on the wake-up prediction point. Here, the wake-up prediction point may be determined based on the sleep onset point identified through the second sleep state information. Specifically, the receiving control unit 416 can identify the user's sleep onset point through the second sleep state information indicating that the user is asleep. The receiving control unit 416 can determine the wake-up prediction point based on the sleep onset point identified through the second sleep state information. For example, the receiving control unit 416 can determine the wake-up prediction point as 8 hours after the sleep onset point, which is considered an appropriate sleep duration. Specifically, if the user's sleep onset point is 11 PM, the receiving control unit 416 can determine the wake-up prediction point as 7 AM. The specific numerical descriptions of each point mentioned above are merely exemplary and do not limit the present invention. That is, the receiving control unit 416 can determine the wake-up prediction point based on the user's sleep onset point.

In yet another embodiment, the wake-up prediction point may be determined based on the user's sleep stage information. For example, a user can wake up most refreshed when waking during the REM stage. During a night's sleep, the user may cycle through light sleep, deep sleep, light sleep, and REM sleep, and waking during the REM sleep stage can result in the most refreshed awakening.

Accordingly, the receiving control unit 416 can determine the user's wake-up prediction time through the sleep stage information related to the user's sleep stages. For a specific example, the receiving control unit 416 can determine the time when the user transitions from the REM stage to another sleep stage as the wake-up prediction time. That is, the receiving control unit 416 can determine the wake-up prediction time based on the sleep stage information (i.e., the REM sleep stage) where the user can wake up most refreshed.

As described above, the receiving control unit 416 can determine the user's wake-up prediction time based on at least one of the sleep plan information, sleep onset time, and sleep stage information acquired from the user terminal. Additionally, when the receiving control unit 416 determines the wake-up prediction time, which is the time the user wishes to wake up, it can determine the wake-up induction time based on this wake-up prediction time. For example, the receiving control unit 416 can determine the wake-up induction time as 30 minutes before the time the user wishes to wake up. Specifically, if the wake-up prediction time set by the user is 7:00 AM, the receiving control unit 416 can determine 6:30 AM as the wake-up induction time. The specific description of the aforementioned times is merely exemplary and does not limit the present invention.

That is, the receiving control unit 416 can identify the wake-up induction time by recognizing the wake-up prediction time when the user's wake-up is anticipated, and generate third environment adjustment information to gradually increase the supply of 3000K white light from 0 lux to 250 lux from the wake-up induction time until the wake-up time (e.g., until the user actually wakes up). The receiving control unit 416 can decide to transmit this third environment adjustment information to the environment adjustment unit 415, and accordingly, the environment adjustment unit 415 can perform light-related adjustment operations in the space where the user is located based on the third environment adjustment information. For example, the environment adjustment unit 415 can gradually increase the 3000K white light from 0 lux to 250 lux starting 30 minutes before wake-up.

According to one embodiment of the present invention, the receiving control unit 416 can acquire living environment sound information and, based on this sound information, acquire sleep sound information.

According to one embodiment of the present invention, the receiving control unit 416 can perform pre-processing on the sleep sound information. The pre-processing of sleep sound information may involve noise removal. Specifically, the receiving control unit 416 can classify the sleep sound information into one or more sound frames with predetermined time units.

Additionally, the receiving control unit 416 can identify the minimum sound frame with the lowest energy level based on the energy level of each of the one or more sound frames. The receiving control unit 416 can perform noise removal on the sleep sound information based on the minimum sound frame.

For a specific example, the receiving control unit 416 can classify 30 seconds of sleep sound information into one or more very short sound frames of 40 ms size. Additionally, the receiving control unit 416 can compare the size of each of the multiple sound frames related to the 40 ms size to identify the minimum sound frame with the lowest energy level. The receiving control unit 416 can remove the identified minimum sound frame component from the entire sleep sound information (i.e., 30 seconds of sleep sound information). For instance, as the minimum sound frame component is removed from the sleep sound information, pre-processed sleep sound information can be acquired. That is, the receiving control unit 416 can identify the minimum sound frame as a background noise frame and remove it from the original signal (i.e., sleep sound information) to perform pre-processing related to noise removal.

Additionally, as shown in FIG. 6(a), the receiving control unit 416 can generate a spectrogram 300 corresponding to the sleep sound information 210. Here, the sleep sound information 210 may refer to the pre-processed sleep sound information. That is, the receiving control unit 416 can generate a spectrogram corresponding to the pre-processed sleep sound information. Regarding the generation of the spectrogram, redundant explanations will be avoided as detailed above.

According to one embodiment of the present invention, the spectrogram generated by the receiving control unit 416 corresponding to the sleep sound information 210 may include a Mel spectrogram. The receiving control unit 416 can acquire a Mel spectrogram through a Mel-Filter Bank applied to the spectrogram.

Generally, the human cochlea may have different vibrating areas depending on the frequency of voice data. Additionally, the human cochlea has the characteristic of detecting frequency changes well in the low-frequency band but not in the high-frequency band. Accordingly, a Mel spectrogram can be acquired from the spectrogram using a Mel-Filter Bank to have a recognition ability similar to the characteristics of the human cochlea regarding voice data. That is, the Mel-Filter Bank may apply fewer filter banks in the low-frequency band and wider filter banks as it goes to the high-frequency band. In other words, the receiving control unit 416 can acquire a Mel spectrogram by applying a Mel-Filter Bank to the spectrogram to recognize voice data similarly to the characteristics of the human cochlea. The Mel spectrogram may include frequency components reflecting human auditory characteristics. That is, the spectrogram generated corresponding to the sleep sound information in the present invention, which is subject to analysis using a neural network, may include the aforementioned Mel spectrogram.

Additionally, the receiving control unit 416 can process the spectrogram 300 as input to the sleep analysis model to acquire sleep stage information. Here, the sleep analysis model is a model for acquiring sleep stage information related to changes in the user's sleep stages, and it can output sleep stage information by using sleep sound information acquired during the user's sleep as input. In the embodiment, the sleep analysis model may include a neural network model configured through one or more network functions. Regarding network functions, redundant explanations will be avoided as detailed above.

As described above, the receiving control unit 416 can acquire a spectrogram based on the sleep sound information. In this case, the conversion to a spectrogram may be intended to facilitate the analysis of breathing or movement patterns related to relatively small sounds. Additionally, the receiving control unit 416 can generate sleep stage information based on the acquired spectrogram by utilizing a sleep analysis model, which includes a feature extraction model and a feature classification model. In this case, the sleep analysis model can perform sleep stage prediction by inputting spectrograms corresponding to multiple epochs to consider both past and future information, thereby outputting more accurate sleep stage information.

That is, the receiving control unit 416 can output sleep stage information corresponding to the sleep sound information by utilizing the sleep analysis model as described above. According to an embodiment, the sleep stage information may relate to information about the changing sleep stages of a user during sleep. For example, the sleep stage information may indicate how the user's sleep changed to light sleep, normal sleep, deep sleep, or REM sleep at each point during the user's 8-hour sleep last night. The specific description of the aforementioned sleep stage information is merely exemplary and does not limit the present invention.

According to one embodiment of the present invention, the receiving control unit 416 can perform data augmentation based on pre-processed sleep sound information. This data augmentation is intended to enable the sleep analysis model to robustly output sleep state information (e.g., sleep stage information) even for sounds measured in various domains (e.g., different bedrooms, different microphones, different placement positions). In the embodiment, data augmentation may include at least one of pitch shifting, Gaussian noise, loudness control, dynamic range control, and spec augmentation.

According to one embodiment, the receiving control unit 416 can perform data augmentation related to pitch shifting based on the sleep sound information. For example, the receiving control unit 416 can perform data augmentation by adjusting the pitch of the sound, such as raising or lowering the pitch at predetermined intervals.

According to the present invention, the receiving control unit 416 can perform data augmentation not only through pitch shifting but also through Gaussian noise, which involves correction related to noise, loudness control, which corrects the sound to maintain sound quality even when the volume is changed, dynamic range control, which adjusts the logarithmic ratio of the maximum and minimum amplitudes of the sound measured in dB, and spec augmentation related to the increase in sound specifications.

That is, through data augmentation of the sound information (i.e., sleep sound information) that forms the basis of the analysis of the present invention, the receiving control unit 416 can enhance the accuracy of sleep stage prediction by enabling the sleep analysis model to perform robust recognition corresponding to sleep sounds acquired in various environments.

According to one embodiment of the present invention, the receiving control unit 416 can acquire fourth environment adjustment information based on third sleep state information. The fourth environment adjustment information is the same as described in relation to the operation of the processor 130 in the embodiment of FIG. 1(a), so redundant description will be omitted.

According to one embodiment of the present invention, the receiving control unit 416 can decide to transmit environment adjustment information to the environment adjustment unit 415. Specifically, the receiving control unit 416 can generate environment adjustment information related to illumination adjustment and decide to transmit the corresponding environment adjustment information to the environment adjustment unit 415, thereby controlling the illumination adjustment operation of the environment adjustment unit 415.

According to an embodiment, light or air quality may be one of the representative factors that can affect the quality of sleep. For example, the quality of sleep can be positively or negatively affected depending on the illumination, color, and exposure level of light. Additionally, the quality of sleep is greatly influenced by the type/concentration of fine dust, the type/concentration of harmful gases, the presence of allergenic substances, and the temperature or humidity of the air. Accordingly, the receiving control unit 416 can adjust the illumination or air quality to improve the user's sleep quality. For example, the receiving control unit 416 can monitor the situation before or after falling asleep and perform illumination adjustment to effectively wake the user. That is, the receiving control unit 416 can automatically adjust the illumination or air quality by identifying the sleep state (e.g., sleep stage) to maximize sleep quality.

In one embodiment, the receiving control unit 416 can receive sleep plan information from the user terminal 10. The sleep plan information is information generated by the user through the user terminal 10 and may include, for example, desired sleep time information and desired wake-up time information. The receiving control unit 416 can generate external environment adjustment information based on the sleep plan information. For a specific example, the receiving control unit 416 can identify the user's sleep time through the sleep plan information and generate environment adjustment information based on the identified sleep time.

Additionally, the receiving control unit 416 can receive sleep planning information from the user terminal 10 and, based on this, generate first environment adjustment information to control the smart home-appliance according to an embodiment of the present invention, from the time predicted as the user preparing for sleep (e.g., sleep induction time) to the time the user falls asleep (i.e., the time when the second sleep state information is acquired).

Furthermore, for example, the receiving control unit 416 can identify the time the user enters sleep, i.e., the sleep entry time, through the second sleep state information, and based on this, generate second environment adjustment information.

In an embodiment, the receiving control unit 416 can generate environment adjustment information based on sleep stage information. In this embodiment, the sleep stage information may include information on the user's sleep stage changes acquired in a time-series manner through the analysis of sleep sound information.

Additionally, the receiving control unit 416 can generate environment adjustment information to provide appropriate illumination according to the user's sleep stage changes during sleep.

Moreover, for example, the receiving control unit 416 can identify the user's desired wake-up time through sleep planning information, generate a wake-up prediction time based on the desired wake-up time, and accordingly generate environment adjustment information.

Furthermore, the receiving control unit 416 can decide to transmit the environment adjustment information to the environment adjustment unit 415. That is, the receiving control unit 416 can generate environment adjustment information based on sleep planning information to facilitate the user falling asleep or waking up naturally at bedtime or wake-up time, and by controlling the environment adjustment operation of the environment adjustment unit 415 through this environment adjustment information, the quality of the user's sleep can be improved.

In an additional embodiment, the receiving control unit 416 can generate recommended sleep planning information based on sleep stage information.

According to one embodiment, the receiving control unit 416 can update the environment adjustment information by comparing the user's actual wake-up time with the desired wake-up time information.

According to the present invention, the receiving control unit 416 performs a comparison of the desired wake-up time information and the actual wake-up time information, and if the comparison result shows that the information differs, it can update the environment adjustment information. Here, the actual wake-up time information compared with the desired wake-up time information may include information on actual wake-up times accumulated over a certain number of occurrences. For example, the actual wake-up time information may include information on the user's actual wake-up times over a week.

In an embodiment, the receiving control unit 416 can analyze the difference between the desired wake-up time and the accumulated actual wake-up time to update the environment adjustment information. Specifically, if the actual wake-up time is later than the desired wake-up time, the receiving control unit 416 can gradually increase the maximum brightness of white light supplied at the wake-up time to advance the user's circadian rhythm. For instance, if the actual wake-up time is later than the desired wake-up time, the environment adjustment information can be updated so that the maximum brightness of white light supplied at the user's wake-up time is higher than the previous day. Conversely, if the actual wake-up time is earlier than the desired wake-up time, the receiving control unit 416 can decrease the maximum brightness of white light supplied at the wake-up time to delay the user's wake-up time. For example, if the actual wake-up time is earlier than the desired wake-up time, the environment adjustment information can be updated so that the maximum brightness of white light supplied at the user's wake-up time is lower than the previous day. In other words, the receiving control unit 416 can compare the user's actual wake-up time with the desired wake-up time and update the environment adjustment information to alter the user's circadian rhythm based on the comparison result. Consequently, an optimized sleep environment can be created for the user, thereby further enhancing sleep efficiency.

According to an embodiment of the present invention, the receiving control unit 416 may drive the sound collection unit through at least one of a manual sleep measurement mode and an automatic sleep measurement mode to collect sound information, and calculate sleep state information based on the collected sound information.

In the embodiment, the manual sleep measurement mode may mean that the measurement mode is passively initiated as a sleep input signal is generated by the user.

For example, the user may generate a sleep input signal by applying physical pressure to a sleep input button formed on the exterior of the sleep environment adjustment device 400, or by utilizing a user terminal. When the sleep input signal is generated, the sleep environment adjustment device 400 (i.e., the receiving module) acquires sound information related to the space at that point in time, and the user's sleep state information can be acquired based on the sound information. That is, through the manual sleep measurement mode, the user can directly determine the point in time to initiate the measurement of their sleep state.

Furthermore, according to the embodiment, the automatic sleep measurement mode may mean that sleep measurement is automatically initiated without the need for a separate user action to generate a sleep input signal. The automatic sleep measurement mode may be characterized by the initiation of the measurement mode automatically when, after detecting the occurrence of user movement within a space through the first sensor unit, the user is identified as being located in a predefined area through the second sensor unit. A detailed description of the automatic sleep measurement mode will be provided later with reference to FIG. 13. Additionally, descriptions of matters overlapping with the previously explained content will be omitted.

FIG. 13 is a flowchart exemplarily illustrating the process of generating sleep state information through the automatic sleep measurement mode related to an embodiment of the present invention. The steps shown in FIG. 13 may be reordered as necessary, and at least one step may be omitted or added. That is, the aforementioned steps are merely one embodiment of the present invention, and the scope of rights of the present invention is not limited thereto.

According to one embodiment, the receiving control unit 416 can detect the occurrence of user movement within a space through the first sensor unit (S110).

The first sensor unit may be equipped to include at least one of a PIR sensor and an ultrasonic sensor. The PIR sensor can detect user movement within the detection range by sensing the change in infrared radiation emitted from the user's body. For example, the PIR sensor can detect user movement in the bedroom by identifying infrared radiation of 8 μm~14 μm emitted from the user's body.

The ultrasonic sensor can detect the movement of an object by emitting sound waves and sensing the signals reflected back from a specific object. For instance, the ultrasonic sensor can emit sound waves within the bedroom space and detect the occurrence of user movement inside the bedroom through the sound waves reflected from the user's body as the user enters the bedroom.

According to one embodiment, the receiving control unit 416 can identify that the user is located in a predefined area through the second sensor unit (S120).

According to one embodiment, the receiving control unit 416 can drive the sound collection unit 414 to collect sound information related to the space (S130). That is, the receiving control unit 416 can detect the occurrence of user movement within the space through the first sensor unit, and when the user is identified as moving within the predefined area through the second sensor unit, it can automatically collect sound information related to the space through the sound collection unit 414.

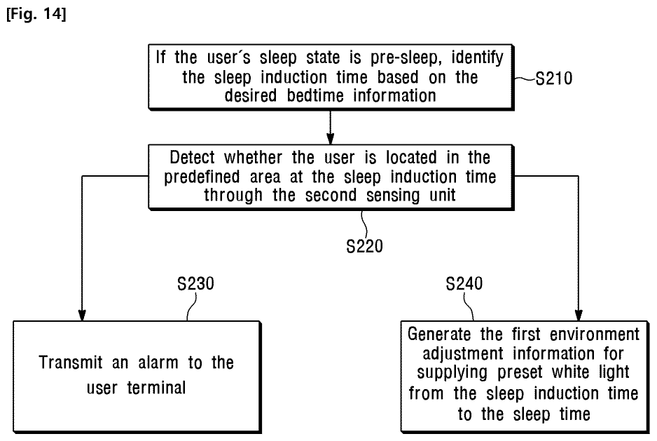

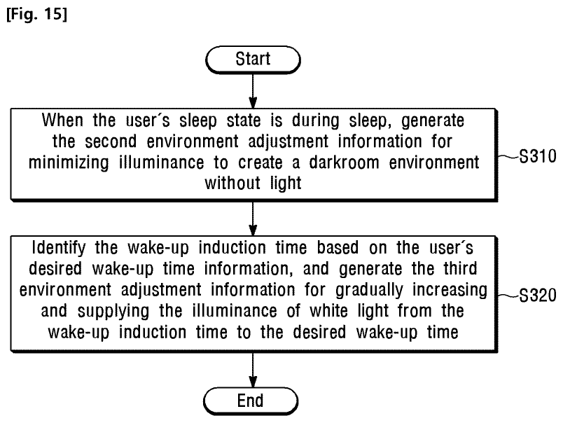

FIG. 14 is a flowchart illustratively showing a process of creating an environment to induce a user's sleep initiation according to an embodiment of the present invention. The steps shown in FIG. 14 may be reordered as necessary, and at least one step may be omitted or added. That is, the aforementioned steps are merely one embodiment of the present invention, and the scope of rights of the present invention is not limited thereto.

According to one embodiment, the receiving module 410 can identify a sleep induction time based on the desired sleep time information when the user's sleep state is pre-sleep (S210). Specifically, the user can set the time they wish to sleep and wake up through the user terminal 10 to generate sleep plan information, which can then be transmitted to the receiving module 410. In this case, the sleep plan information may include desired sleep time information and desired wake-up time information. The receiving module 410 can identify the sleep induction time based on the desired sleep time information. For example, the receiving module 410 may determine a time 20 minutes before the user's intended sleep time (i.e., desired sleep time) as the sleep induction time. Detailed examples related to the sleep induction time have been previously described and will be omitted here.

Additionally, the receiving module 410 can detect whether the user is located in a predefined area at the sleep induction time through the second sensor unit (S220).

In the embodiment, if it is detected that the user is not located in the predefined area, the receiving module 410 can send a notification to the user terminal (S230). Specifically, if the sleep induction time is imminent but the user is not located in the predefined area, a notification can be sent to the user terminal to prompt the user to prepare for sleep.

Furthermore, in the embodiment, if it is detected that the user is located in the predefined area, the receiving module 410 can generate first environment adjustment information to supply predefined white light from the sleep induction time to the sleep time (S240). That is, the first environment adjustment information can be generated only when the user is located in the predefined area corresponding to the sleep induction time.

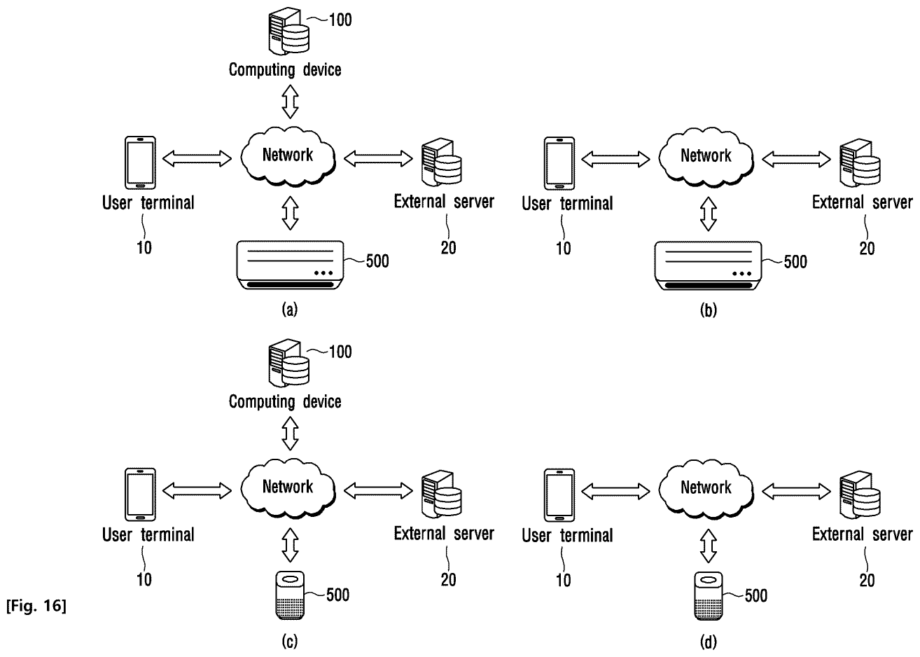

FIG. 15 is a flowchart illustratively showing a process of changing the user's sleep environment during sleep and just before waking according to an embodiment of the present invention. The steps shown in FIG. 15 may be reordered as necessary, and at least one step may be omitted or added. That is, the aforementioned steps are merely one embodiment of the present invention, and the scope of rights of the present invention is not limited thereto.

According to one embodiment, the receiving module 410 can generate second environment adjustment information to create a darkroom environment with minimized illumination when the user's sleep state is during sleep (S310).

That is, when the receiving module 410 detects that the user has entered sleep (or a sleep stage) (i.e., acquires second sleep state information), it can generate control information, namely, second environment adjustment information, to prevent light from being supplied. As a result, the probability of the user achieving deep sleep increases, thereby improving the quality of sleep.

According to one embodiment, the receiving module 410 can identify a wake-up induction time based on the desired wake-up time information and generate third environment adjustment information to gradually increase and supply the illumination of white light from the wake-up induction time to the desired wake-up time (S320). For instance, the third environment adjustment information may be characterized by control information to gradually increase the illumination of 3000K white light from 0 lux to 250 lux from the wake-up induction time to the wake-up time.

That is, when the user's sleep state is during sleep, the receiving module 410 can generate third environment adjustment information to gradually increase and supply the illumination of white light from the wake-up induction time to the user's wake-up time. For example, the third environment adjustment information may relate to control information for gradually increasing the illumination starting 30 minutes before the user's wake-up time (i.e., wake-up induction time).

Furthermore, the detailed description related to the environment adjustment information and the operation of the smart home-appliance corresponding thereto is identical to that described through the processor 130 of FIG. 1(a), and thus, a detailed description will be omitted.

Additionally, although the configuration of the sleep environment adjustment device and its various operations have been described above, the aforementioned operations are not limited to being performed by the sleep environment adjustment device. For instance, in an embodiment such as FIG. 1(c), at least one of the electronic devices depicted in FIG. 1(c) may perform at least one of the operations of the aforementioned sleep environment adjustment device.

### Configuration of Smart Home-Appliance According to Some Embodiments of the Present Invention

### Air Conditioner

### Operation of the Air Conditioner

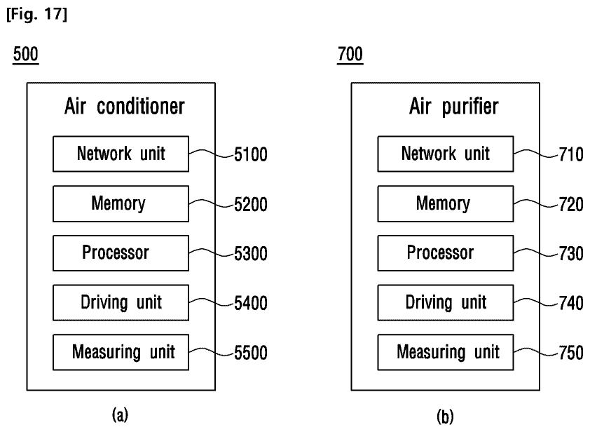

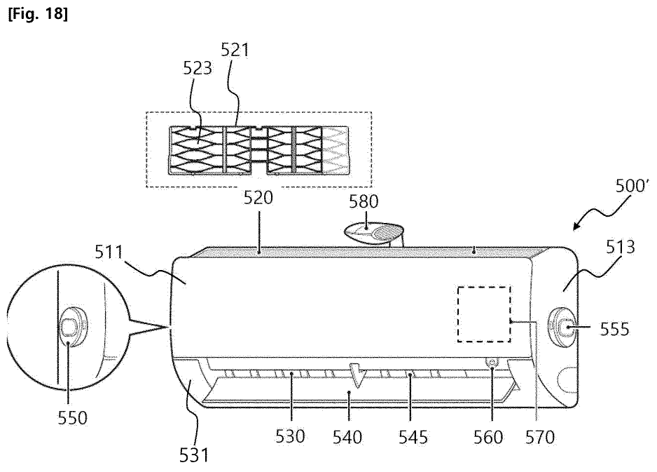

Hereinafter, the air conditioner according to the present invention will be described in detail. FIGS. 16(a) and 16(b) are conceptual diagrams for explaining the operation of the air conditioner according to the present invention.

Specifically, FIG. 16(a) is a schematic diagram where the environment adjustment device 30 of FIG. 1(a) is implemented as an air conditioner 500, and FIG. 16(b) is a schematic diagram showing the air conditioner 500 operating in conjunction with a user terminal 10.

As shown in FIG. 16(a), the air conditioner 500 according to the present invention can operate in conjunction with the user terminal 10 and the computing device 100.

The computing device 100 may include a network unit 110, a memory 120, and a processor 130 (see FIG. 2). The network unit 110 transmits and receives data with the user terminal 10, the external server 20, and the air conditioner 500. The network unit 110 can transmit and receive data for performing a sleep environment adjustment method according to sleep state information in one embodiment of the present invention with other computing devices, servers, etc. That is, the network unit 110 can provide communication functions between the computing device 100, the user terminal 10, the external server 20, and the air conditioner 500. For example, the network unit 110 can receive sleep examination records and electronic health records for multiple users from a hospital server. In another example, the network unit 110 can receive environment sensing information related to the space where the user is active from the user terminal 10. In yet another example, the network unit 110 can transmit environment adjustment information related to temperature and/or humidity to adjust the environment of the space where the user is located to the air conditioner 500.

Here, the operation method, hardware configuration, and software configuration of the network unit 110 and the memory 120 are the same as described above, so redundant descriptions will be omitted.

According to the present invention, the processor 130 can provide a sleep analysis model according to an embodiment of the present invention by reading a computer program stored in the memory 120. In one embodiment of the present invention, the processor 130 can perform calculations to derive environment adjustment information based on sleep state information. In another embodiment of the present invention, the processor 130 can perform calculations to train the sleep analysis model. The specific details of the sleep analysis model are as described above.

According to the present invention, the processor 130 can acquire the user's sleep state information and environment sensing information, as described above. The processor 130 can generate the first environment adjustment information through the nth environment adjustment information. Specifically, if the user's state is pre-sleep, the processor 130 can generate the first environment adjustment information to control the air conditioner from the predicted time when the user is preparing to sleep (e.g., sleep induction time) until the time the user falls asleep (i.e., when the second sleep state information is acquired). Specifically, the processor 130 can generate the first environment adjustment information to control the air conditioner to optimize the indoor temperature and/or humidity seasonally or per user until a predetermined time before sleep (e.g., 20 minutes prior).

Alternatively, the first environment adjustment information may include controlling the air conditioner to induce a level of noise (white noise) that can lead to sleep just before sleep, adjusting the blowing strength to a predefined level or lower, reducing the intensity of the LED, or converting direct airflow to indirect airflow. Additionally, the first environment adjustment information may include information to control the air conditioner to perform dehumidification/humidification based on the temperature and/or humidity information within the sleeping space.

According to one embodiment of the present invention, the processor 130 can generate the second environment adjustment information to control the air conditioner by lowering the brightness of the display unit of the air conditioner, turning off the display unit, operating the air conditioner at a noise level below a predefined level, adjusting the blowing strength to a predefined level or lower, setting the blowing temperature within a predefined range, maintaining the humidity within the sleeping space at a predetermined value, or maintaining indirect airflow based on the second sleep state information.

Furthermore, the processor 130 can generate the third environment adjustment information and the fourth environment adjustment information based on the third sleep state information and the fourth sleep state information.

According to the present invention, the processor 130 can decide to transmit the environment adjustment information to the air conditioner 500. That is, by generating external environment adjustment information that allows the user to easily fall asleep or wake up naturally at bedtime or wake-up time, the processor 130 can improve the quality of the user's sleep.

As shown in FIG. 16(b), the air conditioner 500 according to the present invention can operate in conjunction with the user terminal 10. That is, the system according to the embodiment shown in FIG. 16(b) may include the air conditioner 500, the user terminal 10, an external server 20, and a network. In this embodiment, the air conditioner 500 according to the present invention includes the configuration of the computing device 100 shown in FIG. 16(a) and additional configurations for operating as an air conditioner.

FIG. 17(a) is a block diagram showing the configuration of the air conditioner according to the present invention. As shown in FIG. 17(a), the air conditioner 500 according to the present invention may include a network unit 5100, memory 5200, processor 5300, drive unit 5400, and measurement unit 5500.

According to the present invention, the air conditioner 500 may be implemented as a wall-mounted air conditioner or a wall-mounted heating and cooling air conditioner fixedly installed on the wall of a building, apartment, or house, a system air conditioner embedded in the ceiling, a stand air conditioner placed on one side or corner of an indoor space, or a portable air conditioner that is easy to carry and move.

According to the present invention, the functions, operations, hardware configuration, and software configuration of the network unit 5100, memory 5200, and processor 5300 of the air conditioner 500 are as described above. The first through nth environment adjustment information generated by the processor 5300 can be delivered to the drive unit 5400. The drive unit 5400 operates various hardware elements provided in the air conditioner 500.

According to the present invention, the measurement unit 5500 may include one or more sensors for sensing temperature, humidity, dust concentration, and the condition of air conditioner components within a sleep space. Specifically, the measurement unit may include a dust sensor for detecting invisible particulate matter such as PM1.0, PM2.5, and PM10, an illuminance sensor for detecting indoor illuminance, a temperature sensor for measuring indoor temperature, and a humidity sensor for measuring indoor humidity.

Additionally, the measurement unit 5500 may include a human-body detection sensor. The processor 5300 can detect the user through the human-body detection sensor and perform control to send cool or warm air to the space where the user is located (direct airflow) or to a space where the user is not located (indirect airflow).

Furthermore, the measurement unit 5500 may further include a voice-recognition sensor for recognizing the user's voice.

Although not shown in the drawings, the air conditioner 500 may be composed of a housing equipped with a discharge port and an inlet, a filter unit, a blower fan, a sterilization unit, a humidification unit, a heating unit, a cooling unit, and a measurement unit. The housing can be designed in various ways depending on the implementation method of the air conditioner 500, such as a wall-mounted air conditioner, a system air conditioner, or a stand-type air conditioner. The filter unit can be selected to correspond to methods such as dust-collection filter type or adsorption filter type. The blower fan may be connected to a motor that rotates by power supplied from the power supply unit. The sterilization unit has the function of sterilizing the inhaled air using chemical or electrical methods. The humidification unit has the function of humidifying and discharging the inhaled air, and the heating and cooling units have the function of heating or cooling the inhaled air to a predetermined temperature.

The hardware elements of the air conditioner 500 described above are merely one embodiment, and some of them may be integrated into a single configuration, some configurations may be omitted, and various configurations for performing air purification functions not described above may be added.

Meanwhile, environment sensing information can be acquired through the user terminal 10. The environment sensing information may be sleep sound information acquired from the bedroom where the user is sleeping.

Additionally, the environment sensing information may be temperature and/or humidity or air quality information within the sleep space acquired from the measurement unit 5500 provided in the air conditioner 500. The environment sensing information acquired through the user terminal 10 or the measurement unit 5500 may serve as foundational information for acquiring the user's sleep state information in the present invention.

For a specific example, sleep state information related to whether the user is before sleep, during sleep, or after sleep can be acquired through environment sensing information obtained in relation to the user's activities. Additionally, information related to the surrounding air quality before, during, and after the user's sleep can be acquired.

According to the present invention, the processor 5300 can acquire sleep state information based on the environment sensing information obtained through the user terminal 10 and/or the measurement unit 5500.

Specifically, the processor 5300 can identify singularities where information of a predefined pattern in the environment sensing information is detected. Here, the information of a predefined pattern may relate to breathing and movement patterns related to sleep. For example, in the wake state, all nervous systems are activated, resulting in irregular breathing patterns and frequent body movements. Additionally, because the relaxation of the neck muscles does not occur, breathing sounds may be minimal.

On the other hand, when a user is sleeping, the autonomic nervous system stabilizes, resulting in regular changes in breathing and reduced body movement, and the breathing sound may become louder. Specifically, the processor 5300 can identify the point in time when acoustic information related to a predefined pattern, such as regular breathing, minimal body movement, or minimal breathing sound, is detected in the environment sensing information as a singularity. Furthermore, the processor 5300 can acquire sleep sound information based on the environment sensing information obtained around the identified singularity. The processor 5300 can identify singularities related to the user's sleep onset in the time-series environment sensing information and acquire sleep sound information based on these singularities.

Additionally, the temperature and/or humidity, and/or air quality measured through the measurement unit 5500 significantly affect the user's sleep. According to studies analyzing the relationship between temperature and/or humidity and sleep, there are differences in the frequency of awakenings and the proportion of deep sleep during sleep, adversely affecting the user's work efficiency the next day. Studies analyzing the relationship between air quality and sleep have confirmed that sleep disorders show a statistically significant association with air pollution. For example, in an experiment comparing CO2 levels 800 ppm, 1700 ppm) and temperatures 24° C., 28° C.), it was confirmed that sleep efficiency and next-day work efficiency decreased when sleeping in a 28° C. chamber, and the air felt more stuffy and hot at 800 ppm CO2.

Furthermore, in an experiment comparing sleep conditions at 32° C. (relative humidity 80%) and 26° C. (relative humidity 50%), it was confirmed that the frequency of awakenings increased, and the proportion of deep sleep decreased at 32° C. (relative humidity 80%). Meanwhile, exposure to PM10 can make it difficult to maintain sleep, and it was found that the probability of sleep disorders is highest when males are exposed to PM1. Additionally, for females, the likelihood of sleep disorders is highest when exposed to PM1 and PM2.5. It was also confirmed that the probability of sleep disturbances related to wheezing is highest when SO2 and O3 levels are high. Moreover, it was confirmed that if pregnant women are exposed to PM2.5 between 31 and 35 weeks of pregnancy, the likelihood of the newborn having a shorter sleep duration is highest. Various studies have been conducted on the association between AHI and air quality measurement indices, and although results vary slightly between studies, the high correlation between air quality and sleep remains consistent.

According to the present invention, the air conditioner 500 can acquire sleep state information based on environment sensing information, generate environment adjustment information, and perform appropriate actions for the sleep stage using this information.

Specifically, if the processor 5300 of the air conditioner 500 determines that the user's state is pre-sleep, it can generate first environment adjustment information to control the air conditioner from the predicted time when the user is preparing for sleep (e.g., sleep induction time) until the time the user falls asleep (i.e., when the second sleep state information is acquired). The first environment adjustment information can be generated by reflecting the PM concentration, harmful gas concentration, CO2 concentration, SO2 concentration, O3 concentration, humidity, temperature, etc., measured by the measurement unit 5500.

The first environment adjustment information may include information to control the air conditioner to optimize the indoor temperature and/or humidity until a predetermined time before the user's sleep (e.g., 20 minutes prior), to induce sleep by generating a level of noise (white noise) just before sleep, to adjust the blowing strength to a predefined level or lower, to lower the intensity of the LED, to convert direct airflow to indirect airflow, or to execute dehumidification/humidification based on the temperature and humidity information within the sleep space.

Additionally, based on the second sleep state information, the processor 5300 can generate second environment adjustment information to lower the brightness of the air conditioner's display unit, turn off the display unit, operate the air conditioner at a noise level below a predefined level, adjust the blowing strength to a predefined level or lower, set the blowing temperature within a predefined range, maintain the humidity in the sleep space at a predetermined temperature, or maintain indirect airflow.

According to the present invention, the second environment adjustment information is based on the second sleep state information and may include control information to lower the brightness of the air conditioner's display unit, turn off the display unit, operate the air conditioner at a noise level below a predefined level, adjust the blowing strength to a predefined level or lower, set the blowing temperature within a predefined range, maintain the humidity in the sleep space at a predetermined temperature, or maintain indirect airflow. The user can be induced to sleep with optimized temperature and humidity in the sleep space just before sleep, with airflow, white noise, etc., and can have a sound sleep with optimal temperature, humidity, etc., controlled after falling asleep.

### Configuration of the Air Conditioner According to the Embodiment

FIG. 18 is a diagram for explaining an example of the air conditioner shown in FIGS. 16 and 17.

Referring to FIG. 18, an air conditioner according to an example of the present invention includes an indoor unit 500′). For instance, the indoor unit 500′) may be a wall-mounted indoor unit installed on the wall of an indoor space.

The indoor unit 500′) includes a casing that forms its exterior. The casing includes a front unit 511 forming the front exterior of the indoor unit and side units 513 provided on both sides of the front unit 511 and extending rearward from the front unit 511 toward the wall.

Additionally, the casing further includes a rear unit (not shown) disposed at the rear side of the side units 513. The rear unit (not shown) is positioned between the side units 513 and can be coupled to the wall of the indoor space.

According to one embodiment of the present invention, the front unit 511, side units 513, and rear unit (not shown) form an internal space, within which multiple components provided in the indoor unit 500′) can be accommodated. These components may include a heat exchanger (not shown), a fan (not shown), a processor (not shown), and others.

According to the present invention, the indoor unit 500′) further includes a filter assembly 520 disposed on the upper part of the casing. The filter assembly 520 can form the upper surface of the casing. The filter assembly 520 is provided with multiple filter inlets 523 that draw air from the indoor space into the indoor unit 500′). The air drawn through the filter inlets 523 can be cooled or heated as it passes through the heat exchanger.

The filter assembly 520 is formed on the upper surface of the casing and is positioned at the inlet where air is drawn in to filter the air. The inlet may be an opening formed by the upper surfaces of the front unit 511, side units 513, and rear unit (not shown) of the casing.

The filter assembly 520 may include a filter member 521. The filter member 521 is a filter that removes yellow dust or ultrafine particles and may have an antibacterial function applied to an antibacterial microfilter.

According to one embodiment of the present invention, the filter member 521 may be configured with multiple filter inlets 523, and in this case, the filter member 521 may be composed of multiple frames for forming the filter inlets 523. Additionally, a mesh may be arranged in the filter inlets 523 to filter the air being drawn in.

The indoor unit 500′) further includes a discharge panel 531 disposed at the lower part of the casing, having a discharge port 530 through which the air drawn into the indoor unit 500′) is expelled. The discharge port 530 may be provided with an up-and-down air-direction controller 540 that is movably provided to adjust the discharge direction or airflow of the air expelled from the discharge port 530. For example, the up-and-down air-direction controller 540 may be provided to rotate forward and backward around hinge axes provided at both ends of the air-direction controller 540 to adjust the direction of the wind upward or downward. Additionally, the discharge port 530 may be provided with a left-and-right air-direction controller 545 that is movably provided to adjust the discharge direction or airflow of the air expelled from the discharge port 530.

When the fan operates, the air in the indoor space is drawn into the interior of the indoor unit 500′) through the filter assembly 520 and undergoes heat exchange in the heat exchanger. The heat-exchanged air can then be expelled through the discharge port 530.

In this embodiment, it is described that the filter assembly 520 for air intake by the indoor unit is positioned at the upper part of the indoor unit, and the discharge port 530 for air exhaust is positioned at the lower part of the indoor unit 500′). However, conversely, the filter assembly 520 may be located at the lower part of the indoor unit, and the discharge port 530 may be located at the upper part of the indoor unit. As another example, an additional filter assembly or discharge unit may be formed on the front unit 511 of the casing.

In summary, the indoor unit 500′) according to the present invention can be configured to generate airflow patterns such as upper intake and lower discharge, lower intake and upper discharge, front intake and lower discharge, or upper intake and front discharge.

A sensor device 550 capable of detecting the amount of dust contained in the air of the indoor space may be provided on the side unit 513 of the casing. By installing the sensor device 550 on the side unit 513, it can detect the amount of dust by inhaling only a small amount of air without being affected by the intake and discharge airflow through the indoor unit 500′).

According to the present invention, a voice-recognition sensor 555 for recognizing a user's voice may be disposed on the side unit 513 of the casing.

A forced-operation button 560 that can be used to forcibly turn on or off the air conditioner, or during trial operation, may be disposed on the front unit 511 of the casing.

A display unit 570 that allows checking the desired temperature and the operation status of additional functions when the air conditioner is operating may be disposed on the front unit 511 of the casing. Here, a sensor for receiving remote control signals may be disposed in the display unit 570.

The ion generator 580 is disposed on the upper side of the indoor unit 500′) and is a means capable of diffusing ions to both sides. Specifically, the ion generator 580 is coupled to the frame inside the casing and is a means capable of diffusing ions to both sides.

The ion generator 580 generates high voltage to both sides, thereby ionizing molecules in the air, and as a result, dust is charged by the ionized molecules, and the charged dust can be effectively collected by the filter assembly 520.

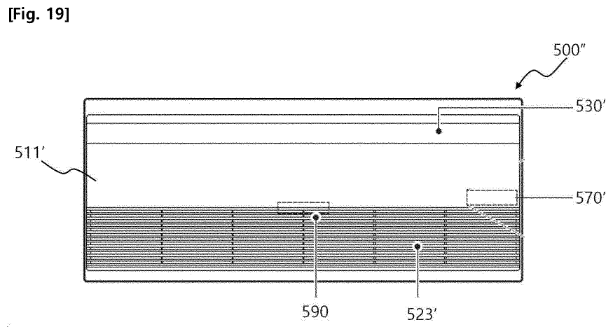

FIG. 19 is a diagram for explaining another example of the air conditioner shown in FIGS. 16 and 17.

Referring to FIG. 19, the air conditioner according to another example of the present invention includes an indoor unit 500″). For example, the indoor unit 500″) may be an indoor unit for a system air conditioner installed on the ceiling of an indoor space.

In another embodiment of the present invention, the indoor unit 500″) includes a casing. The casing may include a front unit 511′). Although not shown in the drawings, the casing may further include other parts besides the front unit 511′).

The front unit 511′) may be the part visible to a user when looking at the ceiling.

The front unit 511′) may be formed with an inlet 523′) through which indoor air is drawn in, and a discharge port 530′) through which cold or hot air is expelled.

The front unit 511′) may be equipped with a display unit 570′) that allows the operational status of the air conditioner to be checked. The display unit 570′) may include a receiving unit for receiving signals from a wireless remote control. Additionally, a forced-operation button may also be arranged.

The front unit 511′) may be equipped with an air-purification indicator 590 that displays various colors according to the indoor air condition.

A predetermined internal space is formed inside the casing, and a plurality of components provided in the indoor unit 500″) may be accommodated within the internal space.

The plurality of components may include a heat exchanger (not shown), a fan (not shown), a processor (not shown), and others.

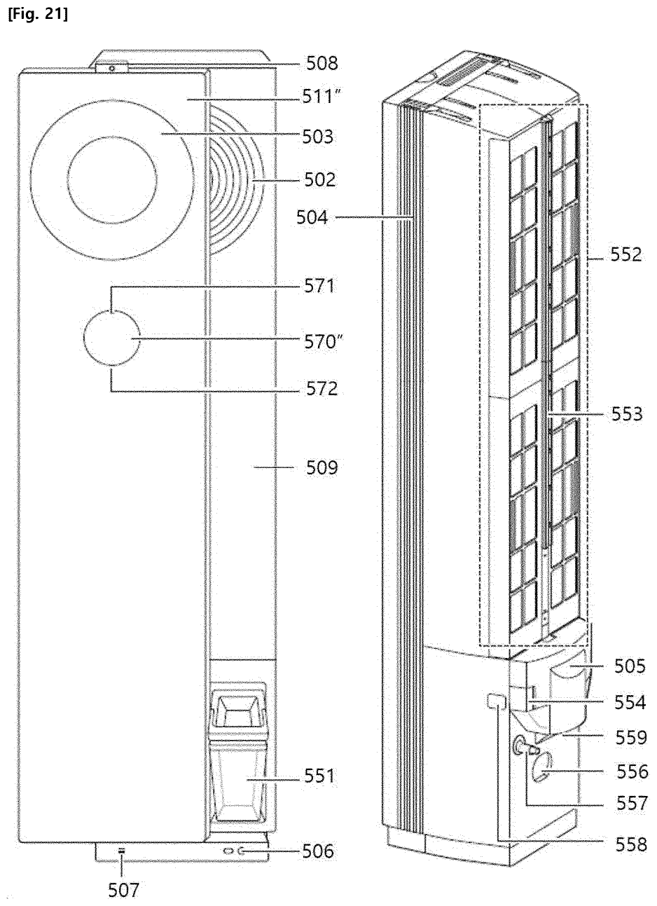

FIGS. 20 to 21 are diagrams for explaining another embodiment of the air conditioner shown in FIGS. 16 and 17.

Referring to FIGS. 20 to 21, another embodiment of the present invention includes an indoor unit 500″′). For example, the indoor unit 500″′) may be a stand-type indoor unit installed upright on the floor of an indoor space.

The indoor unit 500″′) is provided indoors and may be connected to an outdoor unit (not shown) disposed outdoors through a refrigerant piping (not shown).

The indoor unit 500″′) may include a front unit 511″) forming the exterior of the front and a circulator door 503 arranged on the front unit 511″) that moves vertically to open and close.

The indoor unit 500″) may include a base 516, a cabinet 517, and a front unit 511″). The front unit 511″) forms the front exterior of the indoor unit 500″′), and the cabinet 517 may be installed positioned above the base 516.

According to the present invention, a circulator door 503 may be installed on the front unit 511″).

The indoor unit 500″′) includes an air inlet and an air outlet, and can discharge air through the air outlet after conditioning the air drawn in through the air inlet. For example, an inlet may be formed on the rear of the indoor unit 500″), and an outlet may be formed on the upper front of the indoor unit 500″′). Alternatively, the air inlet may be arranged on the outdoor unit. Here, the inlet and outlet may be formed at different locations of the indoor unit 500″′). For example, an outlet may be formed on the side of the lower part of the indoor unit 500″). Additionally, it is possible for multiple outlets to be formed on the upper front and the side of the lower part of the indoor unit 500″′). The inlet may be formed at one or more positions among the rear, lower front, and sides of the main body. A filter unit (not shown) for filtering foreign substances such as dust contained in the drawn-in air may be installed at the inlet.

As shown in FIG. 21, a cleaning module 505 for cleaning the moving filter 552 may be disposed in the indoor unit 500″).

A circulator module 502 may be provided behind the circulator door 503 in its closed state. The circulator module 502 can generate blowing force to draw in air through the inlet and discharge air through the outlet.

As shown in FIG. 21, the circulator module 502 is installed inside the indoor unit 500″′) and can discharge air through the outlet exposed by the opening of the circulator door 503 during operation.

According to the present invention, the circulator module 502 can move forward and operate through the outlet opened by the circulator door 503. For example, at least a part of the circulator module 502 can move forward to pass through a circular outlet opened by the downward movement of the circulator door 503, after which the circulator fan of the circulator module 502 can rotate and operate.

As described above, in this specification, the outlet may refer to an opening through which at least a part of the circulator module 502, which is a discharge unit for discharging air, passes.

The circulator door 503 can open and close the outlet. The circulator door 503 opens and closes the main outlet and may be provided to discharge air processed by the air conditioner, such as heat-exchanged air or purified air, to the outside.

The circulator door 503 opens during the operation of the main body to expose the circulator module 502 to the outside, allowing air to be discharged through the outlet, and closes to seal the outlet when the operation ends. A space for accommodating the circulator door 503 when the outlet is opened may be provided on the inner side or rear of the front unit 511″).

An unillustrated moving means for moving the circulator door 503 may be installed on the inner surface of the front unit 511″). For example, the inner surface of the front unit 511″) may include a circulator door motor, a gear member for moving the circulator door 503 in the upward or downward direction according to the rotation of the circulator door motor, and a rail member. Meanwhile, a step motor, which is cost-effective and easy to control, may be used as the circulator door motor. In this case, the circulator door motor may be referred to as a circulator door step motor.

The circulator door 503 may be configured to open by moving in the upward or downward direction from the inside of the indoor unit 500″′). Since the circulator door 503 is positioned at the upper side of the front unit 511″) of the indoor unit 500″), it is more preferable from a space utilization perspective for the circulator door 503 to be configured to open by moving in the downward direction.

Alternatively, the circulator door 503 may be configured to open by moving backward into the indoor unit 500″′) and then moving in the upward or downward direction. Even in this case, it is more preferable from a space utilization perspective for the circulator door 503 to be configured to open by moving backward into the indoor unit 500″′) and then moving in the downward direction.

Hereinafter, an example where the circulator door 503 opens and closes by moving in the vertical direction will be described, but the circulator door 503 may move backward in the inward direction and then open by moving in the downward direction, and the circulator door 503 may close by moving in the upward direction and then advancing in the forward direction.

When the circulator door 503 opens, the circulator module 502 can advance in the forward direction toward the front unit 511″) to discharge air. Additionally, when the operation is completed, the circulator module 502 can move backward into the indoor unit 500″′) and close the outlet by the movement of the circulator door 13.

In some cases, an additional blower fan (not shown) to assist the blowing force may be further installed inside the main body.

The indoor unit 500″′) may further include multiple blower fans in addition to the circulator module 502. For example, a plurality of blower fans may be arranged below the circulator module 502.

Meanwhile, an auxiliary outlet 504 may be further installed on the side unit of the cabinet 517. Additionally, an air-direction controller for adjusting the wind direction of the discharged air may be arranged at the auxiliary outlet 504.

By providing the circulator module 502 at the top of the indoor unit 500″′), it becomes easier to send air over long distances.

Furthermore, since the circulator module 502 is positioned at the final stage of the air discharge path, it can directly discharge heat-exchanged and purified air over long distances.

After the circulator door 503 is opened, the discharge unit, which is the circulator module 502, can be configured to rotate in a two-dimensional manner. For example, the circulator module 502 may include a rotating unit configured with a dual joint and gear rack structure for two-axis rotation, allowing it to rotate freely in various directions. Accordingly, the circulator module 502 can rotate to the desired location of the user, enabling airflow control.

Once the entire circulator module 502 has rotated, it can direct air to the desired location for focused cooling, thereby enhancing the user's comfort and satisfaction.

According to the present invention, a display unit 570″) may be disposed on the front unit 511″). The display unit 570″) displays the operation state and setting information of the indoor unit 500″′) and is configured as a touchscreen to receive user commands. Depending on the embodiment, the front unit 511″) may be equipped with an operation unit (not shown) that includes at least one input means such as a switch, button, or touchpad.

On one side of the display unit 570″), a proximity sensor 571 and a remote control receiving unit 572 may be provided. In some embodiments, when a proximity signal corresponding to the user's approach is input from the proximity sensor 571, the display unit 570″) can be activated to display operation information, and at least one light provided in the indoor unit 500″) can be operated.

The display unit 570″) may further include one or more lights.

As shown in FIG. 21, an auto-door-open sensor 506 may be installed on the base 516. The auto-door-open sensor 506 detects the user's approach to the indoor unit 500″′) and allows the front unit 511′) to open and close. Meanwhile, the auto-door-open sensor 506 may also be disposed in a predetermined area at the lower part of the front unit 511″).

As illustrated, a microphone and/or speaker 507 may be disposed on the base 516.

Through the microphone and/or speaker 507, the user's voice can be recognized, and information can be delivered to the user via voice.

As shown in FIG. 20, the indoor unit 500″′) may further include a human-body detection sensor 508. The human-body detection sensor 508 may be disposed at the upper part of the front unit 511″). By detecting a person through the human-body detection sensor 508, the airflow can be controlled to direct air towards or away from the person depending on the operation mode. Meanwhile, a vision module including at least one camera may be installed at the upper part of the front unit 511′).

The indoor unit 500″′) may include a heat exchanger (not shown) inside, which exchanges heat with the refrigerant for the air that is drawn in.

The front unit 511′) can slide to the left or right. Therefore, the front unit 511′) may also be referred to as a sliding door.

The front unit 511′) is mounted by sliding means formed on the cabinet 517 and can move left and right. The movement of the front unit 511″) may expose a portion of the inner panel 509 to the outside.

The cabinet 517 may include a sliding door step motor, a gear member for moving the front unit 511″) in the left or right direction according to the rotation of the sliding door step motor, a rail member, and the like.

As shown in FIG. 21, the inner panel 509 may accommodate a circulator module 502, and moving means (not shown) for moving the circulator module 502 may be installed.

According to an embodiment, the circulator module 502 may include a circulator fan (not shown), a circulator rotation unit (not shown) capable of rotating to change the direction in which at least the circulator fan (not shown) is directed, and a circulator moving unit (not shown) capable of moving at least the circulator fan (not shown).

As shown in FIG. 21, a humidification water tank 551 of a humidification module may be installed at the lower part of the inner panel 509. The water tank 551 may be exposed to the outside as the front unit 511″) moves in the left or right direction to open.

According to the present invention, a predetermined area of the humidification water tank 551 may have an inlet formed for filling water. According to an embodiment, the inlet may be open, or a cover capable of opening and closing at least a portion of the inlet may be disposed.

The humidification water tank 551 of the present invention may have a moving shaft formed at the lower part and may be connected to the cabinet 517. The upper part of the humidification water tank 551 may be moved to protrude forward based on the lower moving shaft, thereby forming the inlet to be open. The humidification water tank 551 may be tilted forward so that the upper part forms a predetermined angle with the inner panel 509.

Additionally, the humidification water tank 551 may be detachable from the indoor unit 500″′). Inside the indoor unit 500″′), a sensor for detecting whether the water tank 551 is mounted may be provided.

The humidification water tank 551 may automatically move to open the inlet when user approach is detected by the proximity sensor 571 according to the proximity signal. The humidification water tank 551 may move to open the inlet as a handle (not shown) is pulled toward the front. The humidification water tank 551 may move forward to open the inlet as a fixing part (not shown) is released by being pressed inward. The humidification water tank 551 may automatically rotate to open the inlet as the front unit 511″) slides open.

A water level indicator (not shown) for displaying the water level of the tank may be provided on a portion of the inner panel 509 or the humidification water tank 551.

The humidification water tank 551 may be configured to allow verification of the water level inside. For example, the humidification water tank 551 may be formed of a transparent material at the front. The tank may have a portion of its front formed of a transparent material. Additionally, the entire humidification water tank 551 may be formed of a transparent material.

The indoor unit 500″′) includes a moving filter 552. The moving filter 552 may be positioned at the rear of the indoor unit 500″′).

The indoor unit 500″′) includes an indoor-temperature sensor 553. The indoor-temperature sensor 553 senses the indoor temperature and may be positioned at the rear of the indoor unit 500″′).

As illustrated in FIG. 21, the indoor unit 500″′) may include a dust box 554 for storing dust collected by the cleaning module 505.

As illustrated, the indoor unit 500″′) may further include a humidity sensor 559. The humidity sensor 559 senses the indoor humidity and may be positioned at the rear of the indoor unit 500″′).

As illustrated, the indoor unit 500″′) may further include a piping hole 556 and a drain hole 557. The piping hole 556 and the drain hole 557 may be positioned at the rear of the indoor unit 500″′).

The indoor unit 500″′) may include a PM1.0 sensor 558. The PM1.0 sensor 558 senses fine dust and may be positioned at the rear of the indoor unit 500″).

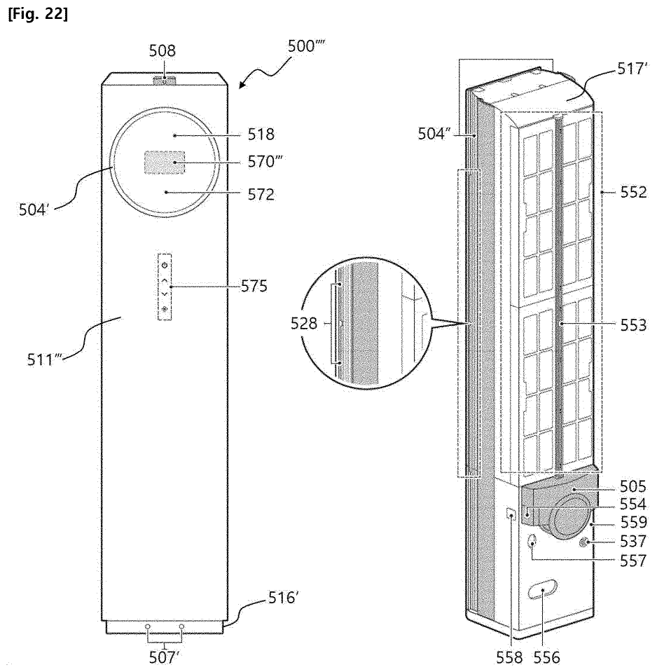

FIG. 22 is a diagram for explaining another example of the air conditioner illustrated in FIGS. 16 and 17.

Referring to FIG. 22, an air conditioner according to another example of the present invention includes an indoor unit 500″′). For instance, the indoor unit 500″′) may be a stand-type indoor unit installed upright on the floor of an indoor space.

The indoor unit 500″″) includes a front unit 511″′). The front unit 511″′) forms the front exterior of the indoor unit 500″″).

The indoor unit 500″′) may include a circulator 518. The circulator 518 can be positioned in a circular opening formed in the front unit 511″′).

The indoor unit 500″″) may include a display unit 570″′). The display unit 570″′) can be positioned on the front unit 511″′). The display unit 570″′) may be arranged within the circulator 518. The display unit 570″) is capable of displaying operation states and setting information. Here, the display unit 570″′) may be configured as a touchscreen to receive user commands.

The indoor unit 500″″) may include a remote control receiving unit 572. The remote control receiving unit 572 can be positioned within the circulator 518 and may be located on one side of the display unit 570″).

The indoor unit 500″′) may include an indoor unit button unit 575. The indoor unit button unit 575 can be positioned on the front unit 511″′). The indoor unit button unit 575 allows the user to turn the power on and off or set the temperature and wind intensity without a remote control.

The indoor unit 500″″) may have outlets 504′, 504′) for discharging air. The first outlet 504′) is formed on the front unit 511″) and may have a ring shape surrounding the circulator 518. The first outlet 504′) may be an opening between the front unit 511″) and the circulator 518. The second outlet 504″) serves as the left and right outlets of the indoor unit 500″′), allowing air to be sent into the room from both sides to adjust the indoor temperature to the user's desired or pre-set temperature.

The indoor unit 500″″) may include an air guard 528. The air guard 528 is positioned on both sides of the indoor unit 500″) and can adjust the direction of the air discharged from the second outlet 504″).

The indoor unit 500″″) may include a microphone 507′). The microphone 507′) can be positioned in the base 516′) constituting the lower part of the indoor unit 500″).

The indoor unit 500″″) may include a speaker 537. The speaker 537 can be positioned in the cabinet 517′) of the indoor unit 500″).

The indoor unit 500″″) may include a filter 552. The filter 552 can be positioned at the rear of the indoor unit 500″).

The indoor unit 500″″) may include an indoor-temperature sensor 553. The indoor-temperature sensor 553 senses the indoor temperature and can be positioned at the rear of the indoor unit 500″′).

The indoor unit 500″′) may include a dust box 554 for storing dust collected by the cleaning module 505.

The indoor unit 500″′) may further include a humidity sensor 559. The humidity sensor 559 senses indoor humidity and may be positioned at the rear of the indoor unit 500″).

The indoor unit 500″′) may further include a piping hole 556 and a drain hole 557. The piping hole 556 and the drain hole 557 may be positioned at the rear of the indoor unit 500″″).

The indoor unit 500″″) may include a PM1.0 sensor 558. The PM1.0 sensor 558 senses fine dust and may be positioned at the rear of the indoor unit 500″″).

The indoor units 500′, 500″, 500″′, 500″″) shown in FIGS. 18 to 22 have display units 570, 570′, 570″, 570″′).

Since the indoor units 500′, 500″) shown in FIGS. 18 and 19 are installed at the top of a wall or on the ceiling, it is not easy for a user to control the indoor units 500′, 500″) through the display units 570, 570′). Therefore, the display units 570, 570′) are used to indicate that the indoor units 500′, 500″) have been controlled via a remote control or user terminal, or to display the current status of the indoor units 500′, 500″). The display unit 570″) of the indoor unit 500″′) shown in FIG. 22 is also used to indicate that the indoor unit 500″′) has been controlled via a remote control or user terminal, or to display the current status of the indoor unit 500″′).

Meanwhile, the display unit 570″) shown in FIGS. 20 to 21, unlike other display units 570, 570′, 570″), is configured as a touchscreen that can directly receive user commands and display a screen according to the input commands. Additionally, the display unit 570″) can be used, like other display units 570, 570′, 570″), to indicate that the indoor unit 500″′) has been controlled via a remote control or user terminal, or to display the current status of the indoor unit 500″′).

As described above, the indoor units 500′, 500″, 500″′, 500″″) shown in FIGS. 18 to 22 can have their power, operation mode, and other settings controlled via a remote control, user terminal, and display unit. Control signals input through the remote control, user terminal, and display unit are input to the processor 5300 shown in FIG. 17, and the processor 5300 can control the drive unit 5400 based on the input control signals.

The operation modes of the indoor units 500′, 500″, 500″, 500″″) may include various operation modes. For example, they may include cooling mode, automatic mode, dehumidification mode, heating mode, blowing mode, air purification mode, and energy-saving mode.

The air conditioner including the indoor units 500′, 500″, 500″′, 500″″) shown in FIGS. 18 to 22 may further include ‘sleep mode’ as an operation mode. Sleep mode is a mode that controls the temperature and/or humidity of the indoor space to enhance the quality of the user's sleep.

Based on the first environment adjustment information to the nth environment adjustment information, or the A environment adjustment information to the H environment adjustment information generated by the computing device 100 of FIG. 16(a) or the air conditioner 500 of FIG. 16(b), the air conditioner 500 can optimize the temperature and/or humidity within the sleep space for sleep.

For example, the air conditioner 500 can adjust the indoor temperature and/or humidity for falling asleep up to a predetermined time before the user sleeps (e.g., 20 minutes prior) according to the first environment adjustment information. Alternatively, it can convert direct airflow to indirect airflow, generate noise (white noise) conducive to sleep just before sleeping, adjust the blowing intensity to below a predefined level, or reduce the brightness of the display unit 570, 570′, 570″, 570″′) as shown in FIGS. 18 to 22.

The air conditioner 500 can further reduce the brightness of the display unit 570, 570′, 570″, 570″′) or turn it off, operate at noise levels below a predefined level, adjust the blowing intensity to below a predefined level, set the indoor temperature within a predefined range, maintain the humidity in the sleep space at a predetermined level, or maintain indirect airflow according to the second environment adjustment information. The air conditioner 500 can adjust the indoor temperature and/or humidity for waking, reduce the blowing intensity and noise at the waking time, generate white noise to gradually induce waking, maintain noise below a predefined level, or operate in conjunction with the predicted waking time or recommended waking time according to the third environment adjustment information. The air conditioner 500 can control at least one of the indoor temperature, indoor humidity, blowing intensity, or noise level and acquire, analyze, and reflect user data according to the fourth environment adjustment information.

For example, the air conditioner 500 can be turned on based on the A environment adjustment information, change the airflow to below a specific intensity or reduce the current airflow to below the specific intensity, convert direct airflow to indirect or no airflow, or reduce the brightness of the display unit to below a predetermined brightness based on the B environment adjustment information. The temperature and humidity set based on the C environment adjustment information can be changed to optimal temperature and humidity. The humidity or temperature set based on the D environment adjustment information can be increased, or direct or indirect airflow can be converted to no airflow. The bedroom temperature or humidity can be changed to optimized temperature or humidity based on the E environment adjustment information. The set temperature and humidity can be changed to the preferred temperature or humidity that the user mainly set when falling asleep in the past based on the F environment adjustment information. The set temperature or humidity can be changed to the specific temperature or humidity most preferred by the user based on the G environment adjustment information. The set temperature or humidity can be changed to the specific temperature or humidity most preferred by the user based on the H environment adjustment information.

In the system configuration of FIG. 16(a), to operate the air conditioner 500 in sleep mode, a device connection process for connecting the air conditioner 500 with the computing device 100 through a network can be performed.

In the system configuration of FIG. 16(b), to operate the air conditioner 500 in sleep mode, a connection process for interfacing the air conditioner 500 with the user terminal 10 can be performed. Here, the air conditioner 500 and the user terminal 10 can be wirelessly connected. For example, the air conditioner 500 and the user terminal 10 can be directly connected through a local network.

### Method for Operating the Air Conditioner in Sleep Mode

The operation of the air conditioner, including the indoor unit 500′, 500′, 500″ 500″′) shown in FIGS. 18 to 22, in sleep mode can be performed either by the user's selection to operate the indoor unit 500′, 500″, 500″′, 500″″) in sleep mode or automatically.

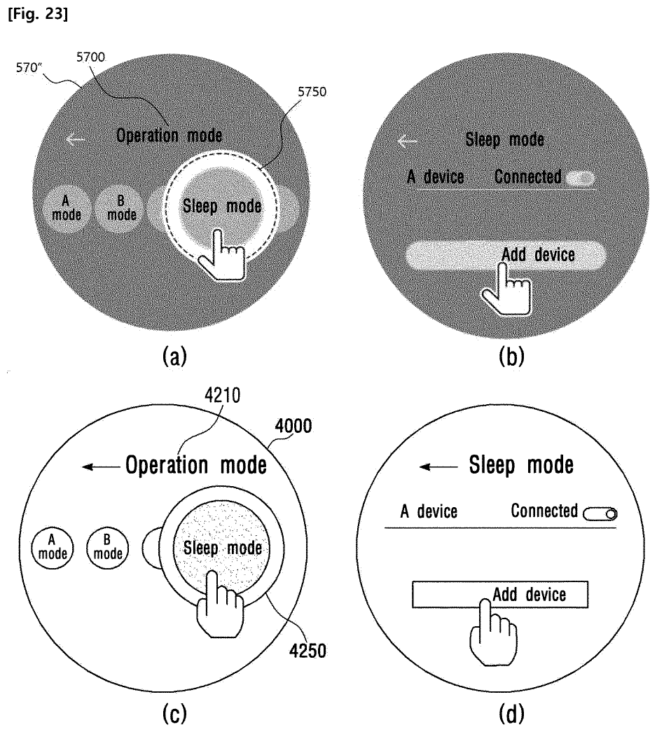

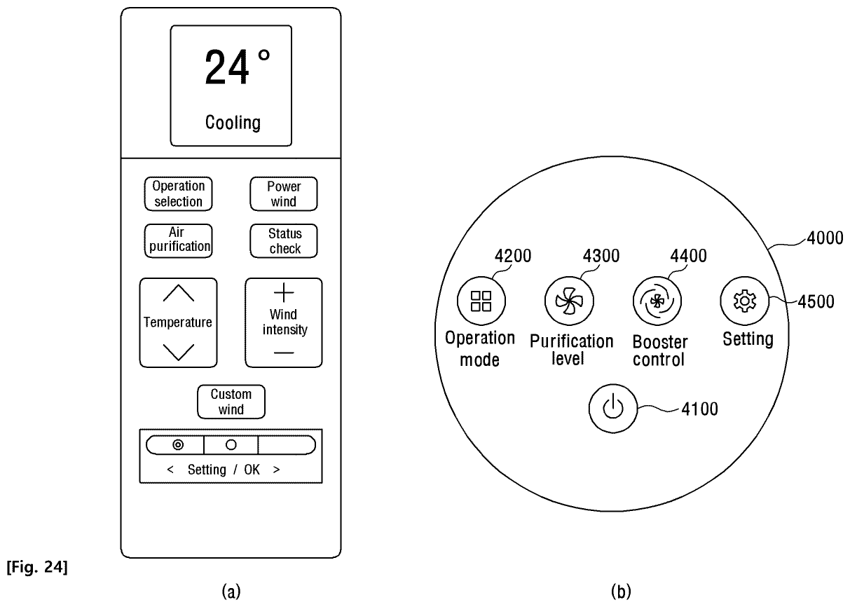

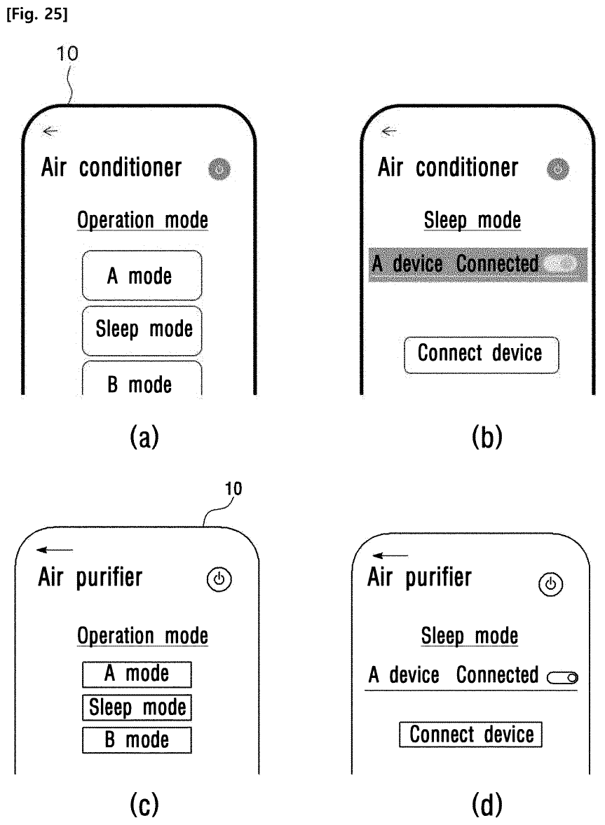

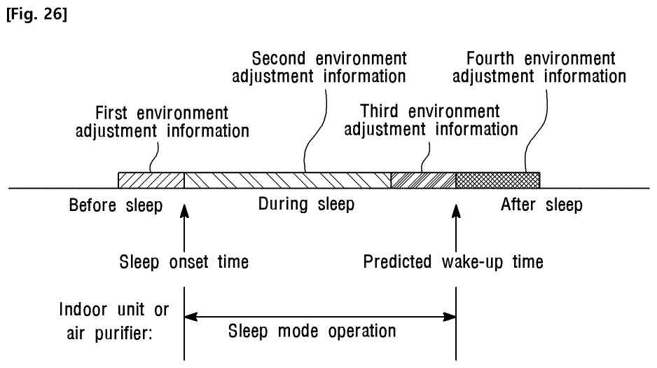

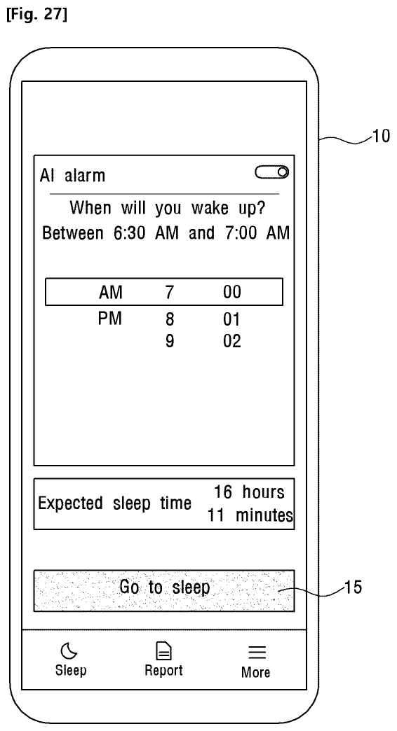

First, the method of operating the indoor unit 500′, 500″, 500″′, 500″″) in sleep mode by the user's selection will be described with reference to FIGS. 23 to 25.

FIGS. 23(a) and (b) are diagrams for explaining the method of operating the indoor unit 500″) in sleep mode through the display unit 570″) of the indoor unit 500″) shown in FIGS. 20 to 21.

Referring to FIG. 23(a), when the user directly touches the display unit 570″) to enter the operation mode 5700, the display unit 570′) can display the sleep mode 5750 and various other modes. In this case, the user can operate the indoor unit 500″) in sleep mode by selecting the sleep mode 5750.

Meanwhile, if there is no device wirelessly connected to the indoor unit 500″), as shown in FIG. 23(b), a screen for adding a device (e.g., user terminal) to be wirelessly connected to the indoor unit 500″) may be displayed on the display unit 570″), and the user can touch the ‘Add Device’ button to connect the desired device.

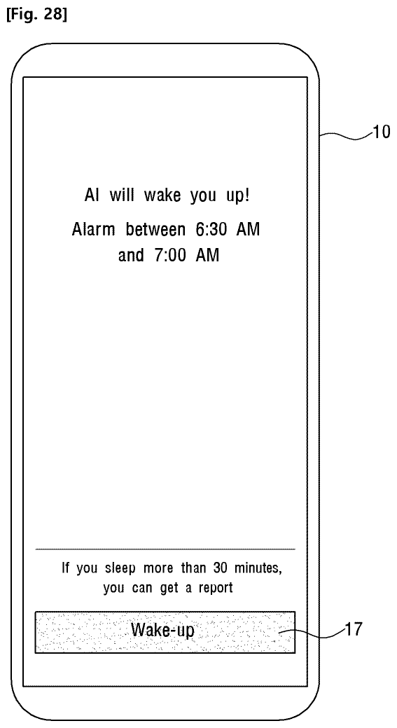

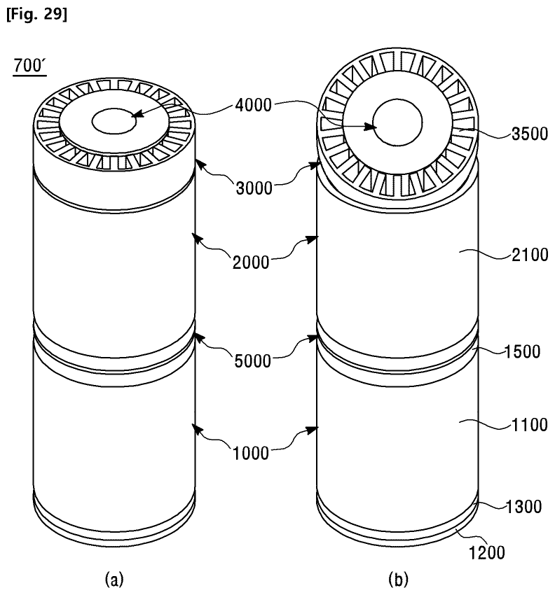

FIG. 24(a) is a diagram for explaining a method of operating the indoor unit 500′, 500″, 500″′, 500″″) shown in FIGS. 18 to 22 in sleep mode using a remote control 600.

Referring to FIG. 24(a), the air conditioner including the indoor unit 500′, 500″, 500″′, 500″″) shown in FIGS. 18 to 22 may further include a remote control 600 capable of controlling the indoor unit 500′, 500″, 500″′, 500″″).

The remote control 600 includes a display unit 601 that displays the currently selected function and operation status, and a power button 602 for turning the power on and off. The remote control 600 may further include various buttons. For example, it may include an operation selection button 603 for selecting the desired operation mode, an air purification button 604 for making the indoor air clean and pleasant, a temperature control button 605 for adjusting the desired temperature, a wind customization button 606 for selecting wind suitable for the situation and space, a power wind button 607 for quickly adjusting the indoor temperature by emitting strong wind, a status check button 608 for checking the operation status of the indoor unit and the indoor environment, a wind intensity button 609 for setting the wind intensity, and a setting unit 610 for setting various functions.

The remote control 600 may further include an AI sleep button 615 capable of operating the sleep mode. The user can press the AI sleep button 615 to operate the indoor unit 500′, 500″, 500″′, 500″″) in sleep mode.

FIGS. 25(a) and (b) are diagrams for explaining a method of operating the indoor unit 500′, 500″, 500″′, 500″″) shown in FIGS. 18 to 22 in sleep mode through a user terminal 10. Specifically, FIGS. 25(a) and (b) show the screen of a first application for remotely controlling the indoor unit 500′, 500″, 500″′, 500″″) from the user terminal 10.

Referring to FIG. 25(a), a first application for controlling the indoor unit 500′, 500″, 500″′, 500″″) may be installed and stored on the user terminal 10. The user can control the operation of the indoor unit 500′, 500′, 500″′, 500″″) through the first application installed on the user terminal 10.

The first application may provide a screen capable of controlling the operation mode of the indoor unit 500′, 500″, 500″′, 500″″), and the user can select the desired operation mode (A mode, sleep mode, B mode, etc.) through the user terminal 10.

If the user selects the sleep mode in FIG. 25(a), the indoor unit 500′, 500″, 500″, 500″″) can operate in sleep mode. Here, if the indoor unit 500′, 500″, 500″′, 500″″) is not yet connected to the user terminal 10, a screen for specifically setting or controlling the sleep mode of the indoor unit 500′, 500″, 500″′, 500″″) may be displayed as shown in FIG. 25(b). Through this, the user can select the user terminal (A device) to connect with the indoor unit 500′, 500″, 500″′, 500″″), and connect other terminals to the indoor unit 500′, 500″, 500″′, 500″″).

The operation method of the sleep mode illustrated in FIGS. 23 to 25 is configured to operate based on the user's selection; however, it is not limited thereto. An air conditioner according to another embodiment of the present invention may be configured to automatically operate in sleep mode without the user's selection. This will be explained with reference to FIG. 26.

FIG. 26 is a diagram for explaining a method by which an indoor unit or air purifier automatically operates in sleep mode.

Referring to FIG. 26, the air conditioner 500 including the indoor unit 500′, 500″, 500″′, 500″″) can automatically operate in sleep mode based on environment adjustment information generated from the previously described sleep state information and/or sleep stage information, without a separate user selection.

For example, if the processor 130 of the computing device 100 in FIG. 16(a) or the processor 5300 of the air conditioner in FIG. 16(b) and FIG. 17(a) identifies the user's sleep onset time through second sleep state information and generates second environment adjustment information, the indoor unit 500′, 500″, 500″′, 500″″) can be configured to automatically start sleep mode.

Additionally, if the processor 130 of the computing device 100 in FIG. 16(a) or the processor 5300 of the air conditioner in FIG. 16(b) and FIG. 17(a) identifies the wake-up prediction time and generates third environment adjustment information, the indoor unit 500′, 500″, 500′ 500″′) can be configured to automatically terminate the sleep mode at the wake-up prediction time.

Contrary to what is shown in FIG. 26, the start and end times of the sleep mode may vary. For example, the start time of the sleep mode may be a sleep induction time before the sleep onset time.

As another example, as shown in FIG. 25(a), the start time of the sleep mode may be immediately after the sleep mode of the first application is selected by the user or after a predetermined time has elapsed following the selection of the sleep mode. Alternatively, as shown in FIG. 25(b), it may be immediately after a predetermined device (Device A) is connected to the indoor unit 500′, 500″, 500″′, 500″″) or after a predetermined time has elapsed following the connection.

Other examples of potential start times for the sleep mode will be explained with reference to FIGS. 27 to 28.

FIGS. 27 to 28 are diagrams for explaining the start times of the sleep mode operation of the indoor unit or air purifier shown in FIG. 26.

As illustrated in FIG. 27, the initiation point of the sleep mode may be when the ‘Go to Sleep (15)’ button is selected via a second application installed on the user terminal 10.

Here, the second application may be an application that measures environment sensing information (e.g., the user's breathing sound) and automatically outputs an AI-based sleep report. The second application can interact with the first application depicted in FIGS. 25 (a) and (b) to utilize each other's data.

Alternatively, referring to FIG. 27, the initiation point of the sleep mode may occur after a predetermined time has elapsed following the selection of the ‘Go to Sleep (15)’ button via the second application. Here, the predetermined time may be the duration during which the result measured by the accelerometer sensor provided in the user terminal 10 remains constant.

Again, referring to FIG. 26, the termination point of the sleep mode may be, for example, after a predetermined time has elapsed following the wake-up prediction time.

In another example, referring to FIGS. 25 (a) and (b), the termination point of the sleep mode may be immediately after the connection between the first application installed on the user terminal 10 and the air conditioner is severed.

In yet another example, referring to FIG. 28, the termination point of the sleep mode may be when the ‘Wake Up (17)’ button is selected via the second application installed on the user terminal 10.

Additionally, the initiation and termination points of the sleep mode can be variously modified by the user or manufacturer of the indoor unit 500′, 500″, 500″′, 500″″).

For the continuous operation of the sleep mode, in the system configuration of FIG. 16 (a), it is preferable that the user terminal 10 is connected to the computing device 100 via a network, and the computing device 100 is connected to the air conditioner 500 via a network. Meanwhile, in the system configuration of FIG. 16 (b), it is preferable that the user terminal 10 is connected to the air conditioner 500 via a network or short-range wireless communication.

### Air Purifier

### Operation of the Air Purifier

Hereinafter, the air purifier according to the present invention will be described in detail. FIGS. 16 (c) and (d) are conceptual diagrams for explaining the operation of the air purifier according to the present invention. Specifically, FIG. 16 (c) is a schematic diagram where the environment adjustment device 30 of FIG. 1 (a) is implemented as an air purifier 700, and FIG. 16 (d) is a schematic diagram showing the air purifier 700 operating in conjunction with the user terminal 10.

As illustrated in FIG. 16(c), the air purifier 700 according to the present invention can operate in conjunction with the user terminal 10 and the computing device 100.

The computing device 100 may include a network unit 110, a memory 120, and a processor 130 (see FIG. 2). The network unit 110 transmits and receives data with the user terminal 10, the external server 20, and the air purifier 700. The network unit 110 can transmit and receive data, such as data for performing a sleep environment adjustment method according to sleep state information, with other computing devices, servers, etc., according to an embodiment of the present invention. Specifically, the network unit 110 can provide communication functions between the computing device 100 and the user terminal 10, the external server 20, and the air purifier 700. For example, the network unit 110 can receive sleep examination records and electronic health records for multiple users from a hospital server. In another example, the network unit 110 can receive environment sensing information related to the space where the user is active from the user terminal 10. In yet another example, the network unit 110 can transmit air quality-related environment adjustment information to the air purifier 700 to adjust the environment of the space where the user is located.

Here, the operation method, hardware configuration, and software configuration of the network unit 110, memory 120, and processor 130 are the same as described above, and thus redundant descriptions are omitted.

Additionally, the operation method, hardware configuration, software configuration, and sleep analysis model of the processor 130 are the same as described above, and thus redundant descriptions are omitted. The processor 130 can acquire the user's sleep state information and environment sensing information, as described above.

The processor 130 can generate first environment adjustment information to nth environment adjustment information. Specifically, it can generate first environment adjustment information to control the air purifier to pre-remove fine dust and harmful gases. The first environment adjustment information may include information to control the air purifier to induce sleep by generating noise (white noise) to a degree that can induce sleep just before sleep, adjust the blowing intensity to a predefined intensity or lower, or reduce the intensity of the LED. Additionally, the first environment adjustment information may include information to control the air purifier to perform dehumidification/humidification based on temperature and humidity information within the sleep space.

Furthermore, the processor 130 can generate second environment adjustment information to turn off the LED of the air purifier based on the second sleep state information, operate the air purifier at a noise level below a predefined level, adjust the blowing intensity to a predefined intensity or lower, set the blowing temperature within a predefined range, or maintain the humidity within the sleep space at a predetermined temperature.

Moreover, the processor 130 can generate third environment adjustment information and fourth environment adjustment information based on the third sleep state information and fourth sleep state information, as previously described.

As illustrated in FIG. 16(d), the air purifier 700 according to an embodiment of the present invention can operate in conjunction with the user terminal 10, and the air purifier 700 according to an embodiment of the present invention may include additional configurations to operate as an air purifier in conjunction with the configuration of the computing device 100 in FIG. 16(c).

FIG. 17(b) is a block diagram showing the configuration of the air purifier according to the present invention. As illustrated in FIG. 17(b), the air purifier 700 according to the present invention may include a network unit 710, a memory 720, a processor 730, a drive unit 740, and a measurement unit 750.

As illustrated in FIG. 17, the air purifier 700 may be implemented as an air purification device embedded in the ceiling or outer wall of a building, apartment, or house, as a fixed air purifier fixed to one side of an indoor space, as a portable and easily movable air purifier, as an in-vehicle air purification device placed in a vehicle, or as a wearable air purifier worn on the body to purify the air quality around the user.

As illustrated in FIG. 17, the air purifier 700 can be implemented in various types, such as a dust-collecting filter-type air purifier that removes dust using pre-filters and HEPA filters, an adsorption filter type that absorbs harmful gases using activated carbon, a wet type that removes dust or harmful gases using water, an electrostatic precipitator type that removes dust using high voltage, a negative ion type that removes dust by generating and supplying negative ions into the air using high voltage, a plasma type that removes harmful gases by generating positive/negative ions with plasma, and a UV photocatalyst type that removes odors and harmful gases through oxidation/reduction of OH radicals and active oxygen generated by UV irradiation on TiO. It may also be a composite air purifier employing two or more of these methods.

As shown, the functions, operations, hardware configuration, and software configuration of the network unit 710, memory 720, and processor 730 of the air purifier 700 are the same as described above. The first to nth environment adjustment information generated by the processor 730 can be transmitted to the drive unit 740. The drive unit 740 can operate various hardware elements provided in the air purifier 700.

As depicted, the measurement unit 750 may include one or more sensors for sensing air components, illumination, and the status of air purifier components within the space. Specifically, the measurement unit may include a dust sensor for detecting invisible airborne particles such as PM1.0, PM2.5, PM10, a gas sensor for detecting indoor harmful gases or odors, an illumination sensor for sensing indoor illumination, a TVOC sensor for measuring the total concentration of over 300 types of volatile organic compounds in indoor air, a CO2 sensor for measuring the concentration of carbon dioxide in indoor air, a radon sensor for measuring the concentration of radon, a pressure sensor for measuring the filter differential pressure according to the lifespan of the filter to indicate the filter replacement time, and a temperature sensor for measuring indoor temperature.

Although not shown in the drawings, the air purifier 700 may be composed of a housing equipped with a discharge port and an inlet, a filter unit, a blower fan, a sterilization unit, a humidification unit, a heating unit, a cooling unit, and a measurement unit. The housing can be designed in various ways depending on the implementation method of the air purifier 700, such as built-in, fixed, mobile, vehicle-mounted, or wearable types. The filter unit can be selected to correspond to air purification methods such as dust-collecting filter type, adsorption filter type, wet type, electrostatic precipitator type, negative ion type, plasma type, and UV photocatalyst type. The blower fan can be connected to a motor that rotates by power supplied from the power supply unit. The sterilization unit has the function of sterilizing the inhaled air using chemical or electrical methods. The humidification unit has the function of humidifying and discharging the inhaled air, and the heating unit and cooling unit have the function of heating or cooling the inhaled air to a predetermined temperature.

The hardware elements of the air purifier 700 described above are merely one embodiment, and some of them may be integrated into a single configuration, some configurations may be omitted, and various configurations for performing air purification functions not described above may be added.

Meanwhile, environment sensing information can be acquired through the user terminal 10. The environment sensing information may be sleep sound information acquired from a bedroom where the user is sleeping.

Additionally, the environment sensing information may be air quality information within the sleep space acquired from the measurement unit 750 provided in the air purifier 700. The environment sensing information acquired through the user terminal 10 or the measurement unit 750 may serve as foundational information for acquiring the user's sleep state information in the present invention.

For a specific example, sleep state information related to whether the user is before sleep, during sleep, or after sleep can be acquired through environment sensing information obtained in relation to the user's activities. Furthermore, information related to the surrounding air quality before, during, and after the user's sleep can be acquired.

According to one embodiment of the present invention, the processor 730 can acquire sleep state information based on the environment sensing information obtained through the user terminal 10 and/or the measurement unit 750.

Specifically, the processor 730 can identify singularities where information of a predefined pattern is detected in the environment sensing information. Here, the information of a predefined pattern may relate to breathing and movement patterns associated with sleep. For instance, in the wake state, all nervous systems are activated, resulting in irregular breathing patterns and frequent body movements. Additionally, due to the lack of relaxation of the neck muscles, breathing sounds may be minimal.

On the other hand, when a user is sleeping, the autonomic nervous system stabilizes, resulting in regular changes in breathing and reduced body movement, and the breathing sound may increase. Specifically, the processor 730 can identify the point in time when acoustic information of a predefined pattern related to regular breathing, minimal body movement, or minimal breathing sound is detected in the environment sensing information as a singularity. Furthermore, the processor 730 can acquire sleep sound information based on the environment sensing information obtained with reference to the identified singularity. The processor 730 can identify singularities related to the user's sleep time from the time-series environment sensing information and acquire sleep sound information based on these singularities.

Additionally, the air quality measured through the measurement unit 750 significantly affects the user's sleep. According to studies analyzing the relationship between air quality and sleep, it has been confirmed that sleep disorders show a statistically significant correlation with air pollution. For example, exposure to PM10 can make it difficult to maintain sleep, and it has been particularly noted that the probability of sleep disorders is highest when males are exposed to PM1.

Furthermore, it has been confirmed that for females, the likelihood of sleep disorders is highest when exposed to PM1 and PM2.5. It has also been found that when SO2 and 03 levels are high, the probability of sleep disturbances related to wheezing is highest. Moreover, it has been confirmed that if pregnant women are exposed to PM2.5 between 31 and 35 weeks of pregnancy, the likelihood of the newborn having a shorter sleep duration is highest. Various studies have been conducted on the correlation between AHI and air quality measurement indices, and although results vary slightly between studies, the high correlation between air quality and sleep remains consistent.

According to an embodiment of the present invention, the air purifier 700 can acquire sleep state information based on environment sensing information, generate environment adjustment information, and perform operations appropriate for the sleep stage using this information.

Specifically, if the processor 730 of the air purifier 700 determines that the user's state is pre-sleep, it can generate first environment adjustment information to control the air purifier from the predicted time when the user is preparing for sleep (e.g., sleep induction time) until the time the user falls asleep (i.e., when the second sleep state information is acquired). The first environment adjustment information can be generated by reflecting the PM concentration, harmful gas concentration, CO2 concentration, SO2 concentration, O3 concentration, humidity, and temperature measured by the measurement unit 750.

The first environment adjustment information may include information to control the air purifier to pre-remove fine dust and harmful gases until a predetermined time before the user's sleep (e.g., 20 minutes prior), control the air purifier 700 to induce sleep with a level of noise (white noise) just before sleep, adjust the blowing intensity to a predefined level or lower, reduce the intensity of the LED, or control the air purifier to perform dehumidification/humidification based on the temperature and humidity information within the sleeping space.

Additionally, the processor 730 can generate second environment adjustment information to control the air purifier by turning off the LED, operating the air purifier 700 at a noise level below a predefined level, adjusting the blowing intensity to a predefined level or lower, setting the blowing temperature within a predefined range, or maintaining the humidity within the sleeping space at a predetermined temperature based on the second sleep state information.

According to the present invention, the second environment adjustment information is based on the second sleep state information and may include control information to turn off the air purifier's LED, operate the air purifier 700 at a noise level below a predefined level, adjust the blowing intensity to a predefined level or lower, set the blowing temperature within a predefined range, or maintain the humidity within the sleeping space at a predetermined temperature. The user can be induced to sleep with air flow and white noise in a sleeping space where fine dust and harmful gases have been removed just before sleep, and can enjoy a deep sleep in an optimally controlled temperature and humidity environment after falling asleep.

### Configuration of the Air Purifier According to the Embodiment

FIGS. 29 (a) and (b) are diagrams for explaining an example of the air purifier shown in FIGS. 16 and 17.

Referring to FIGS. 29(a) and (b), an air purifier 700′) according to an embodiment of the present invention includes a blower 1000, 2000 that generates an airflow and a flow conversion device 3000 that switches the discharge direction of the airflow generated by the blower 1000, 2000.

The blower 1000, 2000 includes a first blower 1000 that generates a first airflow and a second blower 2000 that generates a second airflow. Hereinafter, the blower 1000, 2000 may also be referred to as an ‘air purification module.’

The first blower 1000 and the second blower 2000 may be arranged in a vertical direction. For example, the second blower 2000 may be positioned above the first blower 1000. In this case, the first airflow forms a flow that draws in indoor air present on the lower side of the air purifier 700′), and the second airflow forms a flow that draws in indoor air present on the upper side of the air purifier 700′).

The air purifier 700′) includes a cover 1100, 2100 that forms the exterior.

The cover 1100, 2100 includes a first cover 1100 that forms the exterior of the first blower 100. The first cover 1100 may have a cylindrical shape. Additionally, the upper part of the first cover 1100 may be configured to have a smaller diameter than the lower part. That is, the first cover 1100 may have a truncated conical shape.

The first cover 1100 may be composed of at least two or more parts. These parts can be coupled or separated from each other. When at least one of the parts rotates, the first cover 1100 opens and can be detached from the air purifier 700′). A locking device may be provided at the junction where the parts are coupled. The locking device may include a locking protrusion or a magnetic member. By opening the first cover 1100, the internal components of the first blower 1000 can be replaced or repaired.

The first cover 1100 may have a first inlet through which air is drawn in. The first inlet includes a through-hole formed by penetrating at least a portion of the first cover 1100. Multiple first inlets may be formed.

The multiple first inlets may be evenly formed in the circumferential direction along the outer peripheral surface of the first cover 1100 so that air can be drawn in from any direction relative to the first cover 1100. That is, air can be drawn in from a 360-degree direction based on the vertical centerline passing through the internal center of the first cover 1100.

As such, by configuring the first cover 1100 in a cylindrical shape and forming multiple first inlets along the outer peripheral surface of the first cover 1100, the amount of air intake can be increased.

The air drawn in through the first inlet can flow approximately radially from the outer peripheral surface of the first cover 1100. Directions are defined. Based on FIG. 29, the vertical direction is referred to as the axial direction, and the horizontal direction is defined as the radial direction. The axial direction may correspond to the direction of the central axis of the fan, which is disposed inside the air purifier 700′) to generate airflow, i.e., the direction of the fan's motor axis. The radial direction is understood as the direction perpendicular to the axial direction. The circumferential direction is understood as the virtual circular direction formed when rotating around the axial direction with the distance in the radial direction as the rotation radius.

The first blower 1000 further includes a base 1200 provided beneath the first cover 1100 and placed on the ground. The base 1200 is positioned spaced downward from the lower end of the first cover 1100. Additionally, a base inlet 1300 may be formed in the spaced area between the first cover 1100 and the base 1200. Air can be drawn in through the base inlet 1300, and the drawn air can flow into the first blower 1000.

The first blower 1000 may have multiple inlets, such as the first inlet and the base inlet 1300. Air present in the lower part of the indoor space can easily flow into the first blower 1000 through the multiple inlets. Therefore, the amount of air intake can be increased.

An upper part of the first blower 1000 may be formed with a first outlet 1500. Air discharged through the first outlet 1500 can flow axially upward.

The cover 1100, 2100 may include a second cover 2100 forming the exterior of the second blower 2000. The second cover 2100 may be cylindrical. Furthermore, the upper part of the second cover 2100 may be configured to have a smaller diameter than the lower part. That is, the second cover 2100 may have a truncated conical shape.

The second cover 2100 may be composed of at least two or more parts. These parts can be coupled or separated from each other. When at least one of the parts rotates, the second cover 2100 opens and can be detached from the air purifier 700′). A locking device may be provided at the junction where the parts are coupled. The locking device may include a locking protrusion or a magnetic member. By opening the second cover 2100, internal components of the second blower 2000 can be replaced or repaired.

The lower end diameter of the second cover 2100 may be formed smaller than the upper end diameter of the first cover 1100. Therefore, from the perspective of the overall shape of the covers 1100, 2100, the lower cross-sectional area of the covers 1100, 2100 is formed larger than the upper cross-sectional area, thereby allowing the air purifier 700′) to be stably supported on the ground.

The second cover 2100 may be formed with a second inlet through which air is drawn. The second inlet includes a through-hole formed by penetrating at least a portion of the second cover 2100. Multiple second inlets may be formed.

The multiple second inlets are evenly formed in the circumferential direction along the outer peripheral surface of the second cover 2100 to allow air intake from any direction relative to the second cover 2100. That is, air can be drawn from a 360-degree direction based on the vertical centerline passing through the internal center of the second cover 2100.

As such, by configuring the second cover 2100 in a cylindrical shape and forming multiple second inlets along the outer peripheral surface of the second cover 2100, the amount of air intake can be increased.

Air drawn through the second inlet can flow approximately radially from the outer peripheral surface of the second cover 2100.

The air purifier 700′) includes a partition device 5000 provided between the first blower 1000 and the second blower 2000. By means of the partition device 5000, the second blower 2000 can be positioned spaced apart above the first blower 1000.

The partition device 5000 can guide the air discharged from the first outlet 1500. For example, the partition device 5000 can guide the air discharged axially from the first outlet 1500 to be discharged in the transverse direction perpendicular to the axial direction.

The flow conversion device 3000 can be installed above the second blower 2000. Based on the air flow, the air passage of the second blower 2000 can be in communication with the air passage of the flow conversion device 3000. The air passing through the second blower 2000 can be discharged to the outside via the air passage of the flow conversion device 3000 and through the second outlet 3500. The second outlet 3500 can be formed at the upper end of the flow conversion device 3000.

The flow conversion device 3000 can be provided to be movable. Specifically, as shown in FIG. 29(a), the flow conversion device 3000 can be in a lying state (first position) or, as shown in FIG. 29(b), in an inclined upright state (second position). Hereinafter, the flow conversion device 3000 may also be referred to as a ‘circulator.’

At the upper part of the flow conversion device 3000, a display unit 4000 that includes an operation unit for displaying the operation information of the air purifier 700′) and controlling the operation of the air purifier 700′) can be disposed. The display unit 4000 can move together with the flow conversion device 3000.

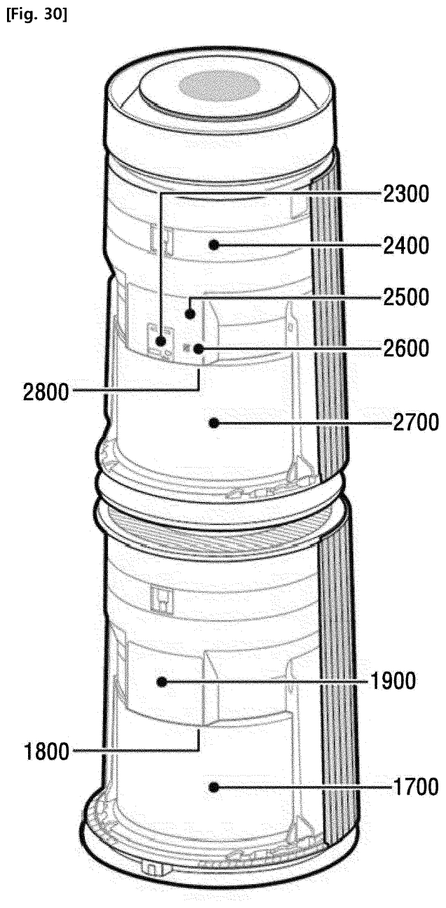

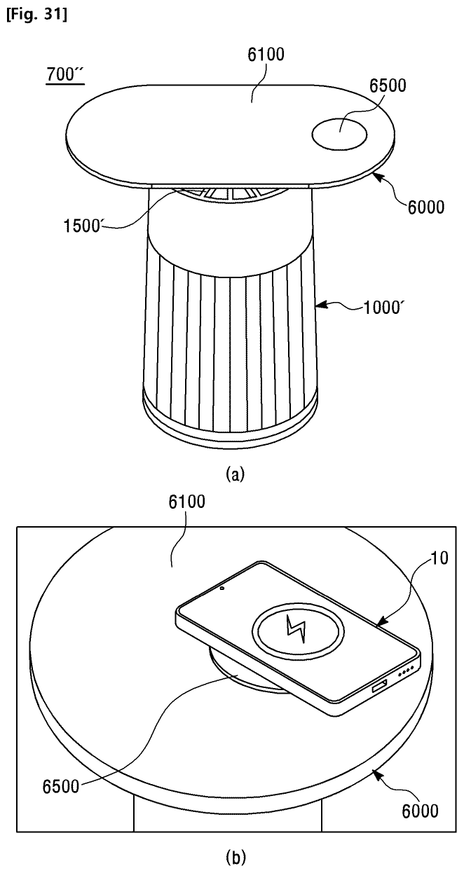

FIG. 30 is a diagram showing a state where some parts of the covers 1100, 2100 of the air purifier 700′) shown in FIG. 29 are removed.

Referring to FIGS. 29 and 30, the air purifier 700′) includes a sensor 2300 for detecting the concentration of dust. The sensor 2300 can be disposed in the second blower 2000, but is not limited thereto and may also be disposed in the first blower 1000. The sensor 2300 may include a sensor for sensing ultrafine particles (PM1.0.

The air purifier 700′) may include a smart diagnosis unit 2400. The smart diagnosis unit 2400 can check the product status through smart diagnosis when the air purifier 700′) malfunctions or breaks down.

The air purifier 700′) may include an AI-Sensor communication module 2500. The AI-Sensor communication module 2500 can be linked with an AI sensor (not shown) that can be communicatively connected to the air purifier 700′) to detect pollution locations.

The air purifier 700′) may include a gas sensor 2600. The gas sensor 2600 can detect gas or odors.

The air purifier 700′) includes filters 1700, 2700 for purifying air. The filters 1700, 2700 may be disposed in each of the first blower 1000 and the second blower 2000.

As shown in FIG. 30, the air purifier 700′) may include a filter-status detection sensor 1900. The filter-status detection sensor 1900 can detect the replacement timing of the filters 1700, 2700.

The air purifier 700′) may include UV light-emitting elements 1800, 2800 for sterilizing the fans disposed inside. Each of the first and second blowers 1000, 2000 may be equipped with a fan, and the UV light-emitting elements 1800, 2800 may be positioned to illuminate the fans inside each blower 1000, 2000.

FIG. 24(b) is a diagram showing an example of the display unit 4000 of the air purifier 700′) according to an embodiment of the present invention. Referring to FIG. 24(b), the display unit 4000 may include multiple operation units 4100, 4200, 4300, 4400, 4500.

As shown in FIG. 24, the multiple operation units 4100, 4200, 4300, 4400, 4500 may include an operation/stop button 4100 for starting or stopping the air purifier 700′), an operation-mode button 4200 for selecting the operation mode, a purification-level button 4300 for adjusting the wind intensity of the air purifier 700′), a booster-control button 4400 for adjusting the intensity and rotation settings of the booster, and a setting button 4500 for managing and setting notifications of the air purifier 700′). These multiple operation units 4100, 4200, 4300, 4400, 4500 may be touch sensors or mechanical buttons.

Control signals input through the multiple operation units 4100, 4200, 4300, 4400, 4500 of the display unit 4000 are input to the processor 730 shown in FIG. 17(b), and the processor 730 can control the drive unit 740 based on the input control signals.

As shown in FIG. 23, the operation mode 4210 may include various operation modes. For example, it may include an AI mode, pet mode, clean booster mode, dual purification mode, and single purification mode. Here, the AI mode is a mode that automatically adjusts the operation mode and purification level according to the comprehensive cleanliness of the air purifier 700′) and the AI sensor; the pet mode is a dedicated mode for users with pets; the clean booster mode is a mode that uses the booster provided in the second blower 2000 to quickly send purified air over a long distance to circulate indoor air; the dual purification mode is a mode where the first blower 1000 and the second blower 2000 operate simultaneously to quickly purify indoor air; and the single purification mode is a mode that purifies indoor air with the second blower 2000.

The air purifier 700′) according to an embodiment of the present invention may further include a sleep mode as the operation mode 4210. This is explained with reference to FIGS. 23(c) and (d).

FIGS. 23(c) and (d) are diagrams of the display unit 4000 for explaining the sleep mode of the air purifier 700′) according to an embodiment of the present invention.

Referring to FIG. 23(c), the operation mode 4210 may include a sleep mode 4250. The sleep mode 4250 is a mode that automatically adjusts the air quality within the user's sleep space by detecting the user's sleep state. The sleep mode 4250 can be controlled by the processor of the air purifier 700′).

Based on the first environment adjustment information to the nth environment adjustment information generated by the computing device 100 in FIG. 16(c) or the air purifier 700′) in FIG. 16(d), the air purifier 700′) can perform air quality control within the sleep space.

For example, the air purifier 700′) can preemptively remove fine dust and harmful gases according to the first environment adjustment information until a predetermined time before the user's sleep (e.g., 20 minutes prior). Alternatively, it can generate noise (white noise) to a degree that induces sleep just before sleeping, adjust the blowing intensity to be below a predefined intensity, or reduce the brightness of the display unit 4000. According to the second environment adjustment information, the air purifier 700′) can turn off the display unit 4000, operate with noise below a predefined level, adjust the blowing intensity to be below a predefined intensity, set the blowing temperature within a predefined range, or maintain the humidity within the sleep space at a predetermined temperature. According to the third environment adjustment information, the air purifier 700′) can reduce the blowing intensity and noise at the waking time, generate white noise to gradually induce waking, maintain noise below a predefined level, or operate in conjunction with a predicted waking time or recommended waking time. According to the fourth environment adjustment information, the air purifier 700′) can control at least one of the blowing intensity, noise level, and alarm.

In the system configuration of FIG. 16(c), to operate the air purifier 700′) in sleep mode 4250, a device connection process for connecting the air purifier 700′) to the computing device 100 via a network can be performed.

In the system configuration of FIG. 16(d), to operate the air purifier 700′) in sleep mode 4250, a connection process for interfacing with the user terminal 10 as shown in FIG. 23(d) can be performed. The air purifier 700′) and the user terminal 10 can be wirelessly connected. For example, the air purifier 700′) and the user terminal 10 can be directly connected via a network as shown in FIG. 16(d).

### Configuration of the Air Purifier According to Another Embodiment

FIG. 31(a) is a diagram for explaining another example of the air purifier shown in FIGS. 16 and 17.

The air purifier 700″) shown in FIG. 31(a) may be an air purifier used in individual spaces such as a bedroom or study.

The air purifier 700″) includes a blower 1000′) and a table 6000.

The blower 1000′) corresponds to the first blower 1000 shown in FIG. 29 and includes configurations for purifying air quality inside, similar to the first blower 1000. Therefore, the detailed description of the blower 1000′) is replaced by the description of the first blower 1000 shown in FIG. 29.

As shown in FIG. 31, the table 6000 can be placed on the blower 1000′).

As illustrated, the table 6000 may include a lower surface (not shown) that guides the air discharged from the outlet 1700′) positioned on the upper part of the blower 1000′), an upper surface 6100 on which items can be placed, and a wireless charging unit 6500 disposed on a portion of the upper surface 6100. A lighting unit capable of emitting light in various colors may also be disposed on the lower surface of the table 6000.

The air purifier 700″) can operate in a sleep mode that automatically adjusts the air quality within the sleep space, similar to the air purifier 700′) shown in FIG. 29.

The sleep mode of the air purifier 700″) can perform air quality adjustment within the sleep space based on the first to nth environment adjustment information generated by the air purifier 700′) as shown in FIG. 16(c) or the computing device 100 in FIG. 16(d).

For example, the air purifier 700″) can pre-remove fine dust and harmful gases according to the first environment adjustment information until a predetermined time before the user's sleep (e.g., 20 minutes prior). Alternatively, it can induce sleep by generating a level of noise (white noise) just before sleep, adjust the blowing intensity to below a predefined level, or reduce the brightness of the lighting unit (not shown). According to the second environment adjustment information, the air purifier 700″) can turn off the lighting unit (not shown), operate with noise below a predefined level, adjust the blowing intensity to below a predefined level, set the blowing temperature within a predefined range, or maintain the humidity within the sleep space at a predetermined temperature.

According to the third environment adjustment information, the air purifier 700″) can reduce the blowing intensity and noise at the wake-up time, generate white noise to gradually induce waking, maintain noise below a predefined level, or operate in conjunction with a wake-up prediction or recommendation time. According to the fourth environment adjustment information, the air purifier 700″) can control at least one of the blowing intensity, noise level, and alarm.

Unlike the air purifier 700′) shown in FIG. 29, the air purifier 700″) may not be equipped with a display unit.

Therefore, according to the system configuration of FIGS. 16(c) and (d), the air purifier 700″) can be remotely controlled by the user terminal 10 in FIG. 16 to operate in sleep mode.

It can be remotely controlled by the user terminal 10 in FIG. 16. This will be explained with reference to FIG. 25.

FIG. 25(c) shows the screen of a first application for remotely controlling the air purifier 700″) from the user terminal 10, and FIG. 25(d) shows the screen of an application for controlling the sleep mode of the air purifier 700″).

Referring to FIG. 25(c), an application for controlling the air purifier 700″) may be installed on the user terminal 10. The air purifier 700″) can be controlled through the application.

The first application can provide a screen capable of controlling the operation mode of the air purifier 700″), allowing the user to select a desired operation mode (e.g., A mode, sleep mode, B mode).

If the user selects the sleep mode as shown in FIG. 25(c), a screen allowing specific control of the sleep mode of the air purifier 700″) may be displayed as shown in FIG. 25(d).

Through this, the user can select a user terminal to connect with the air purifier 700″) and can connect other terminals to the air purifier 700″).

The air purifier 700″) can perform a connection process to interoperate with the user terminal 10 as illustrated in FIGS. 25(c) and (d). The air purifier 700′) and the user terminal 10 can be connected wirelessly. For example, the air purifier 700′) and the user terminal 10 can be connected through a network as shown in FIG. 16(d).

Meanwhile, the control of the air purifier 700′) through the first application shown in FIG. 25 can be directly applied to the air purifier 700 shown in FIG. 29.

### Method for Operating the Sleep Mode of the Air Purifier

The aforementioned air purifiers 700′, 700″) are configured to operate in sleep mode according to the user's selection, but are not limited thereto. In another embodiment of the present invention, the air purifier may be configured to automatically operate in sleep mode without the user's selection. This will be explained with reference to FIG. 26.

FIG. 26 is a diagram for explaining the operation of the air purifier 700″) according to another embodiment of the present invention.

Referring to FIG. 26, the air purifier 700″′) can automatically operate in sleep mode without a separate user selection based on environment adjustment information generated from sleep state information and/or sleep stage information.

For example, if the processor 130 of the computing device 100 in FIG. 16(c) or the processor 730 of the air purifier in FIG. 16(d) and FIG. 17(b) generates second environment adjustment information by identifying the user's sleep onset time through second sleep state information, the air purifier 700″′) can be configured to automatically start the sleep mode.

Additionally, if the processor 130 of the computing device 100 in FIG. 16(c) or the processor 730 of the air purifier in FIG. 16(d) and FIG. 17 identifies the wake-up prediction time to generate third environment adjustment information, the air purifier 700″′) can be configured to automatically terminate the sleep mode at the wake-up prediction time.

Unlike what is depicted in FIG. 26, the start and end points of the sleep mode may vary. For example, the start point of the sleep mode may be a sleep induction point prior to the sleep onset point.

In another example, as shown in FIG. 25(c), the start point of the sleep mode may be immediately after the sleep mode of the first application is selected by the user or after a predetermined time has elapsed following the selection of the sleep mode. Alternatively, as shown in FIG. 25(d), it may be immediately after a predetermined device (Device A) is connected to the air purifier or after a predetermined time has elapsed following the connection.

Further examples of potential start points for the sleep mode will be described with reference to the drawings.

FIGS. 27 and 31(b) are diagrams for explaining the start point of the sleep mode operation of the air purifier 700″′) shown in FIG. 26.

As shown in FIG. 31(b), the start point of the sleep mode may be immediately after the user terminal 10 is placed on the wireless charging unit 6500 and begins charging, or after a predetermined time has elapsed following the start of charging.

Referring to FIG. 31(b), the start point of the sleep mode may be after a predetermined time has elapsed following the placement of the user terminal 10 on a predetermined portion of the upper surface 6100 of the table 6000. Here, the predetermined time may be the duration for which the result measured by the accelerometer sensor provided in the user terminal 10 remains constant.

As shown in FIG. 27, the start point of the sleep mode may be the point at which the ‘Go to Sleep (15)’ button is selected through the second application installed on the user terminal 10. Here, the second application may be an application that automatically outputs an AI-based sleep report by measuring environment sensing information (e.g., the user's breathing sound).

The second application may be interlinked with the first application shown in FIGS. 25(c) and (d), allowing them to utilize each other's data.

Alternatively, referring to FIG. 27, the start point of the sleep mode may be after a predetermined time has elapsed following the selection of the ‘Go to Sleep (15)’ button through the second application. Here, the predetermined time may be the duration for which the result measured by the accelerometer sensor provided in the user terminal 10 remains constant.

Referring again to FIG. 26, the end point of the sleep mode may be, for example, after a predetermined time has elapsed following a predefined time after the wake-up prediction point.

In another example, referring to FIGS. 25(c) and (d), the endpoint of the sleep mode may occur immediately after the disconnection between the first application installed on the user terminal 10 and the air purifier.

In yet another example, referring to FIG. 31(b), the endpoint of the sleep mode may occur immediately after the user terminal 10 is removed from the wireless charging unit 6500 and wireless charging is interrupted, or after a predetermined time has elapsed following the interruption. Alternatively, it may occur immediately after the user terminal 10 is removed from the table 6000 or after a predetermined time has elapsed following the removal.

In another example, referring to FIG. 28, the endpoint of the sleep mode may be the point in time when the ‘Wake Up (17)’ button is selected through the second application installed on the user terminal 10.

Additionally, the start and endpoint of the sleep mode can be variously modified by the user or manufacturer of the air purifier 700″′).

For the continuous operation of the sleep mode, in the system configuration of FIG. 16(c), it is preferable that the user terminal 10 is connected to the computing device 100 through a network, and the computing device 100 is connected to the air purifier 700″′) through the network. Meanwhile, in the system configuration of FIG. 16(d), it is preferable that the user terminal 10 is connected to the air purifier 700″′) through a network or short-range wireless communication.

The air purifier 700″) may have the same hardware configuration as the air purifier 700′, 700″) shown in FIG. 29 or FIG. 31(a).

### Operation of the Air Purifier or Air Purification Fan in Sleep Mode and Wake-Up Mode According to the Embodiment

Additionally, the operation of a table-type air purifier that adjusts air quality and light, and an air purification fan that adjusts air quality and temperature in sleep mode and wake-up mode will be described.

First, the operation of the table-type air purifier in sleep mode is as follows.

The table-type air purifier may operate independently, but if premium air care is required, it may operate in an integrated package format with an air conditioner, humidifier, and/or dehumidifier.

During the preparation for sleep stage, the table-type air purifier can wirelessly charge the smartphone 900 when it is placed on the table during a specific time period when the user is not using the smartphone 900, and can recognize the sleep management app installed on the smartphone 900 to initiate the measurement of the user's sleep.

The air conditioner can adjust the indoor temperature suitable for the user's sleep environment, and the humidifier and/or dehumidifier can adjust the indoor humidity suitable for the sleep environment.

During the falling asleep stage, the table-type air purifier can turn off the indirect lighting, and during the sleep stage, the Sleep Track app and the sleep management app can interoperate to measure the user's sleep situation in real-time.

Next, the operation of the table-type air purifier in the wake-up mode is as follows.

In the pre-wake-up stage, the table-type air purifier can perform a morning alarm function by generating intentional noise in the power air purification mode, activate the smart alarm installed in the healthcare app, or adjust the lighting intensity so that the sleep light gradually brightens.

In the wake-up stage, when the confirmation of the sleep state is detected on the user's smartphone 900, the sleep measurement is terminated, and in the post-wake-up stage, the healthcare app can display the analyzed user's sleep report on the user's smartphone 900.

Meanwhile, the operation of the air purification fan in the sleep mode is as follows.

The air purification fan, like the table-type air purifier, can operate independently, but if premium air care is required, it can operate in an integrated package format with the humidifier and/or dehumidifier.

During the preparation for sleep stage, the air purification fan can be manually activated through the healthcare app to adjust the temperature suitable for sleep using fan and warm air, and the humidifier and/or dehumidifier can adjust the indoor humidity suitable for the sleep environment.

During the sleep stage, the Sleep Track app and the sleep management app can interoperate to measure the user's sleep situation in real-time.

Subsequently, the operation of the air purification fan in the wake-up mode, i.e., during the pre-wake-up stage, wake-up stage, and post-wake-up stage, is identical to the operation of the table-type air purifier in the wake-up mode. Therefore, the explanation will be omitted here.

### Detailed Scenarios of Smart Home-Appliances by Sleep Stage

FIG. 41 is a table illustrating the operation of the preparation-for-sleep stage in the detailed scenario of smart home-appliances operating in a time-series manner by the user's sleep stage using the sleep analysis method according to the present invention.

FIG. 42 is a table illustrating the operation of the stage from post-sleep onset to pre-deep sleep, connected in time-series succession to FIG. 41 in the scenario.

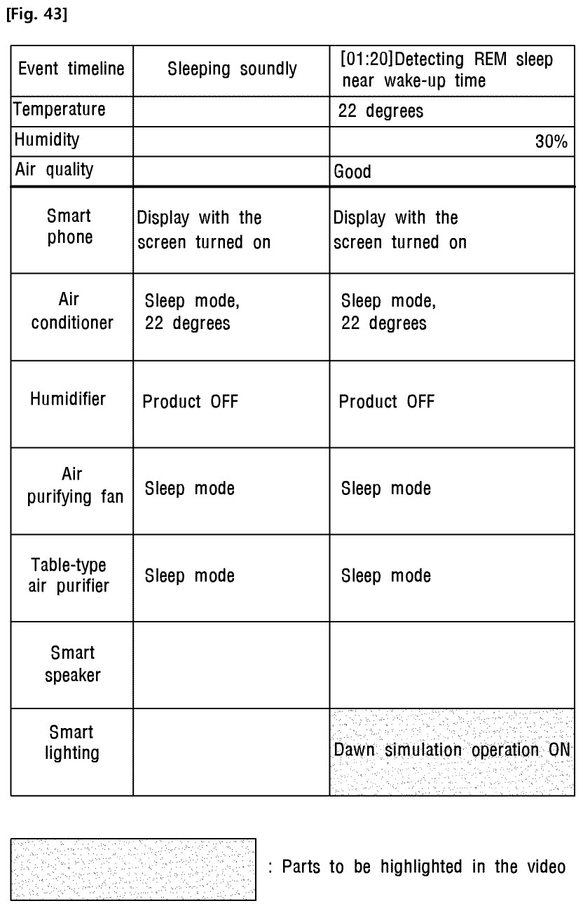

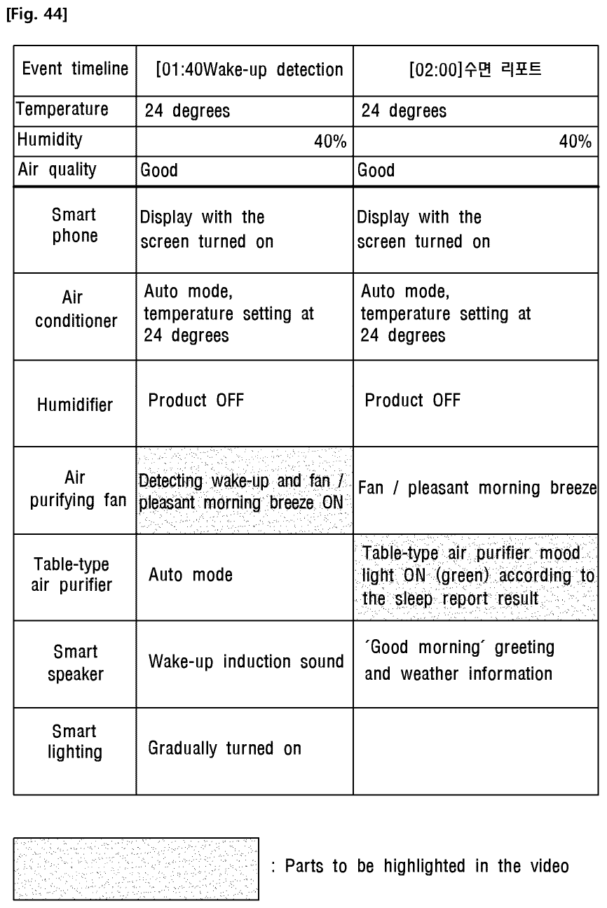

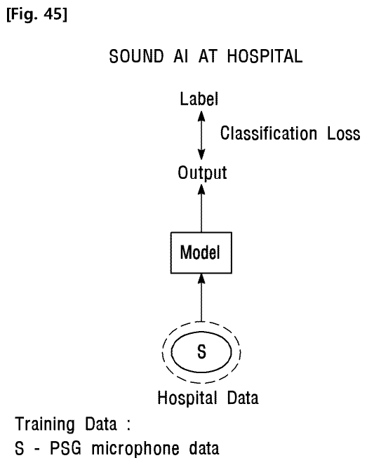

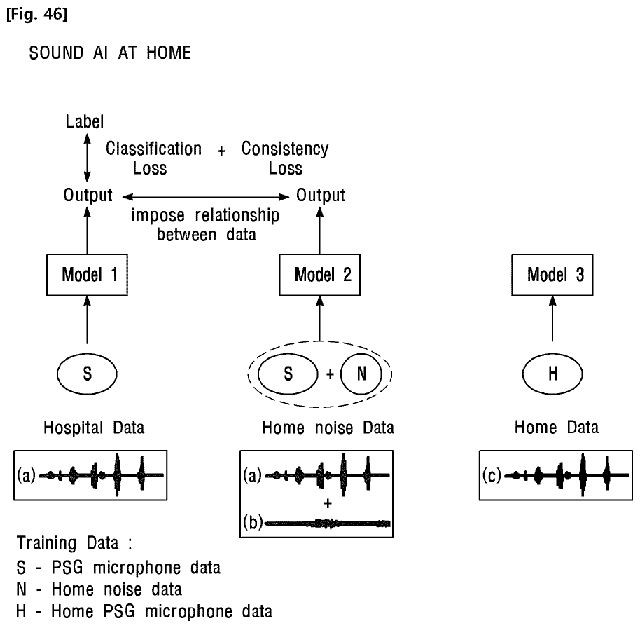

FIG. 43 is a table illustrating the operation of the stage from post-deep sleep to pre-wake-up detection, connected in time-series succession to FIG. 42 in the scenario.

FIG. 44 is a table illustrating the operation of the wake-up stage, connected in time-series succession to FIG. 43 in the scenario.

Referring to FIGS. 50 and 51, and FIGS. 41 to 44, the operation of the detailed scenario of smart home-appliances operating in a time-series manner by the user's sleep stage using the sleep analysis method of the present invention is described as follows. However, the scenario described below is merely an example according to the present invention, and the scope of the present invention is not limited thereto.

First, it is assumed that the time the user enters the bedroom is midnight, and the wake-up time is 01:40 AM.

Additionally, it is assumed that when the user enters the bedroom, the initial temperature is 29 degrees Celsius, and the initial humidity is 30%, indicating a night where the temperature is high but the humidity is not, with the air quality assumed to be ‘moderate.’

As shown in FIG. 41, at the time the user enters the bedroom (00:00, the smartphone 900 may be displayed with the screen turned on (the same applies to subsequent stages).

Additionally, the air conditioner, air purification fan, table-type air purifier, and smart lighting may be turned on, while the humidifier and smart speaker 804 may remain in the off state.

Meanwhile, the air conditioner may be set to an operating temperature of 24 degrees Celsius.

Furthermore, the air purification fan and table-type air purifier may be set to automatic mode.

At the time (00:10 when the user lies down on the bed and places the smartphone 900 on the table of the table-type air purifier, an image of the smartphone 900 being placed on the table-type air purifier may be displayed on the screen of the smartphone 900.

Additionally, the air purification fan and table-type air purifier may be switched to sleep mode. The air purification fan may be set to adjust to the optimal air purification state according to the user's recent sleep records.

Moreover, the smart speaker 804 may turn on sleep-inducing sounds. The smart lighting may be set to gradually dim and then turn off.

Meanwhile, due to the operation of the air conditioner set to an operating temperature of 24 degrees Celsius, the indoor temperature of the bedroom may decrease from the initial temperature of 29 degrees Celsius towards the operating temperature of 24 degrees Celsius.

As shown in FIG. 42, at the time (00:20 when the user's sleep onset state is detected, the air conditioner may be reset to the optimal temperature (e.g., 22 degrees Celsius) according to the user's recent sleep records and may be switched to sleep mode.

Additionally, since there is no longer a need to induce the user's sleep, the smart speaker 804 may turn off the sleep-inducing sounds.

Meanwhile, as the air conditioner operates with the operating temperature reset to the optimal temperature of 22 degrees Celsius, the indoor temperature of the bedroom may decrease from 24 degrees Celsius towards the optimal temperature of 22 degrees Celsius, and the air quality may transition to a ‘good’ state due to the continued operation of the air purification fan and table-type air purifier, which were set to the user's optimal air purification state.

If, at the time point (00:40, it is assumed that the user experiences snoring and/or sleep apnea during sleep, the sound sensor installed inside the humidifier can detect changes in the user's breathing sounds, and the processor 830 within the humidifier can turn on the power of the humidifier to protect the user's nose. Consequently, the initial humidity of 30% can increase to 50%.

Meanwhile, the smart speaker 804 can turn on sleep-inducing sounds to guide the user back to sleep.

As shown in FIG. 43, through the user's breathing sounds detected by the sound sensors installed inside each of the plurality of smart home-appliances 800, the embedded processor can determine that the user has entered a deep sleep state. In this case, the current state of each smart home-appliance 800 can be maintained.

If, at the time point (01:20, when the scheduled wake-up time (01:40 is approaching, it is assumed that the REM sleep stage of the user's sleep stages is detected, the smart lighting can turn on the dawn simulation operation.

Finally, as shown in FIG. 44, if, at the scheduled wake-up time (01:40, it is assumed that the wake-up stage of the user's sleep stages is detected, the air conditioner can switch to automatic mode and reset the operating temperature to 24 degrees Celsius.

Additionally, the air purification fan can be set to the fan and/or pleasant morning breeze mode. The table-type air purifier can be set to automatic mode.

Furthermore, the smart speaker 804 can turn on wake-up inducing sounds to guide the user's awakening. The smart lighting can be set to turn off as it is no longer needed in the bedroom after the dawn simulation operation.

Moreover, if, at the time point (02:00, it is assumed that the user's wake-up state is detected, the color mood light within the table-type air purifier can turn on in green.

Additionally, the smart speaker 804 can audibly provide the user with the weather information for the day along with a ‘Good Morning’ greeting.

### Wearable Device

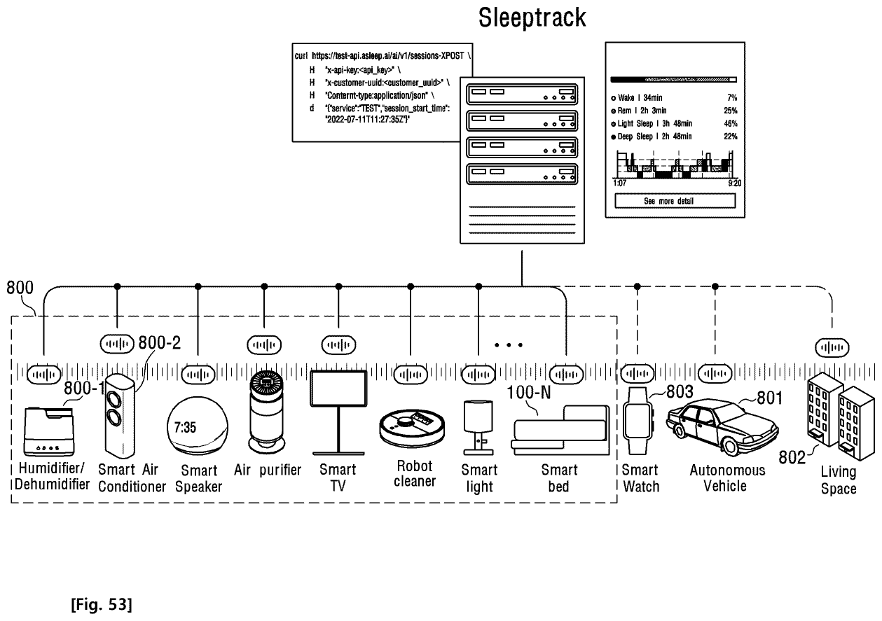

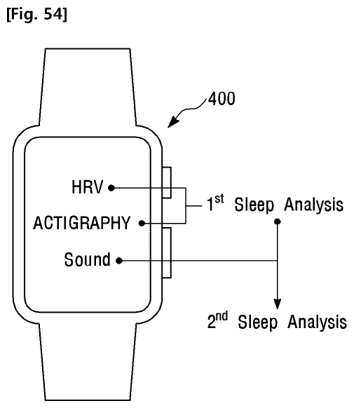

FIG. 53 is a block diagram for explaining the operation of a system implementing the method of providing environment adjustment information according to the present invention, including one or more smart home-appliances 800, a sleep analysis (Sleep track) app, a smart watch 803, an autonomous vehicle 801, and a living space 802.

As illustrated in FIG. 54, the wearable device according to the present invention, specifically the smart watch 803, performs a two-stage sleep analysis. Specifically, the first sleep analysis is conducted based on user biometric information acquired by the sensing unit provided in the wearable device 803. The user biometric information may include HRV (Heart Rate Variability) and Actigraphy (body movement signals). However, it is not limited thereto, and the first sleep analysis may utilize various biometric signals capable of identifying the user's sleep state.

According to one embodiment of the present invention, in the first sleep analysis, the user's sleep onset time or wake-up time can be detected based on Actigraphy information immediately before and after the user falls asleep. Specifically, based on Actigraphy information, the user's sleep onset can be identified. For example, if the user's movements gradually decrease and remain below a predefined threshold (e.g., three times) for a predetermined period, it can be determined that sleep has occurred.

Conversely, if the user's movements are detected above a specified threshold (e.g., three times) and gradually increase over a certain period, it can be determined that sleep has ended. The first sleep analysis analyzes sleep onset time, wake-up time, total sleep time, and various information related to sleep stages, such as hypnogram, sleep time per sleep stage, and sleep cycle. As will be described in more detail below, sleep stages can be divided into NREM (non-REM) sleep and REM (Rapid Eye Movement) sleep, and NREM sleep can be further divided into multiple stages (e.g., two stages of Light and Deep, or four stages from N1 to N4. The setting of sleep stages may be defined by general sleep stage classification methods but can also be defined in various ways by the designer. Through sleep analysis, not only the quality of sleep but also sleep disorders (e.g., sleep apnea) and their underlying causes (e.g., snoring) can be predicted.

In the first sleep analysis, changes in sleep stages are analyzed, and a hypnogram can be generated to identify the analyzed changes in sleep stages, thereby identifying the user's sleep cycle.

Meanwhile, in the first sleep analysis, sleep stages can be classified based on user biometric information such as HRV and/or Actigraphy information. HRV refers to physiological changes occurring according to the intervals between heartbeats (RR interval). Heart rate is determined by the autonomic nervous system affecting the inherent spontaneity of the sinoatrial node, related to the interaction between the sympathetic and parasympathetic nerves.

This interaction changes moment by moment according to changes in the internal or external environment, resulting in changes in the user's heart rate. For example, in healthy individuals, heart rate variability appears large and complex, but in disease or stress states, the complexity can significantly decrease. Since the heart continues to function even during sleep, HRV allows for the prediction of sleep state and evaluation of sleep quality. For instance, if sleep quality deteriorates, sympathetic nerve activity to the heart is activated, and parasympathetic nerve control ability to the heart decreases.

Therefore, HRV, reflecting sleep quality and autonomic nervous system state, has an organic correlation, allowing for the evaluation of sleep quality through HRV. Another example is analyzing the ratio of sympathetic/parasympathetic nervous system activity in HRV, where the wakefulness stage appears higher compared to NREM stages 2, 3, and 4, and NREM stage 4 appears lower compared to REM and NREM stage 1. By utilizing the ratio of sympathetic/parasympathetic nervous system activity, sleep stages can be accurately determined. Various other methods can be used to perform sleep analysis based on HRV.

Additionally, in the first sleep analysis, Obstructive Sleep Apnea (OSA) can be screened. For example, based on user biometric information (e.g., heart rate, heart rate variability, oxygen saturation, etc.) received through the sensing unit, the user's breathing during sleep can be identified. Furthermore, based on Actigraphy information or user posture information, Obstructive Sleep Apnea can be screened in stages.

In the second sleep analysis, the quality of sleep, sleep stages, and the presence of sleep apnea are analyzed based on the results of the first sleep analysis and sleep sound information. Sleep sound information may refer to audio information related to movements or breathing occurring during the user's sleep.

The second sleep analysis involves pre-processing the user's sleep sound information and analyzing the user's sleep stages through an AI algorithm, with the specific analysis method to be described in more detail below.

According to the present invention, the wearable device 803 is in contact with the user's body to acquire biometric signals and perform a primary sleep analysis (contact-based sleep analysis). Subsequently, the microphone embedded in the wearable device 803 acquires the user's sleep sound information and performs a secondary sleep analysis (non-contact-based sleep analysis) using this information, thereby enabling more precise sleep analysis.

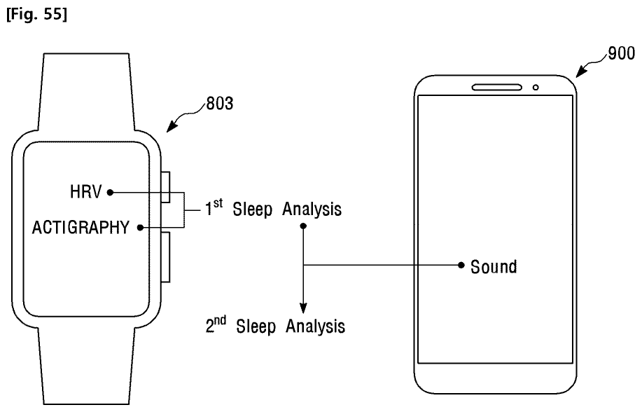

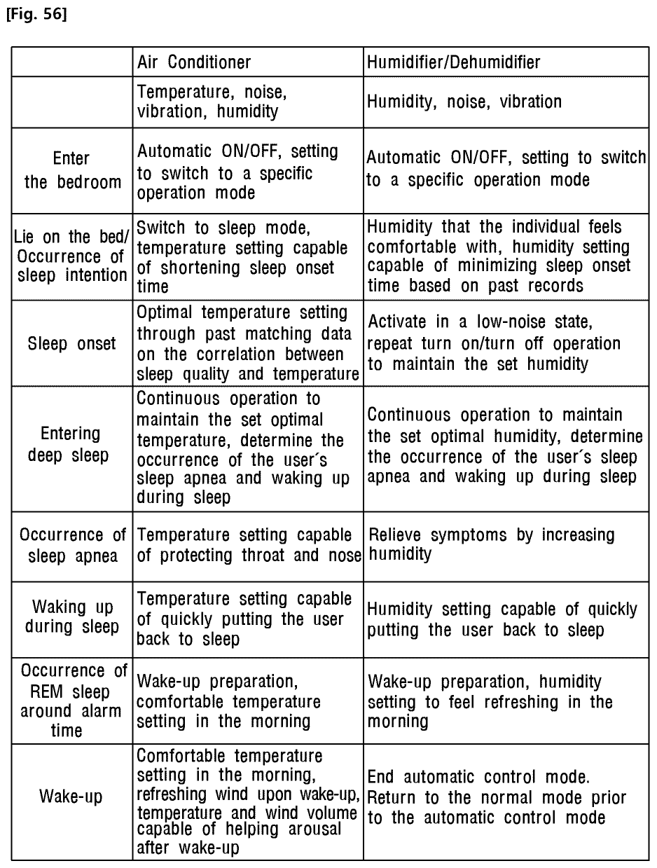

Unlike the embodiment shown in FIG. 54, in the embodiment shown in FIG. 55, the wearable device 803 and the smartphone 900 work in conjunction to perform the user's sleep analysis. The wearable device 803 and the smartphone 900 can be paired via Bluetooth or connected through other wireless communication methods.

In the embodiment shown in FIG. 55, the primary sleep analysis 1st Sleep Analysis) is conducted by the wearable device 803, while the secondary sleep analysis 2nd Sleep Analysis) is conducted by the smartphone 900.

According to the present invention, the primary sleep analysis performed by the wearable device 803 is as described above. The results of the primary sleep analysis are transmitted wirelessly to the smartphone 900, which then performs the secondary sleep analysis based on the primary sleep analysis results and the user's sleep sound information.

At this time, the user's sleep sound information may be acquired from the wearable device 803 and transmitted to the smartphone 900, or it may be independently acquired through the microphone embedded in the smartphone 900.

That is, in the embodiment shown in FIG. 55, the contact-based sleep stage analysis is performed through the wearable device 803, and the non-contact-based sleep stage analysis is performed through the smartphone 900. The user can check the final sleep stage analysis results derived from the smartphone 900 on the smartphone's screen.

Additionally, the final sleep stage analysis results derived from the smartphone 900 are transmitted to the wearable device 803, and the user can check them on the screen of the wearable device 803.

Furthermore, the final sleep stage analysis results derived from the smartphone 900 are transmitted to the wearable device 803, and the user can check them on the screen of the wearable device 803.

Meanwhile, it is preferable for the wearable device 803 to be worn by the user. If the user does not wear the wearable device 803, it is necessary for the wearable device 803 to be appropriately positioned around the user to receive at least part of the input signals for the primary sleep analysis (e.g., Actigraphy information) or the input signals for the secondary sleep analysis (sleep sound information). Particularly, to extract Actigraphy information, it is advisable to place the device in an area capable of detecting the user's movements (e.g., under the pillow, on top of the mattress). Therefore, even if the wearable device 803 is not worn by the user, the sleep stage analysis described above can be performed if it is appropriately positioned within the user's sleep space.

In one embodiment, if the wearable device 803 is not worn by the user, the device can emit a predetermined signal to prompt the user to place the wearable device 803 close to them, allowing it to receive part of the input signals for the primary sleep analysis (e.g., Actigraphy information) or the input signals for the secondary sleep analysis (sleep sound information). The predetermined signal may be in the form of vibration, alarm, text, LED, etc.

The distance between the user and the wearable device 803 may be extracted by the wearable device 803 or by the smartphone 900. That is, since the user's sleep space is fixed, the position of the wearable device 803 can be tracked to determine whether the wearable device 803 is placed in an appropriate position. Alternatively, the wearable device 803 can recognize the distance between bedding (such as pillows, mattresses, etc.) by itself to determine whether it is placed in an appropriate position. In this case, the bedding (such as pillows, mattresses, etc.) may include a sensing unit for distance measurement, and the sensing unit can be connected to the wearable device 803 in various ways (such as communication network, optical connection, electrical connection, etc.) to determine the distance between them.

### Embodiment of the Method for Providing Environment Adjustment Information for Sleep

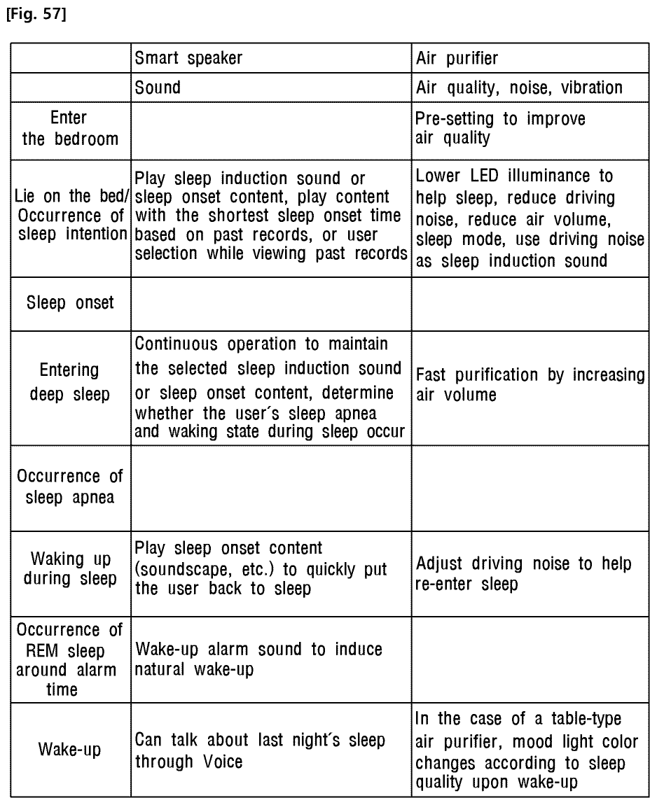

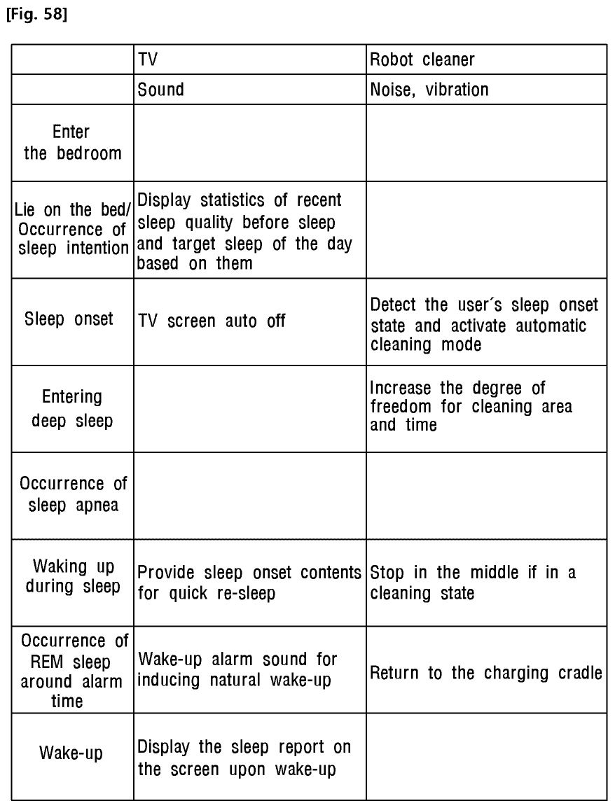

FIG. 57 is a table showing the operation of an air conditioner and a humidifier/dehumidifier among the major smart home-appliances that frequently switch operations according to the user's sleep stage flow using the method for providing environment adjustment information for sleep of the present invention.

FIG. 58 is a table showing the operation of a smart speaker and an air purifier among the major smart home-appliances according to the present invention.

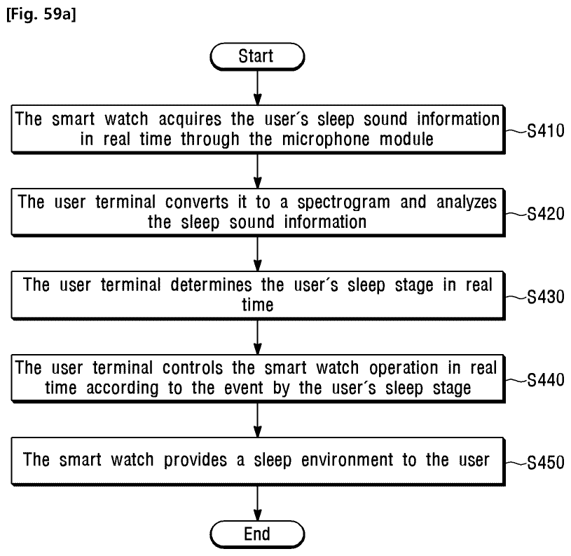

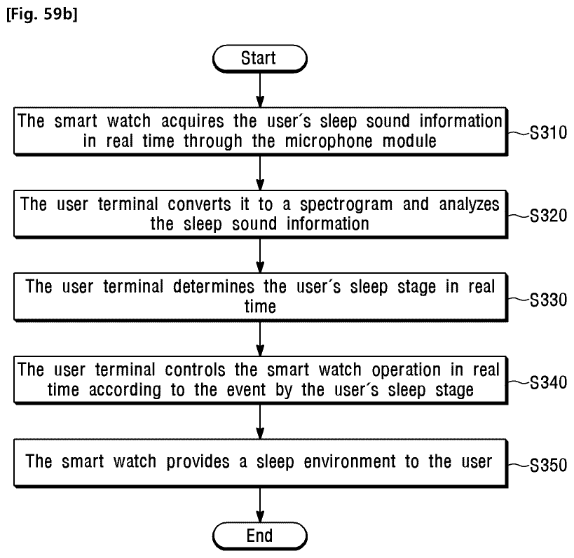

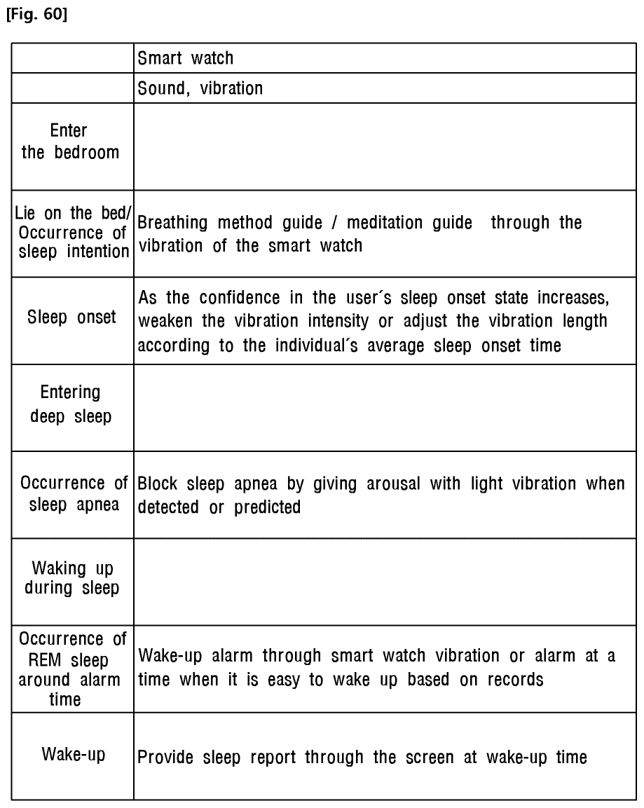

FIG. 59 is a table showing the operation of a smart TV and a robot vacuum cleaner among the major smart home-appliances according to the present invention.

The user's sleep stage may include detection of entering the bedroom, lying on the bed, falling asleep, entering deep sleep, occurrence of sleep apnea, waking during sleep, occurrence of REM sleep around alarm time, and waking up.

In particular, the detection of lying on the bed may include the stage where the user lies on the bed, the stage where the sleep button is pushed, and the stage where the user's intention to fall asleep is estimated by the user terminal.

Additionally, in FIGS. 57 to 59, blank spaces assume that no operation switch occurs from the immediately preceding state, and appropriate operations are performed whenever each event is detected.

Furthermore, the sleep environment composed by each of the one or more smart home-appliances 800 is as follows.

The air conditioner 800-2 may include temperature, noise, vibration, humidity; the humidifier/dehumidifier 800-1 may include humidity, noise, vibration; the smart speaker may include sound; the air purifier may include air quality, noise; the smart TV may include sound; blinds/curtains/lighting may include light; the smart bed may include temperature, position of the user's head and body; the clothing care device may include scent, deodorization, drying, and sterilization of clothing; the smart diffuser may include scent; the robot vacuum cleaner may include floor cleaning, vibration, noise; the washing machine/dryer may include vibration, noise; the water purifier may include customized water dispensing; and the refrigerator may include sleep report display, vibration, noise, etc.

Overall, the method for providing environment adjustment information according to an embodiment of the present invention may include: a step S410 in which one or more smart home-appliances 800 acquire sleep sound information related to the user's sleep in real-time through a microphone module; a step S420 in which the user terminal receives the acquired sleep sound information and performs conversion and analysis into a spectrogram to determine the sleep stage in real-time S430; and a step S440 in which the user terminal outputs a control signal to control the operation of one or more smart home-appliances 800 in real-time according to events occurring at each determined sleep stage.

At this time, the step of outputting the control signal may include a step S450 in which one or more smart home-appliances 800 respond to the control signal to provide the user with an optimal sleep environment.

Hereinafter, all stages for each major smart home-appliance and all states of the smart watch, all data set or configured, and all past records or data can be set or stored by: 1 using a medically proven expert system, 2 using statistical data of an unspecified large number of people, or 3 setting or storing based on the specific user's individual past sleep data.

### Air Conditioner

FIG. 56 is a table showing the operation of the air conditioner and humidifier/dehumidifier, which are among the major smart home-appliances with frequent operation transitions according to the user's sleep stage flow using the method for providing environment adjustment information of the present invention.

As shown in FIG. 50, when it is detected that the user enters the bedroom, the air conditioner 800-2 can be set to automatically turn on/off and switch to a specific operation mode.

Here, the ‘specific operation mode’ may include modes in which airflow, air direction, and set temperature are configured.

In this case, the control method for the configured operation includes: 1 using a medically proven expert system, 2 using statistical data of an unspecified large number of people, or 3 controlling based on the specific user's individual sleep data.

When it is detected that the user lies down on the bed, the operation mode of the air conditioner 800-2 can be switched to sleep mode.

That is, the airflow and the brightness of the display unit can be changed to sleep mode. The type of wind can be switched to indirect wind.

In this case, the control method for the operation that is changed or switched includes: 1 using a medically proven expert system, 2 using statistical data from an unspecified large number of people, and 3 controlling based on the specific user's individual sleep data.

The temperature can be set to reduce the time to fall asleep according to the user's past personal sleep records.

In this case, the control method for the operation being set includes controlling based on the specific user's individual sleep data.

Additionally, in this case, when the user presses the sleep button while lying in bed, the user's intention to fall asleep is estimated.

When the user's intention to fall asleep is detected, the air conditioner 800-2 can set the optimal temperature that enhances sleep quality through past matching data on the correlation between the user's sleep quality and temperature.

In this case, the control method for the operation being set includes: 1 using a medically proven expert system, 2 using statistical data from an unspecified large number of people, and 3 controlling based on the specific user's individual sleep data.

When the user's entry into deep sleep is detected, the air conditioner 800-2 can continue to operate to maintain the temperature set when the intention to fall asleep was detected. It can determine the occurrence of the user's sleep apnea or awakening state during sleep.

In this case, the control method for determining the operation includes: 1 using a medically proven expert system, 2 using statistical data from an unspecified large number of people, and 3 controlling based on the specific user's individual sleep data.

When the occurrence of sleep apnea is detected, the air conditioner 800-2 can be set to a temperature that protects the user's neck, nose, and respiratory system.

That is, when snoring or the occurrence of sleep apnea is detected, it is common for the user to sleep with their mouth open, which can cause the upper airway and inside of the mouth to become dry, making them vulnerable to bacterial infections.

For this reason, the air conditioner 800-2 of the present invention is set to a temperature that protects the user's body part.

In this case, the control method for the setting operation includes: 1 using a medically proven expert system, 2 using statistical data of an unspecified large number of people, and 3 controlling based on the individual sleep data of a specified user.

If awakening during sleep is detected, the air conditioner temperature can be set to allow the user to quickly re-enter sleep.

In this case, the control method for the setting operation includes: 1 using a medically proven expert system, 2 using statistical data of an unspecified large number of people, and 3 controlling based on the individual sleep data of a specified user.

If the occurrence of REM sleep is detected around the alarm time, the air conditioner 800-2 can be set to prepare the user for waking. It can be set to a comfortable temperature in the morning.

In this case, the control method for the setting operation includes: 1 using a medically proven expert system, 2 using statistical data of an unspecified large number of people, and 3 controlling based on the individual sleep data of a specified user.

When waking is detected, the air conditioner 800-2 can be set to a comfortable temperature in the morning. It can provide a refreshing breeze upon the user's waking. It can be set to a temperature and airflow that assist in post-waking alertness.

In this case, the control method for the setting operation includes: 1 using a medically proven expert system, 2 using statistical data of an unspecified large number of people, and 3 controlling based on the individual sleep data of a specified user.

### Humidifier/Dehumidifier

FIG. 56 is a table illustrating the operation of an air conditioner and a humidifier/dehumidifier, which are among the primary smart home-appliances with frequent operation transitions, in one embodiment using the method of providing environment adjustment information for sleep according to the user's sleep stage flow in the present invention.

When entry into the bedroom is detected, the humidifier/dehumidifier 800-1 shown in FIG. 53 can be automatically set to turn on/off and switch to a specific operation mode.

Here, the ‘specific operation mode’ may include modes where settings such as air volume, air direction, and target humidity can be configured.

In this case, the control methods for the set operation include: 1 using a medically proven expert system, 2 using statistical data from an unspecified large number of people, and 3 controlling based on specific user's individual sleep data.

When lying on the bed is detected, the humidity can be set to the level that the individual user finds most comfortable, or to a humidity level that minimizes the time to fall asleep based on the user's past sleep records.

In this case, the control methods for the set operation include: 1 using a medically proven expert system, 2 using statistical data from an unspecified large number of people, and 3 controlling based on specific user's individual sleep data.

When falling asleep is detected, the humidifier/dehumidifier 800-1 is activated in a low-noise state. It may repeat the turn on/off operation to maintain the set humidity detected when lying on the bed.

In this case, the control methods for maintaining the set humidity include: 1 using a medically proven expert system, 2 using statistical data from an unspecified large number of people, and 3 controlling based on specific user's individual sleep data.

When deep sleep entry is detected, the humidifier/dehumidifier 800-1 can continue to operate to maintain the optimal humidity set when lying on the bed is detected.

In this case, the control methods for maintaining the set humidity include: 1 using a medically proven expert system, 2 using statistical data from an unspecified large number of people, and 3 controlling based on specific user's individual sleep data.

Upon detection of sleep apnea occurrence, the humidifier/dehumidifier 800-1 increases the bedroom humidity to alleviate the symptoms.

Specifically, when snoring or sleep apnea occurrence is detected, it is common for the user to sleep with their mouth open, which causes the upper airway and inside of the mouth to become dry, making them susceptible to bacterial infections.

For this reason, the humidifier/dehumidifier 800-1 of the present invention is set to a humidity level that protects the user's aforementioned body parts.

In this case, the control methods for increasing humidity include: 1 using a medically proven expert system, 2 using statistical data from an unspecified large number of people, and 3 controlling based on specific user's individual sleep data.

If awakening during sleep is detected, the humidity of the humidifier/dehumidifier 800-1 can be set to allow the user to quickly re-enter sleep.

In this case, the control methods for setting the operation include: 1 using a medically proven expert system, 2 using statistical data from an unspecified large number of people, and 3 controlling based on specific user's individual sleep data.

If REM sleep occurrence is detected around the alarm time, the humidifier/dehumidifier 800-1 can prepare the user for waking. It can be set to a humidity level that feels refreshing in the morning.

In this case, the control methods for setting the operation include: 1 using a medically proven expert system, 2 using statistical data from an unspecified large number of people, and 3 controlling based on specific user's individual sleep data.

When waking is detected, the humidifier/dehumidifier 800-1 can terminate the automatic control mode and revert to the normal mode prior to the automatic control mode.

In this case, the control methods for termination or reversion of the operation include: 1 using a medically proven expert system, 2 using statistical data from an unspecified large number of people, and 3 controlling based on specific user's individual sleep data.

### Smart Speaker

FIG. 57 is a table illustrating the operation of the smart speaker and air purifier among the main smart home-appliances according to an embodiment of the present invention.

When lying on the bed is detected, the smart speaker plays sleep-inducing sounds or sleep onset content. It can play the content that resulted in the shortest sleep onset time for the user based on the user's past personal sleep records, or the user can select sleep-inducing sounds or sleep onset content by referring to their past records.

In this case, the control methods for the play or selection operation include: 1 using a medically proven expert system, 2 using statistical data from an unspecified large number of people, and 3 controlling based on the specific user's individual sleep data.

When sleep onset is detected, the selected sleep-inducing sounds or sleep onset content can continue to operate to be maintained if lying on the bed is detected.

In this case, the control methods for maintaining the operation include: 1 using a medically proven expert system, 2 using statistical data from an unspecified large number of people, and 3 controlling based on the specific user's individual sleep data.

When deep sleep entry is detected, the smart speaker can consider white noise and monaural beats for brainwave synchronization, especially if the sleep-inducing sounds or sleep onset content are sound-based, taking into account the masking effect.

In this case, the control methods for considering the operation include: 1 using a medically proven expert system, 2 using statistical data from an unspecified large number of people, and 3 controlling based on the specific user's individual sleep data.

When the occurrence of sleep apnea is detected, the smart speaker can maintain the operation of the previous stage.

In this case, the control methods for maintaining the operation include: 1 using a medically proven expert system, 2 using statistical data from an unspecified large number of people, and 3 controlling based on the specific user's individual sleep data.

When awakening is detected during sleep, the smart speaker plays sleep-inducing content that can quickly help the user fall back asleep. In this case, it is preferable to use a form without voice or in the form of a soundscape.

In this scenario, the control methods for the playback operation include: 1 using a medically proven expert system, 2 using statistical data from an unspecified large number of people, and 3 controlling based on specific user's individual sleep data.

If the occurrence of REM sleep is detected around the alarm time, the smart speaker can provide a wake-up alarm sound to induce the user's natural awakening.

In this scenario, the control methods for the providing operation include: 1 using a medically proven expert system, 2 using statistical data from an unspecified large number of people, and 3 controlling based on specific user's individual sleep data.

When awakening is detected, the smart speaker can provide information about the previous night's sleep to the user through voice.

In this scenario, the control methods for the providing operation include: 1 using a medically proven expert system, 2 using statistical data from an unspecified large number of people, and 3 controlling based on specific user's individual sleep data.

### Air Purifier

FIG. 57 is a table illustrating the operation of the smart speaker and air purifier among the main smart home-appliances according to an embodiment of the present invention.

Since the air purifier typically operates for an average of 6 to 8 hours out of 24 hours a day, when entry into the bedroom is detected, air quality improvement such as the removal of fine dust/harmful gases can be preset to ensure the user's sound sleep.

When lying on the bed is detected, the air purifier lowers the brightness of the LED to aid the user's sleep. It reduces noise and airflow generated during operation. Additionally, since it switches to sleep mode, it is also possible to appropriately utilize the operational noise as white noise, similar to sleep-inducing sounds from the smart speaker.

In this case, the control methods for adjusting illumination, noise, and airflow include: 1 using a medically proven expert system, 2 utilizing statistical data from an unspecified large number of individuals, and 3 controlling based on the specific user's individual sleep data.

When sleep onset is detected, the air purifier may maintain the operation of the previous stage.

In this case, the control methods for maintaining the operation include: 1 using a medically proven expert system, 2 utilizing statistical data from an unspecified large number of individuals, and 3 controlling based on the specific user's individual sleep data.

When deep sleep entry is detected, since the user has already entered deep sleep, the air purifier can increase the airflow to enable rapid purification.

In this case, the control methods for adjusting airflow include: 1 using a medically proven expert system, 2 utilizing statistical data from an unspecified large number of individuals, and 3 controlling based on the specific user's individual sleep data.

When the occurrence of sleep apnea is detected, the air purifier may maintain the operation of the previous stage.

In this case, the control methods for maintaining the operation include: 1 using a medically proven expert system, 2 utilizing statistical data from an unspecified large number of individuals, and 3 controlling based on the specific user's individual sleep data.

When awakening during sleep is detected, as the user needs to re-enter sleep, the air purifier can adjust the operational noise to assist the user in falling back asleep, similar to when lying on the bed is detected.

In this case, the control methods for adjusting noise include: 1 using a medically proven expert system, 2 utilizing statistical data from an unspecified large number of individuals, and 3 controlling based on the specific user's individual sleep data.

When the occurrence of REM sleep around the alarm time is detected, the air purifier may maintain the operation of the previous stage.

In this case, the control method for maintaining the operation includes: 1 a method using a medically proven expert system, 2 a method using statistical data from an unspecified large number of people, and 3 a method based on the individual sleep data of a specified user.

If the air purifier is a table-type air purifier, upon detecting awakening, the color of the mood light can be changed according to the quality of the user's sleep upon awakening.

In this case, the control method for the color change operation includes: 1 a method using a medically proven expert system, 2 a method using statistical data from an unspecified large number of people, and 3 a method based on the individual sleep data of a specified user.

### Smart TV

FIGS. 27 to 28 are diagrams for explaining the timing of the sleep mode operation shown in FIG. 26.

When lying on the bed is detected, the smart TV can provide a service that displays statistics of the user's recent sleep quality before sleep and the target sleep for the day based on that.

In this case, the control method for the service operation includes: 1 a method using a medically proven expert system, 2 a method using statistical data from an unspecified large number of people, and 3 a method based on the individual sleep data of a specified user.

When falling asleep is detected, the smart TV screen can automatically turn off.

In this case, the control method for the turn-off operation includes: 1 a method using a medically proven expert system, 2 a method using statistical data from an unspecified large number of people, and 3 a method based on the individual sleep data of a specified user.

It is possible to maintain the operation when deep sleep entry is detected, when sleep apnea occurrence is detected, and when falling asleep is detected.

In this case, the control method for maintaining the operation includes: 1 utilizing a medically proven expert system, 2 using statistical data from an unspecified large number of people, and 3 controlling based on the specific user's individual sleep data.

When awakening during sleep is detected, it necessitates the user's rapid re-entry into sleep; in this case, the smart TV can provide re-sleep content to help the user quickly fall back asleep.

In this case, the control method for the content provision operation includes: 1 utilizing a medically proven expert system, 2 using statistical data from an unspecified large number of people, and 3 controlling based on the specific user's individual sleep data.

If the occurrence of REM sleep is detected around the alarm time, the smart TV can provide an awakening alarm sound to induce the user's natural awakening.

In this case, the control method for the sound provision operation includes: 1 utilizing a medically proven expert system, 2 using statistical data from an unspecified large number of people, and 3 controlling based on the specific user's individual sleep data.

When awakening is detected, the smart TV can display a sleep report on the screen at the user's awakening time.

In this case, the control method for the display operation includes: 1 utilizing a medically proven expert system, 2 using statistical data from an unspecified large number of people, and 3 controlling based on the specific user's individual sleep data.

Meanwhile, the smart TV may include means for controlling the smart TV, such as a processor, through which the smart TV can be controlled.

For example, the means for controlling the smart TV can perform any one of the following according to the generated control information: turning the smart TV on, turning the smart TV off, entering sleep mode, exiting sleep mode, turning the display on, turning the display off, display output, changing display mode, reducing display blue light, display dimming, rolling up the display, rolling down the display, changing display position, turning the lighting on, turning the lighting off, changing lighting mode, changing lighting orientation angle, changing lighting position, changing lighting color, changing lighting color temperature, increasing lighting brightness, decreasing lighting brightness, reducing lighting blue light, lighting dimming, lighting flashing, speaker output, increasing speaker volume, decreasing speaker volume, turning speaker sound on, turning speaker sound off, and changing speaker sound mode.

Additionally, the means for controlling the smart TV can output guided-imagery information to at least one of the display and speaker of the smart TV according to the generated control information, to create a sleep environment for the user, such as inducing sleep onset, deep sleep, or awakening.

Additionally, the means for controlling the smart TV can generate control information to control at least one of the power, display, speaker, microphone, and actuator of the smart TV to create the user's sleep environment.

Furthermore, the means for controlling the smart TV can perform algorithmic operations to create the user's sleep environment based on sleep state information and generate smart TV control information based on the results of the performed algorithmic operations.

Additionally, the generated control information may be control information generated after the user's sleep stage included in the sleep state information has remained in the wake stage for more than a threshold time.

Moreover, the generated control information may be control information for outputting the sleep state information to at least one of the display and speaker of the smart TV.

Additionally, the server 20 of the system associated with the smart TV is configured to include a first server that infers the user's sleep state information and a second server different from the first server. The first server can transmit the user's sleep state information inference completion event information or the inferred sleep state information to the callback URL of the second server. In this case, the second server can receive the inferred sleep state information from the first server and transmit it to the smart TV.

Meanwhile, the smart TV according to an embodiment of the present invention includes a housing; a rollable display equipped with a flexible display such as an organic light-emitting panel (OLED panel); a drive unit including at least one actuator that varies the size of the display exposed outside the housing; a user input interface unit that receives control signals from remote control devices such as a smartphone or remote control; and a control unit that controls components such as the display and speaker of the smart TV.

Here, the control unit can control at least one of the power, display, speaker, microphone, and/or actuator of the smart TV, such as by controlling the drive unit to roll up or roll down the display through at least one actuator based on the sleep state information including the user's sleep stage information inferred through the AI sleep analysis model.

### Robot Vacuum Cleaner

FIGS. 27 to 28 are diagrams for explaining the timing of the sleep mode operation shown in FIG. 26.

When sleep onset is detected, the robot vacuum cleaner verifies whether the user is falling asleep and operates in automatic cleaning mode.

In this case, the control method for the operation includes: 1 a method using a medically proven expert system, 2 a method using statistical data from an unspecified large number of people, and 3 a method based on the individual sleep data of a specified user.

When the entry into deep sleep is detected, since deep sleep poses less risk of waking the user, the degree of freedom for the cleaning area and cleaning time of the robotic cleaner can be increased.

In this case, the control method for increasing the degree of freedom includes: 1 a method using a medically proven expert system, 2 a method using statistical data from an unspecified large number of people, and 3 a method based on the individual sleep data of a specified user.

If the occurrence of sleep apnea is detected, the robotic cleaner can maintain the operation as if the entry into deep sleep had been detected.

In this case, the control method for maintaining the operation includes: 1 a method using a medically proven expert system, 2 a method using statistical data from an unspecified large number of people, and 3 a method based on the individual sleep data of a specified user.

If awakening during sleep is detected, the robotic cleaner can temporarily pause its operation even if it was in the middle of cleaning.

In this case, the control method for the temporary pause operation includes: 1 a method using a medically proven expert system, 2 a method using statistical data from an unspecified large number of people, and 3 a method based on the individual sleep data of a specified user.

If the occurrence of REM sleep around the alarm time is detected, since the probability of the user waking up soon is high, the robotic cleaner can return to the charging dock.

In this case, the control method for the return operation includes: 1 a method using a medically proven expert system, 2 a method using statistical data from an unspecified large number of people, and 3 a method based on the individual sleep data of a specified user.

If awakening is detected, the robotic cleaner can maintain the operation as if the occurrence of REM sleep around the alarm time had been detected.

In this case, the control methods for maintaining the operation include: 1 using a medically proven expert system, 2 using statistical data from an unspecified large number of people, and 3 controlling based on the individual sleep data of a specified user.

### Other Smart Home-Appliances

Referring to FIG. 53, the operation of the other smart home-appliances among one or more smart home-appliances 800 operating in a time-series manner using the method of providing environment adjustment information of the present invention will be described for each case where the user's sleep behavior transition occurs as follows.

When lying on the bed is detected, the blinds/curtains/lighting are set so that the brightness in the bedroom gradually dims. The lighting is set to gradually dim and then turn off, and the operation time of the blinds/curtains and the maintenance time of the lighting can be set according to the user's average sleep onset time based on the user's past individual sleep records.

In this case, the control methods for each setting operation include: 1 using a medically proven expert system, 2 using statistical data from an unspecified large number of people, and 3 controlling based on the individual sleep data of a specified user.

When sleep onset is detected, the washing machine and dryer do not operate until the user enters deep sleep, and they can start operating from the point when the user's deep sleep is detected.

In this case, the control methods for initiating operation include: 1 using a medically proven expert system, 2 using statistical data from an unspecified large number of people, and 3 controlling based on the individual sleep data of a specified user.

Meanwhile, since sleep apnea occurs most frequently when the user is lying in a supine position on the bed, if the occurrence of the user's apnea is detected, it can operate to change the posture of the smart bed user (e.g., head and body position) when the occurrence of sleep apnea is detected.

In this case, the control methods for operation include: 1 using a medically proven expert system, 2 using statistical data from an unspecified large number of people, and 3 controlling based on the individual sleep data of a specified user.

If awakening during sleep is detected, the washing machine and dryer can temporarily pause operation to block vibration and noise. At this time, events during the user's sleep can be detected through the user's movement information and sleep sound information via a smartphone.

In this case, the control method for the pause operation includes: 1 using a medically proven expert system, 2 using statistical data from an unspecified large number of people, and 3 controlling based on the individual sleep data of a specified user.

If the occurrence of REM sleep is detected around the alarm time, the blinds/curtains/lights can induce the user's natural awakening through a dawn simulation operation.

Additionally, the smart bed is inclined to allow the user to wake up naturally around the time of awakening.

In this case, the control method for the awakening induction operation and incline operation includes: 1 using a medically proven expert system, 2 using statistical data from an unspecified large number of people, and 3 controlling based on the individual sleep data of a specified user.

Furthermore, the washing machine operates to complete washing in time with the detection of REM sleep occurrence, and the dryer can operate to dry immediately in the morning.

In this case, the control method for the operation includes: 1 using a medically proven expert system, 2 using statistical data from an unspecified large number of people, and 3 controlling based on the individual sleep data of a specified user.

When awakening is detected, the clothing manager can perform an operation to diffuse different scents according to the quality of the user's sleep after awakening. It can provide the user's sleep report on the display unit. It can be set to different notification melodies according to the quality of the sleep.

Additionally, the smart diffuser can perform an operation to diffuse different scents according to the quality of the user's sleep after awakening.

Furthermore, the water purifier can supply a glass of water to the user in the morning and provide guidance on the sleep report via voice. It can supply cold water, purified water, or hot water according to the sleep state of the previous night.

Additionally, the refrigerator can provide a sleep report through the display unit. If it is a mood-up refrigerator, it can offer a service to change the color of the refrigerator according to the quality of sleep.

In this case, the control methods for direction, water supply, report provision, and color change operations include: 1 using a medically proven expert system, 2 using statistical data from an unspecified large number of people, and 3 controlling based on the individual sleep data of a specified user.

### Smart Watch

FIG. 59 is a flowchart illustrating the operation of a smart watch within the system shown in FIG. 53 according to another embodiment of the present invention. FIG. 60 is a table showing the operation of the smart watch according to another embodiment of the present invention.

As shown in FIG. 59, overall, the method of providing environment adjustment information according to another embodiment of the present invention includes: a step S310 in which the smart watch acquires sleep sound information related to the user's sleep in real-time through a microphone module; a step S320 in which the user terminal receives the acquired sleep sound information, converts it into a spectrogram, and performs analysis to determine the user's sleep stage in real-time S330; and a step S340 in which the user terminal outputs a control signal to control the operation of the smart watch in real-time according to events occurring in each determined sleep stage.

At this time, the step of outputting the control signal may include a step S350 in which the smart watch responds to the control signal to provide the user with an optimal sleep environment.

As in one embodiment of the present invention, in FIG. 60, it can be assumed that the blank spaces represent stages where appropriate actions are taken without a transition of operation when the immediately preceding case is detected.

First, when lying on the bed is detected, the smart watch can provide services for falling asleep, such as a breathing guide based on the user's sleep time and a meditation guide, through vibration.

In this case, the control methods for the service provision operation include: 1 using a medically proven expert system, 2 using statistical data from an unspecified large number of people, and 3 controlling based on the individual sleep data of a specified user.

When falling asleep is detected, the smart watch can weaken the intensity of the vibration as the confidence of detecting the user's falling asleep increases when the user enters the bedroom and lies on the bed, or adjust the vibration duration to match the average individual falling asleep time.

In this case, the control methods for the vibration adjustment operation include: 1 using a medically proven expert system, 2 using statistical data from an unspecified large number of people, and 3 controlling based on the individual sleep data of a specified user.

When the entry into deep sleep is detected, the operation detected during the onset of sleep can be maintained.

In this case, the control methods for maintaining the operation include: 1 using a medically proven expert system, 2 using statistical data from an unspecified large number of people, and 3 controlling based on the specific user's individual sleep data.

If the occurrence of sleep apnea is detected, the smart watch can provide a gentle vibration to the user when sleep apnea is detected or predicted to occur, thereby awakening the user to prevent sleep apnea.

In this case, the control methods for the vibration-providing operation include: 1 using a medically proven expert system, 2 using statistical data from an unspecified large number of people, and 3 controlling based on the specific user's individual sleep data.

If awakening during sleep is detected, the smart watch can maintain the operation detected in the case of sleep apnea occurrence.

In this case, the control methods for maintaining the operation include: 1 using a medically proven expert system, 2 using statistical data from an unspecified large number of people, and 3 controlling based on the specific user's individual sleep data.

If the occurrence of REM sleep is detected around the alarm time, the smart watch can provide a wake-up alarm through vibration or provide an alarm at a time when the user is more likely to wake up, based on the user's past individual sleep records.

In this case, the control methods for the alarm-providing operation include: 1 using a medically proven expert system, 2 using statistical data from an unspecified large number of people, and 3 controlling based on the specific user's individual sleep data.

When awakening is detected, the smart watch can provide a predetermined sleep report through the screen as soon as the user wakes up.

In this case, the control methods for providing the sleep report operation include: 1 using a medically proven expert system, 2 using statistical data from an unspecified large number of people, and 3 controlling based on the specific user's individual sleep data.

Meanwhile, the present invention displays 0 weight/blood pressure and sleep apnea, insomnia, @exercise and insomnia as sleep measurement records, which can motivate users to change their behavior to improve their health. That is, the present invention can very naturally enhance the compliance of user behavior changes.

For example, if a user is overweight, sleep apnea frequently occurs, and since weight loss can help improve sleep apnea, the present invention can be linked with diet, exercise, and weight tracking in a healthcare app.

In other words, the history of sleep apnea allows for behavioral intervention with real-time sleep apnea detection and accuracy provided by the present invention.

Additionally, since sleep apnea can cause hypertension, if the periods of respiratory instability are regular, the present invention enables blood pressure tracking and management.

That is, since weight loss helps lower blood pressure in the human body, upon successful weight loss, the PSQI can be used to objectively compare the quality of sleep before and after.

Furthermore, exercise (excluding within 3 hours before bedtime) helps alleviate insomnia, and extended exposure to natural light through outdoor activities can improve the user's mood.

Additionally, it becomes possible to receive recommendations for various exercise programs from a healthcare app.

Moreover, the present invention can show users the correlation between \textcircled{1} stress levels and sleep, \textcircled{2} premenstrual syndrome and insomnia, which can lead users to re-recognize their health status.

That is, by indicating the correlation between stress levels and sleep quality, an element of interest is added, and depending on the user's stress levels and degree of depression, it becomes possible to fill out psychiatric-related questionnaires provided by the healthcare app.

Additionally, in cases where insomnia is complained of as one of the symptoms of premenstrual syndrome, the present invention allows for the comparison of sleep data within the calendar of the menstrual cycle tracking function, enabling users to check their health status related to physiological phenomena by displaying sleep efficiency.

Meanwhile, one of the important aspects in sleep stage analysis is determining whether a user experiences awakenings during sleep and whether genuine awakening occurs. Specifically, it is crucial to accurately analyze the WAKE stage, and sleep sound information can be a very useful factor in detecting whether the user is in a genuine WAKE stage.

In conventional polysomnography, the measurement of brain waves merely confirmed changes in brain waves when the user was awake. However, the sleep sound information used in the sleep stage analysis of the present invention indicates precursor signals (such as sound patterns and movement patterns) before the user wakes up (before reaching the WAKE stage), allowing for the prediction and detection of the WAKE stage.

According to the AI sleep stage analysis model trained with a large amount of data, the determination of the WAKE stage, particularly based on sleep sound information, becomes more precise. Additionally, while a user may awaken due to their body's biorhythm, they may also be influenced by external factors such as ambient noise or disturbances.

The present invention enables the AI sleep stage analysis model to be constructed by learning various ambient noises, including routine noises in the surrounding space and abnormal or intermittent noises, thereby allowing for a clearer and more reliable prediction and detection of the WAKE stage.

### Overall Configuration

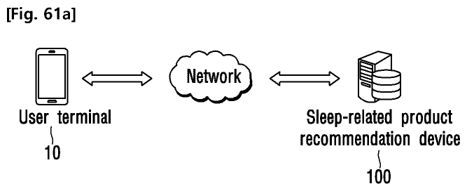

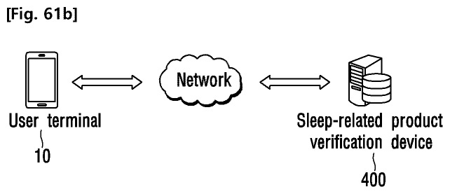

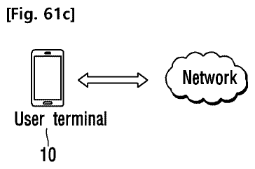

FIG. 61a illustrates a conceptual diagram of a sleep-related product recommendation system according to the present invention. FIG. 61b illustrates a conceptual diagram of a sleep-related product verification system according to the present invention. FIG. 61c illustrates a conceptual diagram of a sleep-related product recommendation/verification system according to another embodiment of the present invention.

FIG. 61a shows a conceptual diagram of a system in which various aspects of a sleep-related product recommendation device 40 can be implemented based on sleep state information related to an embodiment of the present invention. The system according to embodiments of the present invention may include a sleep-related product recommendation device 40, a user terminal 10, and a network.

Meanwhile, FIG. 61b shows a conceptual diagram of a system in which various aspects of a sleep-related product verification device 50 can be implemented according to another embodiment of the present invention. The system according to embodiments of the present invention may include a sleep-related product verification device 50, a user terminal 10, and a network.

Meanwhile, FIG. 61c illustrates a conceptual diagram of a system where the recommendation and/or verification of sleep-related products is implemented on a user terminal 10 according to another embodiment of the present invention. As shown in FIG. 61c, the recommendation and verification of sleep-related products may be performed on the user terminal 10 without a separate recommending device 40 and/or verifying device 50.

Additionally, FIG. 61d illustrates a conceptual diagram of a system in which various aspects of various electronic devices related to another embodiment of the present invention can be implemented.

The electronic devices depicted in FIG. 61d are capable of performing at least one of the operations executed by various devices according to embodiments of the present invention.

For instance, the operations performed by various electronic devices according to embodiments of the present invention may include acquiring environment sensing information, conducting learning for sleep analysis, performing inference for sleep analysis, and acquiring sleep state information.

Alternatively, for example, the operations may include receiving information related to a user's sleep, transmitting or receiving environment sensing information, determining environment sensing information, extracting sound information from environment sensing information, processing or handling data, processing or providing services, constructing a learning dataset based on environment sensing information or information related to a user's sleep, storing acquired data or multiple data inputs to a neural network, transmitting or receiving various information, and mutually transmitting and receiving data for systems according to embodiments of the present invention through a network.

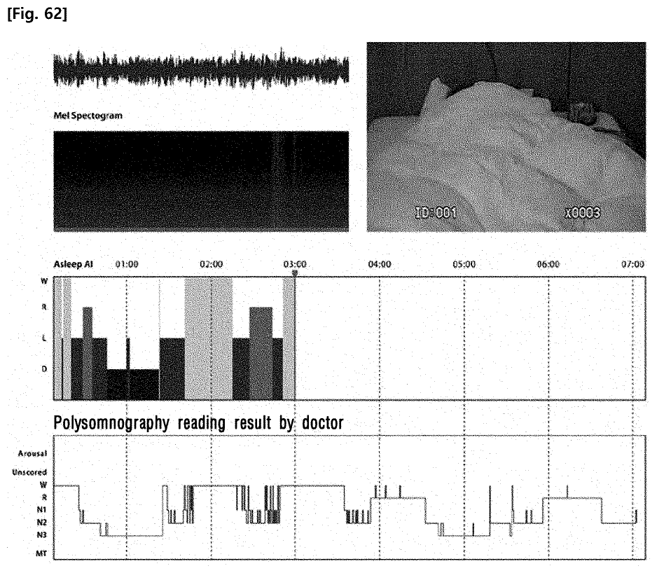

FIG. 62 illustrates an experimental process for verifying the performance of the sleep analysis method according to the present invention.

As shown in FIG. 62, the user's sleep video and sleep sound are acquired in real-time, and the acquired sleep sound information is immediately converted into a spectrogram. During this process, pre-processing of the sleep sound information may be performed. The spectrogram is input into the sleep analysis model, allowing for immediate analysis of the sleep stage.

Additionally, in one embodiment of the present invention, the operation of employing a CNN or Transformer-based deep learning model in the feature classification model may be performed as follows.

According to one embodiment of the present invention, a spectrogram containing time-series information is input into a CNN-based deep learning model, which can output a lower-dimensional vector. This lower-dimensional vector is then input into a Transformer-based deep learning model, which can output a vector encapsulating time-series information.

In one embodiment of the present invention, the output vector from the Transformer-based deep learning model may be input into a 1D CNN 1D Convolutional Neural Network) to apply an average pooling technique, thereby performing a process of converting it into an N-dimensional vector encapsulating time-series information through averaging operations on the time-series information. In this case, the N-dimensional vector encapsulating time-series information corresponds to data that still includes time-series information, albeit with a resolution difference from the input data.

According to one embodiment of the present invention, a multi-epoch classification on the combination of N-dimensional vectors encapsulating time-series information can be performed to predict various sleep stages. In this case, the output vectors from the Transformer-based deep learning models may be used as inputs to multiple FC (Fully Connected) layers to perform predictions of continuous sleep state information.

Furthermore, in one embodiment of the present invention, the operation of employing a ViT or Mobile ViT-based deep learning model in the feature classification model may be performed as follows.

According to an embodiment of the present invention, a spectrogram containing time-series information can be used as input to a Mobile ViT-based deep learning model, which can output a vector with reduced dimensionality.

Additionally, in accordance with an embodiment of the present invention, features can be extracted from each spectrogram as output from the Mobile ViT-based deep learning model.

According to an embodiment of the present invention, by using the vector with reduced dimensionality as input to an Intermediate Layer, a vector encapsulating time-series information can be output. The Intermediate Layer model may include at least one of the following stages: a linearization stage for encapsulating the vector's information, a layer normalization stage for inputting mean and variance, or a dropout stage for deactivating some nodes.

By performing the process of outputting a vector encapsulating time-series information using the vector with reduced dimensionality as input to the Intermediate Layer, overfitting can be prevented according to an embodiment of the present invention.

According to an embodiment of the present invention, by using the output vector from the Intermediate Layer as input to a ViT-based deep learning model, sleep state information can be output. In this case, sleep state information corresponding to information on the frequency domain containing time-series information, a spectrogram, or a mel spectrogram can be output.

Furthermore, according to an embodiment of the present invention, sleep state information corresponding to a series of configurations of information on the frequency domain containing time-series information, a spectrogram, or a mel spectrogram can be output.

Meanwhile, the feature extraction model or feature classification model according to an embodiment of the present invention may employ various deep learning models in addition to the aforementioned AI models to perform learning or inference, and the specific description related to the types of the aforementioned deep learning models is merely exemplary and not limiting to the present invention.

### Performance of the Sleep Analysis Method According to the Present Invention

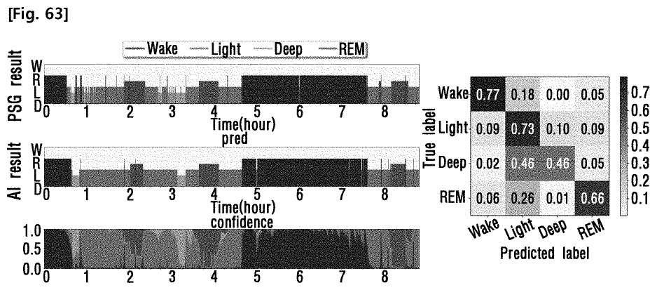

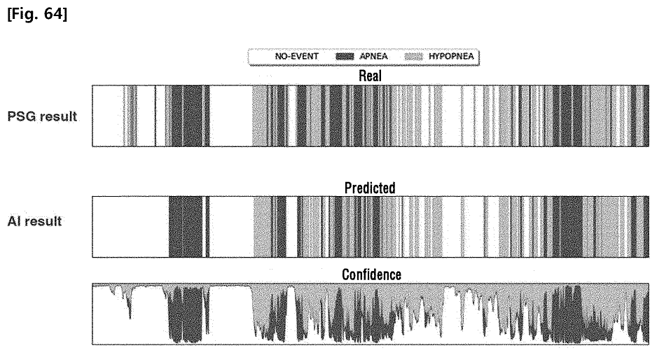

FIG. 62 illustrates the experimental process for verifying the performance of the sleep analysis method according to the present invention. FIG. 63 is a graph verifying the performance of the sleep analysis method according to the present invention, comparing the polysomnography (PSG) results with the analysis results using the AI algorithm according to the present invention.

As illustrated in FIG. 63, the sleep analysis results obtained according to the present invention not only closely align with polysomnography but also include more precise and meaningful information related to sleep stages (Wake, Light, Deep, REM).

The hypnogram depicted at the bottom of FIG. 62 represents the probability of belonging to one of the four classes (Wake, Light, Deep, REM) every 30 seconds when predicting sleep stages based on user sleep sound information. Here, the four classes respectively signify the states of being awake, lightly asleep, deeply asleep, and in REM sleep.

FIG. 64 is a graph verifying the performance of the sleep analysis method according to the present invention, comparing the results of polysomnography (PSG result) with the analysis results using the AI algorithm of the present invention (AI result) concerning sleep apnea and hypopnea.

The hypnogram shown at the bottom of FIG. 64 indicates the probability of belonging to one of the two conditions (sleep apnea, hypopnea) every 30 seconds when predicting sleep disorders based on user sleep sound information. Utilizing the sleep analysis of the present invention, as shown in FIG. 64, the sleep state information obtained according to the present invention not only closely aligns with polysomnography but also includes more detailed analysis information related to apnea and hypopnea.

The present invention can identify the points at which sleep disorders (sleep apnea, sleep hypopnea, sleep hyperpnea) occur while analyzing the user's sleep in real-time. Providing stimuli (tactile, auditory, olfactory, etc.) to the user at the moment a sleep disorder occurs can temporarily alleviate the disorder. That is, the present invention can interrupt the user's sleep disorder and reduce its frequency based on accurate event detection related to sleep disorders.

### Sleep-Related Products

Sleep-related products according to embodiments of the present invention may include items such as mattress covers, pads, mattresses, yoga mattresses, latex mattresses, mattress coolers, mattress covers, bed skirts, flat sheets, comforters, quilts, blankets, electric blankets, weighted blankets, duvet covers, pillows, memory foam pillows, smart pillows, vibrating pillows, body pillows, pillow covers, pajamas, underwear, waist belts, leggings, sleep robes, sleep socks, sleep caps, eye masks, earplugs, noise-canceling earplugs, earphones, slippers, bedroom decorations, bed trays, bed frames, bed canopies, bed headboards, under-bed storage, negative pressure bedroom systems, stress balls, toys, dolls, tents, baby tents, travel sleep kits, curtains, smart blinds, bedside tables, snack pads, sleep lenses, accessories, and nasal hair removal strips.

Additionally, sleep-related products according to embodiments of the present invention may include items such as aromatherapy, diffusers, therapy diffusers, sleep sprays, sprays for dry mouth, pillow sprays, pillow mists, air sprays, essential oils, candles, perfumes, cosmetics, bathroom products (cleansing products), laundry products, fabric softeners, fabric softener sheets, lotions, skin care products, sleep packs, and mask packs.

Furthermore, sleep-related products according to embodiments of the present invention may include consumables such as nutritional supplements, general foods, health functional foods, special nutritional foods, foods for special medical purposes, pharmaceuticals, sleep aids, food additives, beverages, tea, decaffeinated beverages, and sleepy time tea.

Moreover, sleep-related products according to embodiments of the present invention may include items such as tableware, interior building materials, bathtubs, building components, soundproof panels, electronic devices, temperature control devices, alarm clocks, ion generators, humidifiers, dehumidifiers, air purifiers, air conditioners, exercise equipment, yoga balls, nap chairs, home appliances, mobile devices, wearable devices, lighting devices, audio devices, music CDs, music players, smart plugs controlling bedside electronic devices, white noise devices, pet products, beam projectors, ceiling star projectors, positive airway pressure devices, sleep disorder treatment devices, and night medication dispensers.

Additionally, sleep-related products according to embodiments of the present invention may include applications for measuring sleep, such as sleep trackers. Alternatively, they may include applications or services for sleep counseling, items providing information to alleviate sleep disorders, or products assisting a child's sleep.

Furthermore, sleep-related products according to embodiments of the present invention may include guided-imagery information comprising at least one of text information, audio information, or visual information, or devices/applications for providing guided-imagery information.

### Sleep-Related Product Verification Device and Method

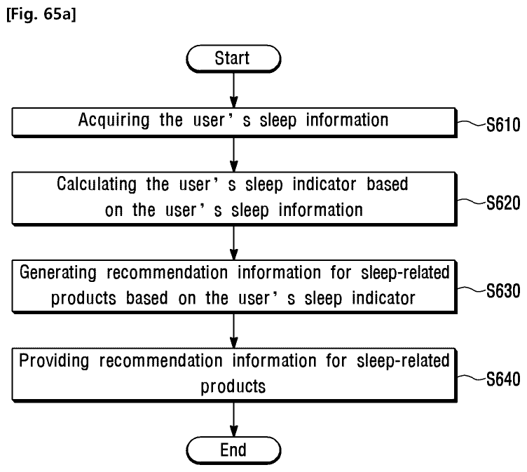

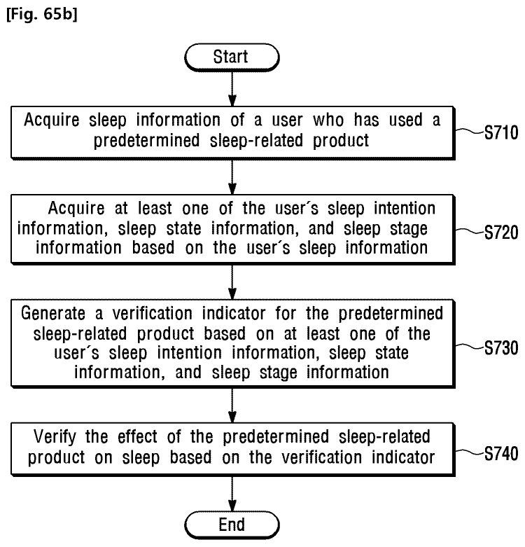

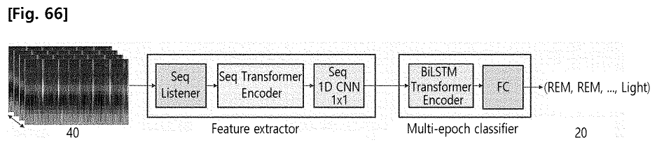

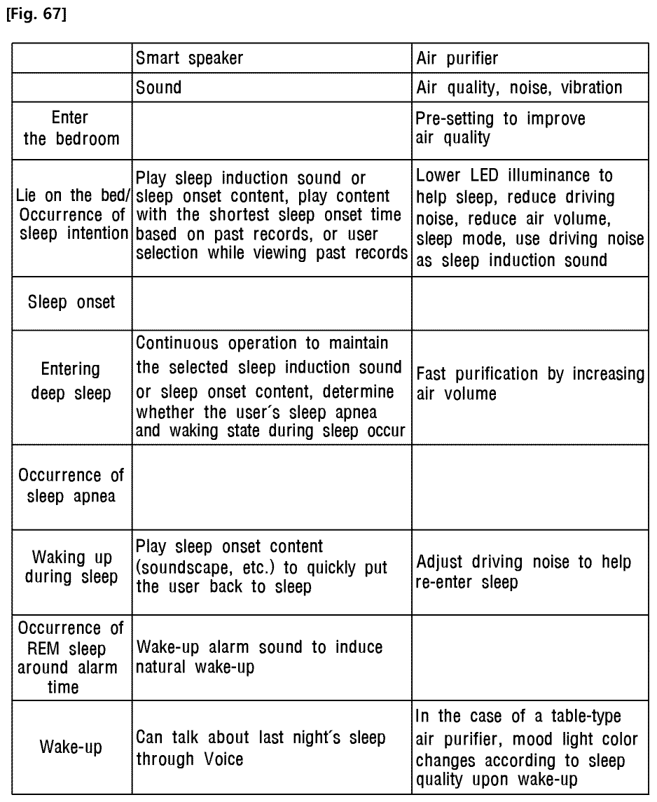

FIG. 65(b) is a flowchart illustrating a method for verifying sleep-related products according to the present invention.

According to the sleep-related product verification method of the present invention, it is possible to verify the impact of the sleep-related product under verification on sleep quality. Specifically, by analyzing the sleep information of a user who has used the sleep-related product and fallen asleep, it can be objectively confirmed and analyzed whether the user's sleep latency has decreased, whether sleep disorders have improved, and whether stable and deep sleep has been achieved. Here, when using various sleep-related products according to embodiments of the present invention, it is assumed that the products can be used not only before falling asleep but also during sleep, at the time of waking, or after waking, depending on the type of product.

The sleep-related product verification method according to the present invention includes, as shown in FIG. 65(b), the steps of acquiring the sleep information of a user who has used a predetermined sleep-related product (S710), acquiring at least one of the user's sleep intention information, sleep state information, and sleep stage information based on the user's sleep information (S720), generating a verification indicator for the predetermined sleep-related product based on at least one of the user's sleep intention information, sleep state information, and sleep stage information (S730), and verifying the impact of the predetermined sleep-related product on sleep based on the verification indicator (S740).

Here, in the step of acquiring the user's sleep information (S710), the user's sleep information can be acquired from one or more sensor devices.

Additionally, the step of acquiring at least one of the user's sleep intention information, sleep state information, and sleep stage information based on the user's sleep information (S720) may include generating an inference model trained with sleep information as input, and extracting the sleep state information as an output by inputting the user's sleep information into the inference model.

First, the user uses a predetermined sleep-related product before falling asleep. The predetermined sleep-related product becomes the subject of sleep impact verification. According to one embodiment of the present invention, to generate an indicator for verifying the sleep-related product, the user may be arbitrarily recommended or suggested to use the sleep-related product.

The user uses the recommended or suggested sleep-related product, and accordingly, a verification indicator may be generated.

Sleep information or environment sensing information related to the user using the sleep-related product is received from the user terminal. The sleep information or environment sensing information has been described in detail above.

Subsequently, based on the environment sensing information, the user's sleep state information and sleep stage information are acquired. The sleep state information includes information related to whether the user is sleeping and may include at least one of the first sleep state information indicating the user is before sleep, the second sleep state information indicating the user is during sleep, and the third sleep state information indicating the user is after sleep. This sleep state information is acquired based on the environment sensing information, which is obtained in a non-contact manner by the user terminal. The sleep stage information can also be extracted based on the environment sensing information, and the sleep stages can be classified into NREM (non-REM) sleep and REM (Rapid Eye Movement) sleep. NREM sleep can further be divided into multiple stages (e.g., two stages of Light and Deep, or four stages from N1 to N4, but is not limited thereto. Through sleep stage analysis, not only the quality of sleep related to sleep but also sleep disorders (e.g., sleep apnea) and their underlying causes (e.g., snoring) can be predicted.

Subsequently, using at least one of the sleep intention information, sleep state information, and sleep stage information, a verification indicator for the predetermined sleep-related products can be generated. The verification indicator for the predetermined sleep-related products according to embodiments of the present invention may be a lookup table or a score that numerically records the analysis of sleep.

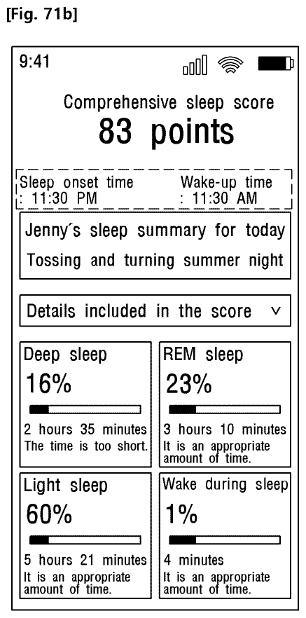

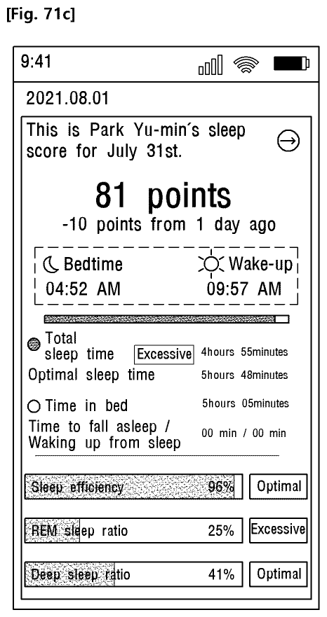

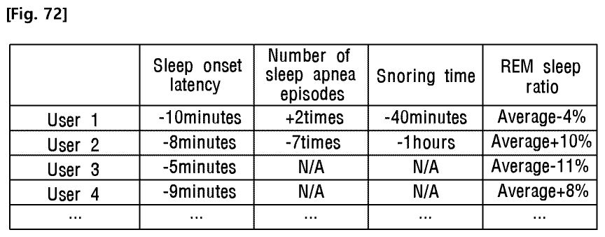

FIG. 71a is a table for explaining a case where the verification indicator for sleep-related products is displayed as a score. FIGS. 71b and 71c are exemplary diagrams for explaining a case where the verification indicator for sleep-related products is displayed as a numerical evaluation of the user's sleep according to an embodiment of the present invention.

According to embodiments of the present invention, when the verification indicator is a score, it may be displayed as shown in the table of FIG. 71a, or provided through a graphical user interface including numerical sleep evaluation as shown in FIGS. 71b and 71c. However, the specific numbers, table forms, or display methods of the graphical user interface shown in FIGS. 71a to 71c are merely simple examples for explaining the present invention, and the present invention is not limited thereto.

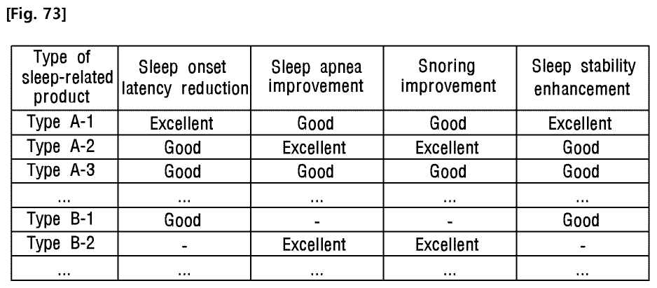

Alternatively, as shown in the table of FIG. 73, a verification indicator for sleep-related products can be generated as a lookup table, and by assigning weights or scores such as 5 points for Excellent, 4 points for Good, etc., a numerical indicator can be calculated based on this lookup table. The specific numbers for the aforementioned scores are merely examples and are not limited thereto.

Hereinafter, a case where the verification indicator is a score that numerically records the analysis of sleep according to an embodiment of the present invention will be described in detail.

According to an embodiment of the present invention, a sleep score, which is a type of verification indicator, can be calculated based on at least one of the user's sleep onset latency, sleep onset time, wake-up time, total sleep time, and sleep time per sleep stage when using the predetermined sleep-related products. The user's total sleep time may be the absolute value of the difference between the bedtime and wake-up time. The sleep time per sleep stage of the user may be represented as a ratio or time of the measured user's sleep stages (e.g., awake, deep sleep, light sleep, REM sleep, etc.).

According to an embodiment of the present invention, the sleep score may be a comprehensive score of sleep with a maximum score of 100, providing a numerical evaluation of sleep. The sleep score can be calculated by a predefined sleep score calculation formula.

For example, scores corresponding to each sleep stage (e.g., REM sleep, deep sleep, light sleep, awake, etc.) can be assigned, and by substituting them into a predefined sleep score calculation formula, a final score can be obtained. The final score divided by the maximum possible score and multiplied by 100 can be the comprehensive sleep score. By providing the sleep score as an evaluation in the form of a score, an intuitive evaluation of sleep can be provided.

According to the present invention, if the sleep score of a user who used the sleep-related products is high, it can be interpreted that the relevant sleep-related products had a positive impact on the user's sleep experience. Conversely, if the sleep score is low, it may be interpreted that the user's sleep experience was not positively impacted.

Meanwhile, according to one embodiment of the present invention, a method for generating verification indicators for sleep-related products based on a user's subjective judgment indicators may be provided. Alternatively, according to one embodiment of the present invention, verification indicators for sleep-related products may be generated based on the user's subjective judgment indicators and the aforementioned numerical indicators.

According to one embodiment of the present invention, the method for generating verification indicators for sleep-related products based on a user's subjective judgment indicators may include receiving a string value or numerical value for the user's subjective judgment indicators and generating verification indicators based on that. For example, when receiving a string value such as “excellent,” a positive verification indicator for the sleep-related product may be generated. Meanwhile, the specific description of the aforementioned string value is merely exemplary and not limited thereto.

Additionally, according to one embodiment of the present invention, when receiving a user's subjective judgment, it may be received through various input actions in addition to receiving string values or numerical values as described above.

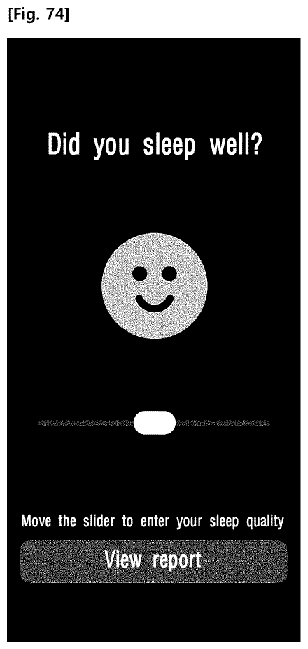

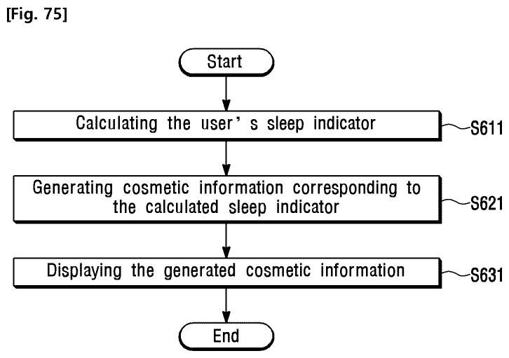

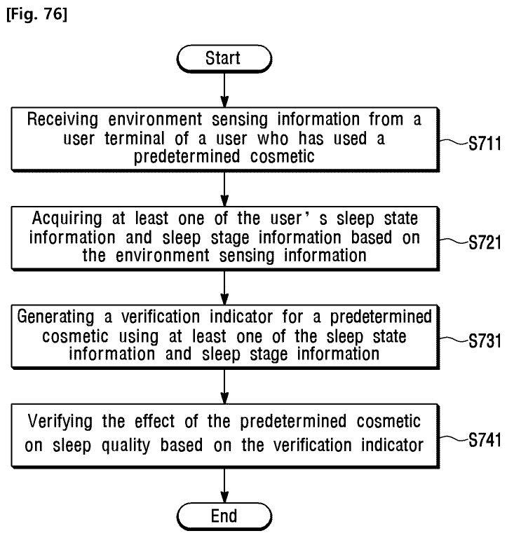

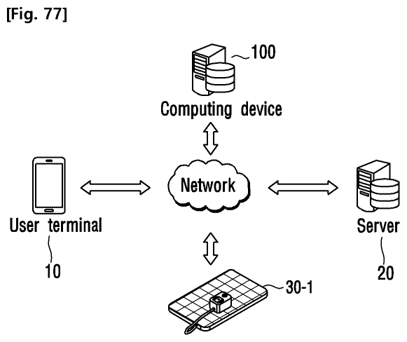

FIG. 74 is a diagram illustrating a graphical user interface for receiving a user's input actions to calculate subjective judgment indicators according to one embodiment of the present invention.

For example, as shown in FIG. 74, a user may input subjective judgment by swiping the input screen of subjective indicators displayed on the display. When a graphical user interface including a slider is displayed on the input screen as shown in FIG. 74, the user may input their subjective judgment of using and experiencing the sleep-related product by moving the slider bar left or right. Meanwhile, a graphical user interface including a slider bar that can move up and down may be provided, and the form of the slider is not limited to the form shown in FIG. 74. Various examples of user input actions may exist beyond this.

Meanwhile, the sleep-related product verification method according to the present invention may be used by multiple users for the same verification-target sleep-related product. In this case, based on the environment sensing information received from multiple users, each user's sleep state information and sleep stage information may be derived, and verification indicators for multiple users may be generated as shown in the table in FIG. 72.

Based on the verification indicators of Table 7 confirmed by multiple users, it can be confirmed that the verification-target sleep-related product is effective in reducing sleep onset latency, and therefore, it may be selected as a recommended sleep-related product for users with relatively high sleep onset latency and recommended to them.

Through the aforementioned process, sleep evaluation statistics for multiple users regarding multiple sleep-related products can be obtained, and a lookup table as shown in the table in FIG. 73 can be obtained, which can be used to generate a lookup table for recommended sleep-related products in the sleep-related product recommendation method described below.

At this time, sleep-related products may be classified by type, by target user such as female/male, or by scent such as lavender or rosemary. Of course, in another embodiment, the type of sleep-related products may be set in a manner different from the above.

### Sleep-Related Product Recommendation Device and Method

FIG. 65a is a flowchart illustrating a method for recommending sleep-related products according to the present invention.

The method for recommending sleep-related products according to the present invention includes, as shown in FIG. 65a, the steps of acquiring user sleep information 5610, calculating a user's sleep indicator based on the user sleep information 5620, generating recommendation information for sleep-related products based on the user's sleep indicator 5630, and providing the recommendation information for sleep-related products 5640.

Here, in the step of acquiring user sleep information 5610, the user sleep information can be acquired from one or more sensor devices.

The step of calculating the user's sleep indicator can be performed based on the sleep state information and sleep stage information described in detail above.

Briefly, the sleep state information includes information related to whether the user is asleep, and may include at least one of the first sleep state information indicating the user is about to fall asleep, the second sleep state information indicating the user is asleep, and the third sleep state information indicating the user is post-sleep.

This sleep state information is acquired based on environment sensing information, which is obtained in a non-contact manner by the user terminal. Sleep stage information can also be extracted based on environment sensing information, and sleep stages can be classified into NREM (non-REM) sleep and REM (Rapid Eye Movement) sleep. NREM sleep can further be divided into multiple stages (e.g., two stages of Light and Deep, or four stages from N1 to N4, but is not limited thereto. Through sleep stage analysis, not only the quality of sleep but also sleep disorders (e.g., sleep apnea) and their underlying causes (e.g., snoring) can be predicted.

According to embodiments of the present invention, recommendations for sleep-related products can be made by comparing the sleep indicator calculated based on the user's sleep state information with the verification indicators of sleep-related products described above. In this case, based on the verification indicators of various sleep-related products, a recommendation can be made for a sleep-related product that has a verification indicator deemed suitable for the user's sleep indicator.

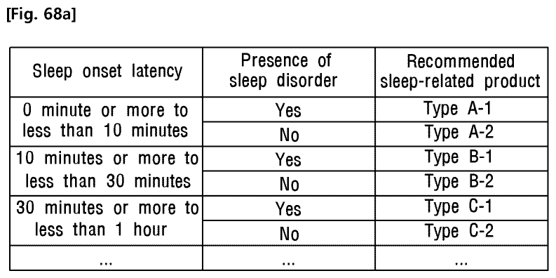

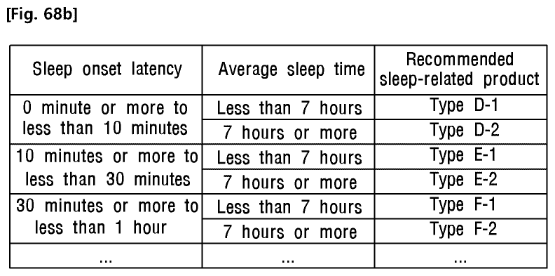

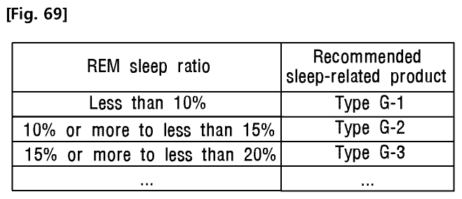

FIGS. 68 to 69 are tables for explaining a lookup table in which information on sleep-related products recommended in response to sleep indicators is recorded.

Meanwhile, the step of generating information on sleep-related products can generate recommendation information for sleep-related products based on a lookup table in which information on sleep-related products corresponding to sleep indicators is recorded. The lookup table can be configured as shown in the tables of FIGS. 17 to 19, but this is merely an example, and the lookup table can be configured in various ways.

At this time, the method for recommending sleep-related products can generate a sleep-related product recommendation model and generate information on sleep-related products based on it. The sleep-related product recommendation model can be generated by the learning method described above, and the sleep indicators input into the input layer of the learning model can be information acquired based on sleep state information and sleep stage information. Specifically, the input sleep indicators can be sleep stage information such as the first to nth sleep state information, NREM (non-REM) sleep, REM (Rapid Eye Movement), two stages of Light and Deep, or four stages from N1 to N4. Additionally, the input sleep indicators can include sleep onset latency, average sleep time, REM sleep cycle, NREM sleep pattern, presence of snoring, presence of sleep disorders, etc., which can be extracted from sleep state information and sleep stage information.

According to one embodiment of the present invention, the sleep onset latency can be extracted based on the user's intended sleep time and the actual time of falling asleep, the average sleep time can be extracted based on the user's sleep onset time and wake-up time, the REM sleep cycle and ratio can be extracted based on the sleep stage information, and the NREM sleep pattern can be extracted based on the pattern, cycle, and time of LIGHT/DEEP sleep or based on the pattern, cycle, and time of N1 to N4 sleep.

For example, if a sleep indicator indicating that the REM sleep ratio is relatively low is calculated based on the user's sleep state information, a method may be implemented to recommend a product with a verification indicator deemed helpful in increasing the REM sleep ratio among sleep-related products to the user.

Alternatively, if a sleep indicator indicating that the sleep onset latency is relatively long is calculated based on the user's sleep state information, a method may be implemented to recommend a product with a verification indicator deemed helpful in reducing the sleep onset latency among sleep-related products to the user. The description of the sleep indicators mentioned above is merely an example to aid in understanding the present invention, and the invention is not limited thereto, as various scenarios can be envisaged.

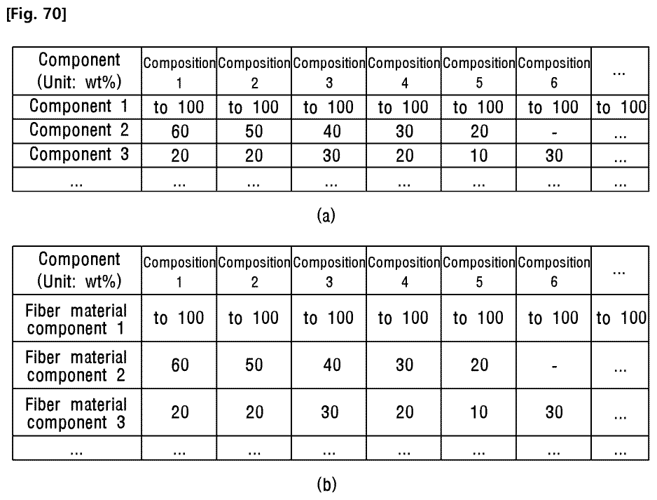

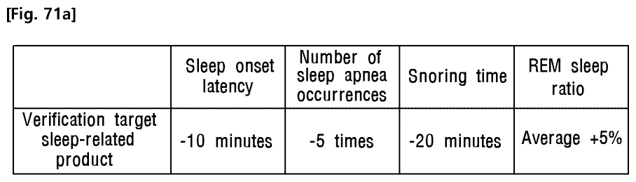

FIG. 70a is a table illustrating a lookup table in which composition information is recorded when the sleep-related product is a composition.

According to embodiments of the present invention, if the recommended sleep-related product is a composition related to fragrance or beauty, such as a diffuser, candle, perfume, cosmetic, bathroom product (cleaning product), laundry product, fabric softener, fabric softener sheet, lotion, skin, sleep pack, mask pack, or a composition that can be ingested by the user, such as a nutritional supplement, general food, health functional food, special nutritional food, special medical-purpose food, pharmaceutical, food additive, etc., it can be manufactured by selecting one composition from the compositions (composition 1 to 6 of the sleep-related product as shown in the table of FIG. 70a. However, the compositions and compositions in FIG. 70a are merely examples, and various compositions and compositions can be used.

FIG. 70b is a table illustrating a lookup table in which fiber component information is recorded when the sleep-related product is composed of fiber components.

According to embodiments of the present invention, if the recommended sleep-related product is an item such as a mattress cover, pad, mattress, mattress cover, blanket, quilt, comforter, duvet cover, pillow, pillow cover, pajamas, sleep socks, sleep cap, eye mask, etc., it can be manufactured by selecting one product from the fiber components (product 1 to 6 of the sleep-related product as shown in the table of FIG. 21. However, the fiber components and products in FIG. 21 are merely examples, and various fiber components and products can be used.

Meanwhile, according to embodiments of the present invention, if the sleep-related product is an item such as a mattress cover, pad, mattress, mattress cover, blanket, quilt, comforter, duvet cover, pillow, pillow cover, pajamas, sleep socks, sleep cap, eye mask, etc., the fiber components of the product may include natural fibers such as seed fibers (cotton, kapok, coir, etc.), bast fibers (linen, ramie, hemp, jute, kenaf, etc.), leaf fibers (sisal, Manila hemp, etc.), animal fibers (silk, wool, cashmere, angora, mohair, alpaca, etc.), and mineral fibers.

Additionally, according to embodiments of the present invention, the fiber components of the sleep-related product may include at least one of synthetic fibers (nylon, polyester, acrylic, modacrylic, polyurethane, vinylon, polyolefin, vinylidene, polyvinyl chloride, aramid, polybenzimidazole, polybenzoxazole, polycarbonate, polyimide, etc.), regenerated fibers (rayon, regenerated protein fibers), and semi-synthetic fibers (acetate, triacetate, etc.).

The step of displaying recommendation information on sleep-related products may be performed on the user terminal 10 or on a display means provided in the sleep-related product recommendation device. Specifically, when the method for recommending sleep-related products according to the present invention is entirely executed on the user terminal, the generation and display of recommendation information on sleep-related products can be performed on the user terminal. In another embodiment, the generation of recommendation information in the method for recommending sleep-related products may be carried out by a sleep-related product recommendation device (a separate device, server, or cloud), and the recommendation information may be displayed via the user terminal.

Particularly, the recommended sleep-related products may be those for which verification indicators have been generated by the sleep-related product verification device and method described in detail above. That is, the lookup table for recommending sleep-related products can be generated based on the verification indicators for each sleep-related product derived by the sleep-related product verification device and method.

Meanwhile, according to one embodiment of the present invention, in the method for recommending sleep-related products, a recommendation method based on user input actions may be provided. Here, the user's input actions may include checking sleep-related products through swiping, entering preferred keywords, or selecting preferred keywords.

Meanwhile, according to one embodiment of the present invention, the method may also include a process where the user checks products with which they have good memories while swiping through various products displayed on the electronic device, such as a device recommending sleep-related products.

Alternatively, during the process of swiping sleep-related products, if the user spends a relatively long time on the content of a particular product, it may be recognized that the user prefers that product.

According to one embodiment of the present invention, if an electronic device, such as a device recommending sleep-related products, asks the user to “Enter a product you found comfortable to use” or “Enter keywords for preferred products,” a method for recommending sleep-related products based on the text information or keyword information entered by the user may be provided.

Meanwhile, according to one embodiment of the present invention, if an electronic device, such as a device recommending sleep-related products, asks the user to “Select keywords you find interesting,” a method for recommending sleep-related products based on the text information or keyword information selected by the user may be provided.

Meanwhile, according to the embodiments of the present invention, a method for recommending sleep-related products based on existing statistical data, which indicates relatively high preference, may be provided. Additionally, in this case, a method for recommending products that are both highly preferred and deemed appropriate for the user's sleep indicators based on the verification indicators of the product may be provided.

Alternatively, according to the embodiments of the present invention, statistics based on attributes of different consumer groups may be generated through the sleep-related product verification method and/or recommendation method according to the present invention, and a method for recommending sleep-related products in light of these statistics may be provided. Here, the attributes of different consumer groups may include various attributes or environmental/non-environmental factors such as gender, age group, occupation, living area, race, and the presence of pets.

For example, if, according to the present invention, the recommendation of sleep-related products to a consumer group of males in their 20s results in a high preference for a certain product or a significant improvement in sleep quality, a method for preferentially recommending that product to the consumer group of males in their 20s may be provided. The aforementioned age and gender are merely examples for understanding the present invention, and the present invention is not limited thereto. The attributes of different consumer groups may include various attributes or environmental/non-environmental factors.

Meanwhile, according to one embodiment of the present invention, it is possible to compare the sleep indicator calculated based on the user's sleep state information with the verification indicator of the sleep-related products described above, while further considering subjective indicators related to products preferred by the user. In this case, the recommendation of sleep-related products can be made by appropriately distributing the weight for the comparison between the sleep indicator and the verification indicator, and the weight for the subjective indicator, as a ratio from 0 to 1.

For example, if the weight for the comparison between the sleep indicator and the verification indicator is 1, and the weight for the subjective indicator is 0, the recommendation for sleep-related products can be made solely based on the comparison of the sleep indicator and the verification indicator. Conversely, if the weight for the comparison between the sleep indicator and the verification indicator is 0.5, and the weight for the subjective indicator is 0.5, the recommendation for sleep-related products can be made by considering both the comparison between the sleep indicator and the verification indicator and the subjective indicator.

Additionally, if the weight for the comparison between the sleep indicator and the verification indicator is 0, and the weight for the subjective indicator is 1, the recommendation for sleep-related products can be made solely based on the subjective indicator. The specific numerical values for the weights mentioned above are merely examples and do not limit the present invention.

The aforementioned method for recommending sleep-related products and the method for verifying sleep-related products can be combined and summarized into the following steps.
(Step 1) The user uses a sleep-related product.
(Step 2) The user's sleep indicator is measured.
(Step 3) The sleep indicator for the used sleep-related product is recorded and collected.
(Step 4) An evaluation of the impact of the used sleep-related product on the user's sleep is calculated.
(Step 5) Statistics on sleep evaluations from multiple users for multiple sleep-related products are obtained.
(Step 6) Based on the user's sleep indicator and the verification indicator of the sleep-related products, a recommendation for sleep-related products is made.

Meanwhile, the content summarized in the above steps is merely one embodiment of the present invention and is not limited thereto. One or more steps may be added, modified, or deleted as long as they can be easily conceived by those skilled in the art in light of the detailed description of the present invention described above. For example, in the step of recommending sleep-related products, a method may be provided that considers the user's input action to recommend sleep-related products, or a method may be provided that recommends sleep-related products based solely on the sleep indicator of an individual who used the sleep-related product, rather than statistics on sleep evaluations from multiple users for multiple sleep-related products.

According to the present invention, by utilizing sleep state information and sleep stage information extracted based on environment sensing information obtained from a user terminal for the verification of sleep-related products, the objectivity and reliability of the verification concerning the impact on sleep can be ensured. Furthermore, by recommending sleep-related products based on verification indicators possessing objectivity and reliability, it is possible to guide the user's sleep, prevent sleep disorders, and simultaneously improve the quality of sleep.

### Contents of the Cosmetic Recommendation Device

FIG. 13 is a flowchart illustratively showing the process of acquiring sleep state information through an automatic sleep measurement mode related to an embodiment of the present invention. The steps shown in FIG. 13 may be reordered as needed, and at least one step may be omitted or added. That is, the aforementioned steps are merely one embodiment of the present invention, and the scope of rights of the present invention is not limited thereto.

According to one embodiment, it is possible to detect the occurrence of a user's movement within a space through the first sensing unit (S110). The first sensing unit may be equipped with at least one of a PIR sensor and an ultrasonic sensor. The PIR sensor can detect changes in infrared radiation emitted from the user's body to detect the user's movement within the detection range. For example, the PIR sensor can identify infrared radiation of 8 μm~14 μm emitted from the user's body to detect the user's movement within the bedroom. The ultrasonic sensor can generate sound waves and detect signals reflected from a specific object to detect the object's movement. For instance, the ultrasonic sensor can generate sound waves within the bedroom space and detect the user's movement within the bedroom through sound waves reflected from the user's body as the user enters the bedroom.

According to one embodiment, it is possible to identify that the user is located in a predefined area through the second sensing unit (S120). The second sensing unit can receive transmitted wireless signals and detect whether the user is located in the predefined area based on the received wireless signals. In the embodiment, the predefined area relates to the area within a space where the user lies down to sleep, for example, an area equipped with a bed. Specifically, in the present invention, the space may refer to the interior space of a bedroom, and the predefined area may refer to the space where the bed is located.

In the embodiment, the second sensing unit may be characterized by being provided in a position opposite the transmitting module based on the predefined area. For example, the transmitting module and the second sensing unit may be provided on each side of the bed where the user sleeps. In this case, the cosmetic recommendation device/cosmetic verification device of the present invention can acquire information on whether the user is located in the predefined area and object state information regarding the user's movement or breathing based on wifi-based OFDM signals transmitted and received through the transmitting module and receiving module.

According to one embodiment, it is possible to collect sound information related to a space (S130). That is, upon detecting the occurrence of a user's movement within a space through the first sensing unit and identifying the user's movement in the predefined area through the second sensing unit, it is possible to automatically collect sound information related to the space.

According to one embodiment, it is possible to calculate sleep state information based on the collected sound information (S140). Sleep state information related to whether the user is before sleep or during sleep can be acquired based on singularities identified from the sound information. Specifically, if no singularity is identified, it can be determined that the user is before sleep, and if a singularity is identified, it can be determined that the user is during sleep after the singularity. Furthermore, after identifying a singularity, if a predefined pattern is not observed at a certain point (e.g., waking point), it can be determined that the user has woken up after sleep.

That is, based on whether a singularity is identified in the sound information and whether a predefined pattern is continuously detected after identifying a singularity, sleep state information related to whether the user is before, during, or after sleep can be acquired.

Additionally, in the embodiment, it is possible to acquire a spectrogram based on sleep sound information. In this case, the conversion to a spectrogram may be intended to facilitate the analysis of breathing or movement patterns related to relatively small sounds. Moreover, by utilizing a sleep analysis model configured to include a feature extraction model and a feature classification model, it is possible to generate sleep stage information based on the acquired spectrogram. In this case, the sleep analysis model can perform sleep stage prediction by inputting spectrograms corresponding to multiple epochs to consider both past and future information, thereby outputting more accurate sleep stage information.

### Cosmetic Recommendation Device and Method

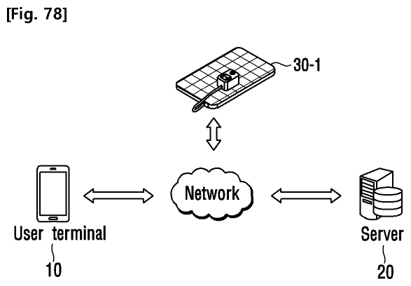

FIG. 75 is a block diagram illustrating a cosmetic recommendation method according to an embodiment of the present invention.

According to the cosmetic recommendation method of the present invention, when the recommended cosmetics are used before sleep, it can stabilize the mind and body, reduce sleep latency, and improve sleep efficiency, which leads to an enhancement in sleep quality.

The cosmetic recommendation method according to the present invention includes, as shown in FIG. 75, the steps of calculating the user's sleep indicator S611, generating cosmetic information corresponding to the calculated sleep indicator S621, and displaying the generated cosmetic information S631.

The step of calculating the user's sleep indicator can be based on the sleep state information and sleep stage information described in detail above.

Briefly, the sleep state information includes information related to whether the user is sleeping and may include at least one of the first sleep state information indicating the user is before sleep, the second sleep state information indicating the user is during sleep, and the third sleep state information indicating the user is after sleep.

This sleep state information is obtained based on environment sensing information, which is acquired in a non-contact manner by the user terminal. Sleep stage information can also be extracted based on environment sensing information, and sleep stages can be divided into NREM (non-REM) sleep and REM (Rapid Eye Movement) sleep. NREM sleep can further be divided into multiple stages (e.g., two stages of Light and Deep, or four stages from N1 to N4, but is not limited thereto. Through sleep stage analysis, not only the quality of sleep related to sleep but also sleep disorders (e.g., sleep apnea) and their underlying causes (e.g., snoring) can be predicted.

Meanwhile, the step of generating cosmetic information can generate recommended cosmetic information based on a lookup table in which cosmetic information corresponding to the sleep indicator is recorded. The lookup table can be configured as shown in Tables 1 to 3 below, but this is merely one embodiment and can be configured in various ways.

TABLE 1

| | Presence of | Recommended |
|---|---|---|
| Sleep Latency | Sleep Disorder | Cosmetics |
| 0 to 10 | Yes | Type A-1 |
| minutes | No | Type A-2 |
| 11 to 30 | Yes | Type B-1 |
| minutes | No | Type B-2 |
| 31 minutes | Yes | Type C-1 |
| to 1 hour | No | Type C-2 |
| . . . | . . . | . . . |

TABLE 2

| Sleep Onset | Average Sleep | Recommended |
|---|---|---|
| Latency | Duration | Cosmetics |
| 0 to 10 | 7 hours or less | Type D-1 |
| minutes | 7 hours or more | Type D-2 |
| 11 to 30 | 7 hours or less | Type E-1 |
| minutes | 7 hours or more | Type E-2 |
| 31 minutes | 7 hours or less | Type F-1 |
| to 1 hour | 7 hours or more | Type F-2 |
| . . . | . . . | . . . |

TABLE 3

| REM Sleep | Recommended |
|---|---|
| Ratio | Cosmetics |
| 10% | Type G-1 |
| 20% | Type G-2 |
| 30% | Type G-3 |
| . . . | . . . |

At this time, the cosmetic recommendation method can generate a cosmetic recommendation model and, based on this, generate cosmetic information. The cosmetic recommendation model can be generated by the learning method described above, and the indicators input into the input layer of the learning model may be information acquired based on sleep state information and sleep stage information. Specifically, the input indicators may include the first to nth sleep state information, and sleep stage information of two stages (NREM (non-REM) sleep, REM (Rapid eye movement), Light, Deep) or four stages N1 to N4. Additionally, the input indicators may include sleep onset latency, average sleep time, REM sleep cycle, and NREM sleep pattern, which can be extracted from sleep state information and sleep stage information. Sleep onset latency can be extracted based on the user's intended sleep time and actual sleep time, average sleep time can be extracted based on the user's sleep and wake times, REM sleep cycle and ratio can be extracted based on sleep stage information, and NREM sleep pattern can be extracted based on the pattern, cycle, and time of LIGHT/DEEP sleep or the pattern, cycle, and time of N1 to N4 sleep.

Here, the recommended cosmetic can be manufactured by selecting one composition from among the cosmetic compositions (Composition 1 to 6 as shown in [Table 4] below and mixing it using the emulsification method, which is a conventional emulsion manufacturing method. However, the compositions below are merely one embodiment, and various compositions can be used. Furthermore, the cosmetics can include all types of cosmetics such as lotions, skin products, sleep packs, and mask packs.

TABLE 4

| Component | Composition | Composition | Composition | Composition | Composition | Composition |
|---|---|---|---|---|---|---|
| (Unit: weight %) | 1 | 2 | 3 | 4 | 5 | 6 |
| Purified Water | to 100 | to 100 | to 100 | to 100 | to 100 | to 100 |
| Cyclopentasiloxane | 6.30 | 6.30 | 6.30 | 6.30 | 6.30 | 6.30 |
| Butylene Glycol | 6.00 | 6.00 | 6.00 | 6.00 | 6.00 | 6.00 |
| Glycerin | 2.10 | 2.10 | 2.10 | 2.10 | 2.10 | 2.10 |
| Trehalose | 2.00 | 2.00 | 2.00 | 2.00 | 2.00 | 2.00 |
| Dimethicone/Vinyl | 0.10 | 0.10 | 0.10 | 0.10 | 0.10 | 0.10 |
| Dimethicone | | | | | | |
| Crosspolymer | | | | | | |
| Dimethiconol | 0.20 | 0.20 | 0.20 | 0.20 | 0.20 | 0.20 |
| Disodium EDTA | 0.02 | 0.02 | 0.02 | 0.02 | 0.02 | 0.02 |
| Ammonium | 0.50 | 0.50 | 0.50 | 0.50 | 0.50 | 0.50 |
| Acryloyldimethyltaurate/ | | | | | | |
| VP Copolymer | | | | | | |
| Carbomer | 0.20 | 0.20 | 0.20 | 0.20 | 0.20 | 0.20 |
| Tromethamine | 0.40 | 0.40 | 0.40 | 0.40 | 0.40 | 0.40 |
| Polysorbate 20 | 0.60 | 0.60 | 0.60 | 0.60 | 0.60 | 0.60 |
| Natural Yuzu Oil | 0.50 | 1.00 | 1.50 | 0.50 | — | — |
| Behenyl Alcohol | 0.50 | 1.00 | 1.50 | — | — | — |
| Jojoba Ester | 0.50 | 1.50 | 1.50 | — | — | — |
| Hydrogenated | 2.50 | 5.00 | 7.50 | — | — | — |
| C₆₋₁₄ Polyolefin | | | | | | |
| Lavender Oil | — | — | — | — | — | 0.50 |

The step of displaying recommended cosmetic information may be performed on a user terminal or on a display means provided in a cosmetic recommendation device. Specifically, if the cosmetic recommendation method according to the present invention is entirely performed on the user terminal, the generation of cosmetic recommendation information and the display of recommendation information can be executed on the user terminal. In another embodiment, the generation of recommendation information in the cosmetic recommendation method may be performed by a cosmetic recommendation device (a separate device, server, or cloud), and the recommendation information may be displayed via the user terminal.

In particular, the recommended cosmetics may be those for which a verification indicator has been generated by the cosmetic verification device and method described below. That is, the lookup table for cosmetic recommendation may be generated based on the verification indicators derived for each cosmetic by the cosmetic verification device and method.

### Cosmetic Verification Device and Method

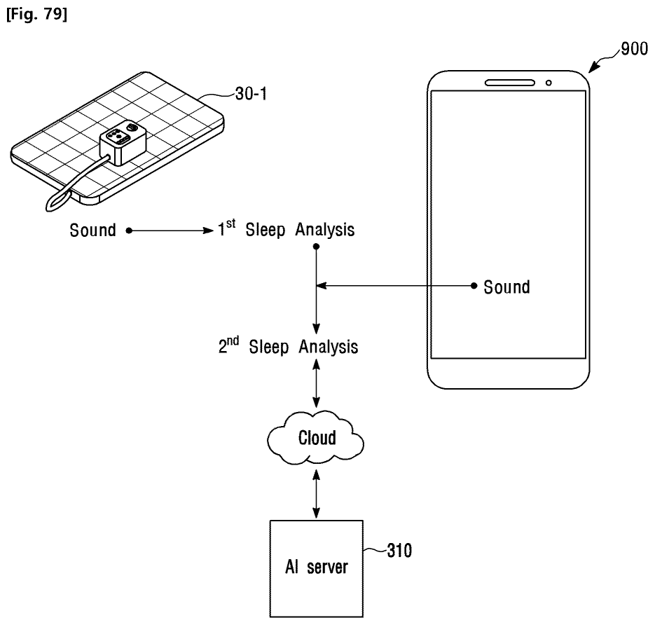

FIG. 76 is a block diagram illustrating a cosmetic verification method according to an embodiment of the present invention.

According to the cosmetic verification method of the present invention, it is possible to verify the impact of the cosmetic under verification on sleep quality. Specifically, by applying the cosmetic under verification before sleep and analyzing the user's sleep information, it becomes possible to objectively confirm and analyze whether the user's sleep latency has decreased, whether sleep disorders have improved, and whether stable and deep sleep has been achieved.

As illustrated in FIG. 76, the cosmetic verification method according to the present invention includes the steps of: receiving environment sensing information from a user terminal of a user who has used a predetermined cosmetic S711; acquiring at least one of the user's sleep state information and sleep stage information based on the environment sensing information S721; generating a verification indicator for the predetermined cosmetic using at least one of the sleep state information and sleep stage information S731; and verifying the effect of the predetermined cosmetic on sleep quality based on the verification indicator S741.

In this context, the step of acquiring at least one of the sleep state information and sleep stage information S721 may include generating an inference model trained with the environment sensing information as input, and extracting the sleep state information as an output value by inputting the environment sensing information received from the user terminal into the inference model.

Initially, the user applies the predetermined cosmetic before falling asleep. The predetermined cosmetic becomes the subject for verifying its impact on sleep. Subsequently, environment sensing information is received from the user's user terminal. The environment sensing information is as detailed above.

Thereafter, based on the environment sensing information, the user's sleep state information and sleep stage information are acquired. The sleep state information includes information related to whether the user is asleep, and may include at least one of the first sleep state information indicating the user is before sleep, the second sleep state information indicating the user is during sleep, and the third sleep state information indicating the user is after sleep. This sleep state information is acquired based on the environment sensing information, which is obtained in a non-contact manner by the user terminal.

Sleep stage information can also be extracted based on the environment sensing information, and sleep stages can be classified into NREM (non-REM) sleep and REM (Rapid Eye Movement) sleep. NREM sleep can be further divided into multiple stages (e.g., two stages of Light and Deep, or four stages from N1 to N4, but is not limited thereto. Through sleep stage analysis, not only the quality of sleep related to sleep can be predicted, but also sleep disorders (e.g., sleep apnea) and their underlying causes (e.g., snoring).

Subsequently, using at least one of the sleep state information and sleep stage information, a verification indicator for the predetermined cosmetic is generated, and the verification indicator can be displayed as a score as shown in Table 5 below, but is not limited thereto.

TABLE 5

| | Sleep | Sleep Apnea | Snoring | REM Sleep |
|---|---|---|---|---|
| | Latency | Count | Time | Ratio |
| Verified | −10 minutes | −5 times | −20 minutes | Average +5% |
| Cosmetic | | | | |

Meanwhile, the cosmetic verification method according to the present invention is used by multiple users for the same cosmetic product to be verified. Based on the environment sensing information received from multiple users, each user's sleep state information and sleep stage information are derived, and a verification indicator for multiple users can be generated as shown in Table 6 below.

TABLE 6

| | Sleep Onset | | Sleep Apnea | Snoring | REM Sleep |
|---|---|---|---|---|---|
| | Latency | | Count | Duration | Ratio |
| User 1 | −10 | minutes | +2 | −40 | Averag −4% |
| | | | occurrences | minutes | |
| User 2 | −8 | minutes | −7 | −1 | Averag +10% |
| | | | occurrences | hour | |
| User 3 | −5 | minutes | N/A | N/A | Averag −11% |
| User 4 | −9 | minutes | N/A | N/A | Averag +8% |

| | | | | |
|---|---|---|---|---|
| . . . | . . . | . . . | . . . | . . . |

Based on the verification indicators in Table 6, confirmed by multiple users, it can be determined that the cosmetic product to be verified is effective in delaying sleep onset latency. Therefore, it can be selected as a recommended cosmetic product for users with relatively longer sleep onset latency and recommended to them.

Through the aforementioned process, sleep evaluation statistics for multiple users regarding multiple cosmetics can be obtained, and a lookup table as shown in Table 7 below can be acquired. This can be used to generate a recommended cosmetic lookup table in the cosmetic recommendation method described above.

TABLE 7

| | Sleep Onset | | | |
|---|---|---|---|---|
| Cosmetic | Latency | Sleep Apnea | Snoring | Sleep Stability |
| Type | Reduction | Improvement | Improvement | Enhancement |
| Type A-1 | Excellent | Good | Good | Excellent |
| Type A-2 | Good | Excellent | Excellent | Good |
| Type A-3 | Good | Good | Good | Good |
| . . . | . . . | . . . | . . . | . . . |
| Type B-1 | Good | — | — | Good |
| Type B-2 | — | Excellent | Excellent | — |
| . . . | . . . | . . . | . . . | . . . |

At this time, the cosmetic type may be classified according to the type such as lotion or mask pack, or according to the target user such as female/male, or according to the fragrance such as lavender or rosemary, or according to the formulation such as emulsion or skin.

Of course, in other embodiments, the cosmetic type may be set in a manner different from the above. By combining the aforementioned cosmetic recommendation method and cosmetic verification method, it can be summarized into the following steps.
(Step 1) The user applies cosmetics before sleep.
(Step 2) The user's sleep indicator information is measured.
(Step 3) The sleep indicator information related to the applied cosmetics is recorded and collected.
(Step 4) An evaluation of the impact of the applied cosmetics on the user's sleep is calculated.
(Step 5) Statistics on sleep evaluations from multiple users for various cosmetics are acquired.
(Step 6) The optimal cosmetics for each user are recommended based on their sleep indicator information.

According to the present invention, by utilizing sleep state information and sleep stage information extracted based on environment sensing information obtained from the user terminal for cosmetic verification, the objectivity and reliability of cosmetic verification related to the impact on sleep can be ensured. Furthermore, by recommending cosmetics based on verification information with objectivity and reliability, it is possible to induce sleep for the user, prevent sleep disorders, and simultaneously improve the quality of sleep.

### Heated-Water Mattress

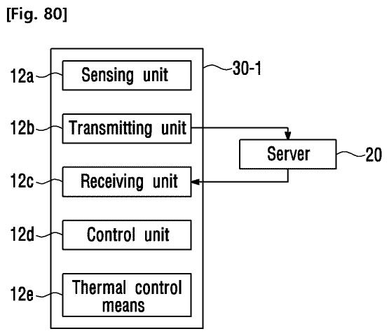

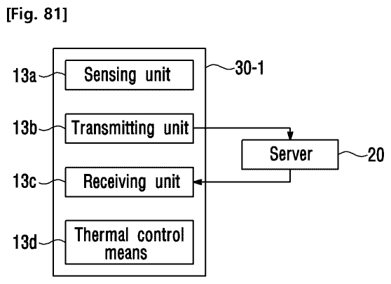

FIG. 77 is a conceptual diagram illustrating a system of a heated-water mattress for creating a sleep environment. The system according to FIG. 77 may include a computing device 100, a user terminal 10, a server 20, a heated-water mattress 30-1, and a network. Meanwhile, FIG. 78 is a conceptual diagram illustrating another embodiment of a system of a heated-water mattress 30-1 for creating a sleep environment. The system according to FIG. 78 may include a user terminal 10, a server 20, a heated-water mattress 30-1, and a network. That is, it includes the heated-water mattress 30-1 directly performing the role of the computing device 100.

As shown in FIG. 77, the present invention allows the computing device 100, user terminal 10, server 20, and heated-water mattress 30-1 to mutually transmit and receive data for the system according to embodiments of the present invention through the network.

According to one embodiment of the present invention, a user terminal 10 is a terminal capable of receiving information related to the user's sleep through information exchange with a computing device 100, and may refer to a terminal possessed by the user. For example, the user terminal 10 may be a terminal related to a user who wishes to improve health through information related to their sleep habits.

The user can acquire monitoring information related to their sleep through the user terminal 10. The monitoring information related to sleep may include, for example, sleep state information related to the time the user fell asleep, the duration of sleep, and the time of waking up, or sleep stage information related to changes in sleep stages during sleep. Specifically, the sleep stage information may refer to information indicating changes in the user's sleep, such as light sleep, normal sleep, deep sleep, or REM sleep, at each point during the user's 8-hour sleep the previous night. The specific description of the sleep stage information mentioned above is merely exemplary and does not limit the present invention.

### Integration of Heated-Water Mattress and Smartphone

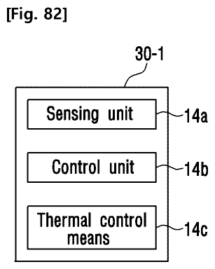

FIG. 79 is a configuration diagram for explaining the operation among components of an AI-based non-contact sleep analysis system according to a heated-water mattress for creating a sleep environment. As shown in FIG. 79, the heated-water mattress 30-1 and the smartphone 900 are integrated to perform sleep analysis for the user. The heated-water mattress 30-1 and the smartphone 900 can be paired via Bluetooth or connected through other wireless communication methods.

The smartphone 900 can perform sleep analysis based on the sleep sound information acquired from the heated-water mattress 30-1. At this time, the user's sleep sound information may be acquired from the heated-water mattress 30-1 and transmitted to the smartphone 900, or it may be acquired independently through a microphone embedded in the smartphone 900.

That is, in the embodiment shown in FIG. 79, sleep stage analysis is performed through the heated-water mattress 30-1 and the smartphone 900 as a non-contact sleep stage analysis. The user can check the sleep stage analysis results derived from the smartphone 900 on the screen of the smartphone 900.

According to the present invention, since sound is transmitted in a radial direction, when using sleep sound information, there is an advantage of being able to collect and analyze information regardless of the user's position, or the distance or angle between the user and the heated-water mattress 30-1.

In one embodiment, the heated-water mattress 30-1 can transmit a predetermined signal to allow the user to place the heated-water mattress 30-1 closer to the user when the heated-water mattress 30-1 is not in contact with the user, so that it can receive input signals (sleep sound information) for sleep analysis. The predetermined signal may be vibration, an alarm, an LED, etc.

Additionally, the heated-water mattress 30-1 may include an acoustic sensor internally to measure various sound information. The heated-water mattress 30-1 can perform a primary sleep analysis using the sound information. The heated-water mattress 30-1 can be paired with the smartphone 900 to transmit the information measured by the heated-water mattress 30-1, or the primary sleep analysis results analyzed by the heated-water mattress 30-1, to the smartphone 900. In this case, the heated-water mattress 30-1 may include a communication module.

Meanwhile, the heated-water mattress 30-1 may include various modules (temperature control module, infrared irradiation module, cooling module) for adjusting the temperature, and the temperature can be adjusted based on the final sleep stage analysis results. This improves the quality of the user's sleep. Specific embodiments of the temperature control of the heated-water mattress 30-1 will be described later.

Meanwhile, the heated-water mattress 30-1 may include a vibration module or an alarm module to alleviate and improve sleep disorders. Specifically, when sleep apnea, snoring, sleep hyperventilation, REM sleep, etc., are detected, the vibration module or alarm module of the heated-water mattress 30-1 can be activated to deliver tactile or auditory stimuli to the user.

### Temperature Control Embodiment of the Heated-Water Mattress

FIG. 79 is a block diagram illustrating the operation among components of an AI-based non-contact sleep analysis system according to the heated-water mattress for creating a sleep environment.

As shown in FIG. 79, the heated-water mattress 30-1 and the smartphone 900 are interlinked to perform sleep analysis of the user. The heated-water mattress 30-1 and the smartphone 900 can be paired via Bluetooth or connected through other wireless communication methods.

According to an embodiment of the present invention, as described above, the heated-water mattress 30-1 or the smartphone 900 can identify the user's sleep stage in real-time. Hereinafter, an embodiment of temperature control of the heated-water mattress in relation to the user's circadian rhythm is described.

According to the present invention, the user's circadian rhythm may refer to the physiological, chemical, and behavioral flow that occurs periodically within all living organisms in accordance with the user's 24-hour day and night changes. Specifically, the user's circadian rhythm may refer to a 24-hour cycle where the user's core body temperature is highest in the evening and lowest just before waking up from sleep in the early morning.

According to an embodiment of the present invention, sleep may commence when the user's core body temperature rapidly declines according to the user's circadian rhythm. Therefore, when the heated-water mattress 30-1 and the smartphone 900 are interlinked to perform sleep analysis of the user, the user's sleep can be detected in real-time during the user's sleep onset, and the temperature of the heated-water mattress 30-1 can be lowered in accordance with the user's sleep onset timing.

Specifically, according to an embodiment of the present invention, when the heated-water mattress 30-1 and the smartphone 900 are interlinked to perform sleep analysis of the user, the temperature of the heated-water mattress 30-1 can be rapidly lowered and then gradually increased during the WAKE state, which corresponds to the user being awake.

According to another embodiment of the present invention, if the user's sleep is detected or the user is in a light sleep state, i.e., corresponding to the Deep stage, the lowering of the temperature, which was decreased during sleep onset, can be halted.

Specifically, according to an embodiment of the present invention, the user's circadian rhythm may indicate that the user's core body temperature drops during NREM and rises gradually during the REM sleep stage. Through this, the heated-water mattress 30-1 or the smartphone 900 can detect sleep in real-time, and if NREM sleep is detected, the temperature of the heated-water mattress 30-1 can be lowered, and in the case of REM sleep, the temperature can be gradually increased.

For instance, the temperature control embodiment of the heated-water mattress 30-1 may be guided such that the temperature difference between the user's core body temperature and the heated-water mattress 30-1 is not significant according to the user's circadian rhythm. However, when the user's core body temperature decreases during NREM sleep, the temperature of the heated-water mattress 30-1 can be increased to maintain a consistent body temperature for the user. This embodiment is not limited thereto.

### Operation of the Heated-Water Mattress and First Environment Adjustment Information Based on Pre-Sleep State

According to one embodiment of the present invention, if the user's state is in the pre-sleep state, the processor 130 can generate first environment adjustment information to lower the temperature of the heated-water mattress 30-1 from the predicted time the user is preparing for sleep (e.g., sleep induction time) until the time the user falls asleep (i.e., the time when the second sleep state information is acquired). The processor 130 can decide to transmit the first environment adjustment information to the heated-water mattress 30-1.

### Operation of the Heated-Water Mattress and Second Environment Adjustment Information Based on the Second Sleep State

According to one embodiment of the present invention, the processor 130 can generate second environment adjustment information based on the second sleep state information. Specifically, if the user's sleep is detected or the user is lightly asleep, corresponding to the Deep stage, the descent of the temperature lowered during sleep onset can be halted.

According to the present invention, as described above, sleep analysis involves analyzing various information such as sleep onset time, wake-up time, and total sleep time. In one embodiment, the processor 130 can extract sleep stage information. The sleep stage information can be extracted based on the user's environment sensing information. Sleep stages can be classified into NREM (non-REM) sleep and REM (Rapid Eye Movement) sleep, with NREM sleep further divided into multiple stages (e.g., 2 stages of Light and Deep, or 4 stages from N1 to N4.

Specifically, according to one embodiment of the present invention, the user's circadian rhythm may indicate that the user's core body temperature decreases during NREM sleep and gradually increases during the REM sleep stage. Through this, the heated-water mattress 30-1 or smartphone 900 can detect sleep in real-time, lowering the temperature of the heated-water mattress 30-1 when NREM sleep is detected, and gently increasing the temperature during REM sleep.

For instance, the temperature control embodiment of the heated-water mattress 30-1 may be guided such that the temperature difference between the user's core body temperature and the heated-water mattress 30-1 is not significant according to the user's circadian rhythm. However, when the user's core body temperature decreases during NREM sleep, the temperature of the heated-water mattress 30-1 can be increased to maintain a consistent body temperature for the user. This embodiment is not limited thereto.

Heated-Water Mattress when Learning and Inference are Performed on a Server

FIG. 80 is a diagram illustrating a heated-water mattress for which learning and inference are performed on a server to create a sleep environment.

As illustrated in FIG. 80, the heated-water mattress 30-1 may include a sensing unit 12a), a transmitting unit 12b), a receiving unit 12c), a control unit 12d), and thermal control means 12e).

Specifically, the sensing unit 12a) acquires the user's sound information, which may be sleep sound information, and the transmitting unit 12b) can transmit the acquired user's sound information to the server 20. More specifically, the transmitting unit 12b) may transmit the user's sound information to the server 20, or it may transmit pre-processed user's sleep sound information to the server 20.

According to the present invention, the server 20 can generate sleep state information based on the transmitted user's sound information. Accordingly, the receiving unit 12c) of the heated-water mattress 30-1 can receive the generated sleep state information from the server 20, and the control unit 12d) can generate thermal control information based on the received sleep state information. Consequently, the thermal control means 12e) can adjust the heat according to the thermal control information generated by the control unit 12d).

According to one embodiment of the present invention, the receiving unit 12c) can receive sleep state information from the server 20 indicating that the user is about to fall asleep, in which case the control unit 12d) can generate temperature control information based on the user-set temperature. For example, if the user-set temperature is 25 degrees, the temperature of the heated-water mattress 30-1 can be adjusted to 25 degrees until the user falls asleep.

According to the present invention, sleep latency may refer to the time it takes for a user to be gradually induced from a fully awake state to the lightest stage of NREM sleep.

According to one embodiment of the present invention, if the user is in sleep latency, the temperature can be gradually lowered based on the user-set temperature. Additionally, the temperature can be gradually increased based on the user-set temperature. For example, preferably, if the user-set temperature is 25 degrees, the control unit 12d) can generate temperature control information to gradually lower the temperature based on 25 degrees during sleep latency.

According to another embodiment of the present invention, if the receiving unit 12c) receives the user-set sleep latency desired by the user, the control unit 12d) can gradually lower or increase the temperature based on the user-set sleep latency. For instance, if the user-set sleep latency is 10 minutes and the user-set temperature is 25 degrees, the control unit 12d) can gradually lower the temperature from the start of sleep measurement until 10 minutes later. Additionally, the temperature can be gradually increased.

According to the present invention, a user body temperature measurement unit (not shown) may be further included. If the receiving unit 12c) receives user body temperature drop information from the user body temperature measurement unit (not shown) during sleep latency, the control unit 12d) can generate temperature control information to maintain the temperature above a predetermined level.

For example, if the user-set temperature is 25 degrees and a body temperature drop is detected during sleep latency, temperature control information to increase the set temperature to 26 degrees can be generated.

According to the present invention, if the server 20 generates sleep state information for a specific user multiple times, it can generate biological rhythm information for that specific user. In this case, if the user is in sleep latency, the control unit 12d) can generate temperature control information based on the generated biological rhythm information.

In a specific example, if the user's biorhythm information indicates that the user's body temperature primarily rises during the sleep latency period, the control unit 12d) may generate temperature control information to lower the temperature below the user-set temperature.

According to one embodiment of the present invention, when the user's first deep sleep is detected, the control unit 12d) may maintain the temperature control information generated during the sleep latency period until that time. Alternatively, it may be maintained for a predetermined time after the first deep sleep is detected.

For instance, if the control unit 12d) generates temperature control information to maintain 24 degrees during the user's sleep latency period, and the first deep sleep is detected 30 minutes after falling asleep, the temperature can be maintained at 24 degrees until 30 minutes after falling asleep. Alternatively, if the first deep sleep ends 50 minutes after falling asleep, the temperature can be maintained at 24 degrees until 50 minutes after falling asleep, but it is not limited thereto.

According to one embodiment of the present invention, when the user's first REM sleep is detected, the control unit 12d) may maintain the temperature control information generated during the sleep latency period until that time. Alternatively, it may be maintained for a predetermined time after the first deep REM sleep is detected.

For instance, if the control unit 12d) generates temperature control information to maintain 24 degrees during the user's sleep latency period, and the first deep REM sleep is detected 30 minutes after falling asleep, the temperature can be maintained at 24 degrees until 30 minutes after falling asleep. Alternatively, if the first REM sleep ends 50 minutes after falling asleep, the temperature can be maintained at 24 degrees until 50 minutes after falling asleep, but it is not limited thereto.

According to one embodiment of the present invention, during REM sleep, changes in autonomic nervous activity may occur, which can manifest as changes in the user's temperature control. That is, since both sympathetic and parasympathetic nervous activations can occur, temperature control during REM sleep can be important. Specifically, if the user is in REM sleep, it is possible to increase the temperature to maintain the gap with the core temperature or to compensate for a lowered body temperature. Conversely, it is also possible to decrease the temperature to maintain the gap with the core temperature or to compensate for an increased body temperature during REM sleep.

Thus, the control unit 12d) can generate temperature control information to induce temperature changes during REM sleep based on changes in the user's sleep state information. Additionally, if the user experiences an awakening state, it is possible to generate new temperature control information to automatically recommend a different temperature by learning whether the set temperature is too high or too low.

According to one embodiment of the present invention, the temperature can be increased from the point when the user is in an awakening state, REM state, or light sleep state until the user's desired awakening time. For example, if the user's desired awakening time is 7:00 AM and the user's REM state is detected at 6:40 AM, the set temperature of the heated-water mattress 30-1 can be gradually increased from 6:40 AM to 7:00 AM.

According to another embodiment of the present invention, the temperature can be gradually increased from the time between the detection of the user's REM state and light sleep state until the user's desired awakening time. For example, if the user's REM state is detected at 6:40 AM and the light sleep state is detected at 6:50 AM, the set temperature of the heated-water mattress 30-1 can be gradually increased from 6:45 AM to the user's desired awakening time of 7:00 AM.

According to the present invention, if the user is in a light sleep state at the desired awakening time, the control unit 12d) can generate first temperature control information, and if the user is in a deep sleep state at the desired awakening time, the control unit 12d) can generate second temperature control information. Specifically, the temperature of the second temperature control information may be higher than that of the first temperature control information.

According to another embodiment of the present invention, if the user's body temperature is detected to be higher than a predetermined temperature by the user body temperature measurement unit (not shown), the control unit 12d) may generate thermal control information to lower the temperature of the heated-water mattress 30-1 below the set temperature at the user's desired wake-up time.

### When Learning, Inference, and Generation of Thermal Control Information are Performed on the Server

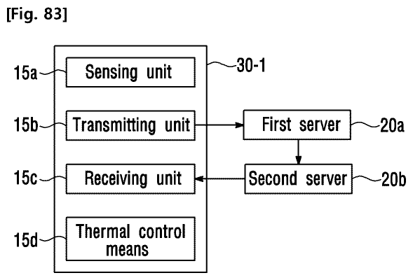

FIG. 81 is a diagram illustrating a heated-water mattress where learning, inference, and temperature control for creating a sleep environment are performed on the server.

According to the present invention, as illustrated, the heated-water mattress 30-1 may include a sensing unit 13a), a transmitting unit 13b), a receiving unit 13c), and thermal control means 13d). Accordingly, the transmitting unit 13b) can transmit the user's sound information acquired by the sensing unit 13a) to the server 20, and based on the user's sound information received by the server 20, sleep state information can be generated. Based on the generated sleep state information, temperature control information can be generated on the server 20.

Specifically, when the server 20 generates temperature control information, if sleep state information indicating that the user is in a pre-sleep state is generated, temperature control information can be generated based on the user-set temperature. Accordingly, the receiving unit 13c) can receive the generated temperature control information.

According to one embodiment of the present invention, if the server 20 generates sleep state information indicating that the user is in a sleep latency period, temperature control information set to a predetermined temperature below the user-set temperature can be generated. Furthermore, if the user-set sleep latency period desired by the user is received, it is also possible to generate temperature control information based on this. In this case, temperature control information can be generated to adjust the temperature to a predetermined temperature below or above the user-set temperature.

Specifically, if the server 20 generates sleep state information indicating that the user is in a sleep latency period and receives user body temperature drop information from the user body temperature measurement unit (not shown), it is also possible to generate temperature control information set to a predetermined temperature above the user-set temperature. In this case, the receiving unit 13c) receives the generated temperature control information, and the thermal control means 13d) can provide the corresponding temperature.

### When Learning, Inference, and Generation of Thermal Control Information are Performed on the Heated-Water Mattress

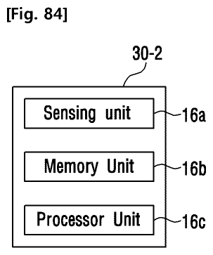

FIG. 82 is a diagram illustrating a heated-water mattress where learning, inference, and temperature control for creating a sleep environment are performed on the heated-water mattress.

According to the present invention, as illustrated, the heated-water mattress 30-1 may include a sensing unit 14a), a control unit 14b), and thermal control means 14c). When the sensing unit 14a) acquires the user's sound information, the control unit 14b) can generate sleep state information based on the acquired sound information and, based on this, generate thermal control information.

In this case, based on the thermal control information generated by the control unit 14b), the thermal control means 14c) can provide heat at the corresponding temperature.

### When Learning and Inference are Performed on the First Server, and Thermal Control Information is Generated on the Second Server.

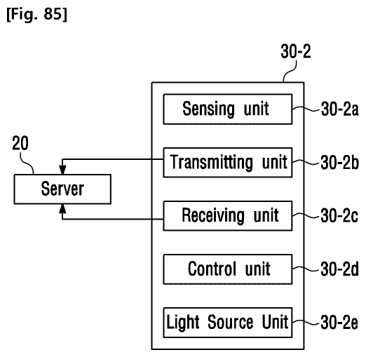

FIG. 83 is a diagram illustrating a heated-water mattress where learning and inference are performed on the first server, and temperature control is performed on the second server to create a sleep environment.

According to the present invention, as illustrated, the heated-water mattress 30-1 may include a sensing unit 15a), a transmitting unit 15b), a receiving unit 15c), and thermal control means 15d). When the transmitting unit 15b) transmits the sound information acquired from the sensing unit 15a) to the first server 20a, the first server 20a transmits the sleep sound information to the second server 20b, and the second server 20b can generate thermal control information.

Accordingly, the receiving unit 15c) receives the thermal control information generated by the second server 20b, allowing the thermal control means 15d) to provide heat accordingly.

### In the Case of a Device for Controlling the Thermal Control Means of a Heated-Water Mattress Having Thermal Control Means.

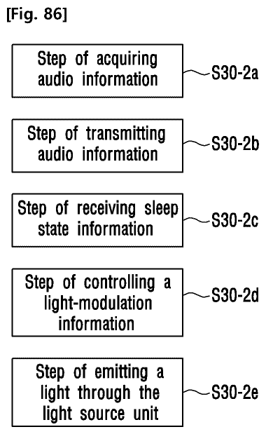

FIG. 84 is a diagram illustrating a device for controlling the thermal control means of a heated-water mattress, which includes a processor unit capable of executing an application.

As illustrated, the device 30-2 for controlling the thermal control means of a heated-water mattress may include a sensing unit 16a), a memory unit 16b), and a processor unit 16c). An application may be recorded in the memory unit 16b), and the application can be executed by the processor unit 16c). In this case, when the processor unit 16c) generates sleep state information based on the sound information acquired from the sensing unit 16a) through the application, thermal control information can be generated according to the generated sleep state information.

According to one embodiment of the present invention, both a first application and a second application may be recorded in the memory unit 16b). In this case, the processor unit 16c) generates sleep state information based on the sound information acquired from the sensing unit 16a) through the first application, and generates temperature control information based on the sleep state information generated by the first application through the second application.

According to one embodiment of the present invention, the processor unit 16c) can transmit the sound information acquired from the sensing unit 16a) to the first server 20a through the application. In this case, the processor unit 16c) receives the sleep state information acquired based on the sound information from the sensing unit 16a) from the first server 20a, and transmits it again to the second server 20b. The processor unit 16c) then receives the temperature control information acquired based on the sleep state information received by the second server 20b, and can transmit the temperature control information to the heated-water mattress 30-1.

### Light-Modulation Device

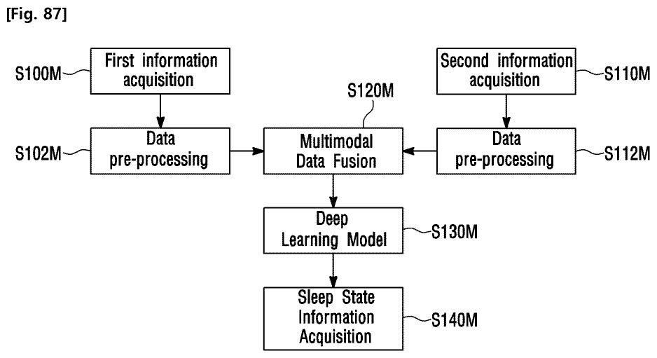

FIG. 85 is a block diagram illustrating a light-modulation device that receives sleep state information from a server according to an embodiment of the present invention.

FIG. 85 pertains to an embodiment of the present invention, where the light-modulation device 30-2 includes a sensing unit 30-2 for acquiring the user's sound information, a transmitting unit 30-2b for transmitting the acquired user's sound information to the server 20, a receiving unit 30-2c for receiving the sleep state information generated by the server 20 based on the transmitted user's sound information, a control unit 30-2d for generating light-modulation information based on the received sleep state information, and a control unit 30-2e for emitting adjusted light based on the generated light-modulation information.

According to one embodiment of the present invention, the server 20 can receive the user's sound information from the sensing unit 30-2.

In this case, the sound information may be environment sensing information, and the server 20 can receive the environment sensing information, but it may also receive pre-processed sleep sound information.

When the server 20 receives pre-processed sleep sound information, it can generate sleep state information by the aforementioned method.

Additionally, when the server 20 receives environment sensing information, it can perform pre-processing at the server 20, acquire sleep sound information, and generate sleep state information.

### Pre-Sleep Dimming

According to one embodiment of the present invention, when the server 20 generates the user's sleep state information, it can generate the user's average sleep onset latency information based on the user's sleep state information.

According to one embodiment of the present invention, before the server 20 generates the user's average sleep onset latency information, the control unit 30-2d can generate light-modulation information based on the set time information.

Specifically, the set time information may include a medically recommended sleep onset delay time.

For example, if the medically recommended sleep onset delay time is 10 minutes, the control unit 30-2d may generate light-modulation information based on 10 minutes.

According to another embodiment, the set time information may include the time desired by the user for sleep onset, which is input by the user.

For instance, if the user inputs a desired sleep onset time of 10 minutes into the receiving unit 30-2c of the light-modulation device 30-2, the control unit 30-2d may generate light-modulation information based on 10 minutes.

According to another embodiment, the set time information may include an average sleep onset delay time known statistically.

For example, the statistically known average sleep onset delay time may include the average sleep onset delay time according to the user's gender, age group, occupation group, or the average sleep onset delay time of all users utilizing the present invention.

For another example, if the user is a male in his 20s and the average sleep onset delay time for males in their 20s is 10 minutes, the control unit 30-2d may generate light-modulation information based on 10 minutes.

According to one embodiment of the present invention, if the server 20 determines that the user has fallen asleep, the control unit 30-2d may generate light-modulation information based on this determination.

For example, if the server 20 determines that the user has fallen asleep, the control unit 30-2d may generate light-modulation information to control the light emission amount of the control unit 30-2e to a predetermined brightness.

The predetermined brightness may mean 0 LUX, but is not limited thereto.

In another example, when the server 20 determines that a predetermined time has elapsed after the user has fallen asleep, i.e., when it is determined that the user has entered a sleep stabilization phase, the control unit 30-2d may generate light-modulation information to control the light emission amount of the control unit 30-2e to a predetermined brightness.

The predetermined brightness may mean 0 LUX, but is not limited thereto.

In another example, if the server 20 detects that the user is currently in a deep sleep state after the user has fallen asleep, the control unit 30-2d may generate light-modulation information to control the light emission amount of the control unit 30-2e to 0 lux.

Additionally, if the user desires to control the light emission amount to a light below a threshold value, rather than 0 lux, after falling asleep, light-modulation information may be generated to adjust the light emission amount to a user-set value, and is not limited thereto.

According to one embodiment of the present invention, when the server 20 generates the user's average sleep onset latency, upon reaching the average sleep onset latency, the control unit 30-2d may generate light-modulation information to control the light amount of the control unit 30-2e to decrease or to a predetermined brightness.

The predetermined brightness may mean 0 LUX, but is not limited thereto.

As described above, it is also possible to generate light-modulation information to adjust the light emission amount to a user-set value.

According to one embodiment of the present invention, when the server 20 generates the user's average sleep onset latency, if the user's sleep onset is detected before reaching the average sleep onset latency, the control unit 30-2d may immediately generate light-modulation information to control the light amount of the control unit 30-2e to below a threshold value.

According to one embodiment of the present invention, when the server 20 generates the user's average sleep onset latency, if the user's sleep onset is detected after reaching the average sleep onset latency, the control unit 30-2d may generate light-modulation information to maintain the light amount of the control unit 30-2e below a first threshold value upon reaching the user's average sleep onset latency, and then control it to below a second threshold value upon the user's sleep onset.

Specifically, the second threshold value is lower than the first threshold value, and the second threshold value may include the predetermined brightness.

The predetermined brightness may signify 0 LUX, but is not limited thereto.

### Circadian Rhythm

According to one embodiment of the present invention, the server 20 can generate the user's circadian rhythm information based on the sleep state information.

The server 20 may also generate this information using not only the sleep state information but also surveys regarding whether the user is a morning or evening person.

Circadian rhythm information refers to the physiological, chemical, and behavioral cycles that occur periodically within all organisms in accordance with the 24-hour changes of day and night.

The control unit 30-2d can generate light-modulation information so that the user's circadian rhythm information generated by the server 20 aligns with predetermined biological time information.

For a specific example, the predetermined biological time information may be absolute biological time information commonly recognized based on sunrise and sunset times, which are determined by latitude, longitude, or date.

More specifically, it may include setting the biological time information such that the server 20 recognizes 6 AM as the sunrise time and 6 PM as the sunset time.

However, setting the biological time information based on sunrise and sunset times can be performed not only by the server 20 but also by the receiving unit 30-2c, and since it is based on latitude or longitude, it can also be done by the sensing unit 30-2, and is not limited thereto.

That is, according to the user's circadian rhythm information generated by the server 20, if the user's wake-up time is 6 AM and sleep time is 10 PM, and if generating light-modulation information to align with absolute biological time information, the control unit 30-2d can generate light-modulation information that guides the user to the optimal wake-up and sleep times when 6 AM is the sunrise time and 6 PM is the sunset time.

According to another embodiment of the present invention, the control unit 30-2d may generate light-modulation information such that the user's biorhythm information generated by the server 20 aligns with predetermined biological time information.

For a specific example, the predetermined biological time information may include the user's desired wake-up time and sleep onset time.

That is, if the user's desired wake-up time is 7:00 AM and the desired sleep onset time is 11:00 PM, but according to the user's biorhythm information generated by the server 20, the user's wake-up time is 6:00 AM and sleep onset time is 10:00 PM, the control unit 30-2d may generate light-modulation information to allow the user to sleep an additional hour or fall asleep an hour later to align with the user's desired predetermined biological time information.

According to another embodiment of the present invention, the predetermined biological time information may be a pattern of biological time information suitable for a user who is a student preparing for an exam.

Additionally, the predetermined biological time information may be biological time information suitable for a user who is an office worker starting work at 8:00 AM.

Furthermore, if the user is in a profession requiring work during early morning hours, the biological time information may be adjusted accordingly, and such biological time information is merely exemplary and not limited thereto.

In other words, the present invention can guide the user's biorhythm information to align with such biological time information, and for this purpose, the control unit 30-2d may generate light-modulation information to change the user's biorhythm to the target biorhythm through the light source unit.

According to another embodiment of the present invention, the predetermined biological time information may be a method of recommending optimal biological time information suitable for the user's living environment.

For a specific example, the predetermined biological time information may be optimized biological time information based on the sunrise, sunset, temperature, or daylight amount due to weather in the user's living environment.

According to one embodiment of the present invention, the display unit (not shown) of the light-modulation device 30-2 may recommend optimized biological time information from predetermined biological time information to the user, allowing the user to select from the recommended biological time information. However, the means for inputting or receiving predetermined biological time information is not limited thereto.

### Advanced Light Alarm Logic

According to one embodiment of the present invention, the receiving unit 30-2c can receive predetermined alarm time information from the user.

Specifically, the predetermined alarm time information may be the time at which the user wishes to wake up.

The user may input the desired wake-up time into the display unit (not shown) of the light-modulation device 30-2 or select from the recommended times, but it is not limited thereto.

According to one embodiment of the present invention, the control unit 30-2d can generate light-modulation information such that the amount of light increases with a predetermined gradient from a set predetermined brightness to a user-set brightness starting from a threshold time before the predetermined alarm time.

For example, if the predetermined alarm time is 7:00 AM, the control unit 30-2d can generate light-modulation information such that the amount of light increases with a predetermined gradient from a set predetermined brightness to a user-set brightness starting at 6:30 AM, which is 30 minutes before 7:00 AM.

The predetermined gradient may have a constant slope in the form of a linear function, but it may also have a curved slope and is not limited thereto.

According to one embodiment of the present invention, if the receiving unit 30-2c receives predetermined alarm time information from the user, and the server 20 detects REM sleep between the predetermined alarm time and the threshold time, the control unit 30-2d can generate light-modulation information such that the amount of light increases with a predetermined gradient from a set predetermined brightness to a user-set brightness after a predetermined time has elapsed following the detection of REM sleep.

Specifically, the predetermined time elapsed after the detection of REM sleep may be a point between the end of REM sleep and the beginning of NI sleep.

Additionally, it may be the point at which REM sleep ends and NI sleep begins, and it may include all stages except the deep (DEEP) sleep stage. It may also include the immediate detection of REM sleep and the point at which REM sleep ends, without limitation.

According to another embodiment, if the receiving unit 30-2c receives predetermined alarm time information from the user, and the server 20 fails to detect REM sleep between the predetermined alarm time and the threshold time, light-modulation information can be generated to increase the amount of light from a predetermined brightness set at the predetermined alarm time to a user-set brightness at a predetermined gradient.

According to one embodiment of the present invention, if the user's wake (WAKE) state stage is not detected for a predetermined time after increasing the amount of light to the user-set brightness, the control unit 30-2d can generate light-modulation information to increase the amount of light beyond a threshold value.

For example, if the server 20 fails to detect REM sleep between the predetermined alarm time and the threshold time, after generating light-modulation information to increase the amount of light from a predetermined brightness set at the predetermined alarm time to a user-set brightness of 150 lux at a constant gradient, and if the wake (WAKE) state stage is not detected for 10 minutes, the control unit 30-2d can generate light-modulation information for the control unit 30-2e to emit light of 250 lux or more.

Additionally, if the WAKE stage is not detected for 0 minutes, the control unit 30-2d can not only generate light-modulation information for the control unit 30-2e to emit light of 250 lux or more but also attempt to change the light or make the light flicker to stimulate the user using the amount of light.

According to one embodiment of the present invention, based on the user's biorhythm information generated by the server 20, the control unit 30-2d can determine a predetermined gradient for the light-modulation information.

For example, in the case of a user sensitive to light where a wake (wake) state occurs even at light levels below the threshold, the control unit 30-2d can start with a gradient below the low threshold for the light-modulation information and adjust to a gradient above the high threshold when reaching the target wake (wake) state point to generate light-modulation information.

FIG. 86 is a conceptual diagram illustrating a light-modulation method for creating a user's sleep environment according to an embodiment of the present invention.

An embodiment of the present invention may include the step of acquiring audio information 530-2a, the step of transmitting audio information 530-2b, the step of receiving sleep state information 530-2c, the step of controlling alight-modulation information 530-2d, or the step of emitting a light through the light source unit 530-2e.

### Method for Analyzing Sleep State Information Using Multimodal Sleep Information

### An Embodiment of the Method for Analyzing Sleep State Information Using Multimodal Sleep Information (Concept-A)

FIG. 87 is a flowchart illustrating a method for analyzing sleep state information, which includes the process of combining sleep sound information and sleep environment information into multimodal data according to an embodiment of the present invention.

According to one embodiment of the present invention, a method for analyzing sleep state information using multimodal sleep sound information and sleep environment information includes: a first information acquisition step S100M of acquiring audio information in the time domain related to the user's sleep; a step S102M of performing data pre-processing on the first information; a second information acquisition step S110M of acquiring user sleep environment information related to the user's sleep; a step S112M of performing data pre-processing on the second information; a combining step S120M of fusing the data into multimodal data; a step S130M of inputting the multimodal data into a deep learning model; and a step S140M of acquiring sleep state information as an output of the deep learning model.

According to an embodiment of the present invention, the first information acquisition step S100M can acquire audio information in the time domain related to the user's sleep from a user terminal 10. The audio information in the time domain related to the user's sleep may include sound source information obtained from the sound source detection unit of the user terminal 10.

According to an embodiment of the present invention, in the step S102M of performing data pre-processing on the first information, the sleep sound information in the time domain can be converted into information including changes over the time axis of frequency components or into information in the frequency domain. Additionally, the information in the frequency domain can be represented as a spectrogram, which may be a mel spectrogram with a mel scale applied. By converting into a spectrogram, user privacy protection and data processing volume can be reduced. Furthermore, the converted information of sleep sound information in the time domain can be visualized, and in such cases, by using it as input for an image processing-based AI model, sleep state information can be acquired through image analysis.

According to an embodiment of the present invention, the step S102M of performing data pre-processing on the first information may further include a step of extracting features based on the audio information. For example, based on the acquired audio information in the time domain, the user's sleep breathing pattern can be extracted. For instance, the acquired audio information in the time domain can be converted into information including changes over the time axis of frequency components, and based on the converted information, the user's breathing pattern can be extracted. Alternatively, the audio information in the time domain can be converted into information in the frequency domain, and based on the audio information in the frequency domain, the user's sleep breathing pattern can be extracted.

In this case, the converted information can be visualized and used as input for an image processing-based AI model to output information such as the user's breathing pattern.

According to an embodiment of the present invention, the step S102M of performing data pre-processing on the first information may include a data augmentation process to obtain a sufficient amount of meaningful data for inputting sleep sound information into a deep learning model. Data augmentation techniques may include pitch shifting augmentation, TUT (Tile UnTile) augmentation, and noise addition augmentation. The aforementioned augmentation techniques are merely examples, and the present invention is not limited thereto.

According to an embodiment of the present invention, the method of adding in the mel scale can reduce the time required for hardware to process the data.

Meanwhile, the specific description regarding the types of noise mentioned above is merely a simple example for explaining the noise addition augmentation of the present invention, and the present invention is not limited thereto.

According to an embodiment of the present invention, the second information acquisition step S110M for acquiring user sleep environment information related to the user's sleep can acquire the user sleep environment information through a user terminal 10, an external server, or a network. The user's sleep environment information may refer to information related to sleep acquired from the space where the user is located. The sleep environment information may be sensing information acquired from the space where the user is located using a non-contact method.

The sleep environment information may include information on respiratory movements and body movements measured through radar. The sleep environment information may include information related to the user's sleep acquired from a smart watch, smart home-appliances, etc. The sleep environment information may be a photoplethysmography (PPG) signal. The sleep environment information may include heart rate variability (HRV) and heart rate obtained through a photoplethysmography (PPG) signal, which can be measured by a smart watch and smart ring. The sleep environment information may be an electroencephalography (EEG) signal. The sleep environment information may be an actigraphy signal measured during sleep.

According to an embodiment of the present invention, the step S112M of proceeding with the pre-processing of the second information may include a data augmentation process to obtain a sufficient amount of meaningful data for inputting the user's sleep environment information data into a deep learning model.

According to an embodiment of the present invention, the step S112M of proceeding with the pre-processing of the second information may include a step of processing the user's sleep environment information data to extract features. For example, if the second information is a photoplethysmography (PPG) signal, heart rate variability (HRV) and heart rate can be extracted from the photoplethysmography signal.

According to an embodiment of the present invention, the step S112M of proceeding with the pre-processing of the second information may include tile untile augmentation and noise addition augmentation if the user's sleep environment information data is obtained as image information. The aforementioned augmentation techniques are merely simple examples of image information augmentation techniques, and the present invention is not limited thereto. The user's sleep environment information may be information in various storage formats. Various methods can be employed for augmenting the user's sleep environment information.

According to an embodiment of the present invention, the step S120M of combining the pre-processed first information and second information into multimodal data is to combine the data for input into a deep learning model.

According to an embodiment of the present invention, the method of combining into multimodal data may involve combining the pre-processed first information and pre-processed second information into data of the same format. Specifically, the first information may be acoustic image information in the frequency domain, and the second information may be heart rate image information in the time domain obtained from a smart watch. In this case, since the first information and the second information are not in the same domain, they can be converted into the same domain for combination.

According to an embodiment of the present invention, the method of combining into multimodal data may involve combining the pre-processed first information and pre-processed second information into data of the same format. Specifically, the first information may be acoustic image information in the frequency domain, and the second information may be heart rate image information in the time domain obtained from a smart watch. In this case, since the first information and the second information are not in the same domain, each piece of data can be labeled as pertaining to the first information and the second information for use as input to the deep learning model.

According to an embodiment of the present invention, the step S120M of combining into multimodal data may proceed with the augmentation of the first information and the augmentation of the second information before combining. For example, the first information may be acoustic information in the time domain of the user, and the second information may be a photoplethysmography (PPG) signal, which can be combined into multimodal data. For example, the first information may be acoustic information in the time domain of the user or a spectrogram obtained by converting acoustic information in the time domain into acoustic information in the frequency domain, and the second information may be a photoplethysmography (PPG) signal, which can be combined into multimodal data.

According to an embodiment of the present invention, the step S120M of combining into multimodal data may proceed with the augmentation of the first information and the extraction of features, and then proceed with the augmentation of the second information for combination. For example, the first information may be a spectrogram obtained by converting acoustic information in the time domain of the user into acoustic information in the frequency domain, and the second information may be heart rate variability (HRV) or heart rate obtained from a photoplethysmography (PPG) signal, which can be combined into multimodal data.

According to an embodiment of the present invention, the step S120M of combining into multimodal data may proceed with the augmentation and feature extraction of the first information, and then proceed with the augmentation of the second information for combination. For example, the first information may be a user breathing pattern extracted based on the user's acoustic information, and the second information may be heart rate variability (HRV) or heart rate obtained from a photoplethysmography (PPG) signal, which can be combined into multimodal data.

According to an embodiment of the present invention, the step S120 of combining multimodal data may proceed with the first information augmentation and feature extraction, and the second information augmentation and feature extraction to combine them. For example, the first information may be a user breathing pattern extracted based on the user's sound information, and the second information may be heart rate variability (HRV) or heart rate obtained from photoplethysmography (PPG), which can be combined into multimodal data.

According to an embodiment of the present invention, the step S130M of inputting multimodal combined data into a deep learning model may process the data into a form required for inputting into the deep learning model.

According to an embodiment of the present invention, the step S140M of acquiring sleep state information as an output of the deep learning model may infer sleep state information by using the multimodal combined data as input to the deep learning model. The sleep state information may be information regarding the user's state of sleep.

According to an embodiment of the present invention, the user's sleep state information may include sleep stage information that represents the user's sleep in stages. The stages of sleep can be divided into NREM (non-REM) sleep and REM (Rapid Eye Movement) sleep, and NREM sleep can be further divided into multiple stages (e.g., two stages of Light and Deep, or four stages from N1 to N4. The setting of sleep stages may be defined as general sleep stages but can also be arbitrarily set to various sleep stages depending on the designer.

According to an embodiment of the present invention, the user's sleep state information may include sleep event information that represents disorders related to sleep or behaviors during sleep occurring in the user's sleep. Specifically, the sleep event information occurring during the user's sleep may include information on sleep apnea and hypopnea due to the user's sleep disorders. Furthermore, specifically, the sleep event information occurring during the user's sleep may include whether the user snores, the duration of snoring, whether the user talks in their sleep, the duration of sleep talking, whether the user tosses and turns, and the duration of tossing and turning. The described user's sleep event information is merely an example for representing events occurring during the user's sleep and is not limited thereto.

### An Embodiment of a Method for Analyzing Sleep State Information Using Multimodal Data (Concept-B)

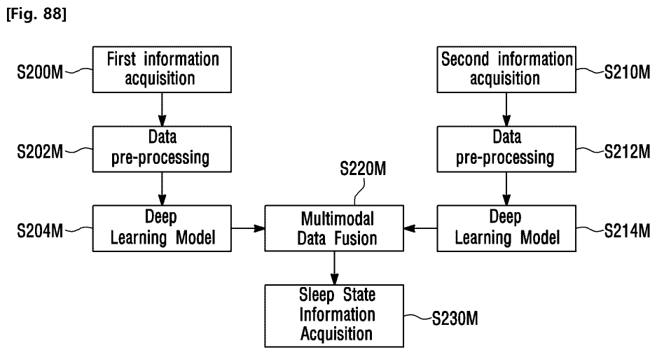

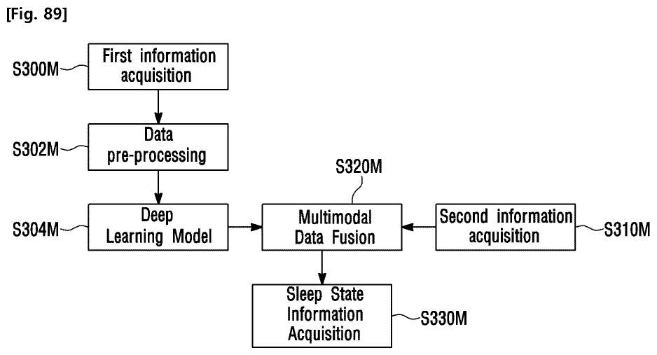

FIG. 88 is a flowchart for explaining a method for analyzing sleep state information, which includes the step of combining sleep sound information and sleep environment information inferred according to an embodiment of the present invention into multimodal data.

To achieve the object of the present invention, according to one embodiment, a method for analyzing sleep state information using multimodal sleep sound information and sleep environment information includes a first information acquisition step S200M of acquiring sound information in the time domain related to the user's sleep, a step S202M of performing pre-processing of the first information, a step S204M of inferring sleep-related information by inputting the first information into a deep learning model, a second information acquisition step S210M of acquiring user sleep environment information related to the user's sleep, a step S212M of performing pre-processing of the second information, a step S214M of inferring sleep-related information by inputting the second information into a deep learning model, a combining step S220M of combining the data into multimodal data, and a step S230M of acquiring sleep state information through the combination of multimodal data.

According to an embodiment of the present invention, the first information acquisition step S200M may acquire sound information in the time domain related to the user's sleep from the user terminal 10. The sound information in the time domain related to the user's sleep may include sound source information obtained from the sound source detection unit of the user terminal 10.

According to an embodiment of the present invention, in the step S202M of performing data pre-processing of the first information, time-domain audio information can be converted into information on frequency components over the time axis or into information in the frequency domain. Additionally, the information in the frequency domain can be represented as a spectrogram, which may be a mel spectrogram with a mel scale applied. By converting into a spectrogram, user privacy can be protected and the amount of data processing can be reduced.

According to an embodiment of the present invention, the step S202 of performing data pre-processing of the first information may include a data augmentation process to obtain a sufficient amount of meaningful data for inputting sleep sound information into a deep learning model. The data augmentation techniques may include pitch shifting augmentation, TUT (Tile UnTile) augmentation, and noise addition augmentation. The aforementioned augmentation techniques are merely simple examples, and the present invention is not limited thereto.

According to an embodiment of the present invention, the method of adding in the mel scale may reduce the time required for hardware to process data.

Meanwhile, the specific description of the types of noise mentioned above is merely a simple example for explaining the noise addition augmentation of the present invention, and the present invention is not limited thereto.

According to an embodiment of the present invention, the second information acquisition step S210M for acquiring user sleep environment information related to the user's sleep can acquire the user sleep environment information through a user terminal 10, an external server 20, or a network. The user's sleep environment information may refer to information related to sleep acquired from the space where the user is located. The sleep environment information may be sensing information acquired from the space where the user is located using a non-contact method. The sleep environment information may include information on respiratory movements and body movements measured through radar. The sleep environment information may be information related to the user's sleep acquired from a smart watch, smart home-appliance, etc. The sleep environment information may include heart rate variability (HRV) and heart rate obtained through photoplethysmography (PPG), and the photoplethysmography signal can be measured by a smart watch and smart ring. The sleep environment information may include electroencephalography (EEG) signals. The sleep environment information may include actigraphy signals measured during sleep.

According to an embodiment of the present invention, the step S212M of pre-processing the second information may include a data augmentation process to obtain a sufficient amount of meaningful data for inputting the user's sleep environment information into a deep learning model.

According to an embodiment of the present invention, the step S212M of pre-processing the second information may include TUT (Tile UnTile) augmentation and noise addition augmentation if the user's sleep environment information data is obtained as image information. The aforementioned augmentation techniques are merely simple examples of image information augmentation techniques, and the present invention is not limited thereto. The user's sleep environment information may be information in various storage formats. Various methods can be employed for augmenting the user's sleep environment information.

According to an embodiment of the present invention, the step S204M of inferring sleep-related information using the pre-processed first information as input to a deep learning model can infer sleep-related information using the input of a pre-trained deep learning model.

According to an embodiment of the present invention, the pre-trained deep learning model can use the inferred data as input for self-learning through the inferred data.

According to an embodiment of the present invention, the deep learning sleep analysis model that infers sleep-related information using the first information on sleep sound as input may include a feature extraction model and a feature classification model.

In an embodiment of the present invention, the feature extraction model of the deep learning sleep analysis model is pre-trained through a one-to-one proxy task, where a single spectrogram is input to predict sleep state information corresponding to the spectrogram. When employing a CNN deep learning model in the feature extraction model according to an embodiment of the present invention, the structure of a Fully Connected Layer (FC) or a Fully Connected Neural Network (FCN) may be adopted for training. When employing a MobileViTV2 deep learning model in the feature extraction model according to an embodiment of the present invention, the structure of an Intermediate Layer may be adopted for training.

In an embodiment of the present invention, the feature classification model of the deep learning sleep analysis model is trained to predict sleep state information for each spectrogram by inputting a plurality of consecutive spectrograms and to analyze the sequence of the plurality of consecutive spectrograms to predict or classify overall sleep state information.

According to an embodiment of the present invention, the step S214M of inferring sleep-related information by using pre-processed second information as input to an inference model can infer sleep-related information by using a pre-trained inference model as input. The pre-trained inference model may be the aforementioned deep learning sleep analysis model, but is not limited thereto, and the pre-trained inference model may be of various types of inference models to achieve the objective. Various methods may be employed for the pre-trained inference model.

According to an embodiment of the present invention, the step S220M of combining first information and second information, which have undergone data pre-processing, into multimodal data involves combining the information to determine sleep state information.

In one embodiment of the present invention, the method of combining into multimodal data may involve combining sleep information inferred through pre-processed first information and information inferred through pre-processed second information into data of the same format.

According to an embodiment of the present invention, the step S230M of acquiring sleep state information through multimodal data fusion involves combining data obtained multimodally to determine the user's sleep state information. The sleep state information may pertain to information about the user's sleep state.

In one embodiment of the present invention, the step S230M of acquiring sleep state information through multimodal data fusion involves combining the user's hypnogram inferred in step S204M using pre-processed first information as input to a deep learning model with the user's hypnogram inferred in step S214M using pre-processed second information as input to an inference model. For example, by overlaying each hypnogram and adopting sleep stage information for matching parts, and determining whether to adopt sleep stage information for non-matching parts by assigning weights, sleep state information can be acquired.

In one embodiment of the present invention, the step S230M of acquiring sleep state information through multimodal data fusion involves combining the user's hypnodensity graph inferred in step S204M using pre-processed first information as input to a deep learning model with the user's hypnodensity graph inferred in step S214M using pre-processed second information as input to an inference model. For example, by substituting the probabilities of each hypnodensity graph into a formula, the sleep stage with the highest confidence at each time can be obtained as the user's sleep stage information. For instance, if the confidence over time in each hypnodensity graph exceeds a predefined confidence threshold, it is adopted as the user's sleep stage information, and if there is no sleep stage information exceeding the predefined confidence threshold, it is adopted as sleep stage information through weighting, thereby acquiring sleep state information.

In one embodiment of the present invention, the step S230M of acquiring sleep state information through multimodal data fusion involves combining the user's hypnogram inferred in step S204M using pre-processed first information as input to a deep learning model with the user's hypnodensity graph inferred in step S214M using pre-processed second information as input to an inference model. For example, if the sleep stage displayed in the hypnogram and the confidence of the hypnodensity graph exceed a predefined threshold, it is adopted as the user's sleep stage, thereby acquiring the user's sleep state information. For instance, if the sleep stage displayed in the hypnogram and the confidence of the hypnodensity graph do not exceed the predefined threshold, it is calculated by assigning weights and adopted as the user's sleep stage, thereby acquiring highly reliable user sleep state information.

According to an embodiment of the present invention, the user's sleep state information may include sleep stage information that represents the user's sleep in stages. The stages of sleep can be classified into NREM (non-REM) sleep and REM (Rapid Eye Movement) sleep, and NREM sleep can be further divided into multiple stages (e.g., two stages of Light and Deep, or four stages from N1 to N4. The setting of sleep stages may be defined as general sleep stages, but can also be arbitrarily set to various sleep stages depending on the designer.

According to an embodiment of the present invention, the user's sleep state information may include sleep stage information that displays the user's sleep in stages. Methods for displaying sleep stages may include a hypnogram that displays sleep stages on a graph and a hypnodensity graph that displays the probability of each sleep stage on a graph, but are not limited to these methods.

According to an embodiment of the present invention, the user's sleep state information may include sleep event information that represents disorders related to sleep or behaviors during sleep occurring in the user's sleep. Specifically, the sleep event information occurring during the user's sleep may include information on sleep apnea and hypopnea due to the user's sleep disorders. Additionally, specifically, the sleep event information occurring during the user's sleep may include whether the user snores, the duration of snoring, whether the user talks in their sleep, the duration of sleep talking, whether the user tosses and turns, and the duration of tossing and turning. The described user's sleep event information is merely an example to represent events occurring during the user's sleep and is not limited thereto.

### An Embodiment of a Method for Analyzing Sleep State Information Using Multimodal Data (Concept-C)

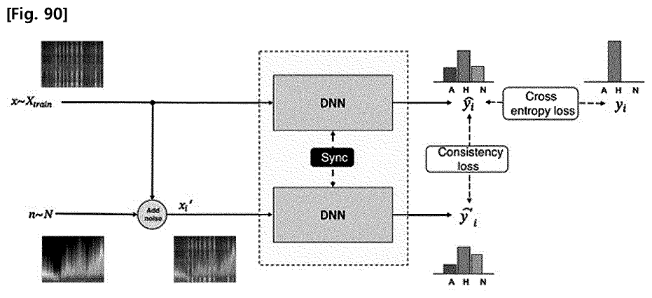

FIG. 89 is a flowchart illustrating a method for analyzing sleep state information, including a step of combining inferred sleep sound information with sleep environment information and multimodal data, according to an embodiment of the present invention.

To achieve the object of the present invention, according to an embodiment, a method for analyzing sleep state information using multimodal sleep sound information and sleep environment information includes a first information acquisition step S300M for acquiring audio information in the time domain related to the user's sleep, a step S302M for performing pre-processing of the first information, a step S304M for inferring sleep-related information using the first information as input to a deep learning model, a second information acquisition step S310M for acquiring user sleep environment information related to the user's sleep, a combining step S320M for fusing data multimodally, and a step S330M for acquiring sleep state information through the fusion of multimodal data.

According to an embodiment of the present invention, the first information acquisition step S300M may acquire audio information in the time domain related to the user's sleep from the user terminal 10. The audio information in the time domain related to the user's sleep may include sound source information obtained from the sound source detection unit of the user terminal 10.

According to an embodiment of the present invention, in the step S302M for performing data pre-processing of the first information, the time-domain audio information can be converted into information in the frequency domain. Additionally, the information in the frequency domain may be represented as a spectrogram, which may be a mel spectrogram with the mel scale applied. By converting to a spectrogram, user privacy protection and data processing volume can be reduced.

According to an embodiment of the present invention, in the step S302M for performing data pre-processing of the first information, a data augmentation process may be included to obtain a sufficient amount of meaningful data for inputting sleep sound information into a deep learning model. Data augmentation techniques may include pitch shifting augmentation, TUT (Tile UnTile) augmentation, and noise addition augmentation. The aforementioned augmentation techniques are merely simple examples, and the present invention is not limited thereto.

According to an embodiment of the present invention, the method of adding in the Mel scale can reduce the time required for hardware to process data.

Meanwhile, the specific description regarding the types of noise mentioned above is merely an example to explain the noise addition augmentation of the present invention, and the present invention is not limited thereto.

According to an embodiment of the present invention, the second information acquisition step S310 for acquiring user sleep environment information related to the user's sleep can obtain the user sleep environment information through a user terminal 10, an external server, or a network. The user's sleep environment information may refer to information related to sleep acquired from the space where the user is located. The sleep environment information may be sensing information acquired from the space where the user is located using a non-contact method.

The sleep environment information may include respiratory movement and body movement information measured through radar. The sleep environment information may be information related to the user's sleep acquired from a smart watch, smart home-appliance, etc. The sleep environment information may include heart rate variability (HRV) and heart rate obtained through photoplethysmography (PPG), and the photoplethysmography signal may be measured by a smart watch and smart ring. The sleep environment information may include electroencephalography (EEG) signals. The sleep environment information may include actigraphy signals measured during sleep. The sleep environment information may be labeling data representing the user's information. Specifically, the labeling data may include the user's age, disease status, physical condition, race, height, weight, and body mass index, which are merely examples of labeling data representing the user's information and are not limited thereto. The aforementioned sleep environment information is merely an example of information that may affect the user's sleep and is not limited thereto.

According to an embodiment of the present invention, the step S304M of inferring information related to sleep by using pre-processed first information as input to a deep learning model can infer information related to sleep by using a pre-trained deep learning model as input.

According to an embodiment of the present invention, the deep learning sleep analysis model that infers information related to sleep by using first information related to sleep sound as input may include a feature extraction model and a feature classification model.

In the deep learning sleep analysis model according to an embodiment of the present invention, the feature extraction model can be pre-trained by a one-to-one proxy task that inputs a spectrogram and is trained to predict sleep state information corresponding to the spectrogram. When employing a CNN deep learning model in the feature extraction model according to an embodiment of the present invention, the structure of a fully connected layer (FC) or a fully connected neural network (FCN) may be adopted for training. When employing a MobileViTV2 deep learning model in the feature extraction model according to an embodiment of the present invention, the structure of an intermediate layer may be adopted for training.

In the deep learning sleep analysis model according to an embodiment of the present invention, the feature classification model can be trained to predict the sleep state information of each spectrogram by inputting a plurality of consecutive spectrograms and to analyze the sequence of the plurality of consecutive spectrograms to predict or classify time-series sleep state information.

According to an embodiment of the present invention, the step S320M of combining the pre-processed first information and second information into multimodal data involves combining the data to input multimodal data into a deep learning model.

According to an embodiment of the present invention, the method of combining into multimodal data may involve combining the sleep information inferred through the pre-processed first information and the information inferred through the pre-processed second information into data of the same format.

According to an embodiment of the present invention, the step S330M of acquiring sleep state information through multimodal data fusion involves combining the data obtained multimodally to determine the user's sleep state information. The sleep state information may be information regarding the state of the user's sleep.

According to an embodiment of the present invention, the user's sleep state information may include sleep stage information that represents the user's sleep in stages. The stages of sleep can be divided into NREM (non-REM) sleep and REM (Rapid Eye Movement) sleep, and NREM sleep can further be subdivided into multiple stages (e.g., two stages of Light and Deep, or four stages from N1 to N4. The setting of sleep stages may be defined as general sleep stages, but can also be arbitrarily set to various sleep stages depending on the designer.

According to an embodiment of the present invention, the user's sleep state information may include sleep event information that represents disorders related to sleep or behaviors during sleep occurring in the user's sleep. Specifically, the sleep event information occurring during the user's sleep may include information on sleep apnea and hypopnea due to the user's sleep disorders. Additionally, specifically, the sleep event information occurring during the user's sleep may include whether the user snores, the duration of snoring, whether the user talks in their sleep, the duration of sleep talking, whether the user tosses and turns, and the duration of tossing and turning. The described user's sleep event information is merely an example to represent events occurring during the user's sleep and is not limited thereto.

### Real-Time Sleep Analysis According to an Embodiment of the Present Invention

According to an embodiment of the present invention, the analysis of sleep state information based on sound information may include a detection stage for sleep events (e.g., apnea, hypopnea, snoring, sleep talking, etc.). However, since the characteristics of sleep sound patterns are reflected over time, it may be difficult to grasp with only short sound data at a specific point in time. Therefore, to model sound information, the analysis should be performed based on the time-series characteristics of the sound information.

Furthermore, sleep events occurring during sleep (e.g., apnea, hypopnea, snoring, sleep talking, etc.) have various characteristics related to sleep events. For example, during an apnea event, there is no sound, but when the apnea event ends, a loud sound may occur as air passes through again, and the characteristics of the apnea event can be learned in a time-series manner to detect sleep events.

### Differences in Deep Neural Networks for Real-Time Sleep Event Detection

According to an embodiment of the present invention, to detect sleep events occurring during sleep, the deep neural network structure for analyzing the aforementioned sleep stages can be modified and used. Specifically, while sleep stage analysis requires time-series learning of sleep sounds, sleep event detection generally occurs between 10 to 60 seconds, so it is sufficient to accurately detect one epoch or two epochs of 30 seconds each. Therefore, the deep neural network structure for analyzing sleep stages according to an embodiment of the present invention can reduce the input and output quantities of the deep neural network structure for sleep stage analysis. For example, if the deep neural network structure for analyzing sleep stages processes 40 mel spectrograms to output 20 epochs of sleep stages, the deep neural network structure for detecting sleep events can process 14 mel spectrograms to output 10 epochs of sleep event labels. Here, the sleep event labels may include no event, apnea, hypopnea, snoring, tossing and turning, etc., but are not limited thereto.

Additionally, the deep neural network structure for detecting sleep events occurring during sleep according to an embodiment of the present invention may include a feature extraction model and a feature classification model. Specifically, the feature extraction model extracts characteristics of sleep events found in each mel spectrogram, and the feature classification model detects multiple epochs to find epochs containing sleep events and analyzes neighboring characteristics to predict and classify the types of sleep events in a time-series manner.

### Class Weights for Real-Time Sleep Event Detection

According to an embodiment of the present invention, the method for detecting sleep events occurring during sleep may assign class weights to solve the class imbalance problem of each sleep event. Specifically, among the sleep events occurring during sleep, “no event” can have a dominant influence on the overall sleep length, which may cause a decrease in sleep event learning efficiency. Therefore, by assigning higher weights to other sleep events than “no event,” learning efficiency and accuracy can be improved. For example, if the sleep event classes are classified into three types: “no event,” “apnea,” and “hypopnea,” to reduce the impact of “no event” on learning, weights of 1.0 for “no event,” 1.3 for “apnea,” and 2.1 for “hypopnea” can be assigned.

### Consistency Training for Real-Time Sleep Event Detection

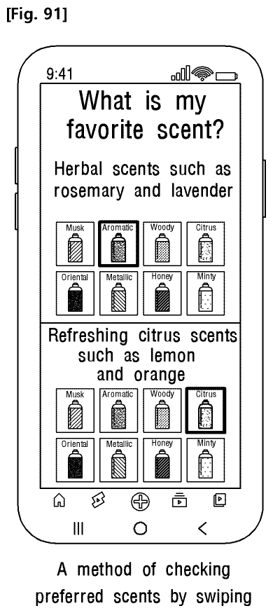

FIG. 90 is a diagram illustrating consistency training according to an embodiment of the present invention.

According to an embodiment of the present invention, the step of detecting sleep events occurring during sleep can utilize consistency training, as shown in FIG. 90, to detect sleep events occurring during sleep in home and noise environments. Consistency training is a type of semi-supervised learning model, and the consistency training according to an embodiment of the present invention may be a method of performing training with data to which noise is intentionally added and data to which noise is not intentionally added.

Additionally, the consistency training according to an embodiment of the present invention may be a method of performing training by generating data of a virtual sleep environment using the noise of the target environment.

According to an embodiment of the present invention, the noise intentionally added may be the noise of the target environment, where the noise of the target environment may be, for example, noise acquired from environments other than polysomnography. Specifically, in detecting sleep events, various noises can be added by adjusting the SNR and the type of noise to make it similar to the actual user's environment. Through this, the types of noise obtained from various laboratories and the noise occurring in actual home environments can be collected and learned.

According to embodiments of the present invention, for convenience, data to which noise is intentionally added is referred to as Corrupted data. Corrupted data preferably means data to which the noise of the target environment is intentionally added.

Also, for convenience, data to which noise is not intentionally added is referred to as Clean data. Here, Clean data may not have noise intentionally added, but it may still substantially include noise.

The Clean data used in consistency training according to an embodiment of the present invention may be data acquired in a specific environment (preferably, a polysomnography environment), and the Corrupted data may be data acquired in another environment or target environment (preferably, an environment other than polysomnography).

The Corrupted data according to an embodiment of the present invention may be data to which noise acquired from another environment or target environment (preferably, an environment other than polysomnography) is intentionally added to the Clean data.

In consistency training, when Clean data and Corrupted data are respectively input into the same deep learning model, a loss function or consistency loss is defined so that each output becomes equal, and training can be performed to promote consistent prediction.

### Home Noise Consistency Training

According to one embodiment of the present invention, the detection of sleep events (e.g., apnea, hypopnea, snoring, sleep talking, etc.) occurring during sleep may include Home Noise Consistency Training. Home Noise Consistency Training can enable the model to operate robustly even with noise present in a home environment. This training allows the model to become noise-resistant by conducting consistency training so that it outputs similar predictions regardless of the presence of noise.

According to one embodiment of the present invention, the detection of sleep events occurring during sleep can proceed with Home Noise Consistency Training. Home Noise Consistency Training may include a consistency loss function. For example, consistency loss can be defined as the mean squared error (MSE) between the prediction of clean sleep breathing sounds and the prediction of their corrupted version.

According to one embodiment of the present invention, Home Noise Consistency Training can generate corrupted sounds by randomly sampling data from training noise and adding noise to clean sleep breathing sounds with a random SNR between −20 and 5.

According to one embodiment of the present invention, Home Noise Consistency Training can be conducted such that the length of the input sequence is 14 epochs, and the total length of sampled noise is at least 7 minutes. Through this, the detection of sleep events according to the present invention can detect information within a shorter time compared to sleep stage analysis according to the present invention, thereby increasing the accuracy of sleep event detection.

Regression Analysis for Estimating AHI Value from Event Detection

FIG. 34 is a diagram for explaining a linear regression analysis function utilized to analyze the Apnea-Hypopnea Index (AHI), which is an index of sleep apnea occurrence, through sleep events occurring during sleep according to one embodiment of the present invention.

According to one embodiment of the present invention, the AHI index, which signifies the number of respiratory events occurring per unit time (e.g., 1 hour), can be analyzed independently of the length of an epoch for sleep stage analysis. Specifically, two or three short sleep events may be included during one epoch, and one long sleep event may be included during multiple epochs. According to one embodiment of the present invention, a regression analysis function can be used to estimate the actual number of events occurring from the number of epochs in which sleep events occurred. For example, a RANSAC (Random Sample Consensus) regression analysis model can be used. The RANSAC regression analysis model is one method of estimating the parameters of a fitting model, which involves randomly sampling data and then selecting the model that maximally fits.

Multi-Task Analysis through Multi-Heads

According to one embodiment of the present invention, a method for analyzing sleep state may include analysis through a deep learning model. The deep learning model according to one embodiment of the present invention can perform multi-task learning and/or multi-task analysis. Specifically, multi-task learning and multi-task analysis can simultaneously learn tasks according to the embodiments of the present invention described above (e.g., multimodal learning, real-time sleep event analysis, sleep stage analysis, etc.).

According to an embodiment of the present invention, a deep learning model for analyzing sleep state information can perform multi-task learning and multi-task analysis.

Specifically, for multi-task learning and analysis, the deep learning model may adopt a structure with multiple heads. Each of the multiple heads can be responsible for a specific task or task (e.g., multimodal learning, real-time sleep event analysis, sleep stage analysis, etc.). For example, the deep learning model may have a structure with a total of three heads: a first head, a second head, and a third head. The first head may perform inference and/or classification on sleep stage information, the second head may perform detection and/or classification of sleep apnea and hypopnea during sleep events, and the third head may perform detection and classification of snoring during sleep events. The specific description of the tasks or tasks of the aforementioned heads is merely an example for explaining the present invention and is not limited thereto. The deep learning model according to the present invention can proceed with multi-task learning and analysis through a structure with multiple heads, and can optimize multiple tasks or specific tasks by increasing data efficiency.

### Effects of the Sleep Analysis Method According to the Present Invention

Compared to the results of polysomnography (PSG), it was confirmed that the results of the sleep analysis model using sleep sound information as input are very accurate.

Existing sleep analysis models predicted sleep stages using inputs such as ECG (Electrocardiogram) or HRV (Heart Rate Variability), but the present invention can perform sleep stage analysis and inference by converting sleep sound information into frequency domain information, spectrogram, or mel spectrogram as input. Therefore, by converting sleep sound information into frequency domain information, spectrogram, or mel spectrogram as input, unlike existing sleep analysis models, it is possible to sense or acquire sleep stages in real-time through the analysis of the singularity of sleep patterns.

The present invention can identify the points where sleep disorders (sleep apnea, sleep hyperpnea, sleep hypopnea) occur while analyzing the user's sleep in real-time. If a stimulus (tactile, auditory, olfactory, etc.) is provided to the user at the moment a sleep disorder occurs, the sleep disorder can be temporarily alleviated. That is, the present invention can interrupt the user's sleep disorder and reduce the frequency of sleep disorders based on accurate event detection related to sleep disorders. Additionally, according to the present invention, performing sleep analysis in a multimodal manner allows for very accurate sleep analysis.

### Method of Providing Scent

According to one embodiment of the present invention, after randomly providing one of the types of scents mentioned above, user modeling can be performed based on user feedback on the provided scent. For example, if a randomly selected “aroma scent” is provided to a user falling asleep or about to fall asleep 100f, and the user's feedback after waking is positive, the user terminal 200f or server 20 can recognize that the user prefers the “aroma scent.”

According to another embodiment of the present invention, if the user terminal 200f or server 20 recognizes that the user prefers a specific scent through the method of emotional modeling described below, user modeling can be performed again on the scent provided by the method of emotional modeling. For example, if an “aroma scent” recognized as preferred by a user falling asleep or about to fall asleep 100 is provided, and the user's feedback after waking is negative, the user terminal 200f or server 20 can recognize that the user does not prefer the “aroma scent.”

According to another embodiment of the present invention, after randomly providing one of the types of scents mentioned above, quantitative modeling based on environment sensing information described below can be performed by the randomly provided scent. For example, if a randomly selected “aroma scent” is provided to a user falling asleep or about to fall asleep 100, and the time taken for the user to wake up after falling asleep (WASO) is relatively short, or the number of times the user wakes up during sleep is relatively high, or the time taken for the user's deep sleep to first appear is relatively long, the user terminal 200f or server 20 can recognize that the user does not prefer the “aroma scent.”

According to another embodiment of the present invention, if the user terminal 200f or server 20 recognizes that the user prefers a specific scent through the method of emotional modeling described below, quantitative modeling based on environment sensing information described below can be performed on the scent provided by the method of emotional modeling.

For example, if an “aroma scent” recognized as preferred by a user falling asleep or about to fall asleep 100 is provided, and the time taken for the user to wake up after falling asleep (WASO) is relatively short, or the number of times the user wakes up during sleep is relatively high, or the time taken for the user's deep sleep to first appear is relatively long, the user terminal 200f or server 20 can recognize that the user does not prefer the “aroma scent.”

### User Modeling

### Emotional Modeling

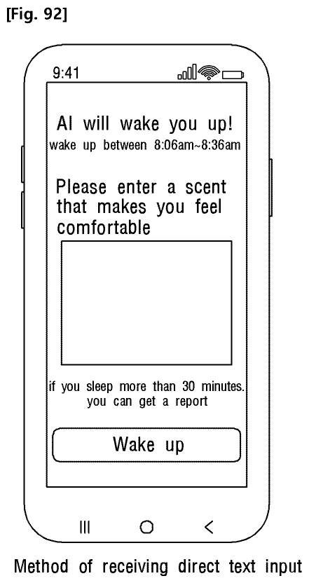

FIG. 91 is a diagram illustrating a user modeling method of emotional modeling according to the present invention, wherein a user checks preferred scents by swiping.

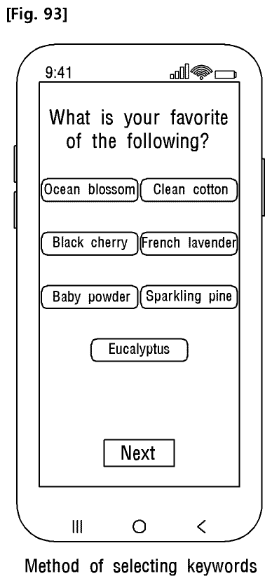

FIG. 92 is a diagram illustrating a user modeling method of emotional modeling according to the present invention, wherein a user inputs text related to preferred scents.

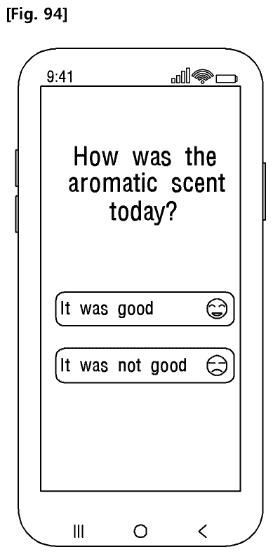

FIG. 93 is a diagram illustrating a user modeling method of emotional modeling according to the present invention, wherein a user selects keywords for preferred scents.

User modeling, in the method of providing scents, may include emotional modeling and quantitative modeling based on environment sensing information. Emotional modeling may include modeling scents associated with good memories or scents that the user finds comforting.

Specifically, as shown in FIG. 91, emotional modeling within user modeling may include a method where the user checks scents associated with good memories or scents that the user finds comforting during the process of swiping through scent types. For example, if the user terminal 10 or server 20 detects that the user lingers on a particular scent type for a relatively long time, it may recognize that the user prefers that scent type. In a specific example, as shown in FIG. 1, the user interface for emotional modeling may provide a phrase like “What is my preferred scent?” at the top, and the user may swipe through the interface for emotional modeling, lingering on “herbal scents like rosemary and lavender” for a relatively long time, or tapping to select “herbal scents like rosemary and lavender.” In this case, the user terminal 10 or server 20 may recognize that the user prefers “herbal scents like rosemary and lavender.” Accordingly, aromatic scents with “herbal scents like rosemary and lavender” may be provided during the user's sleep onset stage, REM sleep stage, and awakening stage.

As shown in FIG. 92, emotional modeling within user modeling may include a method where the user inputs content about preferred scents through text. For example, if the user terminal 10 asks the user, as shown in FIG. 92, “Please input scents that make you feel comfortable,” the user terminal 10 or server 20 may recognize the text input by the user as scents associated with good memories or scents that the user finds comforting. In a specific example, if the user inputs “I liked the smell of tangerines at the tangerine farm during my trip to Jeju Island,” the user terminal 10 or server 20 may recognize the user's emotional modeling as a preference for “refreshing citrus scents like tangerine and orange,” and accordingly, the environment adjustment device 30 may provide citrus scents with “refreshing citrus scents like tangerine and orange” during the user's sleep onset stage, REM sleep stage, and awakening stage.

As shown in FIG. 93, emotional modeling within user modeling may include a method where the user selects keywords for preferred scents. For example, if the user terminal 10 asks the user to “Select your preferred scent,” and the user selects the scent “clean cotton,” the user terminal 10 or server 20 may recognize that the selected “clean cotton” is a scent associated with good memories or a scent that the user finds comforting, and accordingly, “clean cotton” scent may be provided during the user's sleep onset stage, REM sleep stage, and awakening stage.

### Post-Awakening Feedback

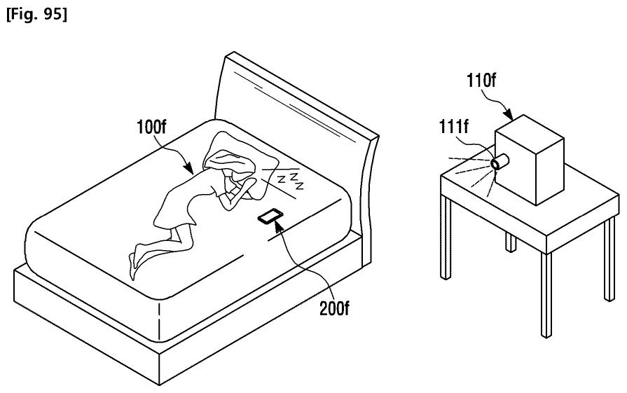

FIG. 94 illustrates a diagram for explaining a user modeling method for receiving feedback after waking up regarding a scent provided to the user as part of the emotional modeling method according to the present invention.

As shown in FIG. 94, feedback can be received after waking up regarding a scent provided by a user modeling method where the user swipes to check preferred scents, a scent provided by a user modeling method where the user inputs text related to preferred scents, a scent provided by a user modeling method where the user selects keywords for preferred scents, or a randomly provided scent.

Specifically, if the scent provided by the user modeling method where the user swipes to check preferred scents, the scent provided by the user modeling method where the user inputs text related to preferred scents, the scent provided by the user modeling method where the user selects keywords for preferred scents, or the randomly provided scent is the “aromatic scent” shown in FIG. 94, a user interface in the form of a sentence such as “How was the aromatic scent today?” can be provided. Alternatively, a user interface in the form of a sentence such as “Did you like the scent while sleeping?” can be provided without offering information about the scent.

Accordingly, a user interface can be provided to allow the user to select between mutually exclusive sentences such as “I liked it” and “Not really,” or between “Like” and “Dislike.” Additionally, a user interface can be provided to allow the selection between a smiling emoji and a non-smiling emoji, and as shown in FIG. 94, a user interface providing both sentences and emojis can also be offered.

For a specific example, according to one embodiment of the present invention, if a user interface in the form of a sentence such as “How was the aromatic scent today?” is provided and the user selects a user interface with a sentence like “Like,” the user terminal 10 or server 20 can recognize that the user prefers the “aromatic scent.” Accordingly, the user can be provided with citrus or “aromatic scent” during the sleep onset stage, REM sleep stage, and waking stage.

Conversely, if a user interface in the form of a sentence such as “How was the aromatic scent today?” is provided and the user selects a user interface with a sentence like “Dislike,” the user terminal 10 or server 20 can recognize that the user does not prefer the “aromatic scent.” Accordingly, a scent other than “aromatic scent” can be provided during the user's sleep onset stage, REM sleep stage, and waking stage.

FIG. 95 illustrates a diagram for explaining the provision of scent to a sleeping user by the scent-providing device according to the present invention.

As illustrated, the user terminal 200f and the scent-providing device 110f can sense the environment sensing information of the environment of a user 100f who is falling asleep or about to fall asleep, analyze the sleep state information of the user 100f who is falling asleep or about to fall asleep, and when the sleep state information of the user 100f who is falling asleep or about to fall asleep is in a WAKE state, Light state, REM state, etc., the scent-providing device 110f can spray scent from the discharge port 111f of the scent-providing device.

For a specific example, if the user terminal 200f or server 20 recognizes that the user 100f who is falling asleep or about to fall asleep prefers an aroma scent according to the emotional modeling described below, the scent-providing device 110f can spray aroma scent from the discharge port 111f of the scent-providing device when the sleep state information of the user 100f who is falling asleep or about to fall asleep is in a WAKE state, Light state, REM state, etc.

### Quantitative Modeling by Environment Sensing Information

In the method of providing a scent, user modeling may include emotional modeling and quantitative modeling based on environment sensing information. Quantitative modeling based on environment sensing information may be a modeling method based on the user's sleep results. Specifically, the user terminal 10 or server 20 can evaluate the quality of sleep when a specific scent is provided, allowing for an assessment of the specific scent provided to the user.

According to one embodiment of the present invention, the user terminal 10 or server 20 can evaluate the quality of sleep when a specific scent is provided, and if the user's sleep quality is poor, a penalty can be imposed on the specific scent. For example, if a specific scent is provided and the time taken for the user to wake after falling asleep (WASO) is relatively short, or if the user wakes up frequently during sleep, or if the time taken for the user's deep sleep to first appear is relatively long, the user terminal 10 or server 20 may impose a penalty on the scent.

Hereinafter, a detailed description is provided for the case where the sleep quality evaluation indicator according to an embodiment of the present invention is a score that numerically records the analysis of sleep.

According to one embodiment of the present invention, a sleep score, which is a type of sleep quality evaluation indicator, can be calculated based on at least one of the user's sleep onset latency, sleep onset time, wake-up time, total sleep time, and sleep time per sleep stage. The user's total sleep time may be the absolute value of the difference between the bedtime and wake-up time. The user's sleep time per sleep stage can be represented as a ratio or time of the measured user's sleep stages (e.g., wake, deep sleep, light sleep, REM sleep, etc.).

According to one embodiment of the present invention, the sleep score may be a comprehensive score of sleep with a maximum score of 100, providing a numerical evaluation of sleep. The sleep score can be calculated using a predefined sleep score calculation formula.

For example, scores corresponding to each sleep stage (e.g., REM sleep, deep sleep, light sleep, wake, etc.) can be assigned, and by substituting these into a predefined sleep score calculation formula, a final score can be obtained. The final score can be divided by the maximum possible score and multiplied by 100 to yield the comprehensive sleep score. By providing an evaluation of sleep as a score, an intuitive assessment of sleep can be offered.

According to the present invention, if the sleep score of a user who received a specific scent is high, it can be interpreted that the scent had a positive impact on the user's sleep experience. Conversely, if the sleep score is low, it may be interpreted that the scent did not have a positive impact on the user's sleep experience.

### Scent-Providing Device

### Closed-Loop Control

According to the present invention, reinforcement learning is a learning method structured as a closed-loop, where after performing a predetermined action in a specific environment, the action is evaluated and updated (or modified) through a reward function. Hereinafter, the closed-loop control or reinforcement learning method of the scent-providing device according to one embodiment of the present invention will be described.

According to the present invention, in a method for controlling an electronic device that provides a predetermined scent in response to predetermined scent-providing information, the method may include generating first scent-providing information based on sleep state information generated over a time corresponding to one or more epochs, and generating second scent-providing information based on sleep state information generated over a time corresponding to one or more epochs after the provision of the first scent has commenced.

Specifically, one or more epochs may be set as data corresponding to 30-second intervals, and may indicate the probability (i.e., sleep stage probability information) of belonging to any of the four classes (Wake, Light, Deep, REM) of sleep stages in periodic units according to one or more epochs.

Through a hypnodensity graph according to an embodiment of the present invention, when predicting sleep stage information, it is possible to indicate the probability (i.e., sleep stage probability information) of belonging to any of the four classes (Wake, Light, Deep, REM) of sleep stages in periodic units according to one or more epochs. Additionally, it is possible to indicate the probability of belonging to any of the five classes (Wake, N1, N2, N3, REM), the probability of belonging to any of the three classes (Wake, Non-REM, REM), and the probability of belonging to any of the two classes (Wake, Sleep). Here, the sleep stage probability information may mean the numerical representation of the extent to which a predetermined sleep stage occupies a predetermined epoch when classifying sleep stages.

According to an embodiment of the present invention, closed-loop control or reinforcement learning is possible based on sleep stage information or sleep stage probability information acquired in epoch cycles (e.g., 30 seconds). For example, sleep state information (e.g., sleep stage information) of the user can be generated over a time corresponding to one or more epochs.

Alternatively, according to an embodiment of the present invention, sleep stage probability information indicating the probability of the user's sleep over a time corresponding to one or more epochs belonging to a certain sleep stage can be calculated.

First, from a user who received the first scent, sleep state information (e.g., sleep stage information or sleep stage probability information, etc.) over a time corresponding to one or more epochs is acquired, and then, after providing the second scent to the user at a specific point in time corresponding to the next epoch, sleep state information of the user who received the second scent can be generated over a time corresponding to one or more epochs. Through this method, learning can be performed so that the scent is provided in a direction that increases or decreases the sleep stage probability information for a specific sleep stage.

For example, if the sleep stage probability information indicating the probability that the user's sleep stage, who received the first scent at the first point in time, corresponds to the Deep sleep stage was x (where x is a value within the range of 0≤x≤1, and when sleep state information is generated while providing the second scent to the user after the first point in time has ended, if the sleep stage probability information indicating the probability of corresponding to the Deep sleep stage is x or more, learning that the second scent can induce the user's Deep sleep is possible.

Even if there is no immediate change in the sleep stage information or sleep stage probability information immediately after receiving the second scent, reinforcement learning of the scent-providing information can be performed based on the time when the sleep stage information or sleep stage probability information changes after receiving the second scent.

Meanwhile, the first scent and the second scent may be different scents, but they may also be the same scent (or scents of the same series). According to another embodiment, the first scent may be “unscented,” and the second scent may be a “scented” scent, and when sleep state information is generated while providing the second scent, if the sleep stage probability information indicating the probability of corresponding to the Deep sleep stage is x or more, learning that the second scent can induce the user's Deep sleep is possible. Alternatively, both the first scent and the second scent may be “unscented,” or the first scent may be a “scented” scent and the second scent may be “unscented.” If at least one of the first scent or the second scent is “unscented,” at least one of the first scent-providing information or the second scent-providing information may be “information indicating no scent is provided.”

Alternatively, for example, if the sleep stage probability information indicating the probability that the user's sleep stage, who received the third scent at the third point in time, corresponds to the Wake sleep stage was y (where y is a value within the range of 0≤y≤1, and when sleep state information is generated while providing the fourth scent to the user after the third point in time has ended, if the sleep stage probability information indicating the probability of corresponding to the Wake sleep stage is y or less, learning that the fourth scent can suppress the user's Wake sleep is possible.

Even if there is no immediate change in the sleep stage information or sleep stage probability information immediately after receiving the fourth scent, reinforcement learning of the scent-providing information can be performed based on the time at which the sleep stage information or sleep stage probability information changes after receiving the fourth scent.

Meanwhile, the third scent and the fourth scent may be different from each other, but they may also be the same scent (or scents of the same series). According to another embodiment, the third scent may be “unscented,” and the fourth scent may be a “scented” scent. When the fourth scent is provided and sleep state information is generated, if the sleep stage probability information indicating the probability of being in the Wake sleep stage is less than or equal to y, it can be learned that the fourth scent is a scent that can suppress the user's Wake sleep. In this manner, reinforcement learning can be performed to induce a Deep sleep stage, suppress the Wake stage during sleep, or form the proportion of REM sleep within an appropriate range throughout the overall sleep period.

Furthermore, as described above, according to one embodiment of the present invention, closed-loop control or reinforcement learning can be performed on an epoch cycle basis (e.g., every 30 seconds), allowing closed-loop control or reinforcement learning of the sleep stage for the scent-providing device 110f at relatively short time intervals (or in real-time). That is, in a method for controlling an electronic device that provides a predetermined scent in response to predetermined scent-providing information, the electronic device can be controlled in real-time based on the generated sleep state information.

Alternatively, according to one embodiment of the present invention, when the time set for the alarm approaches, reinforcement learning can be performed in a direction that increases Wake, allowing the scent-providing device 110f to assist in efficient waking.

Meanwhile, as described above, at least one of the third scent or the fourth scent may be “unscented,” and in such cases, at least one of the third scent-providing information or the fourth scent-providing information may be “information indicating no scent is provided.”

Meanwhile, according to one embodiment of the present invention, reinforcement learning can be performed not only in the direction of controlling sleep stages but also in the direction of controlling sleep events (e.g., snoring, sleep apnea, teeth grinding). In other words, scent-providing information can be generated based on sleep event information indicating that a predetermined sleep event has occurred or sleep event probability information indicating the probability that a predetermined sleep event is judged to have occurred.

That is, in a method for controlling an electronic device that provides a predetermined scent in response to predetermined scent-providing information, the sleep state information may include at least one of sleep stage information, sleep stage probability information, sleep event information, and sleep event probability information.

According to one embodiment of the present invention, in a method for controlling an electronic device that provides a predetermined scent in response to predetermined scent-providing information, the first scent-providing information and the second scent-providing information may be scent attribute information or scent provision control information. For example, scent attribute information may include the humidity of the scent, the intensity of the wind when the scent is sprayed from the discharge port 111f, the type of scent, etc., and scent provision control information may include provision information over time, such as spraying for a relatively long time at a first time point and for a relatively short time at a second time point.

For another example, scent provision control information may include information such as spraying an aroma scent for a relatively long time at a first time point and spraying a citrus scent for a relatively short time at a second time point.

According to one embodiment of the present invention, a specific example of generating scent-providing information based on sleep event probability information is as follows: If the sleep event probability information indicating the probability that a predetermined sleep event has occurred for a user who received the first scent is a (where a is a value within the range of 0≤a≤1, and after providing the first scent, the second scent is provided while generating sleep state information, and the sleep event probability information indicating the probability of the corresponding sleep event is less than or equal to a, it can be learned that the second scent is a scent that can control (including mitigating or suppressing) the user's corresponding sleep event.

Even if the probability information of the sleep event is not immediately adjusted right after receiving the second scent, reinforcement learning of the scent-providing information can be performed based on the time when the probability information of the sleep event is adjusted after receiving the second scent.

Alternatively, according to an embodiment of the present invention, a specific example of generating scent-providing information based on sleep event information is as follows: If information is obtained that a predetermined sleep event occurred for a user who received the first scent, and when the second scent is provided to the user after receiving the first scent, if information is obtained that the sleep event does not correspond to the predetermined event, it is possible to learn that the second scent can adjust (including alleviating or suppressing) the user's sleep event.

Even if the sleep event is not immediately adjusted right after receiving the second scent, reinforcement learning of the scent-providing information can be performed based on the time when the sleep event is adjusted after receiving the second scent.

Meanwhile, although the method of adjusting in the direction of alleviating or suppressing a predetermined sleep event has been described above, conversely, learning to generate information for providing a scent that induces a predetermined sleep event may also be performed.

Here, the first scent and the second scent may be different scents, but they may also be the same scent (or scents of the same series). The present invention is not limited to the specific examples of sleep events described above. For example, if a snoring event occurs during the user's sleep, the second scent provided may be a mint series scent that clears the user's blocked nose or airway, but it is not limited thereto. Additionally, at least one of the first scent or the second scent may be “unscented,” and if at least one of the first scent or the second scent is “unscented,” at least one of the first scent-providing information or the second scent-providing information may be “information indicating no scent is provided.”

Reinforcement learning can be performed in the direction of adjusting the sleep event using such a method. Furthermore, as previously examined, according to an embodiment of the present invention, since closed-loop control or reinforcement learning is possible in units of epochs (e.g., 30 seconds), there is an effect that closed-loop control or reinforcement learning for the sleep event of the scent-providing device 110f can be performed at relatively short time intervals (or in real-time).

According to another embodiment of the present invention, in a method for controlling an electronic device that provides a predetermined scent in response to predetermined scent-providing information, after the electronic device provides the first scent for a first time period, if the second scent-providing information is generated based on the user's sleep state information generated after the electronic device starts providing the first scent, the electronic device can be made to provide the second scent for a second time period. Specifically, the step of generating the second scent-providing information may further include generating the second scent-providing information based on at least one of the user's sleep stage information, sleep stage probability information, sleep event information, and sleep event probability information generated after the electronic device starts providing the first scent.

Specifically, the first time and the second time may be multiples of a predetermined minimum time unit. For example, the minimum time unit may be 1 second, 0.1 seconds, or 0.01 seconds, but is not limited thereto, and the minimum time unit for the electronic device to provide the first scent for the first time period may be the discharge time when the scent is sprayed once from the discharge port 111f.

The specific figures and operations of smart home-appliances described above are merely examples to aid in understanding the content of the present invention, and the present invention is not limited thereto.

The descriptions of the presented embodiments are provided to enable any person skilled in the art to utilize or implement the present invention. Various modifications to these embodiments will be apparent to those skilled in the art, and the general principles defined herein may be applied to other embodiments without departing from the scope of the present invention.

Thus, the present invention should not be limited to the embodiments set forth herein but should be construed in the broadest scope consistent with the principles and novel features disclosed herein.

### DESCRIPTION OF SYMBOLS

1a: electronic device
1b: electronic device
1c: electronic device
1d: electronic device
2a: electronic device
2b: electronic device
10: user terminal
40: sleep-related product recommendation device
50: sleep-related product verification device
11a: Predefined area
12a: Sensing unit
12b: Transmitting unit
12c: Receiving unit
12d: Control unit
12e: Thermal control means
13a: Sensing unit
13b: Transmitting unit
13c: Receiving unit
13d: Thermal control means
14a: Sensing Unit
14b: Control Unit
14c: Thermal Control Means
15a: Sensing Unit
15b: Transmitting Unit
15c: Receiving Unit
15d: Thermal Control Means
16a: Sensing Unit
16b: Memory Unit
16c: Processor Unit
17: Wake-up
20: External server
20a: First server
20b: Second server
30: Environment adjustment device
30-1: Heated-water mattress
30-2: Light-modulation device
30-2a: Sensing unit
30-2b: Transmitting unit
30-2c: Receiving unit
30-2d: Control Unit
30-2e: Light Source Unit
40: Environment Sensing Information Acquisition Sensor
41: Control Unit
41-1: Pre-processing Means
41-2: Sleep State Information Generation Means
41-3: Environment-Adjustment-Unit Control Means
42: Environment Adjustment Unit
46: Communication Unit
50: Home-Appliance Control Device
51: Environment adjustment information acquisition sensor
52: Control unit
52-1: Pre-processing means
52-2: Sleep state information generation means
52-3: Home-appliance control means
56: Communication unit
60: Other electronic device
61: Home-appliance control device
61-1: Sleep state information receiving means
61-2: Home-appliance control device
70: Home-appliance control device
71: Environment adjustment information acquisition sensor
72: Control unit
72-1: Pre-processing means
72-2: Home-appliance control means
76: Communication unit
79: Communication unit
80: Home-appliance control device
81: Environment sensing information acquisition sensor
82: Control unit
82-1: Pre-processing means
82-2: Home-appliance control means
86: Communication unit
100: Computing device
100f: User falling asleep or about to fall asleep
110: Network unit
110f: Scent-providing device
111f: Discharge port
120: Memory unit
130: Processor
200: Environment sensing information
200f: User terminal
201: Singularity
210: Sleep sound information
300: Spectrogram
310: AI server
400: Sleep environment adjustment device
410: Receiving module
411: Network unit
412: Memory unit
413: Sensor Unit
414: Sound Collection Unit
415: Environment Adjustment Unit
416: Receiving Control Unit
420: Transmitting Module
500: Air Conditioner
500′: Indoor Unit
500″: Indoor Unit
500″′: Indoor Unit
500″″: Indoor Unit
502: Circulator Module
503: Circulator Door
505: Cleaning Module
506: Auto-Door-Open Sensor
507: Microphone and/or Speaker
508: Human-Body Detection Sensor
509: Inner Panel
511′: Front Unit
511″: Front Unit
511″′: Front Unit
511″″: Front unit
516: Base
517: Cabinet
513: Side unit
520: Filter assembly
521: Filter member
523: Filter inlet
523′: Inlet
530: Discharge port
530′: Discharge port
531: Discharge panel
540: Air-direction controller
545: Left-right air-direction controller
550: Sensor device
551: Humidification water tank
552: Moving filter
553: Indoor-temperature sensor
554: Dust box
555: Voice-recognition sensor
556: Piping hole
557: Drain hole
558: PM1.0 sensor
559: Humidity sensor
560: Forced-operation button
570: Display unit
571: Proximity sensor
570′: Display unit
570″: Display unit
580: Ion generator
590: Air-purification indicator
700: Air purifier
700′: Air purifier
700″: Air purifier
700″′: Air purifier
710: Network unit
720: Memory
730: Processor
740: Drive unit
750: Measurement unit
800: Smart home-appliance
800-1: Humidifier/Dehumidifier
800-2: Air Conditioner
801: Autonomous Vehicle
802: Living Space
803: Smart Watch
804: Smart Speaker
810: Communication Unit
820: Sensor Unit
830: Processor
840: Memory Unit
850: Alarm unit
900: Smartphone
1000: First blower
1000′: Blower
1100: First cover
1500: First outlet
1700: Filter
1700′: Outlet
1800: UV light-emitting element
1900: Filter-status detection sensor
2000: Second blower
2100: Second cover
2300: Sensor
2400: Smart diagnosis unit
2500: AI-Sensor communication module
2600: Gas sensor
2700: Filter
2800: UV light-emitting element
3000: Flow conversion device
4000: Display unit
4100: Operation/stop button
4200: Operation-mode button
4210: Operation mode
4250: Sleep mode
4300: Purification-level button
4400: Booster-control button
4500: Setting button
5000: Partition device
5100: Network unit
5200: Memory unit
5300: Processor
5400: Drive unit
5500: Measurement unit
6000: Table
6100: Upper surface
6500: Wireless charging unit
S100M: First information acquisition
S102M: Data pre-processing
S110M: Second information acquisition
S112M: Data pre-processing
S120M: Multimodal Data Fusion
S130M: Deep Learning Model
S140M: Sleep State Information Acquisition
S200M: First Information Acquisition
S202M: Data Pre-processing
S204M: Deep Learning Model
S210M: Second Information Acquisition
S212M: Data Pre-processing
S214M: Inference Model
S220M: Multimodal Data Fusion
S230M: Sleep State Information Acquisition
S300M: First Information Acquisition
S302M: Data Pre-processing
S304M: Deep Learning Model
S310M: Second Information Acquisition
S320M: Multimodal Data Fusion
S330M: Sleep State Information Acquisition
S110: Detect the occurrence of user movement within a space through the first sensing unit
S120: Identify the user's presence in a predefined area through the second sensing unit
S130: Operate the sound collection unit to gather sound information related to the space
S140: Calculate sleep state information based on the collected sleep sound information.
S210: If the user's sleep state is pre-sleep, identify the sleep induction time based on the desired sleep time information.
S220: Detect whether the user is located within the predefined area at the sleep induction time through the second sensing unit.
S230: Transmit a notification to the user terminal.
S240: Generate the first environment adjustment information to supply predefined white light from the sleep induction time to the sleep time.
S310: The smart watch acquires sleep sound information related to the user's sleep in real-time through the microphone module.
S320: The user terminal receives the acquired sleep sound information and performs conversion and analysis into a spectrogram.
S330: Determine the user's sleep stage in real-time.
S340: The user terminal outputs a control signal to control the operation of the smart watch in real-time.
S410: The smart watch acquires sleep sound information related to the user's sleep in real-time through the microphone module.
S420: The user terminal receives the acquired sleep sound information and performs conversion and analysis into a spectrogram.
S430: Determine the user's sleep stage in real-time.
S440: The user terminal outputs a control signal to control the operation of the smart watch in real-time.
S450: Provide the user with an optimal sleep environment in response to the control signal.
S610: A step of acquiring the user's sleep information.
S611: A step of calculating the user's sleep indicator.
S620: A step of calculating the user's sleep indicator based on the user's sleep information.
S621: A step of generating cosmetic information corresponding to the calculated sleep indicator.
S630: A step of generating recommendation information for sleep-related products based on the user's sleep indicator.
S631: A step of displaying the generated cosmetic information.
S640: A step of providing recommendation information for sleep-related products
S710: A step of acquiring the sleep information of a user who has used a predetermined sleep-related product
S711: A step of receiving environment sensing information from a user terminal of a user who has used a predetermined cosmetic
S720: A step of acquiring at least one of the user's sleep intention information, sleep state information, and sleep stage information based on the user's sleep information
S721: A step of acquiring at least one of the user's sleep state information and sleep stage information based on the environment sensing information
S730: A step of generating a verification indicator for a predetermined sleep-related product based on at least one of the user's sleep intention information, sleep state information, and sleep stage information
S731: A step of generating a verification indicator for a predetermined cosmetic using at least one of the sleep state information and sleep stage information
S740: A step of verifying the impact of a predetermined sleep-related product on sleep based on the verification indicator
S741: A step of verifying the effect of the predetermined cosmetic on sleep quality based on the verification indicator

What is claimed is:

1. A method for controlling an environment adjustment device, comprising:
an acquisition step of acquiring environment sensing information;
a pre-processing step of performing pre-processing on the acquired environment sensing information;
a generation step of generating sleep state information based on the pre-processed environment sensing information; and
a control step of controlling the environment adjustment device based on the generated sleep state information.

2. The method of claim 1, wherein the control step comprises controlling the environment adjustment device in real time based on the generated sleep state information.

3. The method of claim 1, wherein the generation step further comprises converting the environment sensing information into information that includes changes of the frequency components over the time axis of the environment sensing information.

4. The method of claim 2, wherein the control step comprises:
generating first environment adjustment information based on the generated sleep state information;
enabling the environment adjustment device to adjust the environment based on the first environment adjustment information;
after the environment adjustment device begins adjusting the environment based on the first environment adjustment information, generating second environment adjustment information based on the generated user sleep state information; and
enabling the environment adjustment device to adjust the environment based on the second environment adjustment information.

5. The method of claim 4, wherein:
the first environment adjustment information is generated based on sleep state information generated during a period corresponding to one or more epochs; and
the second environment adjustment information is generated based on sleep state information generated during a period corresponding to one or more epochs after the environment adjustment device begins adjusting the environment based on the first environment adjustment information.

6. An electronic device for controlling an environment adjustment device, comprising:
a sensor configured to acquire environment sensing information; and
a control unit configured to:
perform pre-processing on the acquired environment sensing information;
generate sleep state information based on the pre-processed environment sensing information; and
control the environment adjustment device based on the generated sleep state information.

7. The electronic device of claim 6, wherein the control unit is further configured to control the environment adjustment device in real time based on the generated sleep state information.

8. The electronic device of claim 6, wherein the control unit is further configured to convert the environment sensing information into information that includes changes of the frequency components over the time axis of the environment sensing information.

9. The electronic device of claim 7, wherein, upon generating the sleep state information, the control unit:
generates first environment adjustment information based on the generated sleep state information;
controls the environment adjustment device based on the first environment adjustment information;
after the environment adjustment device begins adjusting the environment based on the first environment adjustment information, generates second environment adjustment information based on the generated user sleep state information; and
controls the environment adjustment device based on the second environment adjustment information.

10. The electronic device of claim 9, wherein:
the first environment adjustment information is generated based on sleep state information generated during a period corresponding to one or more epochs; and
the second environment adjustment information is generated based on sleep state information generated during a period corresponding to one or more epochs after the environment adjustment device begins adjusting the environment based on the first environment adjustment information.

11. An environment adjustment system comprising:
an electronic device including a sensor for acquiring environment sensing information, a control unit, and a communication unit for transmitting and receiving information through a network;
a server configured to generate sleep state information based on the environment sensing information; and
an environment adjustment device;
wherein the control unit is configured to pre-process the acquired environment sensing information and transmit the pre-processed environment sensing information to the server via the communication unit;
the server is configured to generate sleep state information based on the pre-processed environment sensing information received from the electronic device; and
the control unit is configured to receive the generated sleep state information from the server via the communication unit and control the environment adjustment device based on the received sleep state information.

12. The environment adjustment system of claim 11, wherein the control unit is further configured to control the environment adjustment device in real time based on the received sleep state information.

13. The environment adjustment system of claim 11, wherein the control unit is further configured to receive, via the communication unit, information that converts the environment sensing information into information including changes over the time axis of frequency components of the environment sensing information.

14. The environment adjustment system of claim 12, wherein the control unit:
controls the environment adjustment device based on first environment adjustment information generated based on the sleep state information received via the communication unit; and
after the environment adjustment device begins adjusting the environment based on the first environment adjustment information, generates second environment adjustment information based on the user sleep state information received via the communication unit and controls the environment adjustment device based on the second environment adjustment information.

15. The environment adjustment system of claim 14, wherein:
the first environment adjustment information is generated based on sleep state information generated during a period corresponding to one or more epochs; and
the second environment adjustment information is generated based on sleep state information generated during a period corresponding to one or more epochs after the environment adjustment device begins adjusting the environment based on the first environment adjustment information.

16. An environment adjustment system comprising:
an electronic device including a sensor for acquiring environment sensing information, a control unit, and a communication unit for transmitting and receiving information through a network;
a server configured to generate sleep state information based on the environment sensing information and to generate environment adjustment information based on the sleep state information; and
an environment adjustment device controlled based on the environment adjustment information;
wherein the control unit is configured to pre-process the acquired environment sensing information and transmit the pre-processed environment sensing information to the server via the communication unit; and
the server is configured to generate sleep state information based on the pre-processed environment sensing information received from the electronic device, generate environment adjustment information for controlling the environment adjustment device based on the generated sleep state information, and transmit the generated environment adjustment information to the environment adjustment device.

17. The environment adjustment system of claim 16, wherein:
the server comprises a first server and a second server,
the first server being configured to generate sleep state information based on the pre-processed environment sensing information received from the electronic device, and
the second server being configured to generate environment adjustment information for controlling the environment adjustment device based on the generated sleep state information.

18. The environment adjustment system of claim 16, wherein the server is further configured to control the environment adjustment device in real time based on the generated sleep state information.

19. The environment adjustment system of claim 16, wherein the server is configured to convert the environment sensing information received from the electronic device into information that includes changes of the frequency components over the time axis of the environment sensing information.

20. The environment adjustment system of claim 17, wherein the server:
controls the environment adjustment device based on first environment adjustment information generated based on the generated sleep state information and,
after the environment adjustment device begins adjusting the environment based on the first environment adjustment information,
generates second environment adjustment information based on the generated sleep state information, and
controls the environment adjustment device based on the second environment adjustment information.

21. The environment adjustment system of claim 19, wherein:
the first environment adjustment information is generated based on sleep state information generated during a period corresponding to one or more epochs; and
the second environment adjustment information is generated based on sleep state information generated during a period corresponding to one or more epochs after the environment adjustment device begins adjusting the environment based on the first environment adjustment information.
